(19) United States
(12) Patent Application Publication
(10) Pub. No.: US 2023/0321186 A1
(43) Pub. Date: Oct. 12, 2023

(54) Mucin Glycans as Antifungal Agents

(71) Applicants: Massachusetts Institute of Technology, Cambridge, MA (US); Universitat Basel, Basel (CH)

(72) Inventors: Katharina Ribbeck, Cambridge, MA (US); Julie Sant'Anna Takagi, Mountain View, CA (US); Rachel Hevey, Basel (CH)

(21) Appl. No.: 18/189,190

(22) Filed: Mar. 23, 2023

Related U.S. Application Data
(60) Provisional application No. 63/269,825, filed on Mar. 23, 2022.

(51) Int. Cl. A61K 38/17; A61K 47/54; A61P 31/10

(52) U.S. Cl. CPC A61K 38/1735; A61P 31/10; A61K 47/549

(57) ABSTRACT

The disclosure provides, in various embodiments, methods of treating fungal infections in subjects in need thereof, methods of attenuating virulence of fungi (e.g., in subjects in need thereof), and methods of inhibiting formation of fungal biofilms on surfaces (e.g., living or inert surfaces) with mucin glycans, tautomer, stereoisomer and/or pharmaceutically acceptable salts thereof. The disclosure further provides, in various embodiments, mucin glycan compositions, such as synthetic mucin glycans and defined mucin glycan compositions.

### RELATED APPLICATION

This application claims the benefit of U.S. Provisional Application No. 63/269,825, filed on Mar. 23, 2022. The entire teachings of the above application are incorporated herein by reference.

### GOVERNMENT SUPPORT

This invention was made with government support under DMR1419807 and PHY1454673 awarded by the National Science Foundation, and under EB017755, GM124594 and GM103694 awarded by the National Institutes of Health. The government has certain rights in the invention.

### INCORPORATION BY REFERENCE OF MATERIAL IN XML

This application incorporates by reference the Sequence Listing contained in the following eXtensible Markup Language (XML) file being submitted concurrently herewith:
a) File name: 00502370001.xml; created Jan. 18, 2023, 33,541 bytes in size.

### BACKGROUND

Candida albicans is an opportunistic fungal pathogen that asymptomatically colonizes the mucosal surfaces of most healthy humans, particularly on surfaces of the oral cavity, gastrointestinal tract, and female genital tract. Alterations to the mucus barrier and perturbations in the microbiota can lead to C. albicans overgrowth and infection, causing conditions such as oral thrush, vulvovaginal candidiasis, and life-threatening systemic candidiasis. The scarcity of antifungal drug classes, their limited efficacy, toxicity, and the development of resistance contribute to a high mortality rate of ˜40% in deep-seated candidiasis, highlighting a profound and urgent need for the development of alternative treatments for fungal infections.

### SUMMARY

The subject matter disclosed herein is based, in part, on the discovery that mucin glycans, when chemically released from their naturally grafted state, as well as being synthetically produced, can be used as novel therapeutic agents against the fungal pathogen, Candida albicans.

In one aspect, the disclosure provides methods of attenuating virulence of a fungus in a subject in need thereof, the methods comprising administering to the subject a therapeutically effective amount of a mucin glycan, tautomer, stereoisomer and/or a pharmaceutically acceptable salt thereof.

In another aspect, the disclosure provides methods of treating a fungal infection in a subject in need thereof, the methods comprising administering to the subject a therapeutically effective amount of a mucin glycan, tautomer, stereoisomer and/or a pharmaceutically acceptable salt thereof.

In another aspect, the disclosure provides methods of treating a biofilm-related infection in a subject in need thereof, the methods comprising administering to the subject a therapeutically effective amount of a mucin glycan, tautomer, stereoisomer and/or a pharmaceutically acceptable salt thereof.

In another aspect, the disclosure provides methods of maintaining a microbiota in a subject in need thereof, the methods comprising administering to the subject a therapeutically effective amount of a mucin glycan, tautomer, stereoisomer and/or a pharmaceutically acceptable salt thereof.

In another aspect, the disclosure provides methods of repairing or restoring a damaged microbiome or microbiota in a subject in need thereof, comprising administering to the subject a therapeutically effective amount of a mucin glycan, tautomer, stereoisomer and/or a pharmaceutically acceptable salt thereof.

In another aspect, the disclosure provides methods of maintaining a mucus barrier in a subject in need thereof, the methods comprising administering to the subject a therapeutically effective amount of a mucin glycan, tautomer, stereoisomer and/or a pharmaceutically acceptable salt thereof.

In another aspect, the disclosure provides methods of attenuating virulence of a fungus, the methods comprising contacting the fungus with a mucin glycan, tautomer, stereoisomer and/or a pharmaceutically acceptable salt thereof.

In another aspect, the disclosure provides methods of inhibiting formation of a fungal biofilm on a surface, the methods comprising contacting the surface with a mucin glycan, tautomer, stereoisomer and/or a pharmaceutically acceptable salt thereof.

In another aspect, the disclosure provides methods of maintaining a mucus barrier on a surface, the methods comprising contacting the surface with a mucin glycan, tautomer, stereoisomer and/or a pharmaceutically acceptable salt thereof.

In another aspect, the disclosure provides compositions comprising a (i.e., one or more) synthetic mucin glycan.

In another aspect, the disclosure provides compositions comprising a mucin glycan, wherein the purity of the mucin glycan is at least about 30%.

In another aspect, the disclosure provides defined mucin glycan compositions comprising one or more mucin glycans desired mucin glycans, wherein at least 80% of total mucin glycans present in the compositions are of the one or more desired mucin glycans or pharmaceutically acceptable salts thereof.

In another aspect, the disclosure provides defined mucin glycan compositions comprising one or more mucin glycans, wherein at least 80% of total mucin glycans present in the compositions are of the one or more mucin glycans or pharmaceutically acceptable salts thereof.

In some embodiments, a fungus comprises Candida albicans.

In some embodiments, a mucin glycan is a synthetic mucin glycan.

In some embodiments, attenuating virulence of a fungus comprises modulating expression of a virulence-associated gene of a fungus, reducing a fungus's surface adhesion, inhibiting a fungus's morphological transition to an invasive or virulent cell type, inhibiting fungal biofilm formation, reducing a fungus's secretion of a hydrolytic enzyme, or a combination thereof.

In some embodiments, a surface comprises a living surface, an inert surface, or both.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1C Mucins are the bioactive components of mucus that suppress adhesion and filamentation in Candida albicans (C. albicans) independent of growth. FIG. 1A Mucus, which is made of gel-forming mucins (with protein names including MUC), covers all non-keratinized epithelial surfaces of the body. This study investigated MUC5B purified from human saliva, and MUC2 and MUC5AC purified from porcine intestinal and gastric mucus, respectively. FIG. 1B Native mucus across the major sources decreases adherence to polystyrene wells. Bars indicate mean±SEM from n=3 biologically independent replicates using fluorescence images. Significance was assessed using ordinary one-way ANOVA followed by Bonferroni correction for multiple comparisons; ****P<0.0001. FIG. 1C The depletion of mucus components leads to increased fungal adherence. Supplementation of mucus filtrates with exogenous purified mucins lead to decreased adhesion. Bars indicate mean±SEM from n=3 biologically independent replicates using fluorescence images. Significance was assessed using one-way ANOVA followed by Bonferroni correction for multiple comparisons; ****P<0.0001.

FIGS. 2A-2H Mucins across major mucosal surfaces share a conserved function in attenuating C. albicans virulence in vitro and in vivo. FIG. 2A The mucus barrier hosts a diverse range of microorganisms while limiting infections. Candida albicans, an opportunistic fungal pathogen, resides within the mucosa. FIG. 2B Native mucus suppresses fungal adherence to polystyrene wells. Depletion of intestinal mucus components increases fungal adherence. Supplementation of mucus filtrates with purified MUC2 reduces adhesion. Bars indicate mean SEM from n=3 biologically independent replicates using fluorescence images. Significance was assessed using one-way ANOVA followed by Bonferroni multiple comparisons test; ****P<0.0001 **P<0.01. FIG. 2C MUC5AC, MUC5B, and MUC2 elicit global transcriptional responses in C. albicans. FIG. 2D Dendrogram and hierarchical clustering heat map of genome-wide expression profiles from MUC5AC, MUC2, and MUC5B. Clustering was performed first along the sample dimension and then along the individual gene dimension using the Matlab clustergram function with Euclidean distances and Ward's linkage method. Scale bar units are in SDs. FIG. 2E Venn diagrams indicate the number of genes differentially expressed after exposure to mucins. FIG. 2F RNA sequencing data for selected genes belonging to filamentation, biofilm formation, and pathogenesis. FIG. 2G Fungal viability was monitored in a live dermal wound model. FIG. 2H Fungal burden on murine puncture wounds at 5 and 7 d after treatment with MUC2 or a mock-treatment (NT) control. Symbols represent colony-forming units from n=6 biologically independent replicates. The center bars indicate the median, the box limits indicate the upper and lower quartiles, and the whiskers indicate the minimum and maximum values. Significance was assessed using Welch and Brown-Forsythe ANOVA (assumes unequal variance) followed by Dunnett T3 multiple comparisons test; *P=0.016.

FIGS. 3A-3D Mucin glycan-mediated suppression of virulence pathways is independent of specific experimental conditions, including medium and time point.

FIG. 3A Quantification of C. albicans morphology in the presence of mucins at 8 h using phase-contrast images from n=3 (MUC5AC) or n=4 (Media, MUC5B, MUC2) biologically independent replicates. FIG. 3B Quantification of C. albicans morphology in the presence of mucin glycans in RPMI medium at 8 h using phase-contrast images from n=6 (CPH1), n=4 (WT), n=3 (RAS1) biologically independent replicates. prADH1-CPH1 strain abbreviated as CPH1; RAS1^{G13V} strain abbreviated as RAS1. FIG. 3C Quantification of C. albicans morphology in the presence of mucin glycans in Spider medium at 8 h using phase-contrast images from n=3 (WT), n=4 (EFG1⁻) or n=5 (EFG1⁺) biologically independent replicates. PCKpr-efg1-T206E strain abbreviated as EFG1. FIG. 3D Quantification of C. albicans morphology in the presence of synthesized glycans in RPMI medium at 8 h using phase-contrast images from n=6 (MG), n=3 (1, 3, 4, 6) biologically independent replicates. 1, Core 1; 3, Core 1+fucose; 6, Core 2+galactose; 4, Core 1+sialic acid. The percentage of hyphae was obtained by dividing the number of hyphae by the total number of cells from n>100 cells. Bars indicate mean±SEM. MG, mucin glycans.

FIGS. 4A-4G Mucin glycans potently inhibit filamentation in a time- and dose-dependent manner. FIG. 4A Structural diversity and relative abundances of MUC5AC glycans analyzed by NSI-MS. Negative mode NSI-MS detected sulfation on a subset of O-glycans, indicated with the letter S in a circle. NeuAc, N-acetylneuraminic acid. FIG. 4B Relative abundances of glycan features in the MUC5AC glycan pool. LacNAc, N-acetyllactosamine or Galβ1-3/4GlcNAc; GalGal, Galα1-3Gal; LacdiNAc, GalNAcβ1-4GlcNAc; O-Man, mannose linked α1 to serine or threonine. FIG. 4C Functional enrichment analyses reveal key virulence pathways among downregulated genes. Significance of enrichment was calculated using a two-tailed Mann-Whitney U test based on mean log₂-transformed FCs from n=3 biologically independent replicates. The dotted line represents the threshold for significance (FDR-adjusted P<0.05). FIG. 4D MUC5AC glycans inhibit filamentation. Phase-contrast images of WT SC5314 cells grown in RPMI medium alone, the monosaccharide (MS) pool or mucin glycans at 37° C. for 8 h. Scale bar, 20 μm. FIG. 4E Mucin glycans, unlike their monosaccharide components, downregulate signature virulence genes. Bars indicate mean±SEM from n=5 (MS pool), n=10 (MUC5AC glycans) biologically independent replicates. FIG. 4F MUC5AC glycans downregulate virulence gene expression over a prolonged time course. Bars indicate mean SEM from n=5 (0.5 h, 2 h), n=4 (4 h) biologically independent replicates. FIG. 4G Mucin glycans regulate YWP1 expression in a concentration-dependent manner. Data points indicate mean±SEM and are fitted to a nonlinear agonist binding curve from n=3 (0.01%, 0.025%, 0.3%), n=5 (0.05%, 0.1%) biologically independent replicates. For FIGS. 4E-4G, data are log₂-transformed qPCR measurements normalized to a control gene (ACT1).

FIGS. 5A-5B Mucin glycans downregulate virulence traits and alter fungal-bacterial dynamics. FIG. 5A RNA sequencing data for selected genes belonging to the filamentation pathway that are differentially regulated in the presence of MUC5AC glycans at 8 h. FC data are mean measurements from n=3 biologically independent replicates. FIG. 5B qRT-PCR confirms that mucin glycans downregulate the expression of key virulence genes. Exposure to 0.1% MUC5AC glycans decreases the expression of filamentation genes and increases the expression of YWP1 at 8 h. Data are log₂-transformed qPCR measurements of relative gene expression normalized to a control gene (ACT1). Data mean±standard error of the mean and were calculated from n=3 biologically independent replicates.

FIGS. 6A-6C Mucin glycans act via Nrg1 to prevent filamentation and hyphal gene expression. FIG. 6A Expression of NRG1 in wild-type (WT) SC5314 cells after 0.5 h, 2 h, or 4 h in 0.1% MUC5AC glycans (versus growth in medium alone). Gene expression was measured with qRT-PCR and normalized to a control gene (ACT1). Bars indicate the mean±SEM from n=5 (0.5 h, 2 h), n=4 (4 h) biologically independent replicates. FIG. 6B Expression of filamentation-associated genes in WT SC5314 or Δ/Δnrg1 mutant cells after 2 h in MUC5AC glycans (versus growth in medium alone). Gene expression was measured with qRT-PCR and normalized to a control gene (ACT1). Bars indicate the mean±SEM from n=5 (WT), n=3 (ΔΔnrg1) biologically independent replicates. FIG. 6C FC values for gene-expression changes in WT SC5314 or Δ/Δnrg1 mutant cells after 2 h in MUC5AC glycans (versus growth in medium alone).

FIGS. 7A-7G Mucin glycans downregulate virulence cascades and mediate fungal-bacterial dynamics. FIG. 7A Mucin glycans downregulate the expression of adhesion-related genes. Gene expression was measured by qRT-PCR and normalized to a control gene (ACT1). Bars indicate mean±SEM from n=11 (ECE1, ALS3), n=4 (HWP1) biologically independent replicates. MS, monosaccharide. FIG. 7B Quantification of adhesion to polystyrene wells using fluorescence images from n=9 (MS pool, MUC5AC glycans), n=6 (Medium) biologically independent replicates. Bars indicate mean±SEM. Significance was assessed using ordinary one-way ANOVA followed by Bonferroni multiple comparisons test; ****P<0.0001.

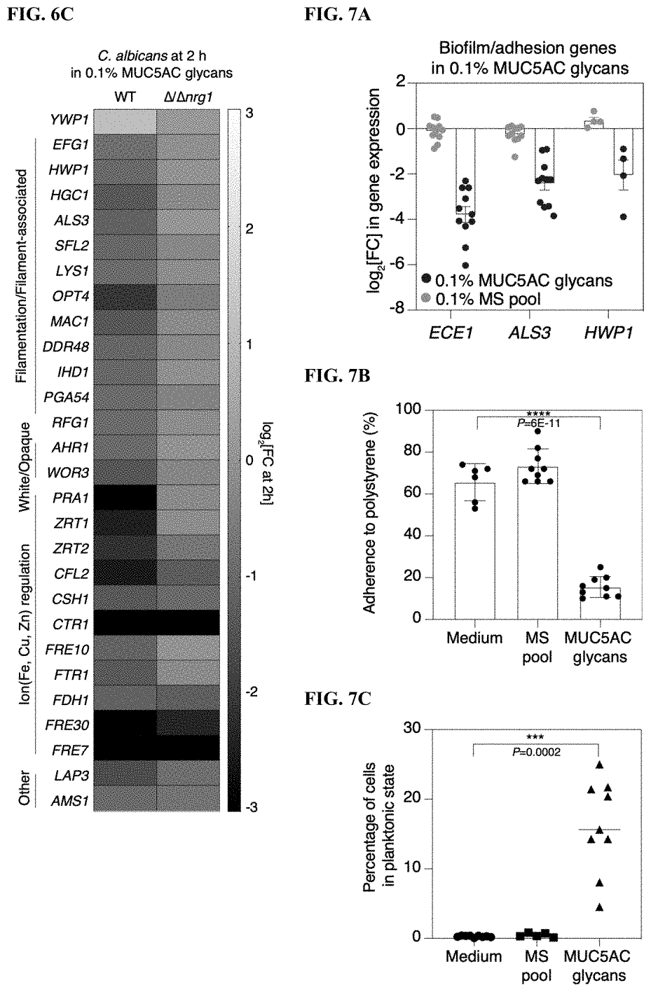

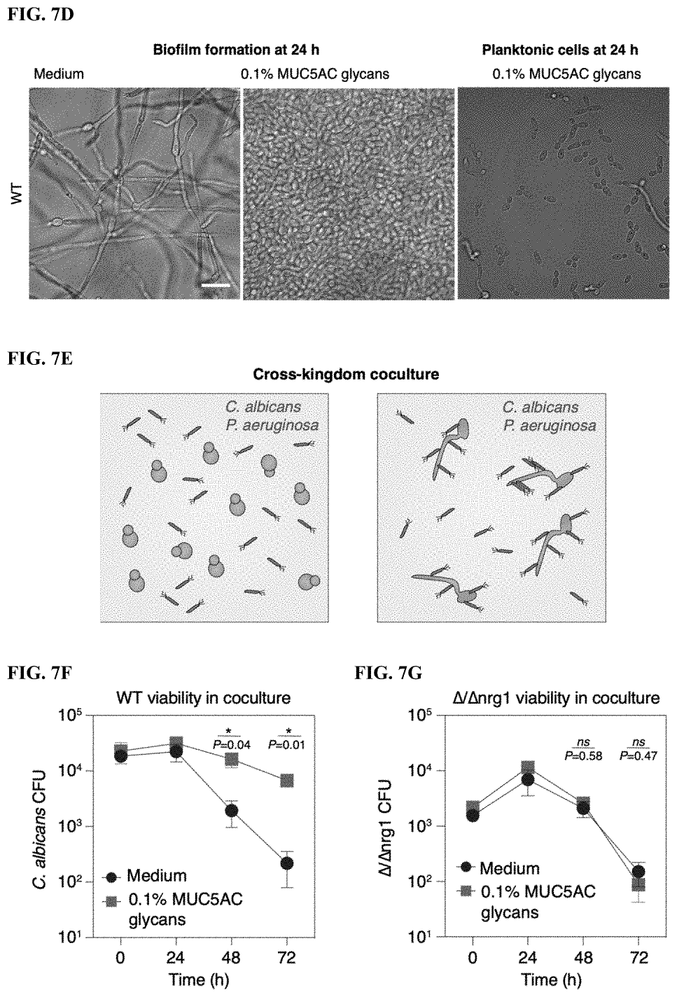

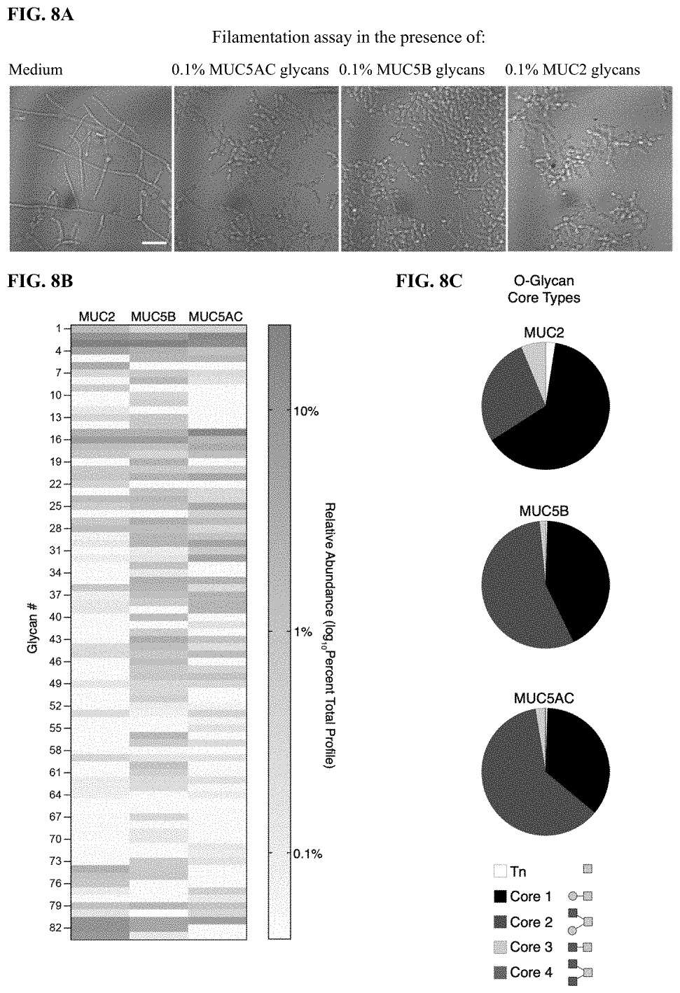

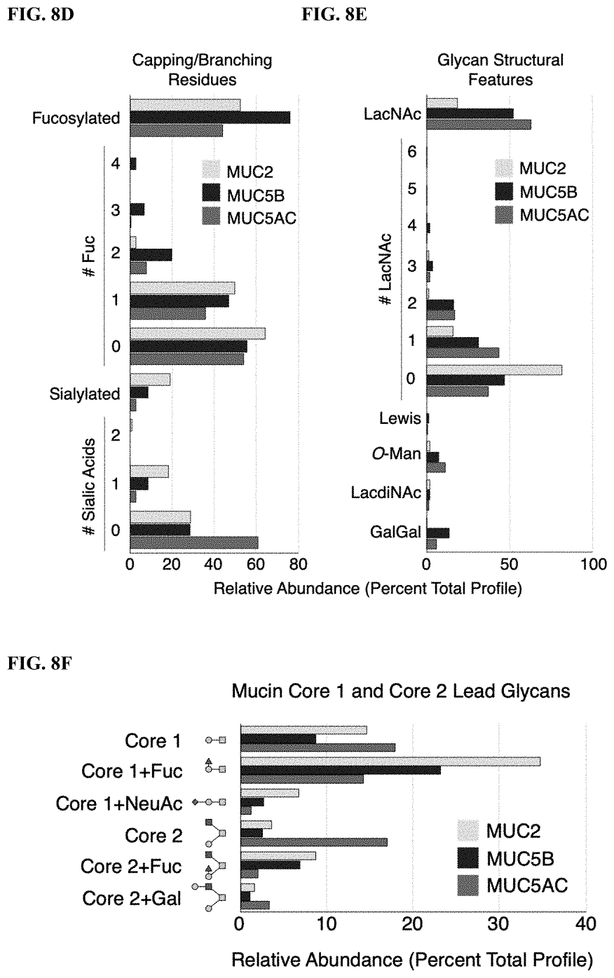

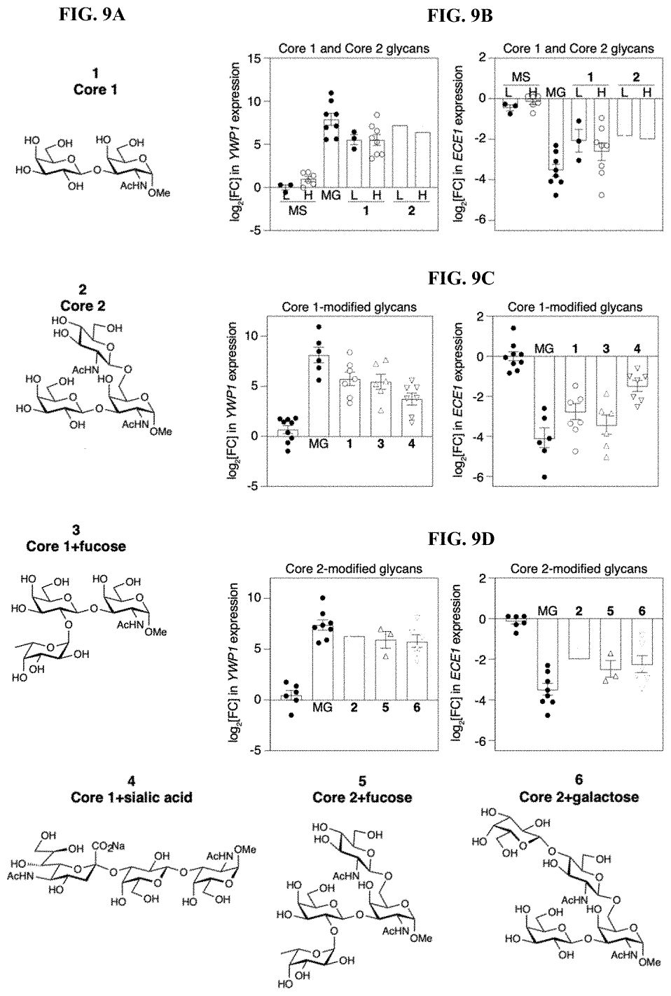

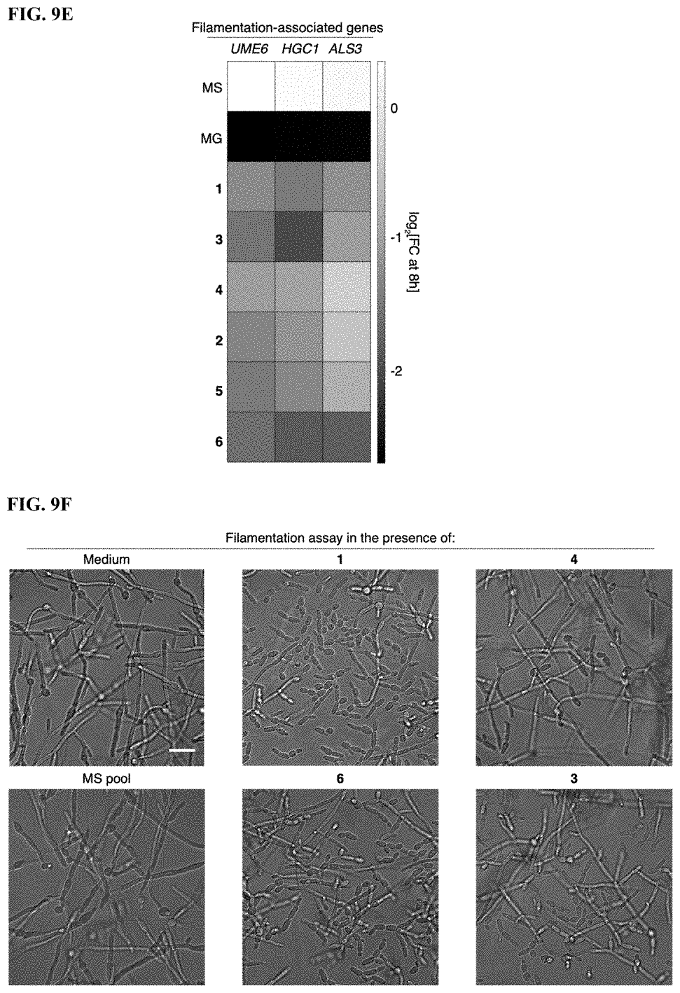

FIG. 7C Quantification of CFUs in the supernatant (planktonic cells) relative to total CFU from adhered cells in the biofilm from n=9 (Medium, MUC5AC glycans) and n=5 (MS pool) biologically independent replicates. Significance was assessed using Brown-Forsythe and Welch ANOVA tests (assumes unequal variance) followed by Dunnett T3 multiple comparisons test; ***P=0.0002. FIG. 7D Phase-contrast images of (left, middle) biofilm and (right) planktonic WT cells grown for 24 h in the (left) absence or (middle, right) presence of MUC5AC glycans. Non-adhered (planktonic) cells in the MUC5AC glycan-exposed biofilms were imaged (right). Scale bar, 20 μm. FIG. 7E Schematics of in vitro P. aeruginosa (bacteria) and C. albicans (yeast) interactions. Left: P. aeruginosa does not effectively kill yeast form C. albicans. Right: In contrast, P. aeruginosa adheres to C. albicans hyphae and secretes toxins, leading to fungal death. FIG. 7F C. albicans yeast cells were diluted into RPMI medium with or without MUC5AC glycans for 4 h. P. aeruginosa cells (OD₆₀₀=0.25) in spent LB were added to the C. albicans cells with or without MUC5AC glycans and cocultured at 37° C. for 72 h. The fungal viable cell population was determined daily by plating. *P=0.04 (48 h); *P=0.014 (72 h).
FIG. 7G Cultures of Δ/Δnrg1 cells were treated as in (g) with or without MUC5AC glycans and cocultured with P. aeruginosa at 37° C. for 72 h. For FIGS. 7F-7G, Data are mean±SEM from n=5 biologically independent replicates. Significance was assessed using two-tailed Student's t-tests with Welch's correction.

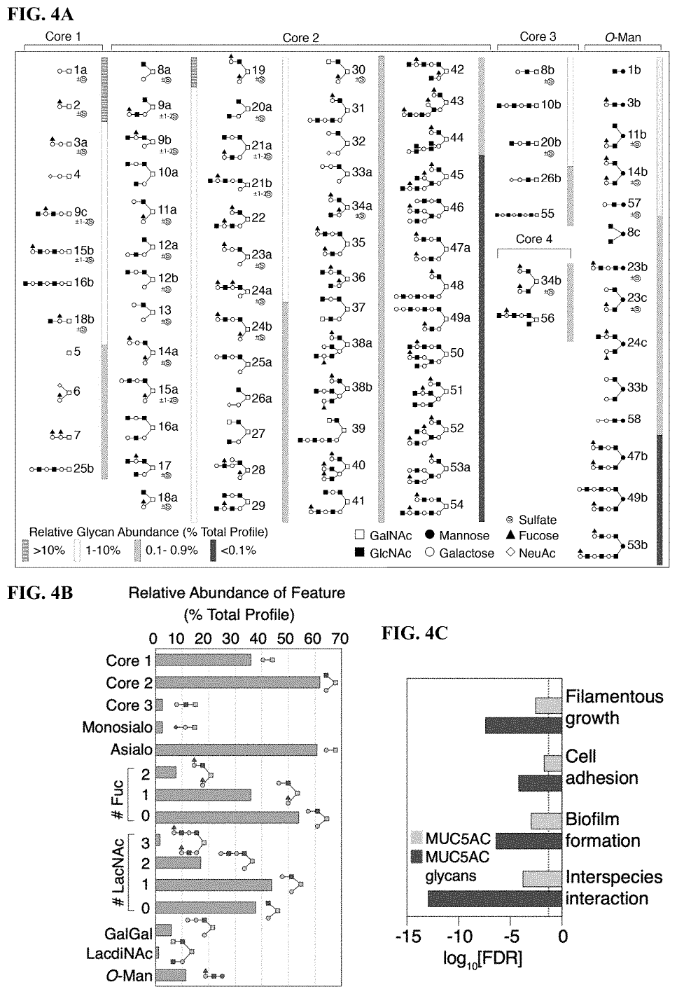

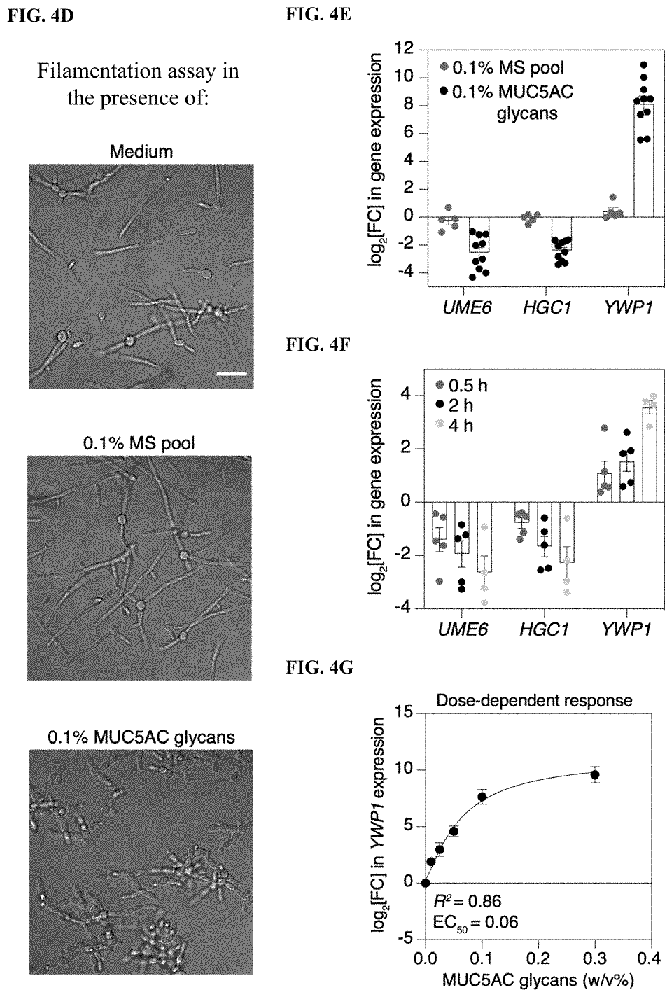

FIGS. 8A-8F Native mucins across microbial niches display a plethora of complex glycan structures with regulatory potential. FIG. 8A Wild-type C. albicans SC5314 cells were diluted into RPMI medium with or without mucin glycan libraries purified from MUC5AC, MUC2, and MUC5B and cultured at 37° C. for 8 h. Phase-contrast images of C. albicans revealed that mucin glycans across three mucin types suppress filamentation. Scale bar, 20 μm. FIG. 8B Heatmap presenting log₁₀ values for the relative abundances of individual glycans released from the three mucins and detected by NSI-MS as permethylated derivatives (Tables 4-6). MUC2, MUC5B, and MUC5AC are dominated by O-GalNAc Core 1 (glycans #3, 4, 5) and Core 2 derived glycan structures (glycans #15, 16, 17). Glycans #81, 82, and 83 represent non-reducing terminal disaccharides of incomplete core structures likely generated through peeling reactions during preparation. FIG. 8C Distribution of O-glycans by GalNAc-initiated core type on each mucin. Minimal core structures are shown in the legend. The relative abundances of each glycan containing a minimal core structure was summed for comparison. FIG. 8D The relative abundances of glycans carrying capping/branching fucose or sialic acid residues were summed for comparison across the three mucins. Glycans with between 0 and 4 fucose residues or between 0 and 2 sialic acid residues were detected. The relative abundances of sialylated and fucosylated glycans was calculated based on the total glycan profile, while the relative abundances of glycans lacking sialic acid or fucose was calculated based on the subset of glycans in the total profile that are structurally amenable to sialylation or fucosylation. FIG. 8E The relative abundances of glycans that possess the indicated structural features or motifs were summed for comparison across the three mucins. Abbreviations for the features are as previously described (FIGS. 4A-4G). The Lewis designation refers to the detection of a fucosylated LacNAc residue. FIG. 8F The relative abundance of the six most prevalent Core 1 and Core 2 glycans shared across all three mucins is presented. These mucin-derived glycans defined structures that served as synthetic targets for generating lead compounds for subsequent functional analysis (FIGS. 9A-9F).

FIGS. 9A-9F Synthetic Core 1- and Core 2-modified glycans are sufficient to suppress C. albicans filamentation. FIG. 9A Depiction of synthesized mucin glycan structures (1-6) that are abundant in the complex mucin glycan pool. FIG. 9B Exposure to low (L; 0.1% w/v) and high (H; 0.4% w/v) concentrations of synthesized glycan structures (left) increase transcription of the yeast-associated gene YWP1 and (right) decreases transcription of the filamentation-associated toxin gene ECE1. Data from n=3 (0.1% MS, 1 and 2), n=4 (0.4% 2), n=6 (0.4% MS) and n=8 (0.1% MG, 0.4% 1) biologically independent replicates.

FIG. 9C Exposure to Core 1-modified glycan structures decreases the expression of the virulence-associated gene, ECE1, and increases the expression of the yeast-associated gene, YWP1. These results are dampened by the addition of sialic acid to Core 1. Data from n=6 (MG, 3), n=7 (4, 1) and n=9 (MS) biologically independent replicates. FIG. 9D Exposure to Core 2-modified glycan structures decreases the expression of the virulence-associated gene, ECE1, and increases the expression of YWP1. Data from n=3 (5), n=4 (2), n=6 (6, MS) and n=8 (MG) biologically independent replicates. FIG. 9E Expression of filamentation-associated genes in the presence of MS, MG, and synthesized Core 1- and Core 2-modified glycan structures. Data indicates the mean from n=3 (6), n=4 (3, 5), n=6 (2) and n=7 (MS, MG, 1, 4) biologically independent replicates. FIG. 9F Phase-contrast images of C. albicans SC5314 cells that were grown in RPMI medium alone, 0.4% monosaccharide (MS) pool or the indicated synthetic glycan structure (0.4%) at 37° C. for 8 h. Scale bar, 20 μm. For FIGS. 9B-9E, gene expression was measured with qRT-PCR and normalized to a control gene (ACT1). Bars indicate mean±SEM. MS, black circles; MG, mucin glycans; FC, fold change.

### DETAILED DESCRIPTION

A description of example embodiments follows.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

When introducing elements disclosed herein, the articles “a,” “an,” “the,” and “said” are intended to mean that there are one or more of the elements. Further, the one or more elements may be the same or different.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

Compounds described herein include those described generally, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

“Alkyl” refers to a branched or straight-chain, monovalent, hydrocarbon radical having the specified number of carbon atoms. Thus, “(C₂-C₈)alkyl” refers to a radical having from 2-8 carbon atoms in a branched or linear arrangement. Typically, alkyl is (C₁-C₂₅)alkyl, e.g., (C₁-C₁₅)alkyl, (C₁-C₁₀)alkyl, (C₁-C₈)alkyl, (C₂-C₈)alkyl, (C₁-C₆)alkyl, (C₂-C₆)alkyl, (C₁-C₅)alkyl, (C₂-C₅)alkyl or (C₂-C₃)alkyl. Examples of alkyl groups include methyl, ethyl, n-propyl, isopropyl, n-butyl, isobutyl, sec-butyl, t-butyl, n-pentyl, isopentyl, neopentyl, 2-methylpentyl, n-hexyl, and the like. In some embodiments, alkyl is optionally substituted, e.g., with one or more substituents described herein.

“Aryl” refers to a monocyclic or polycyclic (e.g., bicyclic, tricyclic), aromatic, hydrocarbon ring system having the specified number of ring atoms, and includes aromatic rings fused to non-aromatic rings, as long as one of the fused rings is an aromatic hydrocarbon. Thus, “(C₆-C₁₅)aryl” refers to a ring system having from 6-15 ring atoms. Examples of aryl include phenyl, naphthyl and fluorenyl. In some embodiments, aryl (e.g., (C₆-C₁₅)aryl) is phenyl, naphthyl or fluorenyl. In some embodiments, aryl is optionally substituted, e.g., with one or more substituents described herein.

“Heteroaryl” refers to a monocyclic or polycyclic (e.g., bicyclic, tricyclic), aromatic, hydrocarbon ring system having the specified number of ring atoms, wherein at least one carbon atom in the ring system has been replaced with a heteroatom selected from nitrogen, sulfur and oxygen. Thus, “(C₅-C₁₅)heteroaryl” refers to a heteroaromatic ring system having from 5-15 ring atoms consisting of carbon, nitrogen, sulfur and oxygen. “Heteroaryl” includes heteroaromatic rings fused to non-aromatic rings, as long as one of the fused rings is a heteroaromatic hydrocarbon. A heteroaryl can contain 1, 2, 3 or 4 (e.g., 1, 2 or 3) heteroatoms independently selected from nitrogen, sulfur and oxygen. In some embodiments, a heteroaryl contains 1, 2 or 3 heteroatoms, each of which is nitrogen. Typically, heteroaryl is (C₅-C₂₀)heteroaryl, e.g., (C₅-C₁₅)heteroaryl, (C₅-C₁₂)heteroaryl, C₅ heteroaryl or C₆ heteroaryl. Monocyclic heteroaryls include, but are not limited to, furan, oxazole, thiophene, triazole, triazene, thiadiazole, oxadiazole, imidazole, isothiazole, isoxazole, pyrazole, pyridazine, pyridine, pyrazine, pyrimidine, pyrrole, tetrazole and thiazole. Bicyclic heteroaryls include, but are not limited to, indolizine, indole, isoindole, indazole, benzimidazole, benzofuran, benzothiazole, purine, quinoline, isoquinoline, cinnoline, phthalazine, quinazoline, quinoxaline, naphthyridine and pteridine. In some embodiments, heteroaryl (e.g., (C₅-C₁₅)heteroaryl) is pyridinyl, pyrimidinyl or carbazolyl. In some embodiments, heteroaryl is optionally substituted, e.g., with one or more substituents described herein.

“Alkoxy” refers to an alkyl radical attached through an oxygen linking atom, wherein alkyl is as described herein. Examples of alkoxy include, but are not limited to, methoxy, ethoxy, propoxy, isopropoxy, and the like.

“Halogen” and “halo” are used interchangeably herein and each refers to fluorine, chlorine, bromine, or iodine. In some embodiments, halo is fluoro, chloro or bromo. In some embodiments, halo is fluoro.

Unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this disclosure. In all provided structures, any hydrogen atom can also be independently selected from deuterium (²H), tritium (³H) and/or fluorine (¹⁸F). Such compounds are useful, for example, as analytical tools, as probes in biological assays, or as therapeutic agents in accordance with the present disclosure.

“Derived from,” as used herein, refers to a chemical structure that is homologous to or structurally similar to a related chemical structure.

The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds described herein can also exist as “solvates” or “hydrates.” A “hydrate” is a compound that exists in a composition with one or more water molecules. A hydrate can include water in stoichiometric quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

Compounds disclosed herein may exist as stereoisomers. For example, compounds disclosed herein may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, or as individual diastereomers or enantiomers.

Unless otherwise indicated, all possible isomers and mixtures thereof, including optical isomers, rotamers, tautomers and cis- and trans-isomers, are included in the present invention.

When a disclosed compound is depicted by structure without indicating the stereochemistry, and the compound has one chiral center, it is to be understood that the structure encompasses one enantiomer or diastereomer of the compound separated or substantially separated from the corresponding optical isomer(s), a racemic mixture of the compound and mixtures enriched in one enantiomer or diastereomer relative to its corresponding optical isomer(s).

When a disclosed compound is depicted by a structure indicating stereochemistry, and the compound has more than one chiral center, the stereochemistry indicates relative stereochemistry, rather than the absolute configuration of the substituents around the one or more chiral carbon atoms. “R” and “S” are used to indicate the absolute configuration of substituents around one or more chiral carbon atoms.

“Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms.

“Racemate” or “racemic mixture,” as used herein, refer to a mixture containing equimolar quantities of two enantiomers of a compound. Such mixtures exhibit no optical activity (i.e., they do not rotate a plane of polarized light).

Methods of obtaining an optical isomer separated or substantially separated from the corresponding optical isomer(s) are known in the art. For example, an optical isomer can be purified from a racemic mixture by well-known chiral separation techniques, such as, but not limited to, normal- and reverse-phase chromatography, and crystallization. An optical isomer can also be prepared by the use of chiral intermediates or catalysts in synthesis. In some cases, compounds having at least some degree of enantiomeric enrichment can be obtained by physical processes, such as selective crystallization of salts or complexes formed with chiral adjuvants.

As used herein, the term “compound of the disclosure” refers to a compound of any structural formula depicted herein (e.g., a compound of structural formula I or a subformula thereof)), as well as isomers, such as stereoisomers (including diastereoisomers, enantiomers and racemates) and tautomers thereof, isotopologues thereof, and inherently formed moieties (e.g., polymorphs and/or solvates, such as hydrates) thereof. When a moiety is present that is capable of forming a salt, then salts are included as well, in particular, pharmaceutically acceptable salts.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

“Treating” or “treatment,” as used herein, refers to taking steps to deliver a therapy to a subject, such as a mammal, in need thereof (e.g., as by administering to a mammal one or more therapeutic agents). “Treating” or “treatment” includes inhibiting the disease or condition (e.g., as by slowing or stopping its progression or causing regression of the disease or condition), and relieving the symptoms resulting from the disease or condition. The term “treating” or “treatment” refers to the medical management of a subject with the intent to improve, ameliorate, stabilize (i.e., not worsen), prevent or cure a disease, pathological condition, or disorder-such as the particular indications exemplified herein. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

“Administering” or “administration,” as used herein, refers to providing a compound, composition, or pharmaceutically acceptable salt thereof described herein to a subject in need of treatment or prevention.

“A therapeutically effective amount” or “an effective amount” refers to an amount effective, at dosages and for periods of time necessary, to achieve a desired therapeutic or biological result (e.g., treatment, healing, inhibition or amelioration of physiological response or condition, etc.). Non-limiting examples of desired therapeutic or biological results include disruption of fungal biofilm formation, growth, and/or maintenance, for example, at or proximate to the surface of an implanted medical device. Effective reductions of signs and/or symptoms associated with fungal infection can be determined by one or more suitable means in the art.

The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of an individual, e.g., a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, or from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

### Methods of the Disclosure

In one aspect, the disclosure provides methods of attenuating virulence of a fungus, the methods comprising contacting the fungus with a mucin glycan, tautomer, stereoisomer and/or a pharmaceutically acceptable salt thereof.

In some embodiments, a fungus comprises a yeast. In some embodiments, a fungus (e.g., yeast) is capable of biofilm growth. Non-limiting examples of fungi capable of biofilm growth include Candida species (e.g., Candida albicans, Candida glabrate, C. parapsilosis, Candida dubiliensis, and Candida tropicalis), Cryptococcus neoformans (e.g., C. neoformans), Trichosporon species (e.g., Trichosporon asahii), Aspergillus fumigatus (e.g., A. fumigatus) and Histoplasma capsulatum. In some embodiments, a yeast is from the CTG clade (Candida clade). In some embodiments, a fungus comprises Candida albicans.

In some embodiments, a fungus comprises a fungus population (e.g., a community of Candida albicans).

“Virulence,” as used herein, refers to a phenotypic state of a fungus associated with infection that may harm its host, for example, the host's epithelial tissues. In some embodiments, attenuating virulence of a fungus (e.g., Candida albicans) comprises modulating (e.g., downregulating) a virulence-associated gene of the fungus, reducing the fungus's surface adhesion (reducing the fungus from attaching to a surface), inhibiting the fungus's morphological transition to an invasive or virulent cell type, inhibiting fungal biofilm formation, reducing the fungus's secretion of a hydrolytic enzyme, or a combination thereof.

In some embodiments, attenuating virulence of a fungus (e.g., Candida albicans) comprises downregulating a gene positively correlated with virulence, upregulating a gene negatively correlated with virulence, or both. Non-limiting examples of virulence-associated gene include genes encoding proteins within the pathways of regulating filamentation and/or adhesion (e.g., encoding adhesins, secreted proteases, cytolytic toxins) and interspecies interactions.

In some embodiments, modulating a virulence-associated gene comprises:
a) downregulating expression of SOD3, SOD5, CSH1, PGA13, or a combination thereof;
b) increasing expression of CHT3 or GLT1, or both; or
both a) and b).

In some embodiments, modulating a virulence-associated gene comprises:
a) downregulating expression of RAS1, EED1, HGC1, UME6, EFG1, BRG1, EFG1, TEC1, ROB1, RFX2, AHR1, ECE1, HYR1, ALS3, HWP1, PRA1, PHR1, GAC1, PTP3, AHR1, YVC1, SAP5, IHD1, RTA4, PGA54, IHD2, DDR48, CSA1, SHE3, or TUP1, or a combination thereof;
b) increasing expression of NRG1, YWP1, or both; or
both a) and b).

In some embodiments, a method disclosed herein reduces expression of a virulence-associated gene by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%. In some embodiments, a method reduces expression of a virulence-associated gene by at least about 30%.

In some embodiments, a method increases expression expression of a virulence-associated gene by at least about 20%, for example, by at least about: 50%, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold or 100-fold. In some embodiments, a method increases expression expression of a virulence-associated gene by at least about 3-fold.

Expression of a virulence-associated gene can be determined by a person of ordinary skill using methods known in the art, for example, at the RNA level using RNA sequencing (RNA-Seq) or a microarray.

In some embodiments, attenuating virulence of a fungus (e.g., Candida albicans) comprises reducing the fungus's surface adhesion. In some embodiments, a method reduces surface adhesion of a fungus (e.g., Candida albicans) by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%. In some embodiments, a method reduces surface adhesion of a fungus (e.g., Candida albicans) by at least about 30%.

The term “surface,” as used herein, refers to an area of upon which fungal biofilm may grow. In some embodiments, a surface comprises a living surface, an inert surface, or both. In some embodiments, a surface comprises a living surface (e.g., a non-keratinized epithelial surface of the body). In some embodiments, a surface comprises an inert surface.

Non-limiting examples of living surfaces include skin and epithelium of the gastrointestinal (GI) tract, oral cavity and reproductive tract. Non-limiting examples of inert surfaces include those of implants and in-dwelling devices, for example contact lenses, dentures, prosthetics (e.g., hip prosthesis, joint prosthesis, voice prosthesis), valves (e.g., mechanical heart valves), pacemakers, catheters (e.g., urinary catheters and central venous catheters), cannulae, vascular access devices, intrauterine devices (IUDs), intravenous lines, endotracheal tubes, enteral feeding tubes, drainage tubes (e.g., wound drains), tracheostomies, instruments (e.g., surgical or examination instruments), laboratory benches, and a material that supports cell growth, replication, and/or maintenance. In some embodiments, a surface is a surface of a device in fluid communication with a subject's circulatory system.

In some embodiments, attenuating virulence of a fungus (e.g., Candida albicans) comprises inhibiting morphological transition to an invasive or virulent cell type. In some embodiments, attenuating virulence of a fungus (e.g., Candida albicans) comprises inhibiting the fungus's morphological transition to a filamentous state (hyphae formation). In some embodiments, a method reduces morphological transition (e.g., hyphae formation) of a fungus (e.g., Candida albicans) by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%. In some embodiments, a method reduces morphological transition (e.g., from yeast to hyphal form) of a fungus (e.g., Candida albicans) by at least about 30%.

In some embodiments, attenuating virulence of a fungus (e.g., Candida albicans) comprises inhibiting fungal biofilm formation. “Biofilm,” as used herein, refers to a structured community of fungi enclosed in a (e.g., self-produced) polymeric matrix that is adherent to a surface. See, e.g., Desai et al., Fungal biofilms, drug resistance, and recurrent infection, Cold Spring Harb Perspect Med. 4(10):a019729 (2014) for additional information on fungal biofilm, the contents of which are incorporated by reference in their entirety. In some embodiments, a method reduces biofilm formation by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%. In some embodiments, a method reduces biofilm formation by at least about 30%.

In some embodiments, attenuating virulence of a fungus (e.g., Candida albicans) comprises reducing the fungus's secretion of a hydrolytic enzyme. In some embodiments, a method reduces secretion of a hydrolytic enzyme by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%. In some embodiments, a method reduces secretion of a hydrolytic enzyme by at least about 30%.

In another aspect, the disclosure provides methods of inhibiting formation of a fungal biofilm on a surface, the methods comprise contacting the surface with a mucin glycan, tautomer, stereoisomer and/or a pharmaceutically acceptable salt thereof. A surface may comprise any one or more of the surfaces described herein.

In another aspect, the disclosure provides methods of maintaining a mucus barrier on a surface, the methods comprise contacting the surface with a mucin glycan, tautomer, stereoisomer and/or a pharmaceutically acceptable salt thereof.

In another aspect, the disclosure provides methods of attenuating virulence of a fungus in a subject in need thereof, the methods comprise administering to the subject a therapeutically effective amount of a mucin glycan, tautomer, stereoisomer and/or a pharmaceutically acceptable salt thereof.

In another aspect, the disclosure provides methods of treating a fungal infection in a subject in need thereof, the methods comprise administering to the subject a therapeutically effective amount of a mucin glycan, tautomer, stereoisomer and/or a pharmaceutically acceptable salt thereof.

In some embodiments, a fungal infection results from a burn, a wound, keratitis, a bioprosthesis or a medical device, e.g., an indwelling medical device. In some embodiments, a fungal infection is in the lung of a subject.

In another aspect, the disclosure provides methods of treating a biofilm-related infection in a subject in need thereof, the methods comprise administering to the subject a therapeutically effective amount of a mucin glycan, tautomer, stereoisomer and/or a pharmaceutically acceptable salt thereof.

In some embodiments, a biofilm-related infection results from a burn, a wound, keratitis, a bioprosthesis or an indwelling medical device. In some embodiments, a biofilm-related infection is in the lung of a subject.

In another aspect, the disclosure provides methods of maintaining a microbiota in a subject in need thereof, the methods comprise administering to the subject a therapeutically effective amount of a mucin glycan, tautomer, stereoisomer and/or a pharmaceutically acceptable salt thereof.

In another aspect, the disclosure provides methods of maintaining a mucus barrier in a subject in need thereof, the methods comprise administering to the subject a therapeutically effective amount of a mucin glycan, tautomer, stereoisomer and/or a pharmaceutically acceptable salt thereof.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some embodiments, a subject is a human. In some embodiments, the subject is male. In some embodiments the subject is female. In some embodiments, the subject (e.g., human) is immunocompromised (e.g., with AIDS, undergoing an anticancer therapy, or undergoing an immunosuppression therapy). In some embodiments, the subject (e.g., human) has an implanted medical device.

“Subject in need thereof,” as used herein, refers to a subject (e.g., a mammalian subject such as a human) diagnosed with or suspected of having a fungal infection, who will be or has been administered a mucin glycan according to a method of the disclosure. “Subject in need thereof” includes those subjects who already have the undesired physiological change or disease as well as those subjects prone to have the physiological change or disease.

In some embodiments, the subject is diagnosed with or is suspected of having a pulmonary disease (e.g., a chronic pulmonary disease), a lung infection, a mucosal infection, a dermal infection, or an infection, for example, caused by a device, e.g., a medical device. In some embodiments, the subject has a wound (e.g., a burn wound). In some embodiments, the subject is diagnosed with or is suspected of having thrush, vaginal yeast infections, diaper rash or hematogenously disseminated candidiasis. In some embodiments, the subject is diagnosed with or is suspected of having hematogenously disseminated candidiasis. In some embodiments, the subject has a chronic fungal infection.

The administration of the compounds (agents, salts, etc.) and compositions may be carried out in any manner, e.g., by parenteral or nonparenteral administration, including by aerosol inhalation, injection, infusions, ingestion, transfusion, implantation or transplantation. For example, the compositions described herein may be administered to a subject trans-arterially, intradermally, subcutaneously, intratumorally, by intramedullar administration, intranodally, intramuscularly, intravenously (e.g., through an IV drip or by intravenous (i.v.) injection), intranasally, intrathecally or intraperitoneally. In some embodiments, the administration is intravenous. In some embodiments, the administration is topical. In some embodiments, the administration is oral. In some embodiments, the administration is by injection, for instance, directly into a tissue, organ, or site of infection. In some embodiments, the administration is ex vivo. In some embodiments, compounds and compositions are administered by routes such as oral, endobronchial, intrathecal, intracisternal, intra-articular, intraperitoneal, ophthalmic (e.g., in an ophthalmic preparation such as eye drops, intraocular injections, ointments), aerosol, irrigant, peritoneal lavage, endobronchial and intrathecal administration. In one embodiment, the subject has a burn wound and the administration is in an ointment.

In some embodiments, a composition is administered topically, orally, intravenously, nasally, ocularly, or transdermally. In some embodiments, a composition is administered topically. In some embodiments, a composition is administered orally. In some embodiments, a composition is administered intravenously. In some embodiments, a composition is administered nasally. In some embodiments, a composition is administered ocularly. In some embodiments, a composition is administered transdermally.

In some embodiments, a composition is provided in a liquid form. In some embodiments, a composition comprises a dose of from about 0.1 g/liter to about 50 g/liter of mucin glycan(s), for example, about: 0.1 g/liter, 0.2 g/liter, 0.3 g/liter, 0.4 g/liter, 0.5 g/liter, 0.6 g/liter, 0.7 g/liter, 0.8 g/liter, 0.9 g/liter, 1 g/liter, 2 g/liter, 3 g/liter, 4 g/liter, 5 g/liter, 6 g/liter, 7 g/liter, 8 g/liter, 9 g/liter, 10 g/liter, 11 g/liter, 12 g/liter, 13 g/liter, 14 g/liter, 15 g/liter, 16 g/liter, 17 g/liter, 18 g/liter, 19 g/liter, 20 g/liter, 21 g/liter, 22 g/liter, 23 g/liter, 24 g/liter, 25 g/liter, 26 g/liter, 27 g/liter, 28 g/liter, 29 g/liter, 30 g/liter, 31 g/liter, 32 g/liter, 33 g/liter, 34 g/liter, 35 g/liter, 36 g/liter, 37 g/liter, 38 g/liter, 39 g/liter, 40 g/liter, 41 g/liter, 42 g/liter, 43 g/liter, 44 g/liter, 45 g/liter, 46 g/liter, 47 g/liter, 48 g/liter, 49 g/liter or 50 g/liter of mucin glycan(s).

In some embodiments, a composition is provided in a dried form.

In some embodiments, the protein component of a composition is less than about 50% by weight, for example, less than about: 25%, 20%, 18%, 15%, 12%, 10%, 9%, 8%, 7%, 6%, 5%, 4%, 3%, 2%, 1%, 0.9%, 0.8%, 0.7%, 0.6%, 0.5%, 0.4%, 0.3%, 0.2% or 0.1%, by weight.

Administration of a compound, composition, or pharmaceutically acceptable salt described herein may be in conjunction with another active ingredient (e.g., an anti-fungal), for example, simultaneously in the same composition, simultaneously in different dosage forms, or sequentially. A compound, composition, or pharmaceutically acceptable salt described herein and another active ingredient may be formulated in a single combination, multiple combinations, or separate compositions.

In some embodiments, an amount (e.g., a therapeutically effective amount) of mucin glycan or pharmaceutically acceptable salt thereof is sufficient to increase rate of fungal (e.g., Candida albicans) clearance in a subject, for example, compared to the same subject were it left untreated. In some embodiments, an amount (e.g., a therapeutically effective amount) of mucin glycan or pharmaceutically acceptable salt thereof is sufficient to increase the rate of fungal (e.g., Candida albicans) clearance by at least about 20%, for example, by at least about: 50%, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold or 100-fold.

In some embodiments, an amount (e.g., a therapeutically effective amount) of mucin glycan or pharmaceutically acceptable salt thereof is sufficient to reduce expression of a virulence-associated gene (e.g., UME6 or HGC1) by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%. In some embodiments, an amount (e.g., a therapeutically effective amount) of mucin glycan or pharmaceutically acceptable salt thereof is sufficient to reduce expression of a virulence-associated gene by at least about 30%.

In some embodiments, an amount (e.g., a therapeutically effective amount) of mucin glycan or pharmaceutically acceptable salt thereof is sufficient to increase expression of a virulence-associated gene (e.g., YWP1) by at least about 20%, for example, by at least about: 50%, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold or 100-fold. In some embodiments, an amount (e.g., a therapeutically effective amount) of mucin glycan or pharmaceutically acceptable salt thereof is sufficient to increase expression of a virulence-associated gene by at least about 3-fold.

In some embodiments, an amount (e.g., a therapeutically effective amount) of mucin glycan or pharmaceutically acceptable salt thereof is sufficient to reduce surface adhesion of a fungus (e.g., Candida albicans) by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%. In some embodiments, an amount (e.g., a therapeutically effective amount) of mucin glycan or pharmaceutically acceptable salt thereof is sufficient to reduce surface adhesion of a fungus (e.g., Candida albicans) by at least about 30%.

In some embodiments, an amount (e.g., a therapeutically effective amount) of mucin glycan or pharmaceutically acceptable salt thereof is sufficient to reduce a morphological transition (e.g., hyphae formation) of a fungus (e.g., Candida albicans) by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%. In some embodiments, an amount (e.g., a therapeutically effective amount) of mucin glycan or pharmaceutically acceptable salt thereof is sufficient to reduce a morphological transition (e.g., from yeast to hyphal form) of a fungus (e.g., Candida albicans) by at least about 30%.

In some embodiments, an amount (e.g., a therapeutically effective amount) of mucin glycan or pharmaceutically acceptable salt thereof is sufficient to reduce biofilm formation by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%. In some embodiments, an amount (e.g., a therapeutically effective amount) of mucin glycan or pharmaceutically acceptable salt thereof is sufficient to reduce biofilm formation by at least about 30%.

In some embodiments, an amount (e.g., a therapeutically effective amount) of mucin glycan or pharmaceutically acceptable salt thereof is sufficient to reduce secretion of a hydrolytic enzyme by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75% 40-75%, 40-70%, 45-70%, 45-65%, 50-65% or 50-60%. In some embodiments, an amount (e.g., a therapeutically effective amount) of mucin glycan or pharmaceutically acceptable salt thereof is sufficient to reduce secretion of a hydrolytic enzyme by at least about 30%.

### Compositions of the Disclosure

In another aspect, the disclosure provides defined or semi-defined mucin glycan compositions, comprising one or more desired mucin glycans or pharmaceutically acceptable salts thereof, wherein at least 80% of total mucin glycans present in the compositions are of the one or more desired mucin glycans or pharmaceutically acceptable salts thereof.

A defined, or semi-defined mucin glycan composition comprises at least 1 desired mucin glycan or pharmaceutically acceptable salt thereof, for example, 2, 3, 4, 5, 6, 7, 8, 9, 10, 15, 20 or more desired mucin glycans or pharmaceutically acceptable salts or 1-20, 1-18, 1-15, 1-12, 1-10, 1-8, 1-5, 1-4, 1-3, 1-2, 2-20, 2-18, 2-15, 2-12, 2-10, 2-8, 2-5, 2-4, 2-3, 3-20, 3-18, 3-15, 3-12, 3-10, 3-8, 3-5, 3-4, 4-20, 4-18, 4-15, 4-12, 4-10, 4-8, 4-5, 5-20, 5-18, 5-15, 5-12, 5-10, 5-8, 8-20, 8-18, 8-15, 8-12 or 8-10 desired mucin glycans or pharmaceutically acceptable salts thereof.

In some embodiments, a defined, or semi-defined mucin glycan composition comprises at least 2 desired mucin glycans or pharmaceutically acceptable salts thereof at a desired ratio. In some embodiments, a desired ratio of a first versus a second desired mucin glycan or pharmaceutically acceptable salt thereof is about: 1:1, 3:4, 2:3, 1:2, 1:3, 1:4, 1:5, 1:6, 1:8, 1:10 or 1:15.

In some embodiments, a mucin glycan composition is defined. In other embodiments, a mucin glycan composition is semi-defined.

In some embodiments, at least 85%, 90%, 95%, 99%, 99.5% or 99.9% of the total mucin glycans or pharmaceutically acceptable salts thereof present in a composition are of the one or more desired mucin glycans or pharmaceutically acceptable salts thereof. In some embodiments, about 80-99.9% of the total mucin glycans or pharmaceutically acceptable salts thereof present in a composition are of the one or more desired mucin glycans or pharmaceutically acceptable salts thereof, for example, about: 85-99.9%, 85-99.5%, 85-99%, 85-98%, 85-95%, 88-99.9%, 88-99.5%, 88-99%, 88-98%, 88-95%, 90-99.9%, 90-99.5%, 90-99%, 90-98%, 90-95%, 92-99.9%, 92-99.5%, 92-99%, 92-98%, 92-95%, 95-99.9%, 95-99.5%, 95-99% or 95-98% of the total mucin glycans present in the composition are of the one or more desired mucin glycans or pharmaceutically acceptable salts thereof.

In some embodiments, a defined, or semi-defined mucin glycan composition is incorporated into a formulation for therapeutic administration (e.g., a pharmaceutical composition). In some embodiments, a pharmaceutical composition further comprises one or more pharmaceutically acceptable carriers or diluents. In some embodiments, a pharmaceutical composition further comprises one or more additional therapeutics, i.e., therapeutic agents (e.g., an antifungal). Pharmaceutical compositions may be formulated into preparations in, fore example, solid, semi-solid, liquid or gaseous forms, such as capsules, gels, granules, microspheres, ointments, powders, solutions, drops, and tablets. Defined, or semi-defined mucin glycan compositions may be formulated for various routes of administration, for example, oral formulations, intravenous formulations, or in the form of a douche. In some embodiments, a defined, or semi-defined mucin glycan composition is formulated into an ointment.

In some embodiments, a defined, or semi-defined mucin glycan composition is incorporated into a coating (e.g., a film) for any one or more of the surfaces described herein (e.g., to inhibit formation of a fungal biofilm). In some embodiments, a coating (e.g., a single- of multi-layer film) is biocompatible.

In another aspect, the disclosure provides compositions comprising a synthetic mucin glycan.

In another aspect, the disclosure provides compositions comprising a mucin glycan, wherein the purity of the mucin glycan is at least about 30%. In some embodiments, the purity of the mucin glycan is at least about: 55%, 60%, 65%, 70%, 75%, 80%, 85%, 90%, 95%, or 99.5%. In some embodiments, the purity of the mucin glycan is at least about 50%. In some embodiments, the purity of the mucin glycan is at least about 99.5%. In some embodiments, the composition comprises at least about 100 mg of a mucin glycan having a purity of at least about 99.5%.

### Mucins

Mucins are heavily O-glycosylated glycoproteins that are found in mucous secretions (secreted mucins) and on the cell surface (membrane-bound (transmembrane) mucins). Secreted mucins include gel-forming mucins and non-gel-forming (soluble) mucins.

Mucin genes are expressed in a tissue- and/or region-specific fashion, for example, in the airway, digestive system, reproductive system, and different regions of the gastrointestinal tract. About 20 different mucin genes have been cloned, including gel-forming mucin genes such as MUC2, MUC5AC, MUC5B, MUC6 and MUC19; soluble mucin genes such as MUC7, MUC8, MUC9 and MUC20; and transmembrane mucin genes such as MUC1, MUC3A, MUC3B, MUC4, MUC12, MUC13, MUC15 and MUC21.

Non-limiting examples of mucin genes include human MUC1 (e.g., GenBank: AAA60019.1, UniProtKB/Swiss-Prot: P15941.3, Gene ID 4582), porcine MUC1 (e.g., NCBI: XP_020945387.1), human MUC2 (e.g., GenBank: AAB95295.1, Gene ID 4583), porcine MUC2 (e.g., NCBI: XP_020938243.1), human MUC5AC (e.g., GenBank: ABV02582.1, UniProtKB/Swiss-Prot: P98088.4, Gene ID 4586), porcine MUC5AC (e.g., NCBI: XP_020938242.1), human MUC5B (e.g., UniProtKB/Swiss-Prot: Q9HC84.3, Gene ID 727897), porcine MUC5B (e.g., NCBI: XP_020938146.1), human MUC6 (e.g., GenBank: AZL49144.1) and porcine MUC6 (e.g., NCBI: XP_020938133.1).

Additional non-limiting examples of mucin genes include MUC2 (e.g., HomoloGene 130504, 131905, 132025, or 133451), MUC5AC (e.g., UniGene IDs 3881294, 1370646, 1774723, 1133368, 441382, and 5878683, HomoloGene 130646; Gene ID 100170143; and reference sequences AAC48526, AAD19833, and AAD19832), MUC5B (e.g., HomoloGene 124413), MUC6 (e.g., HomoloGene 18768), MUC19 (bovine submaxillary mucin (BSM), e.g., Gene ID 100140959; HomoloGene 130967; and reference protein sequence XP_0035861 12.1).

### Mucin Glycans

A mucin protein comprises an amino region and/or a carboxy region that are cysteine-rich and a central region enriched in serine and/or threonine residues. Native mucin glycans are typically built upon an N-acetylgalactosamine that is O-linked via its C-1 hydroxyl to serine or threonine residues of a mucin protein. The monosaccharide unit or series of monosaccharide units that, in a native mucin glycan, would be O-linked via the C-1 hydroxyl of the monosaccharide unit or first monosaccharide unit in the series of monosaccharide units, respectively, to a serine or threonine residue of the mucin protein is also referred to herein as the “glycan core” or “mucin glycan core.”

As used herein, the term “mucin glycan” refers to a compound comprising (e.g., consisting of) a glycan, or a portion thereof, found on a native mucin. “Mucin glycans” can be natural (e.g., derived from purification) or synthetic. In some embodiments, the mucin glycan is a synthetic mucin glycan.

In some embodiments, a mucin glycan is purified from a non-human animal, for example, a domesticated mammal such as a porcine, a bovine. In some embodiments, a mucin glycan is gut-derived. In some embodiments, a mucin glycan is prepared from cell culture (e.g., of a recombinant cell line or a non-recombinant cell line).

In some embodiments, a mucin glycan is released from a mucin clycoprotein, for example, by enzymatic degradation, or a chemical release (reductive or non-reductive).

It has been found that native glycans exhibit certain common cores, such as those described herein as Cores 1-8. Thus, in some embodiments, a mucin glycan comprises a glycan core. In some embodiments, the glycan core comprises GalNAc, e.g., as the sole monosaccharide in the core or as the first monosaccharide unit in a series of monosaccharide units that make up the core. In some embodiments, the glycan core comprises (e.g., consists of) two or more monosaccharide units linked via O-glycosidic linkages (e.g., GalNAc and one or more additional monosaccharide units linked via O-glycosidic linkages).

It will be understood that one or more monosaccharide units of a mucin glycan compound and/or glycan core described herein can be optionally substituted (e.g., unsubstituted; substituted) by one or more (e.g., from one to ten, from one to five, from one to three) independently selected substituents in accordance with this disclosure as, for example, when a monosaccharide in a glycan core is substituted with an additional monosaccharide unit via an O-glycosidic linkage. Typically, when a monosaccharide unit of a glycan core is substituted with an additional monosaccharide unit, the hydrogen atom of a hydroxyl group of the monosaccharide of the glycan core being substituted is replaced with the substituent, such as the additional monosaccharide unit.

It will also be understood that the mucin glycan compound, in addition to comprising a glycan may contain further, non-saccharide substituents. Thus, for example, one or more oxygen atoms of the monosaccharide hydroxyls in the glycan may be independently substituted, as by replacing the hydrogen atom of a monosaccharide hydroxyl with a substituent, such as alkyl (e.g., methyl, ethyl, n-propyl, isopropyl), aryl (e.g., phenyl, biaryl), —C(O)H or —C(O)alkyl (e.g., acetyl). See, for example, Compounds 3 and 6 described herein, which are substituted with methyl at the 1-position of the galactopyranoside. In some embodiments, the oxygen atom of the hydroxyl at the anomeric/C-1 position of the monosaccharide unit or first monosaccharide unit in the series of monosaccharide units of the glycan core is substituted (e.g., with alkyl (e.g., methyl, ethyl, n-propyl, isopropyl), aryl (e.g., phenyl, biaryl), —C(O)H or —C(O)alkyl (e.g., acetyl)). In some embodiments, the oxygen atom of the hydroxyl at the anomeric/C-1 position of the GalNAc residue of Cores 1-8 is substituted (e.g., with alkyl (e.g., methyl, ethyl, n-propyl, isopropyl), aryl (e.g., phenyl, biaryl), —C(O)H or —C(O)alkyl (e.g., acetyl)). In further embodiments, the oxygen atoms(s) of the remaining hydroxyls in the glycan or glycan core are not substituted with a non-saccharide substituent. In further embodiments, the oxygen atom(s) of the remaining hydroxyls in the glycan or glycan core are not substituted.

Combinations of substituents envisioned by this invention are preferably those that result in the formation of stable or chemically feasible compounds.

When a group is substituted herein, the substituted group can have a suitable substituent at each substitutable position of the group and, when more than one position in any given structure may be substituted with more than one substituent selected from a specified group, the substituent can be the same or different at every position.

Suitable substituents include, but are not limited to, halogen, hydroxyl, carbonyl (such as carboxyl, alkoxycarbonyl, formyl, or acyl), thiocarbonyl (such as thioester, thioacetate, or thioformate), alkyl, alkoxy, alkylthio, acyloxy, phosphoryl, phosphate, phosphonate, amino, amido, amidino, imino, cyano, nitro, azido, sulfhydryl, alkylthio, sulfate, sulfonate, sulfamoyl, sulfonamido, sulfonyl, cycloalkyl, heterocyclyl, aryl, biaryl or heteroaryl. It will be understood by those skilled in the art that substituents can themselves be substituted, if appropriate. For instance, substituent(s) of a substituted alkyl may include substituted and unsubstituted forms of hydroxyl, amino, imino, amido, phosphoryl (including phosphonate and phosphinate), sulfonyl (including sulfate, sulfonamido, sulfamoyl and sulfonate) and carbonyls (including ketones, aldehydes, carboxylates, and esters), and the like.

It will also be appreciated by a person of ordinary skill in the art, that a mucin glycan having hydroxyl at the anomeric/C-1 position of the GalNAc residue of Cores 1-8 may equilibrate into various different forms, for example, in aqueous solutions, such as aqueous formulations and in the body. The process by which equilibration occurs is known as glycan mutarotation/tautomerization. The tautomers (e.g., ring-chain tautomers, including both the cyclic (such as, furanose and pyranose) tautomeric forms and the linear or open-chain tautomeric forms) as well as the isomers (e.g., anomers, including both the alpha- and beta-anomers) resulting from glycan mutarotation/tautomerization are within the scope of the present disclosure. Thus, in some embodiments of any of the structural formulas herein, the structural formula includes tautomers (e.g., ring-chain tautomers, including both the cyclic (such as, furanose and pyranose) tautomeric forms and the linear or open-chain tautomeric forms) and isomers (e.g., anomers) thereof, in particular, those resulting from glycan mutarotation/tautomerization.

In some embodiments, a mucin glycan is a primate (e.g., human) mucin glycan. In some embodiments, a mucin glycan is a non-primate (e.g., porcine, bovine, or mouse) mucin glycan.

In some embodiments, a native mucin is expressed in the airway, the digestive system, the reproductive system, or a combination thereof. In some embodiments, a native mucin is expressed in the digestive system. In some embodiments, a mucin glycan comprises a gastric mucin glycan, a salivary gland mucin glycan, an intestinal mucin glycan, or a combination thereof.

In some embodiments, a mucin glycan is a secreted mucin glycan (e.g., of the digestive system). In some embodiments, a mucin glycan is a gel-forming mucin glycan (e.g., of the digestive system).

In some embodiments, a mucin glycan comprises an oligosaccharide of from about 2 to about 10 monosaccharide subunits in length, for example, 2, 3, 4, 5, 6, 7, 8, 9, 10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9 or 9-10 monosaccharide subunits in length. In some embodiments, a mucin glycan comprises an oligosaccharide of from about 2 to about 6 monosaccharide subunits in length. In some embodiments, a mucin glycan comprises an oligosaccharide of from about 4 to about 6 monosaccharide subunits in length. In some embodiments, a mucin glycan comprises an oligosaccharide of from about 6 to about 8 monosaccharide subunits in length. In some embodiments, a mucin glycan comprises an oligosaccharide of from about 6 to about 10 monosaccharide subunits in length. In some embodiments, a mucin glycan comprises an oligosaccharide of from about 8 to about 10 monosaccharide subunits in length.

Non-limiting examples of linear and/or branched oligosaccharides include galactose, N-acetylgalactosamine, fucose, glucose and N-acetylglucosamine, with heterogenous linkages within any one glycan, e.g., a mixture of α2-3, α2-6, α1-2, α1-3, α1-4, β1-3, β1-4, β1-6, etc. The numbering is shown with respect to D-glucopyranose, where “α” refers to the configuration of a cyclic sugar where the oxygen on the anomeric carbon is on the opposite face of the ring relative to the substituent on the other carbon flanking the ring oxygen and “β” refers to the configuration of a cyclic sugar where the oxygen on the anomeric carbon is on the same face of the ring as the substituent on the other carbon flanking the ring oxygen.

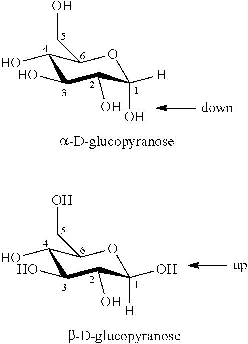

In some embodiments, a mucin glycan comprises one or more N-acetylgalactosamine (GalNAc), N-acetylglucosamine (GlcNAc), mannose (Man), fucose (Fuc), N-acetylneuraminic acid (Neu5Ac), galactose (Gal) or a combination thereof. In some embodiments, a mucin glycan comprises one or more N-acetylgalactosamine (GalNAc), N-acetylglucosamine (GlcNAc), N-acetylneuraminic acid (Neu5Ac), galactose (Gal) or a combination thereof. In some embodiments, a mucin glycan further comprises sialic acid.

In some embodiments, a mucin glycan is unsulfated. In other embodiments, a mucin glycan is sulfated.

### Core 1 Mucin Glycan

In some embodiments, a mucin glycan comprises a glycan core of the following structural formula:

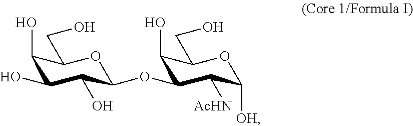

or a tautomer or stereoisomer thereof.

In some embodiments, a mucin glycan comprises a glycan structure selected from the group consisting of 1a, 2, 3a, 4, 9c, 15b, 16b, 18b, 5, 6, 7 and 25b of FIG. 4A. In some embodiments, a Core 1 mucin glycan is unsulfated. In some embodiments, a Core 1 mucin glycan is sulfated. In some embodiments, a sulfated Core 1 mucin glycan comprises a glycan structure selected from the group consisting of 1a, 2, 3a, 9c, 15b and 18b of FIG. 4A.

In some embodiments, a mucin glycan (e.g., Core 1 mucin glycan) further comprises fucose, sialic acid, or a combination thereof. In some embodiments, a mucin glycan comprises the following structural formula:

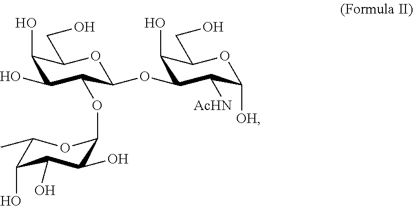

or a tautomer or stereoisomer thereof, or

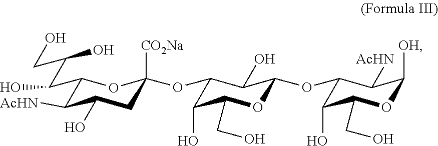

or a tautomer or stereoisomer thereof.

### Core 2 Mucin Glycan

In some embodiments, a mucin glycan comprises a glycan core of the following structural formula:

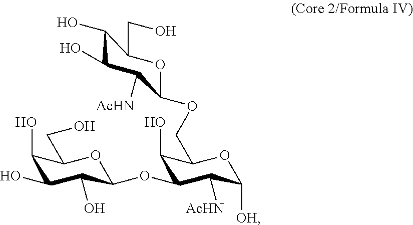

or a tautomer or stereoisomer thereof.

In some embodiments, a mucin glycan comprises a glycan structure selected from the group consisting of 8a, 9a, 9b, 10a, 11a, 12a, 12b, 13, 14a, 15a, 16a, 17, 18a, 19, 20a, 21a, 21b, 22, 23a, 24a, 24b, 25a, 26a, 27, 28, 29, 30, 31, 32, 33a, 34a, 35, 36, 37, 38a, 38b, 39, 40, 41, 42, 43, 44, 45, 46, 47a, 48, 49a, 50, 51, 52, 53a and 54 of FIG. 4A. In some embodiments, a Core 2 mucin glycan is unsulfated. In some embodiments, a Core 2 mucin glycan is sulfated. In some embodiments, a sulfated Core 2 mucin glycan comprises a glycan structure selected from the group consisting of 8a, 9a, 9b, 11a, 12a, 12b, 13, 14a, 15a, 17, 18a, 19, 20a, 21a, 21b, 23a, 24a, 24b, 30 and 34a of FIG. 4A.

In some embodiments, a mucin glycan (e.g., Core 2 mucin glycan) further comprises fucose, galactose, or a combination thereof. In some embodiments, a mucin glycan comprises the following structural formula:

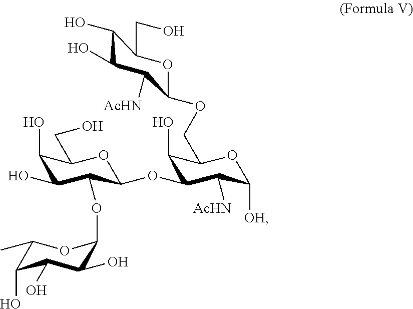

or a tautomer or stereoisomer thereof or

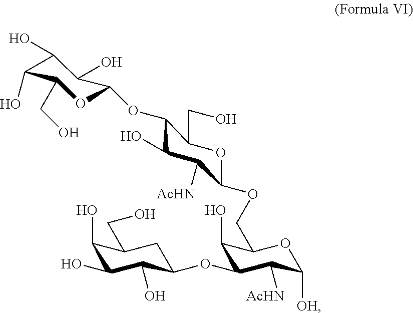

or a tautomer or stereoisomer thereof.

### Core 3 Mucin Glycan

In some embodiments, a mucin glycan comprises a glycan core of the following structural formula:

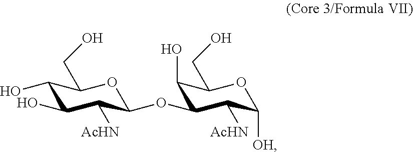

or a tautomer or stereoisomer thereof.

In some embodiments, a mucin glycan comprises a glycan structure selected from the group consisting of 8b, 10b, 20b, 26b and 55 of FIG. 4A. In some embodiments, a Core 3 mucin glycan is unsulfated. In some embodiments, a Core 3 mucin glycan is sulfated. In some embodiments, a sulfated Core 3 mucin glycan comprises a glycan structure selected from the group consisting of 8b and 20b of FIG. 4A.

### Core 4 Mucin Glycan

In some embodiments, a mucin glycan comprises a glycan core of the following structural formula:

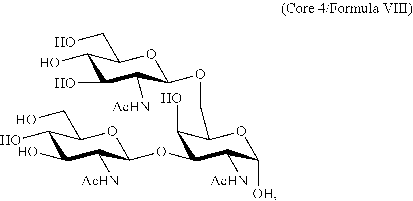

or a tautomer or stereoisomer thereof.

In some embodiments, a mucin glycan comprises a glycan structure selected from the group consisting of 34b and 56 of FIG. 4A. In some embodiments, a Core 4 mucin glycan is unsulfated. In some embodiments, a Core 4 mucin glycan is sulfated. In some embodiments, a sulfated Core 4 mucin glycan comprises a glycan structure of 34b of FIG. 4A.

### O-Man Core Mucin Glycan

In some embodiments, a mucin glycan comprises a glycan core of the following structural formula:

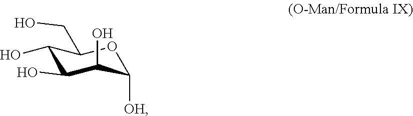

or a tautomer or stereoisomer thereof.

In some embodiments, a mucin glycan comprises a glycan structure selected from the group consisting of 1b, 3b, 11b, 14b, 57, 8c, 23b, 23c, 24c, 33b, 58, 47b, 49b and 53b of FIG. 4A. In some embodiments, an O-Man Core mucin glycan is unsulfated. In some embodiments, an O-Man Core mucin glycan is sulfated. In some embodiments, a sulfated O-Man Core mucin glycan comprises a glycan structure of 11b, 14b, 57, 23b and 23c of FIG. 4A.

### Tn Antigen Mucin Glycan

In some embodiments, a mucin glycan comprises a glycan core of one of the following structural formula:

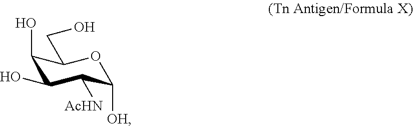

or a tautomer or stereoisomer thereof.

In some embodiments, a mucin glycan comprises a glycan structure of 5 of FIG. 4A.

### Core 5, 6, 7 or 8 Mucin Glycan

In some embodiments, a mucin glycan comprises a glycan core of one of the following structural formula:

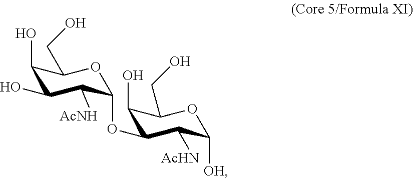

or a tautomer or stereoisomer thereof,

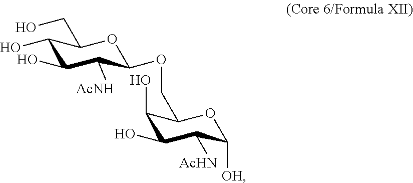

or a tautomer or stereoisomer thereof,

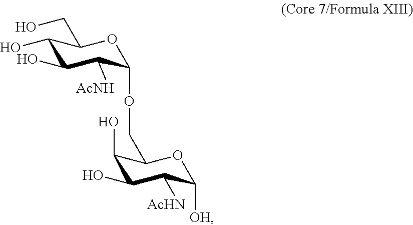

or a tautomer or stereoisomer thereof, or

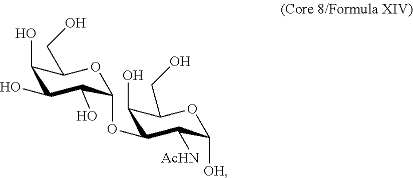

or a tautomer or stereoisomer thereof.

### MUC2, MUC5AC & MUC5B Glycans

In some embodiments, a mucin glycan comprises (e.g., consists of) a MUC2 glycan, a MUC5AC glycan, a MUC5B glycan, or a combination thereof. In some embodiments, a mucin glycan comprises a porcine MUC2 glycan, a porcine MUC5AC glycan, a human MUC5B glycan, or a combination thereof.

In some embodiments, a mucin glycan comprises a formula selected from the group consisting of (HexNAc)1 (e.g., GlyTouCan Accession: G57321FI); (Hex)1 (HexNAc)1 (e.g., G76355TG, G85856KC); (Hex)1 (HexNAc)1 (Deoxyhexose)1 (e.g., G94435QH); (Hex)1 (HexNAc)1 (NeuAc)1 (e.g., G65562ZE); (Hex)2 (HexNAc)1 (Deoxyhexose)1 (e.g., G73318SN, G33986KK); (Hex)1 (HexNAc)1 (NeuGc)1 (e.g., G64527IJ); (Hex)1 (HexNAc)1 (Deoxyhexose)1 (NeuAc)1 (e.g., G47180UC); (Hex)2 (HexNAc)1 (Deoxyhexose)2 (e.g., G68200GL); (Hex)1 (HexNAc)1 (NeuAc)2 (e.g., G01614ZM); (Hex)2 (HexNAc)1 (Deoxyhexose)3 (e.g., G82961CS); (Hex)2 (HexNAc)1 (Deoxyhexose)1 (NeuAc)1 (e.g., G49549VN, G95742RK); (Hex)1 (HexNAc)1 (NeuAc)1 (NeuGc)1 (e.g., G49527BY); (Hex)1 (HexNAc)2 (Deoxyhexose)1 (NeuAc)1 (e.g., G75749JP, G40270LS); (Hex)3 (HexNAc)2 (Deoxyhexose)4 (e.g., G93469SN, G15747RC); (Hex)1 (HexNAc)2 (e.g., G00033MO, G61730RY, G56868BH); (Hex)1 (HexNAc)2 (Deoxyhexose)1 (e.g., G32426JY, G74353FF); (Hex)2 (HexNAc)2 (e.g., G64973KT); (Hex)1 (HexNAc)3 (e.g., G68893BQ, G23438NR); (Hex)1 (HexNAc)2 (Deoxyhexose)2 (e.g., G89748NG, G09520ZQ); (Hex)1 (HexNAc)2 (NeuAc)1 (e.g., G85608AG, G64844ET); (Hex)2 (HexNAc)2 (Deoxyhexose)1 (e.g., G94514IB, G61216ZY); (Hex)1 (HexNAc)2 (NeuGc)1 (e.g., G60426XC); (Hex)3 (HexNAc)2 (e.g., G16404NW, G25323VU); (Hex)1 (HexNAc)3 (Deoxyhexose)1 (e.g., G28878FC); (Hex)2 (HexNAc)3 (e.g., G96915PP, G13483MW); (Hex)1 (HexNAc)4 (e.g., G59229NY); (Hex)2 (HexNAc)2 (Deoxyhexose)2 (e.g., G23119MJ); (Hex)2 (HexNAc)2 (NeuAc)1 (e.g., G46748BU); (Hex)3 (HexNAc)2 (Deoxyhexose)1 (e.g., G15849KC, G81911LP, G41486OC); (Hex)2 (HexNAc)3 (Deoxyhexose)1 (e.g., G91459EI, G26724PP, G52132CU); (Hex)3 (HexNAc)3 (e.g., G86537AD, G89585FG); (Hex)2 (HexNAc)4 (e.g., G34764BK, G12074QJ); (Hex)2 (HexNAc)2 (Deoxyhexose)3 (e.g., G01532FF); (Hex)2 (HexNAc)2 (Deoxyhexose)1 (NeuAc)1 (e.g., G77740PR, G59155GF); (Hex)3 (HexNAc)2 (Deoxyhexose)2 (e.g., G20310DM, G78177LC); (Hex)2 (HexNAc)3 (Deoxyhexose)2 (e.g., G68384KC, G39326PP); (Hex)3 (HexNAc)3 (Deoxyhexose)1 (e.g., G66741QE, G16458JH); (Hex)2 (HexNAc)4 (Deoxyhexose)1 (e.g., G23700TV); (Hex)3 (HexNAc)4 (e.g., G94517VF, G57672ST); (Hex)3 (HexNAc)2 (Deoxyhexose)3 (e.g., G70416EY, G13012GZ); (Hex)2 (HexNAc)5 (e.g., G23048PE); (Hex)3 (HexNAc)2 (Deoxyhexose)1 (NeuAc)1 (e.g., G74607VK, G81461IK); (Hex)3 (HexNAc)3 (Deoxyhexose)2 (e.g., G35949CT, G71094KR, G08426KY); (Hex)2 (HexNAc)4 (Deoxyhexose)2 (e.g., G90965BZ); (Hex)3 (HexNAc)4 (Deoxyhexose)1 (e.g., G02990AF, G29956GF); (Hex)3 (HexNAc)3 (Deoxyhexose)3 (e.g., G23021IW); (Hex)4 (HexNAc)3 (Deoxyhexose)2 (e.g., G68308CM); (Hex)3 (HexNAc)4 (Deoxyhexose)2 (e.g., G93333OF); (Hex)4 (HexNAc)4 (Deoxyhexose)1 (e.g., G94768NG); (Hex)2 (HexNAc)5 (Deoxyhexose)2 (e.g., G25612EW); (Hex)3 (HexNAc)3 (Deoxyhexose)4 (e.g., G84853WN); (Hex)5 (HexNAc)6 (Deoxyhexose)3 (e.g., G65612SS); (Hex)3 (HexNAc)5 (Deoxyhexose)1 (e.g., G05252QE); (Hex)7 (HexNAc)6 (Deoxyhexose)1 (e.g., G61898SS); (Hex)3 (HexNAc)6 (e.g., G62609ZF); (Hex)3 (HexNAc)4 (Deoxyhexose)3 (e.g., G59787TQ); (Hex)4 (HexNAc)4 (Deoxyhexose)2 (e.g., G98518WL); (Hex)6 (HexNAc)7 (Deoxyhexose)2 (e.g., G29852ZH); (Hex)4 (HexNAc)5 (Deoxyhexose)1 (e.g., G92547QZ); (Hex)3 (HexNAc)4 (Deoxyhexose)4 (e.g., G89469SP); (Hex)5 (HexNAc)4 (Deoxyhexose)2 (e.g., G21630AC); (Hex)4 (HexNAc)5 (Deoxyhexose)2 (e.g., G82251ZP, G18603ZQ); (Hex)5 (HexNAc)5 (Deoxyhexose)1 (e.g., G25957KN); (Hex)4 (HexNAc)6 (Deoxyhexose)1 (e.g., G32752FJ); (Hex)6 (HexNAc)5 (Deoxyhexose)1 (e.g., G37901JE, G84713IO); (Hex)4 (HexNAc)6 (Deoxyhexose)2 (e.g., G99804SJ); (Hex)4 (HexNAc)5 (Deoxyhexose)4 (e.g., G11381FO); (Hex)5 (HexNAc)5 (Deoxyhexose)3 (e.g., G90829NZ); (Hex)6 (HexNAc)5 (Deoxyhexose)2 (e.g., G70999YJ, G02681FY); (Hex)5 (HexNAc)6 (Deoxyhexose)2 (e.g., G44467ZE); (Hex)3 (HexNAc)5 (Deoxyhexose)2 (e.g., G28921PH); (Hex)6 (HexNAc)5 (Deoxyhexose)3 (e.g., G18501TC); (Hex)4 (HexNAc)4 (Deoxyhexose)3 (e.g., G66166BF); (HexNAc)2 (e.g., G00041MO, G00057MO); (HexNAc)2 (NeuAc)1 (e.g., G63334FZ); (HexNAc)2 (NeuGc)1 (e.g., G09441IP); (Hex)3 (HexNAc)5 (e.g., G00505CR); (Hex)2 (HexNAc)5 (Deoxyhexose)1 (e.g., G09396HG); (Hex)2 (HexNAc)1 (e.g., G28052FT); (Hex)2 (HexNAc)1 (NeuAc)1 (e.g., G59126YU); (Hex)1 (Deoxyhexose)1 (e.g., G00068MO); (Hex)1 (NeuAc)1 (e.g., G30207PZ, G63069TR); and (Hex)1 (NeuGc)1 (e.g., G38557KR, G59867EM).

“Hex” refers to hexose, a monosaccharide with six carbon atoms, C₆H₁₂O₆. “NAC” refers to N-acetylcysteine. “NeuAc” or “Neu5Ac” refers to N-acetylneuraminic acid. “NeuGc” or “Neu5Gc” refers to N-glycolylneuraminic acid. “HexNAc” refers to N-acetylhexosamine. “Deoxyhexose” refers to any deoxysugar derived from a hexose.

### MUC2 Glycans

In some embodiments, a mucin glycan comprises a porcine MUC2 glycan.

In some embodiments, a mucin glycan comprises a formula selected from the group consisting of (Hex)1 (HexNAc)1 (Deoxyhexose)1 (e.g., G94435QH); (Hex)1 (NeuAc)1 (e.g., G30207PZ, G63069TR); (Hex)1 (Deoxyhexose)1 (e.g., G00068MO); (Hex)1 (HexNAc)1 (e.g., G76355TG, G85856KC); (Hex)1 (NeuGc)1 (e.g., G38557KR, G59867EM); (Hex)1 (HexNAc)2 (Deoxyhexose)1 (e.g., G32426JY, G74353FF); (Hex)1 (HexNAc)1 (NeuAc)1 (e.g., G65562ZE); (Hex)1 (HexNAc)1 (NeuGc)1 (e.g., G64527IJ); (Hex)1 (HexNAc)2 (e.g., G00033MO, G61730RY, G56868BH); (HexNAc)2 (e.g., G00041MO, G00057MO); (HexNAc)1 (e.g., G57321FI); (Hex)1 (HexNAc)3 (Deoxyhexose)1 (e.g., G28878FC); (Hex)2 (HexNAc)2 (NeuAc)1 (e.g., G46748BU); (Hex)2 (HexNAc)2 (e.g., G64973KT); (HexNAc)2 (NeuAc)1 (e.g., G63334FZ); (Hex)2 (HexNAc)1 (e.g., G28052FT); (Hex)2 (HexNAc)2 (Deoxyhexose)1 (e.g., G94514IB, G61216ZY); (Hex)1 (HexNAc)3 (e.g., G68893BQ, G23438NR); (HexNAc)2 (NeuGc)1 (e.g., G09441IP); (Hex)2 (HexNAc)2 (Deoxyhexose)2 (e.g., G23119MJ); (Hex)1 (HexNAc)2 (NeuAc)1 (e.g., G85608AG, G64844ET); (Hex)2 (HexNAc)3 (Deoxyhexose)2 (e.g., G68384KC, G39326PP); (Hex)2 (HexNAc)3 (e.g., G96915PP, G13483MW); (Hex)1 (HexNAc)1 (NeuAc)2 (e.g., G01614ZM); (Hex)1 (HexNAc)2 (NeuGc)1 (e.g., G60426XC); (Hex)1 (HexNAc)1 (Deoxyhexose)1 (NeuAc)1 (e.g., G47180UC); (Hex)1 (HexNAc)2 (Deoxyhexose)1 (NeuAc)1 (e.g., G75749JP, G40270LS); (Hex)4 (HexNAc)5 (Deoxyhexose)1 (e.g., G92547QZ); (Hex)3 (HexNAc)4 (Deoxyhexose)1 (e.g., G02990AF, G29956GF); (Hex)2 (HexNAc)4 (Deoxyhexose)2 (e.g., G90965BZ); (Hex)3 (HexNAc)5 (Deoxyhexose)1 (e.g., G05252QE); (Hex)2 (HexNAc)1 (NeuAc)1 (e.g., G59126YU); (Hex)3 (HexNAc)4 (e.g., G94517VF, G57672ST); (Hex)4 (HexNAc)4 (Deoxyhexose)1 (e.g., G94768NG); (Hex)2 (HexNAc)3 (Deoxyhexose)1 (e.g., G91459EI, G26724PP, G52132CU); (Hex)1 (HexNAc)1 (NeuAc)1 (NeuGc)1 (e.g., G49527BY); (Hex)2 (HexNAc)4 (Deoxyhexose)1 (e.g., G23700TV); (Hex)3 (HexNAc)5 (e.g., G00505CR); (Hex)4 (HexNAc)5 (Deoxyhexose)2 (e.g., G82251ZP, G18603ZQ); (Hex)5 (HexNAc)5 (Deoxyhexose)1 (e.g., G25957KN); (Hex)2 (HexNAc)4 (e.g., G34764BK, G12074QJ); (Hex)3 (HexNAc)3 (Deoxyhexose)1 (e.g., G66741QE, G16458JH); (Hex)4 (HexNAc)6 (Deoxyhexose)1 (e.g., G32752FJ); (Hex)3 (HexNAc)2 (Deoxyhexose)1 (e.g., G15849KC, G81911LP, G41486OC); (Hex)2 (HexNAc)1 (Deoxyhexose)2 (e.g., G68200GL); (Hex)4 (HexNAc)6 (Deoxyhexose)2 (e.g., G99804SJ); (Hex)3 (HexNAc)2 (e.g., G16404NW, G25323VU); (Hex)5 (HexNAc)6 (Deoxyhexose)2 (e.g., G44467ZE); and (Hex)3 (HexNAc)3 (e.g., G86537AD, G89585FG).

In some embodiments, a mucin glycan comprises a formula selected from the group consisting of (Hex)1 (HexNAc)1 (Deoxyhexose)1 (e.g., G94435QH); (Hex)1 (NeuAc)1 (e.g., G30207PZ, G63069TR); (Hex)1 (Deoxyhexose)1 (e.g., G00068MO); (Hex)1 (HexNAc)1 (e.g., G76355TG, G85856KC); (Hex)1 (NeuGc)1 (e.g., G38557KR, G59867EM); (Hex)1 (HexNAc)2 (Deoxyhexose)1 (e.g., G32426JY, G74353FF); (Hex)1 (HexNAc)1 (NeuAc)1 (e.g., G65562ZE); (Hex)1 (HexNAc)1 (NeuGc)1 (e.g., G64527IJ); (Hex)1 (HexNAc)2 (e.g., G00033MO, G61730RY, G56868BH); (HexNAc)2 (e.g., G00041MO, G00057MO); (HexNAc)1 (e.g., G57321FI); (Hex)1 (HexNAc)3 (Deoxyhexose)1 (e.g., G28878FC); (Hex)2 (HexNAc)2 (NeuAc)1 (e.g., G46748BU); (Hex)2 (HexNAc)2 (e.g., G64973KT); (HexNAc)2 (NeuAc)1 (e.g., G63334FZ); and (Hex)2 (HexNAc)1 (e.g., G28052FT).

In some embodiments, a mucin glycan comprises a formula selected from the group consisting of (Hex)1 (HexNAc)1 (Deoxyhexose)1 (e.g., G94435QH); (Hex)1 (NeuAc)1 (e.g., G30207PZ, G63069TR); (Hex)1 (Deoxyhexose)1 (e.g., G00068MO); (Hex)1 (HexNAc)1 (e.g., G76355TG, G85856KC); (Hex)1 (NeuGc)1 (e.g., G38557KR, G59867EM); (Hex)1 (HexNAc)2 (Deoxyhexose)1 (e.g., G32426JY, G74353FF); (Hex)1 (HexNAc)1 (NeuAc)1 (e.g., G65562ZE); and (Hex)1 (HexNAc)1 (NeuGc)1 (e.g., G64527IJ).

In some embodiments, a mucin glycan comprises a formula selected from the group consisting of (Hex)1 (HexNAc)1 (Deoxyhexose)1 (e.g., G94435QH); (Hex)1 (NeuAc)1 (e.g., G30207PZ, G63069TR); (Hex)1 (Deoxyhexose)1 (e.g., G00068MO); and (Hex)1 (HexNAc)1 (e.g., G76355TG, G85856KC).

### MUC5AC Glycans

In some embodiments, a mucin glycan comprises a porcine MUC5AC glycan.

In some embodiments, a mucin glycan comprises a formula selected from the group consisting of (Hex)1 (HexNAc)1 (e.g., G76355TG, G85856KC); (Hex)1 (HexNAc)2 (e.g., G00033MO, G61730RY, G56868BH); (Hex)1 (HexNAc)1 (Deoxyhexose)1 (e.g., G94435QH); (Hex)1 (Deoxyhexose)1 (e.g., G00068MO); (Hex)2 (HexNAc)3 (Deoxyhexose)1 (e.g., G91459EI, G26724PP, G52132CU); (Hex)2 (HexNAc)4 (e.g., G34764BK, G12074QJ); (Hex)2 (HexNAc)2 (Deoxyhexose)1 (e.g., G94514IB, G61216ZY); (Hex)2 (HexNAc)3 (e.g., G96915PP, G13483MW); (Hex)2 (HexNAc)2 (e.g., G64973KT); (Hex)3 (HexNAc)2 (Deoxyhexose)2 (e.g., G20310DM, G78177LC); (Hex)3 (HexNAc)3 (Deoxyhexose)1 (e.g., G66741QE, G16458JH); (Hex)3 (HexNAc)4 (e.g., G94517VF, G57672ST); (Hex)2 (HexNAc)4 (Deoxyhexose)1 (e.g., G23700TV); (Hex)2 (HexNAc)1 (Deoxyhexose)1 (e.g., G73318SN, G33986KK); (Hex)1 (HexNAc)2 (Deoxyhexose)1 (e.g., G32426JY, G74353FF); (Hex)2 (HexNAc)2 (Deoxyhexose)2 (e.g., G23119MJ); (Hex)1 (HexNAc)3 (e.g., G68893BQ, G23438NR); (Hex)3 (HexNAc)4 (Deoxyhexose)1 (e.g., G02990AF, G29956GF); (Hex)1 (HexNAc)1 (NeuAc)1 (e.g., G65562ZE); (Hex)3 (HexNAc)4 (Deoxyhexose)2 (e.g., G93333OF); (Hex)3 (HexNAc)2 (Deoxyhexose)1 (e.g., G15849KC, G81911LP, G41486OC); (Hex)2 (HexNAc)1 (e.g., G28052FT); (Hex)3 (HexNAc)3 (Deoxyhexose)2 (e.g., G35949CT, G71094KR, G08426KY); (Hex)3 (HexNAc)3 (e.g., G86537AD, G89585FG); (Hex)1 (HexNAc)2 (NeuAc)1 (e.g., G85608AG, G64844ET); (Hex)3 (HexNAc)5 (e.g., G00505CR); (HexNAc)1 (e.g., G57321FI); (Hex)1 (HexNAc)4 (e.g., G59229NY); (Hex)4 (HexNAc)3 (Deoxyhexose)2 (e.g., G68308CM); (Hex)3 (HexNAc)5 (Deoxyhexose)1 (e.g., G05252QE); (Hex)1 (HexNAc)3 (Deoxyhexose)1 (e.g., G28878FC); (Hex)2 (HexNAc)1 (NeuAc)1 (e.g., G59126YU); (Hex)4 (HexNAc)4 (Deoxyhexose)1 (e.g., G94768NG); (Hex)2 (HexNAc)2 (NeuAc)1 (e.g., G46748BU); (Hex)3 (HexNAc)2 (e.g., G16404NW, G25323VU); (Hex)2 (HexNAc)3 (Deoxyhexose)2 (e.g., G68384KC, G39326PP); (Hex)1 (HexNAc)1 (Deoxyhexose)1 (NeuAc)1 (e.g., G47180UC); (Hex)4 (HexNAc)4 (Deoxyhexose)2 (e.g., G98518WL); (Hex)2 (HexNAc)4 (Deoxyhexose)2 (e.g., G90965BZ); (Hex)2 (HexNAc)5 (e.g., G23048PE); (Hex)4 (HexNAc)5 (Deoxyhexose)2 (e.g., G82251ZP, G18603ZQ); (Hex)3 (HexNAc)6 (e.g., G62609ZF); (Hex)2 (HexNAc)1 (Deoxyhexose)2 (e.g., G68200GL); (Hex)4 (HexNAc)4 (Deoxyhexose)3 (e.g., G66166BF); (Hex)4 (HexNAc)5 (Deoxyhexose)1 (e.g., G92547QZ); (Hex)3 (HexNAc)5 (Deoxyhexose)2 (e.g., G28921PH); (Hex)2 (HexNAc)5 (Deoxyhexose)1 (e.g., G09396HG); (Hex)6 (HexNAc)5 (Deoxyhexose)3 (e.g., G18501TC); (Hex)4 (HexNAc)6 (Deoxyhexose)1 (e.g., G32752FJ); (Hex)5 (HexNAc)6 (Deoxyhexose)3 (e.g., G65612SS); (Hex)7 (HexNAc)6 (Deoxyhexose)1 (e.g., G61898SS); (Hex)5 (HexNAc)4 (Deoxyhexose)2 (e.g., G21630AC); (Hex)5 (HexNAc)5 (Deoxyhexose)1 (e.g., G25957KN); (Hex)6 (HexNAc)5 (Deoxyhexose)1 (e.g., G37901JE, G84713IO); (Hex)6 (HexNAc)7 (Deoxyhexose)2 (e.g., G29852ZH); (Hex)4 (HexNAc)6 (Deoxyhexose)2 (e.g., G99804SJ); (Hex)5 (HexNAc)5 (Deoxyhexose)3 (e.g., G90829NZ); (Hex)6 (HexNAc)5 (Deoxyhexose)2 (e.g., G70999YJ, G02681FY); and (Hex)5 (HexNAc)6 (Deoxyhexose)2 (e.g., G44467ZE).

In some embodiments, a mucin glycan comprises a formula selected from the group consisting of (Hex)1 (HexNAc)1 (e.g., G76355TG, G85856KC); (Hex)1 (HexNAc)2 (e.g., G00033MO, G61730RY, G56868BH); (Hex)1 (HexNAc)1 (Deoxyhexose)1 (e.g., G94435QH); (Hex)1 (Deoxyhexose)1 (e.g., G00068MO); (Hex)2 (HexNAc)3 (Deoxyhexose)1 (e.g., G91459EI, G26724PP, G52132CU); (Hex)2 (HexNAc)4 (e.g., G34764BK, G12074QJ); (Hex)2 (HexNAc)2 (Deoxyhexose)1 (e.g., G94514IB, G61216ZY); (Hex)2 (HexNAc)3 (e.g., G96915PP, G13483MW); (Hex)2 (HexNAc)2 (e.g., G64973KT); (Hex)3 (HexNAc)2 (Deoxyhexose)2 (e.g., G20310DM, G78177LC); (Hex)3 (HexNAc)3 (Deoxyhexose)1 (e.g., G66741QE, G16458JH); (Hex)3 (HexNAc)4 (e.g., G94517VF, G57672ST); (Hex)2 (HexNAc)4 (Deoxyhexose)1 (e.g., G23700TV); (Hex)2 (HexNAc)1 (Deoxyhexose)1 (e.g., G73318SN, G33986KK); (Hex)1 (HexNAc)2 (Deoxyhexose)1 (e.g., G32426JY, G74353FF); (Hex)2 (HexNAc)2 (Deoxyhexose)2 (e.g., G23119MJ); (Hex)1 (HexNAc)3 (e.g., G68893BQ, G23438NR); (Hex)3 (HexNAc)4 (Deoxyhexose)1 (e.g., G02990AF, G29956GF); and (Hex)1 (HexNAc)1 (NeuAc)1 (e.g., G65562ZE).

In some embodiments, a mucin glycan comprises a formula selected from the group consisting of (Hex)1 (HexNAc)1 (e.g., G76355TG, G85856KC); (Hex)1 (HexNAc)2 (e.g., G00033MO, G61730RY, G56868BH); (Hex)1 (HexNAc)1 (Deoxyhexose)1 (e.g., G94435QH); (Hex)1 (Deoxyhexose)1 (e.g., G00068MO); (Hex)2 (HexNAc)3 (Deoxyhexose)1 (e.g., G91459EI, G26724PP, G52132CU); (Hex)2 (HexNAc)4 (e.g., G34764BK, G12074QJ); (Hex)2 (HexNAc)2 (Deoxyhexose)1 (e.g., G94514IB, G61216ZY); (Hex)2 (HexNAc)3 (e.g., G96915PP, G13483MW); (Hex)2 (HexNAc)2 (e.g., G64973KT); (Hex)3 (HexNAc)2 (Deoxyhexose)2 (e.g., G20310DM, G78177LC); (Hex)3 (HexNAc)3 (Deoxyhexose)1 (e.g., G66741QE, G16458JH); and (Hex)3 (HexNAc)4 (e.g., G94517VF, G57672ST).

In some embodiments, a mucin glycan comprises a formula selected from the group consisting of (Hex)1 (HexNAc)1 (e.g., G76355TG, G85856KC); (Hex)1 (HexNAc)2 (e.g., G00033MO, G61730RY, G56868BH); (Hex)1 (HexNAc)1 (Deoxyhexose)1 (e.g., G94435QH); (Hex)1 (Deoxyhexose)1 (e.g., G00068MO); (Hex)2 (HexNAc)3 (Deoxyhexose)1 (e.g., G91459EI, G26724PP, G52132CU); and (Hex)2 (HexNAc)4 (e.g., G34764BK, G12074QJ).

### MUC5B Glycans

In some embodiments, a mucin glycan comprises a human MUC5B glycan.

In some embodiments, a mucin glycan comprises a formula selected from the group consisting of (Hex)1 (HexNAc)1 (Deoxyhexose)1 (e.g., G94435QH); (Hex)1 (HexNAc)1 (e.g., G76355TG, G85856KC); (Hex)1 (HexNAc)2 (Deoxyhexose)1 (e.g., G32426JY, G74353FF); (Hex)1 (NeuAc)1 (e.g., G30207PZ, G63069TR); (Hex)1 (Deoxyhexose)1 (e.g., G00068MO); (Hex)3 (HexNAc)3 (Deoxyhexose)2 (e.g., G35949CT, G71094KR, G08426KY); (Hex)2 (HexNAc)2 (Deoxyhexose)2 (e.g., G23119MJ); (Hex)1 (HexNAc)1 (NeuAc)1 (e.g., G65562ZE); (Hex)3 (HexNAc)2 (Deoxyhexose)2 (e.g., G20310DM, G78177LC); (Hex)1 (HexNAc)2 (e.g., G00033MO, G61730RY, G56868BH); (Hex)1 (HexNAc)2 (Deoxyhexose)2 (e.g., G89748NG, G09520ZQ); (Hex)2 (HexNAc)2 (Deoxyhexose)1 (e.g., G94514IB, G61216ZY); (Hex)2 (HexNAc)1 (Deoxyhexose)1 (e.g., G73318SN, G33986KK); (Hex)2 (HexNAc)3 (Deoxyhexose)1 (e.g., G91459EI, G26724PP, G52132CU); (Hex)2 (HexNAc)3 (Deoxyhexose)2 (e.g., G68384KC, G39326PP); (Hex)3 (HexNAc)2 (Deoxyhexose)1 (e.g., G15849KC, G81911LP, G41486OC); (Hex)2 (HexNAc)1 (e.g., G28052FT); (Hex)1 (HexNAc)3 (Deoxyhexose)1 (e.g., G28878FC); (Hex)3 (HexNAc)4 (Deoxyhexose)3 (e.g., G59787TQ); (Hex)2 (HexNAc)1 (Deoxyhexose)2 (e.g., G68200GL); (Hex)2 (HexNAc)2 (NeuAc)1 (e.g., G46748BU); (Hex)3 (HexNAc)2 (Deoxyhexose)3 (e.g., G70416EY, G13012GZ); (Hex)2 (HexNAc)4 (Deoxyhexose)2 (e.g., G90965BZ); (Hex)3 (HexNAc)3 (Deoxyhexose)1 (e.g., G66741QE, G16458JH); (Hex)3 (HexNAc)3 (Deoxyhexose)3 (e.g., G23021IW); (Hex)2 (HexNAc)2 (e.g., G64973KT); (Hex)2 (HexNAc)3 (e.g., G96915PP, G13483MW); (Hex)2 (HexNAc)2 (Deoxyhexose)3 (e.g., G01532FF); (Hex)3 (HexNAc)4 (Deoxyhexose)4 (e.g., G89469SP); (Hex)3 (HexNAc)2 (e.g., G16404NW, G25323VU); (Hex)3 (HexNAc)4 (Deoxyhexose)1 (e.g., G02990AF, G29956GF); (HexNAc)2 (NeuAc)1 (e.g., G63334FZ); (Hex)3 (HexNAc)4 (Deoxyhexose)2 (e.g., G93333OF); (Hex)1 (HexNAc)2 (Deoxyhexose)1 (NeuAc)1 (e.g., G75749JP, G40270LS); (Hex)3 (HexNAc)2 (Deoxyhexose)4 (e.g., G93469SN, G15747RC); (Hex)1 (HexNAc)2 (NeuAc)1 (e.g., G85608AG, G64844ET); (Hex)1 (HexNAc)1 (Deoxyhexose)1 (NeuAc)1 (e.g., G47180UC); (Hex)4 (HexNAc)3 (Deoxyhexose)2 (e.g., G68308CM); (Hex)4 (HexNAc)4 (Deoxyhexose)2 (e.g., G98518WL); (HexNAc)2 (e.g., G00041MO, G00057MO); (Hex)4 (HexNAc)4 (Deoxyhexose)3 (e.g., G66166BF); (Hex)2 (HexNAc)4 (Deoxyhexose)1 (e.g., G23700TV); (Hex)3 (HexNAc)2 (Deoxyhexose)1 (NeuAc)1 (e.g., G74607VK, G81461IK); (Hex)2 (HexNAc)1 (Deoxyhexose)1 (NeuAc)1 (e.g., G49549VN, G95742RK); (Hex)3 (HexNAc)3 (Deoxyhexose)4 (e.g., G84853WN); (Hex)5 (HexNAc)4 (Deoxyhexose)2 (e.g., G21630AC); (HexNAc)1 (e.g., G57321FI); (Hex)1 (HexNAc)3 (e.g., G68893BQ, G23438NR); (Hex)2 (HexNAc)1 (Deoxyhexose)3 (e.g., G82961CS); (Hex)2 (HexNAc)5 (Deoxyhexose)2 (e.g., G25612EW); (Hex)4 (HexNAc)4 (Deoxyhexose)1 (e.g., G94768NG); (Hex)4 (HexNAc)5 (Deoxyhexose)4 (e.g., G11381FO); (Hex)4 (HexNAc)5 (Deoxyhexose)2 (e.g., G82251ZP, G18603ZQ); (Hex)5 (HexNAc)5 (Deoxyhexose)1 (e.g., G25957KN); (Hex)2 (HexNAc)4 (e.g., G34764BK, G12074QJ); (Hex)4 (HexNAc)5 (Deoxyhexose)1 (e.g., G92547QZ); (Hex)1 (NeuGc)1 (e.g., G38557KR, G59867EM); (Hex)5 (HexNAc)6 (Deoxyhexose)2 (e.g., G44467ZE); (Hex)6 (HexNAc)5 (Deoxyhexose)2 (e.g., G70999YJ, G02681FY); (Hex)2 (HexNAc)2 (Deoxyhexose)1 (NeuAc)1 (e.g., G77740PR, G59155GF); (Hex)5 (HexNAc)6 (Deoxyhexose)3 (e.g., G65612SS); (Hex)3 (HexNAc)3 (e.g., G86537AD, G89585FG); (Hex)3 (HexNAc)5 (Deoxyhexose)1 (e.g., G05252QE); (Hex)5 (HexNAc)5 (Deoxyhexose)3 (e.g., G90829NZ); (Hex)2 (HexNAc)1 (NeuAc)1 (e.g., G59126YU); (Hex)7 (HexNAc)6 (Deoxyhexose)1 (e.g., G61898SS); and (Hex)3 (HexNAc)4 (e.g., G94517VF, G57672ST).

In some embodiments, a mucin glycan comprises a formula selected from the group consisting of (Hex)1 (HexNAc)1 (Deoxyhexose)1 (e.g., G94435QH); (Hex)1 (HexNAc)1 (e.g., G76355TG, G85856KC); (Hex)1 (HexNAc)2 (Deoxyhexose)1 (e.g., G32426JY, G74353FF); (Hex)1 (NeuAc)1 (e.g., G30207PZ, G63069TR); (Hex)1 (Deoxyhexose)1 (e.g., G00068MO); (Hex)3 (HexNAc)3 (Deoxyhexose)2 (e.g., G35949CT, G71094KR, G08426KY); (Hex)2 (HexNAc)2 (Deoxyhexose)2 (e.g., G23119MJ); (Hex)1 (HexNAc)1 (NeuAc)1 (e.g., G65562ZE); (Hex)3 (HexNAc)2 (Deoxyhexose)2 (e.g., G20310DM, G78177LC); (Hex)1 (HexNAc)2 (e.g., G00033MO, G61730RY, G56868BH); (Hex)1 (HexNAc)2 (Deoxyhexose)2 (e.g., G89748NG, G09520ZQ); (Hex)2 (HexNAc)2 (Deoxyhexose)1 (e.g., G94514IB, G61216ZY); (Hex)2 (HexNAc)1 (Deoxyhexose)1 (e.g., G73318SN, G33986KK); (Hex)2 (HexNAc)3 (Deoxyhexose)1 (e.g., G91459EI, G26724PP, G52132CU); (Hex)2 (HexNAc)3 (Deoxyhexose)2 (e.g., G68384KC, G39326PP); (Hex)3 (HexNAc)2 (Deoxyhexose)1 (e.g., G15849KC, G81911LP, G41486OC); (Hex)2 (HexNAc)1 (e.g., G28052FT); (Hex)1 (HexNAc)3 (Deoxyhexose)1 (e.g., G28878FC); (Hex)3 (HexNAc)4 (Deoxyhexose)3 (e.g., G59787TQ); (Hex)2 (HexNAc)1 (Deoxyhexose)2 (e.g., G68200GL); (Hex)2 (HexNAc)2 (NeuAc)1 (e.g., G46748BU); (Hex)3 (HexNAc)2 (Deoxyhexose)3 (e.g., G70416EY, G13012GZ); (Hex)2 (HexNAc)4 (Deoxyhexose)2 (e.g., G90965BZ); (Hex)3 (HexNAc)3 (Deoxyhexose)1 (e.g., G66741QE, G16458JH); (Hex)3 (HexNAc)3 (Deoxyhexose)3 (e.g., G23021IW); (Hex)2 (HexNAc)2 (e.g., G64973KT); and (Hex)2 (HexNAc)3 (e.g., G96915PP, G13483MW).

In some embodiments, a mucin glycan comprises a formula selected from the group consisting of (Hex)1 (HexNAc)1 (Deoxyhexose)1 (e.g., G94435QH); (Hex)1 (HexNAc)1 (e.g., G76355TG, G85856KC); (Hex)1 (HexNAc)2 (Deoxyhexose)1 (e.g., G32426JY, G74353FF); (Hex)1 (NeuAc)1 (e.g., G30207PZ, G63069TR); (Hex)1 (Deoxyhexose)1 (e.g., G00068MO); (Hex)3 (HexNAc)3 (Deoxyhexose)2 (e.g., G35949CT, G71094KR, G08426KY); (Hex)2 (HexNAc)2 (Deoxyhexose)2 (e.g., G23119MJ); (Hex)1 (HexNAc)1 (NeuAc)1 (e.g., G65562ZE); (Hex)3 (HexNAc)2 (Deoxyhexose)2 (e.g., G20310DM, G78177LC); (Hex)1 (HexNAc)2 (e.g., G00033MO, G61730RY, G56868BH); (Hex)1 (HexNAc)2 (Deoxyhexose)2 (e.g., G89748NG, G09520ZQ); (Hex)2 (HexNAc)2 (Deoxyhexose)1 (e.g., G94514IB, G61216ZY); and (Hex)2 (HexNAc)1 (Deoxyhexose)1 (e.g., G73318SN, G33986KK).

In some embodiments, a mucin glycan comprises a formula selected from the group consisting of (Hex)1 (HexNAc)1 (Deoxyhexose)1 (e.g., G94435QH); (Hex)1 (HexNAc)1 (e.g., G76355TG, G85856KC); (Hex)1 (HexNAc)2 (Deoxyhexose)1 (e.g., G32426JY, G74353FF); (Hex)1 (NeuAc)1 (e.g., G30207PZ, G63069TR); and (Hex)1 (Deoxyhexose)1 (e.g., G00068MO).

### Sulfated Mucin Glycans

In some embodiments, a mucin glycan (e.g., MUC2, MUC5B or MUC5AC glycan) is a sulfated mucin glycan.

In some embodiments, a sulfated mucin glycan comprises a formula selected from the group consisting of S1 (Hex)1 (HexNAc)1 (e.g., G10634LC); S1 (Hex)1 (HexNAc)1 (Deoxyhexose)1 (e.g., G08671QK); S1 (Hex)1 (HexNAc)2 (e.g., G32406CO); S1 (Hex)2 (HexNAc)1 (Deoxyhexose)1 (e.g., G24803MV); S1 (Hex)1 (HexNAc)2 (Deoxyhexose)1 (e.g., G10520JC); S1 (Hex)1 (HexNAc)2 (NeuAc)1 (e.g., G96888OD); S1 (Hex)2 (HexNAc)2 (Deoxyhexose)1 (e.g., G24803MV); S1 (Hex)1 (HexNAc)2 (NeuGc)1 (e.g., G60426XC); S1 (Hex)1 (HexNAc)3 (Deoxyhexose)1 (e.g., G72091WB, G60644GY); and S1 (Hex)2 (HexNAc)3 (Deoxyhexose)1 (e.g., G35891PL).

In some embodiments, a sulfated mucin glycan comprises a sulfated MUC2 glycan, a sulfated MUC5AC glycan, a sulfated MUC5B glycan, or a combination thereof.

In some embodiments, a sulfated MUC2 glycan comprises a formula selected from the group consisting of S1 (Hex)1 (HexNAc)2 (e.g., G32406CO); S1 (Hex)1 (HexNAc)2 (Deoxyhexose)1 (e.g., G10520JC); S1 (Hex)1 (HexNAc)2 (NeuAc)1 (e.g., G96888OD); S1 (Hex)1 (HexNAc)3 (Deoxyhexose)1 (e.g., G72091WB, G60644GY); and S1 (Hex)1 (HexNAc)2 (NeuGc)1 (e.g., G60426XC).

In some embodiments, a sulfated MUC5AC glycan comprises a formula selected from the group consisting of S1 (Hex)1 (HexNAc)2 (Deoxyhexose)1 (e.g., G10520JC); S1 (Hex)2 (HexNAc)2 (Deoxyhexose)1 (e.g., G24803MV); S1 (Hex)2 (HexNAc)1 (Deoxyhexose)1 (e.g., G24803MV); S1 (Hex)2 (HexNAc)3 (Deoxyhexose)1 (e.g., G35891PL); and S1 (Hex)1 (HexNAc)2 (e.g., G32406CO).

In some embodiments, a sulfated MUC5B glycan comprises a formula selected from the group consisting of S1 (Hex)2 (HexNAc)1 (Deoxyhexose)1 (e.g., G24803MV); S1 (Hex)1 (HexNAc)1 (Deoxyhexose)1 (e.g., G08671QK); S1 (Hex)1 (HexNAc)1 (e.g., G10634LC); S1 (Hex)2 (HexNAc)2 (Deoxyhexose)1 (e.g., G24803MV); and S1 (Hex)2 (HexNAc)3 (Deoxyhexose)1 (e.g., G35891PL).

In some embodiments, a mucin glycan comprises a synthesized (e.g., chemically synthesized) mucin glycan, a purified (e.g., native) mucin glycan, or a combination thereof. In some embodiments, a mucin glycan comprises a synthesized mucin glycan. Methods for chemically synthesizing mucin glycans are described herein; methods for purifying mucin glycan are described herein and are known in the art.

Concentrations of a mucin glycan in compositions or methods disclosed herein can vary, for example, depending on the desired use. In some embodiments, a mucin glycan concentration is about: 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8% or 0.9% (w/v) in a solution. In some embodiments, a mucin glycan concentration in a solution is within physiological concentration ranges of the mucin glycan.

Headings used in this application are for convenience only and do not affect the interpretation of this application.

Preferred features of each of the aspects provided by the disclosure are applicable to all of the other aspects of the disclosure mutatis mutandis and, without limitation, are exemplified by the dependent claims and also encompass combinations and permutations of individual features (e.g., elements, including numerical ranges and exemplary embodiments) of particular embodiments and aspects of the disclosure, including the working examples. For example, particular experimental parameters exemplified in the working examples can be adapted for use in the claimed disclosure piecemeal without departing from the disclosure. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

### Example Embodiments

1. A method of treating a fungal infection in a subject in need thereof, the method comprising administering to the subject a therapeutically effective amount of a mucin glycan, tautomer, stereoisomer and/or a pharmaceutically acceptable salt thereof.
2. A method of attenuating virulence of a fungus in a subject in need thereof, the method comprising administering to the subject a therapeutically effective amount of a mucin glycan, tautomer, stereoisomer and/or a pharmaceutically acceptable salt thereof.
3. A method of attenuating virulence of a fungus, the method comprising contacting the fungus with a mucin glycan, tautomer, stereoisomer and/or a pharmaceutically acceptable salt thereof.
4. A method of inhibiting formation of a fungal biofilm on a surface, the method comprising contacting the surface with a mucin glycan, tautomer, stereoisomer and/or a pharmaceutically acceptable salt thereof.
5. The method of any one of embodiments 1-4, wherein the fungus comprises Candida albicans.
6. The method of embodiment 2, 3 or 5, wherein attenuating virulence of a fungus comprises modulating a virulence-associated gene of the fungus, reducing the fungus's surface adhesion, inhibiting the fungus's morphological transition to an invasive or virulent cell type, inhibiting fungal biofilm formation, reducing the fungus's secretion of a hydrolytic enzyme, or a combination thereof.
7. The method of embodiment 6, wherein modulating a virulence-associated gene comprises:
   a) downregulating expression of RAS1, EED1, HGC1, UME6, EFG1, BRG1, EFG1, TEC1, ROB1, RFX2, AHR1, ECE1, HYR1, ALS3, HWP1, PRA1, PHR1, GAC1, PTP3, AHR1, YVC1, SAP5, IHD1, RTA4, PGA54, IHD2, DDR48, CSA1, SHE3, or TUP1, or a combination thereof,
   b) increasing expression of NRG1, YWP1, or both; or
   both a) and b).
8. The method of embodiment 4 or 5, wherein the surface comprises a living surface, an inert surface, or both.
9. The method of embodiment 1, 2 or 5, wherein the subject is immunocompromised.
10. The method of embodiment 1, 2, 5 or 9, wherein the subject has an implanted medical device.
11. The method of any one of embodiments 1-10, wherein the mucin glycan is a secreted gel-forming mucin glycan.
12. The method of any one of embodiments 1-11, wherein the mucin glycan comprises a MUC2 glycan, a MUC5AC glycan, a MUC5B glycan, or a combination thereof.
13. The method of any one of embodiments 1-12, wherein the mucin glycan comprises one or more monosaccharides selected from N-acetylgalactosamine (GalNAc), N-acetylglucosamine (GlcNAc), mannose (Man), fucose (Fuc), N-acetylneuraminic acid (Neu5Ac), or galactose (Gal), or a combination thereof.
14. The method of any one of embodiments 1-13, wherein the mucin glycan comprises a glycan core of the following structural formula.

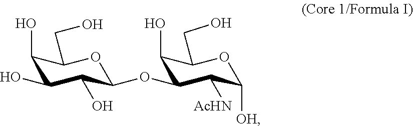

or a tautomer or stereoisomer thereof.
15. The method of embodiment 14, wherein the mucin glycan comprises a glycan structure selected from the group consisting of 1a, 2, 3a, 4, 9c, 15b, 16b, 18b, 6, 7 and 25b of FIG. 4A.
16. The method of embodiment 15, wherein the mucin glycan further comprises fucose, sialic acid, or a combination thereof.
17. The method of embodiment 16, wherein the mucin glycan comprises the following structural formula:

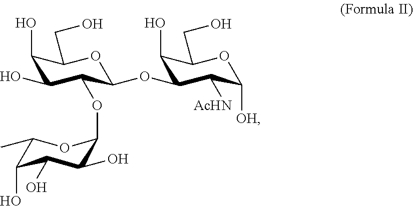

or a tautomer or stereoisomer thereof, or

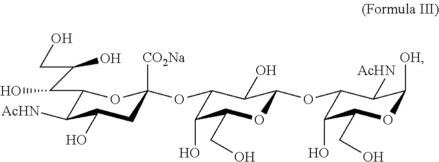

or a tautomer or stereoisomer thereof.
18. The method of embodiment 14, wherein the mucin glycan comprises a glycan core of the following structural formula:

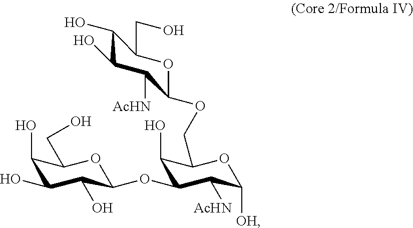

or a tautomer or stereoisomer thereof.
19. The method of embodiment 18, wherein the mucin glycan comprises a glycan structure selected from the group consisting of 8a, 9a, 9b, 10a, 11a, 12a, 12b, 13, 14a, 15a, 16a, 17, 18a, 19, 20a, 21a, 21b, 22, 23a, 24a, 24b, 25a, 26a, 27, 28, 29, 30, 31, 32, 33a, 34a, 35, 36, 37, 38a, 38b, 39, 40, 41, 42, 43, 44, 45, 46, 47a, 48, 49a, 50, 51, 52, 53a and 54 of FIG. 4A.
20. The method of embodiment 18, wherein the mucin glycan comprises fucose, galactose, or a combination thereof.
21. The method of embodiment 20, wherein the mucin glycan comprises the following structural formula:

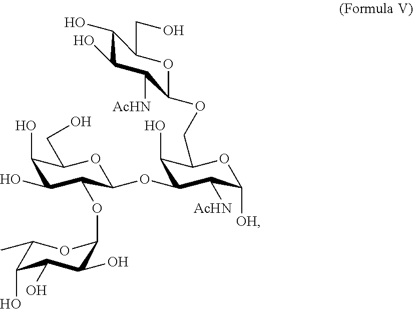

or a tautomer or stereoisomer thereof or

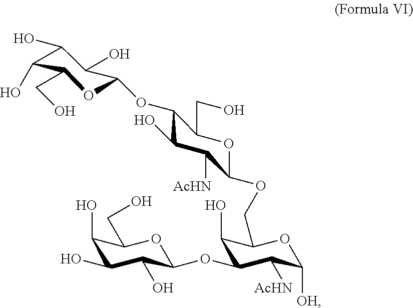

or a tautomer or stereoisomer thereof.
22. The method of any one of embodiments 1-13, wherein the mucin glycan comprises a glycan core of the following structural formula:

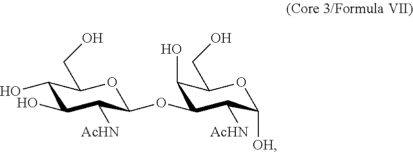

or a tautomer or stereoisomer thereof.
23. The method of embodiment 22, wherein the mucin glycan comprises a glycan structure selected from the group consisting of 8b, 10b, 20b, 26b and 55 of FIG. 4A.
24. The method of any one of embodiments 1-13, wherein the mucin glycan comprises a glycan core of the following structural formula:

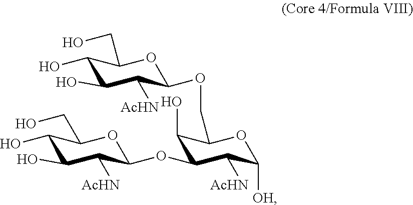

or a tautomer or stereoisomer thereof.
25. The method of embodiment 24, wherein the mucin glycan comprises a glycan structure selected from the group consisting of 34b and 56 of FIG. 4A.
26. The method of any one of embodiments 1-13, wherein the mucin glycan comprises a glycan core of the following structural formula:

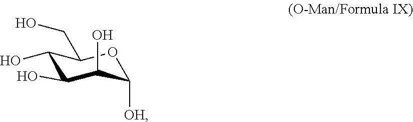

or a tautomer or stereoisomer thereof.
27. The method of embodiment 26, wherein the mucin glycan comprises a glycan structure selected from the group consisting of 1b, 3b, 11b, 14b, 57, 8c, 23b, 23c, 24c, 33b, 58, 47b, 49b and 53b of FIG. 4A.
28. The method of any one of embodiments 1-13, wherein the mucin glycan comprises a glycan core of the following structural formula:

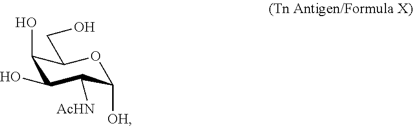

or a tautomer or stereoisomer thereof.
29. The method of embodiment 28, wherein the mucin glycan comprises a glycan structure of 5 of FIG. 4A.
30. The method of any one of embodiments 1-29, wherein the mucin glycan is unsulfated.
31. The method of any one of embodiments 1-29, wherein the mucin glycan is sulfated.
32. The method of embodiment 31, wherein the sulfated mucin glycan comprises the glycan structure selected from:
   a) the group consisting of 1a, 2, 3a, 9c, 15b and 18b of FIG. 4A;
   b) the group consisting of 8a, 9a, 9b, 11a, 12a, 12b, 13, 14a, 15a, 17, 18a, 19, 20a, 21a, 21b, 23a, 24a, 24b, 30 and 34a of FIG. 4A;
   c) 8b or 20b of FIG. 4A;
   d) 34b of FIG. 4A; or
   e) the group consisting of 11b, 14b, 57, 23b and 23c of FIG. 4A.
33. The method of any one of embodiments 1-32, wherein the mucin glycan further comprises N-acetylgalactosamine, N-acetylglucosamine, mannose, fucose, N-acetylneuraminic acid, galactose, sulfate, sialic acid, or a combination thereof.
34. The method of any one of embodiments 1-13, wherein the mucin glycan comprises a formula selected from the group consisting of (HexNAc)1 (G57321FI); (Hex)1 (HexNAc)1 (G76355TG, G85856KC); (Hex)1 (HexNAc)1 (Deoxyhexose)1 (G94435QH); (Hex)1 (HexNAc)1 (NeuAc)1 (G65562ZE); (Hex)2 (HexNAc)1 (Deoxyhexose)1 (G73318SN, G33986KK); (Hex)1 (HexNAc)1 (NeuGc)1 (G64527IJ); (Hex)1 (HexNAc)1 (Deoxyhexose)1 (NeuAc)1 (G47180UC); (Hex)2 (HexNAc)1 (Deoxyhexose)2 (G68200GL); (Hex)1 (HexNAc)1 (NeuAc)2 (G01614ZM); (Hex)2 (HexNAc)1 (Deoxyhexose)3 (G82961CS); (Hex)2 (HexNAc)1 (Deoxyhexose)1 (NeuAc)1 (G49549VN, G95742RK); (Hex)1 (HexNAc)1 (NeuAc)1 (NeuGc)1 (G49527BY); (Hex)1 (HexNAc)2 (Deoxyhexose)1 (NeuAc)1 (G75749JP, G40270LS); (Hex)3 (HexNAc)2 (Deoxyhexose)4 (G93469SN, G15747RC); (Hex)1 (HexNAc)2 (G00033MO, G61730RY, G56868BH); (Hex)1 (HexNAc)2 (Deoxyhexose)1 (G32426JY, G74353FF); (Hex)2 (HexNAc)2 (G64973KT); (Hex)1 (HexNAc)3 (G68893BQ, G23438NR); (Hex)1 (HexNAc)2 (Deoxyhexose)2 (G89748NG, G09520ZQ); (Hex)1 (HexNAc)2 (NeuAc)1 (G85608AG, G64844ET); (Hex)2 (HexNAc)2 (Deoxyhexose)1 (G94514IB, G61216ZY); (Hex)1 (HexNAc)2 (NeuGc)1 (G60426XC); (Hex)3 (HexNAc)2 (G16404NW, G25323VU); (Hex)1 (HexNAc)3 (Deoxyhexose)1 (G28878FC); (Hex)2 (HexNAc)3 (G96915PP, G13483MW); (Hex)1 (HexNAc)4 (G59229NY); (Hex)2 (HexNAc)2 (Deoxyhexose)2 (G23119MJ); (Hex)2 (HexNAc)2 (NeuAc)1 (G46748BU); (Hex)3 (HexNAc)2 (Deoxyhexose)1 (G15849KC, G81911LP, G41486OC); (Hex)2 (HexNAc)3 (Deoxyhexose)1 (G91459EI, G26724PP, G52132CU); (Hex)3 (HexNAc)3 (G86537AD, G89585FG); (Hex)2 (HexNAc)4 (G34764BK, G12074QJ); (Hex)2 (HexNAc)2 (Deoxyhexose)3 (G01532FF); (Hex)2 (HexNAc)2 (Deoxyhexose)1 (NeuAc)1 (G77740PR, G59155GF); (Hex)3 (HexNAc)2 (Deoxyhexose)2 (G20310DM, G78177LC); (Hex)2 (HexNAc)3 (Deoxyhexose)2 (G68384KC, G39326PP); (Hex)3 (HexNAc)3 (Deoxyhexose)1 (G66741QE, G16458JH); (Hex)2 (HexNAc)4 (Deoxyhexose)1 (G23700TV); (Hex)3 (HexNAc)4 (G94517VF, G57672ST); (Hex)3 (HexNAc)2 (Deoxyhexose)3 (G70416EY, G13012GZ); (Hex)2 (HexNAc)5 (G23048PE); (Hex)3 (HexNAc)2 (Deoxyhexose)1 (NeuAc)1 (G74607VK, G81461IK); (Hex)3 (HexNAc)3 (Deoxyhexose)2 (G35949CT, G71094KR, G08426KY); (Hex)2 (HexNAc)4 (Deoxyhexose)2 (G90965BZ); (Hex)3 (HexNAc)4 (Deoxyhexose)1 (G02990AF, G29956GF); (Hex)3 (HexNAc)3 (Deoxyhexose)3 (G23021IW); (Hex)4 (HexNAc)3 (Deoxyhexose)2 (G68308CM); (Hex)3 (HexNAc)4 (Deoxyhexose)2 (G93333OF); (Hex)4 (HexNAc)4 (Deoxyhexose)1 (G94768NG); (Hex)2 (HexNAc)5 (Deoxyhexose)2 (G25612EW); (Hex)3 (HexNAc)3 (Deoxyhexose)4 (G84853WN); (Hex)5 (HexNAc)6 (Deoxyhexose)3 (G65612SS); (Hex)3 (HexNAc)5 (Deoxyhexose)1 (G05252QE); (Hex)7 (HexNAc)6 (Deoxyhexose)1 (G61898SS); (Hex)3 (HexNAc)6 (G62609ZF); (Hex)3 (HexNAc)4 (Deoxyhexose)3 (G59787TQ); (Hex)4 (HexNAc)4 (Deoxyhexose)2 (G98518WL); (Hex)6 (HexNAc)7 (Deoxyhexose)2 (G29852ZH); (Hex)4 (HexNAc)5 (Deoxyhexose)1 (G92547QZ); (Hex)3 (HexNAc)4 (Deoxyhexose)4 (G89469SP); (Hex)5 (HexNAc)4 (Deoxyhexose)2 (G21630AC); (Hex)4 (HexNAc)5 (Deoxyhexose)2 (G82251ZP, G18603ZQ); (Hex)5 (HexNAc)5 (Deoxyhexose)1 (G25957KN); (Hex)4 (HexNAc)6 (Deoxyhexose)1 (G32752FJ); (Hex)6 (HexNAc)5 (Deoxyhexose)1 (G37901JE, G84713IO); (Hex)4 (HexNAc)6 (Deoxyhexose)2 (G99804SJ); (Hex)4 (HexNAc)5 (Deoxyhexose)4 (G11381FO); (Hex)5 (HexNAc)5 (Deoxyhexose)3 (G90829NZ); (Hex)6 (HexNAc)5 (Deoxyhexose)2 (G70999YJ, G02681FY); (Hex)5 (HexNAc)6 (Deoxyhexose)2 (G44467ZE); (Hex)3 (HexNAc)5 (Deoxyhexose)2 (G28921PH); (Hex)6 (HexNAc)5 (Deoxyhexose)3 (G18501TC); (Hex)4 (HexNAc)4 (Deoxyhexose)3 (G66166BF); (HexNAc)2 (G00041MO, G00057MO); (HexNAc)2 (NeuAc)1 (G63334FZ); (HexNAc)2 (NeuGc)1 (G09441IP); (Hex)3 (HexNAc)5 (G00505CR); (Hex)2 (HexNAc)5 (Deoxyhexose)1 (G09396HG); (Hex)2 (HexNAc)1 (G28052FT); (Hex)2 (HexNAc)1 (NeuAc)1 (G59126YU); (Hex)1 (Deoxyhexose)1 (G00068MO); (Hex)1 (NeuAc)1 (G30207PZ, G63069TR); and (Hex)1 (NeuGc)1 (G38557KR, G59867EM).
35. The method of embodiment 34, wherein the mucin glycan comprises a MUC2 glycan.
36. The method of embodiment 35, wherein the MUC2 glycan comprises a formula selected from the group consisting of (Hex)1 (HexNAc)1 (Deoxyhexose)1 (G94435QH); (Hex)1 (NeuAc)1 (G30207PZ, G63069TR); (Hex)1 (Deoxyhexose)1 (G00068MO); (Hex)1 (HexNAc)1 (G76355TG, G85856KC); (Hex)1 (NeuGc)1 (G38557KR, G59867EM); (Hex)1 (HexNAc)2 (Deoxyhexose)1 (G32426JY, G74353FF); (Hex)1 (HexNAc)1 (NeuAc)1 (G65562ZE); (Hex)1 (HexNAc)1 (NeuGc)1 (G64527IJ); (Hex)1 (HexNAc)2 (G00033MO, G61730RY, G56868BH); (HexNAc)2 (G00041MO, G00057MO); (HexNAc)1 (G57321FI); (Hex)1 (HexNAc)3 (Deoxyhexose)1 (G28878FC); (Hex)2 (HexNAc)2 (NeuAc)1 (G46748BU); (Hex)2 (HexNAc)2 (G64973KT); (HexNAc)2 (NeuAc)1 (G63334FZ); (Hex)2 (HexNAc)1 (G28052FT); (Hex)2 (HexNAc)2 (Deoxyhexose)1 (G94514IB, G61216ZY); (Hex)1 (HexNAc)3 (G68893BQ, G23438NR); (HexNAc)2 (NeuGc)1 (G09441IP); (Hex)2 (HexNAc)2 (Deoxyhexose)2 (G23119MJ); (Hex)1 (HexNAc)2 (NeuAc)1 (G85608AG, G64844ET); (Hex)2 (HexNAc)3 (Deoxyhexose)2 (G68384KC, G39326PP); (Hex)2 (HexNAc)3 (G96915PP, G13483MW); (Hex)1 (HexNAc)1 (NeuAc)2 (G01614ZM); (Hex)1 (HexNAc)2 (NeuGc)1 (G60426XC); (Hex)1 (HexNAc)1 (Deoxyhexose)1 (NeuAc)1 (G47180UC); (Hex)1 (HexNAc)2 (Deoxyhexose)1 (NeuAc)1 (G75749JP, G40270LS); (Hex)4 (HexNAc)5 (Deoxyhexose)1 (G92547QZ); (Hex)3 (HexNAc)4 (Deoxyhexose)1 (G02990AF, G29956GF); (Hex)2 (HexNAc)4 (Deoxyhexose)2 (G90965BZ); (Hex)3 (HexNAc)5 (Deoxyhexose)1 (G05252QE); (Hex)2 (HexNAc)1 (NeuAc)1 (G59126YU); (Hex)3 (HexNAc)4 (G94517VF, G57672ST); (Hex)4 (HexNAc)4 (Deoxyhexose)1 (G94768NG); (Hex)2 (HexNAc)3 (Deoxyhexose)1 (G91459EI, G26724PP, G52132CU); (Hex)1 (HexNAc)1 (NeuAc)1 (NeuGc)1 (G49527BY); (Hex)2 (HexNAc)4 (Deoxyhexose)1 (G23700TV); (Hex)3 (HexNAc)5 (G00505CR); (Hex)4 (HexNAc)5 (Deoxyhexose)2 (G82251ZP, G18603ZQ); (Hex)5 (HexNAc)5 (Deoxyhexose)1 (G25957KN); (Hex)2 (HexNAc)4 (G34764BK, G12074QJ); (Hex)3 (HexNAc)3 (Deoxyhexose)1 (G66741QE, G16458JH); (Hex)4 (HexNAc)6 (Deoxyhexose)1 (G32752FJ); (Hex)3 (HexNAc)2 (Deoxyhexose)1 (G15849KC, G81911LP, G41486OC); (Hex)2 (HexNAc)1 (Deoxyhexose)2 (G68200GL); (Hex)4 (HexNAc)6 (Deoxyhexose)2 (G99804SJ); (Hex)3 (HexNAc)2 (G16404NW, G25323VU); (Hex)5 (HexNAc)6 (Deoxyhexose)2 (G44467ZE); and (Hex)3 (HexNAc)3 (G86537AD, G89585FG).
37. The method of embodiment 36, wherein the MUC2 glycan comprises a formula selected from the group consisting of (Hex)1 (HexNAc)1 (Deoxyhexose)1 (G94435QH); (Hex)1 (NeuAc)1 (G30207PZ, G63069TR); (Hex)1 (Deoxyhexose)1 (G00068MO); (Hex)1 (HexNAc)1 (G76355TG, G85856KC); (Hex)1 (NeuGc)1 (G38557KR, G59867EM); (Hex)1 (HexNAc)2 (Deoxyhexose)1 (G32426JY, G74353FF); (Hex)1 (HexNAc)1 (NeuAc)1 (G65562ZE); (Hex)1 (HexNAc)1 (NeuGc)1 (G64527IJ); (Hex)1 (HexNAc)2 (G00033MO, G61730RY, G56868BH); (HexNAc)2 (G00041MO, G00057MO); (HexNAc)1 (G57321FI); (Hex)1 (HexNAc)3 (Deoxyhexose)1 (G28878FC); (Hex)2 (HexNAc)2 (NeuAc)1 (G46748BU); (Hex)2 (HexNAc)2 (G64973KT); (HexNAc)2 (NeuAc)1 (G63334FZ); and (Hex)2 (HexNAc)1 (G28052FT).
38. The method of embodiment 37, wherein the MUC2 glycan comprises a formula selected from the group consisting of (Hex)1 (HexNAc)1 (Deoxyhexose)1 (G94435QH); (Hex)1 (NeuAc)1 (G30207PZ, G63069TR); (Hex)1 (Deoxyhexose)1 (G00068MO); (Hex)1 (HexNAc)1 (G76355TG, G85856KC); (Hex)1 (NeuGc)1 (G38557KR, G59867EM); (Hex)1 (HexNAc)2 (Deoxyhexose)1 (G32426JY, G74353FF); (Hex)1 (HexNAc)1 (NeuAc)1 (G65562ZE); and (Hex)1 (HexNAc)1 (NeuGc)1 (G64527IJ).
39. The method of embodiment 38, wherein the MUC2 glycan comprises a formula selected from the group consisting of (Hex)1 (HexNAc)1 (Deoxyhexose)1 (G94435QH); (Hex)1 (NeuAc)1 (G30207PZ, G63069TR); (Hex)1 (Deoxyhexose)1 (G00068MO); and (Hex)1 (HexNAc)1 (G76355TG, G85856KC).
40. The method of embodiment 34, wherein the mucin glycan comprises a MUC5B glycan.
41. The method of embodiment 40, wherein the MUC5B glycan comprises a formula selected from the group consisting of (Hex)1 (HexNAc)1 (Deoxyhexose)1 (G94435QH); (Hex)1 (HexNAc)1 (G76355TG, G85856KC); (Hex)1 (HexNAc)2 (Deoxyhexose)1 (G32426JY, G74353FF); (Hex)1 (NeuAc)1 (G30207PZ, G63069TR); (Hex)1 (Deoxyhexose)1 (G00068MO); (Hex)3 (HexNAc)3 (Deoxyhexose)2 (G35949CT, G71094KR, G08426KY); (Hex)2 (HexNAc)2 (Deoxyhexose)2 (G23119MJ); (Hex)1 (HexNAc)1 (NeuAc)1 (G65562ZE); (Hex)3 (HexNAc)2 (Deoxyhexose)2 (G20310DM, G78177LC); (Hex)1 (HexNAc)2 (G00033MO, G61730RY, G56868BH); (Hex)1 (HexNAc)2 (Deoxyhexose)2 (G89748NG, G09520ZQ); (Hex)2 (HexNAc)2 (Deoxyhexose)1 (G94514IB, G61216ZY); (Hex)2 (HexNAc)1 (Deoxyhexose)1 (G73318SN, G33986KK); (Hex)2 (HexNAc)3 (Deoxyhexose)1 (G91459EI, G26724PP, G52132CU); (Hex)2 (HexNAc)3 (Deoxyhexose)2 (G68384KC, G39326PP); (Hex)3 (HexNAc)2 (Deoxyhexose)1 (G15849KC, G81911LP, G41486OC); (Hex)2 (HexNAc)1 (G28052FT); (Hex)1 (HexNAc)3 (Deoxyhexose)1 (G28878FC); (Hex)3 (HexNAc)4 (Deoxyhexose)3 (G59787TQ); (Hex)2 (HexNAc)1 (Deoxyhexose)2 (G68200GL); (Hex)2 (HexNAc)2 (NeuAc)1 (G46748BU); (Hex)3 (HexNAc)2 (Deoxyhexose)3 (G70416EY, G13012GZ); (Hex)2 (HexNAc)4 (Deoxyhexose)2 (G90965BZ); (Hex)3 (HexNAc)3 (Deoxyhexose)1 (G66741QE, G16458JH); (Hex)3 (HexNAc)3 (Deoxyhexose)3 (G23021IW); (Hex)2 (HexNAc)2 (G64973KT); (Hex)2 (HexNAc)3 (G96915PP, G13483MW); (Hex)2 (HexNAc)2 (Deoxyhexose)3 (G01532FF); (Hex)3 (HexNAc)4 (Deoxyhexose)4 (G89469SP); (Hex)3 (HexNAc)2 (G16404NW, G25323VU); (Hex)3 (HexNAc)4 (Deoxyhexose)1 (G02990AF, G29956GF); (HexNAc)2 (NeuAc)1 (G63334FZ); (Hex)3 (HexNAc)4 (Deoxyhexose)2 (G93333OF); (Hex)1 (HexNAc)2 (Deoxyhexose)1 (NeuAc)1 (G75749JP, G40270LS); (Hex)3 (HexNAc)2 (Deoxyhexose)4 (G93469SN, G15747RC); (Hex)1 (HexNAc)2 (NeuAc)1 (G85608AG, G64844ET); (Hex)1 (HexNAc)1 (Deoxyhexose)1 (NeuAc)1 (G47180UC); (Hex)4 (HexNAc)3 (Deoxyhexose)2 (G68308CM); (Hex)4 (HexNAc)4 (Deoxyhexose)2 (G98518WL); (HexNAc)2 (G00041MO, G00057MO); (Hex)4 (HexNAc)4 (Deoxyhexose)3 (G66166BF); (Hex)2 (HexNAc)4 (Deoxyhexose)1 (G23700TV); (Hex)3 (HexNAc)2 (Deoxyhexose)1 (NeuAc)1 (G74607VK, G81461IK); (Hex)2 (HexNAc)1 (Deoxyhexose)1 (NeuAc)1 (G49549VN, G95742RK); (Hex)3 (HexNAc)3 (Deoxyhexose)4 (G84853WN); (Hex)5 (HexNAc)4 (Deoxyhexose)2 (G21630AC); (HexNAc)1 (G57321FI); (Hex)1 (HexNAc)3 (G68893BQ, G23438NR); (Hex)2 (HexNAc)1 (Deoxyhexose)3 (G82961CS); (Hex)2 (HexNAc)5 (Deoxyhexose)2 (G25612EW); (Hex)4 (HexNAc)4 (Deoxyhexose)1 (G94768NG); (Hex)4 (HexNAc)5 (Deoxyhexose)4 (G11381FO); (Hex)4 (HexNAc)5 (Deoxyhexose)2 (G82251ZP, G18603ZQ); (Hex)5 (HexNAc)5 (Deoxyhexose)1 (G25957KN); (Hex)2 (HexNAc)4 (G34764BK, G12074QJ); (Hex)4 (HexNAc)5 (Deoxyhexose)1 (G92547QZ); (Hex)1 (NeuGc)1 (G38557KR, G59867EM); (Hex)5 (HexNAc)6 (Deoxyhexose)2 (G44467ZE); (Hex)6 (HexNAc)5 (Deoxyhexose)2 (G70999YJ, G02681FY); (Hex)2 (HexNAc)2 (Deoxyhexose)1 (NeuAc)1 (G77740PR, G59155GF); (Hex)5 (HexNAc)6 (Deoxyhexose)3 (G65612SS); (Hex)3 (HexNAc)3 (G86537AD, G89585FG); (Hex)3 (HexNAc)5 (Deoxyhexose)1 (G05252QE); (Hex)5 (HexNAc)5 (Deoxyhexose)3 (G90829NZ); (Hex)2 (HexNAc)1 (NeuAc)1 (G59126YU); (Hex)7 (HexNAc)6 (Deoxyhexose)1 (G61898SS); and (Hex)3 (HexNAc)4 (G94517VF, G57672ST).
42. The method of embodiment 41, wherein the MUC5B glycan comprises a formula selected from the group consisting of (Hex)1 (HexNAc)1 (Deoxyhexose)1 (G94435QH); (Hex)1 (HexNAc)1 (G76355TG, G85856KC); (Hex)1 (HexNAc)2 (Deoxyhexose)1 (G32426JY, G74353FF); (Hex)1 (NeuAc)1 (G30207PZ, G63069TR); (Hex)1 (Deoxyhexose)1 (G00068MO); (Hex)3 (HexNAc)3 (Deoxyhexose)2 (G35949CT, G71094KR, G08426KY); (Hex)2 (HexNAc)2 (Deoxyhexose)2 (G23119MJ); (Hex)1 (HexNAc)1 (NeuAc)1 (G65562ZE); (Hex)3 (HexNAc)2 (Deoxyhexose)2 (G20310DM, G78177LC); (Hex)1 (HexNAc)2 (G00033MO, G61730RY, G56868BH); (Hex)1 (HexNAc)2 (Deoxyhexose)2 (G89748NG, G09520ZQ); (Hex)2 (HexNAc)2 (Deoxyhexose)1 (G94514IB, G61216ZY); (Hex)2 (HexNAc)1 (Deoxyhexose)1 (G73318SN, G33986KK); (Hex)2 (HexNAc)3 (Deoxyhexose)1 (G91459EI, G26724PP, G52132CU); (Hex)2 (HexNAc)3 (Deoxyhexose)2 (G68384KC, G39326PP); (Hex)3 (HexNAc)2 (Deoxyhexose)1 (G15849KC, G81911LP, G41486OC); (Hex)2 (HexNAc)1 (G28052FT); (Hex)1 (HexNAc)3 (Deoxyhexose)1 (G28878FC); (Hex)3 (HexNAc)4 (Deoxyhexose)3 (G59787TQ); (Hex)2 (HexNAc)1 (Deoxyhexose)2 (G68200GL); (Hex)2 (HexNAc)2 (NeuAc)1 (G46748BU); (Hex)3 (HexNAc)2 (Deoxyhexose)3 (G70416EY, G13012GZ); (Hex)2 (HexNAc)4 (Deoxyhexose)2 (G90965BZ); (Hex)3 (HexNAc)3 (Deoxyhexose)1 (G66741QE, G16458JH); (Hex)3 (HexNAc)3 (Deoxyhexose)3 (G23021IW); (Hex)2 (HexNAc)2 (G64973KT); and (Hex)2 (HexNAc)3 (G96915PP, G13483MW).
43. The method of embodiment 42, wherein the MUC5B glycan comprises a formula selected from the group consisting of (Hex)1 (HexNAc)1 (Deoxyhexose)1 (G94435QH); (Hex)1 (HexNAc)1 (G76355TG, G85856KC); (Hex)1 (HexNAc)2 (Deoxyhexose)1 (G32426JY, G74353FF); (Hex)1 (NeuAc)1 (G30207PZ, G63069TR); (Hex)1 (Deoxyhexose)1 (G00068MO); (Hex)3 (HexNAc)3 (Deoxyhexose)2 (G35949CT, G71094KR, G08426KY); (Hex)2 (HexNAc)2 (Deoxyhexose)2 (G23119MJ); (Hex)1 (HexNAc)1 (NeuAc)1 (G65562ZE); (Hex)3 (HexNAc)2 (Deoxyhexose)2 (G20310DM, G78177LC); (Hex)1 (HexNAc)2 (G00033MO, G61730RY, G56868BH); (Hex)1 (HexNAc)2 (Deoxyhexose)2 (G89748NG, G09520ZQ); (Hex)2 (HexNAc)2 (Deoxyhexose)1 (G94514IB, G61216ZY); and (Hex)2 (HexNAc)1 (Deoxyhexose)1 (G73318SN, G33986KK).
44. The method of embodiment 43, wherein the MUC5B glycan comprises a formula selected from the group consisting of (Hex)1 (HexNAc)1 (Deoxyhexose)1 (G94435QH); (Hex)1 (HexNAc)1 (G76355TG, G85856KC); (Hex)1 (HexNAc)2 (Deoxyhexose)1 (G32426JY, G74353FF); (Hex)1 (NeuAc)1 (G30207PZ, G63069TR); and (Hex)1 (Deoxyhexose)1 (G00068MO).
45. The method of embodiment 34, wherein the mucin glycan comprises a MUC5AC glycan.
46. The method of embodiment 45, wherein the MUC5AC glycan comprises a formula selected from the group consisting of (Hex)1 (HexNAc)1 (G76355TG, G85856KC); (Hex)1 (HexNAc)2 (G00033MO, G61730RY, G56868BH); (Hex)1 (HexNAc)1 (Deoxyhexose)1 (G94435QH); (Hex)1 (Deoxyhexose)1 (G00068MO); (Hex)2 (HexNAc)3 (Deoxyhexose)1 (G91459EI, G26724PP, G52132CU); (Hex)2 (HexNAc)4 (G34764BK, G12074QJ); (Hex)2 (HexNAc)2 (Deoxyhexose)1 (G94514IB, G61216ZY); (Hex)2 (HexNAc)3 (G96915PP, G13483MW); (Hex)2 (HexNAc)2 (G64973KT); (Hex)3 (HexNAc)2 (Deoxyhexose)2 (G20310DM, G78177LC); (Hex)3 (HexNAc)3 (Deoxyhexose)1 (G66741QE, G16458JH); (Hex)3 (HexNAc)4 (G94517VF, G57672ST); (Hex)2 (HexNAc)4 (Deoxyhexose)1 (G23700TV); (Hex)2 (HexNAc)1 (Deoxyhexose)1 (G73318SN, G33986KK); (Hex)1 (HexNAc)2 (Deoxyhexose)1 (G32426JY, G74353FF); (Hex)2 (HexNAc)2 (Deoxyhexose)2 (G23119MJ); (Hex)1 (HexNAc)3 (G68893BQ, G23438NR); (Hex)3 (HexNAc)4 (Deoxyhexose)1 (G02990AF, G29956GF); (Hex)1 (HexNAc)1 (NeuAc)1 (G65562ZE); (Hex)3 (HexNAc)4 (Deoxyhexose)2 (G93333OF); (Hex)3 (HexNAc)2 (Deoxyhexose)1 (G15849KC, G81911LP, G41486OC); (Hex)2 (HexNAc)1 (G28052FT); (Hex)3 (HexNAc)3 (Deoxyhexose)2 (G35949CT, G71094KR, G08426KY); (Hex)3 (HexNAc)3 (G86537AD, G89585FG); (Hex)1 (HexNAc)2 (NeuAc)1 (G85608AG, G64844ET); (Hex)3 (HexNAc)5 (G00505CR); (HexNAc)1 (G57321FI); (Hex)1 (HexNAc)4 (G59229NY); (Hex)4 (HexNAc)3 (Deoxyhexose)2 (G68308CM); (Hex)3 (HexNAc)5 (Deoxyhexose)1 (G05252QE); (Hex)1 (HexNAc)3 (Deoxyhexose)1 (G28878FC); (Hex)2 (HexNAc)1 (NeuAc)1 (G59126YU); (Hex)4 (HexNAc)4 (Deoxyhexose)1 (G94768NG); (Hex)2 (HexNAc)2 (NeuAc)1 (G46748BU); (Hex)3 (HexNAc)2 (G16404NW, G25323VU); (Hex)2 (HexNAc)3 (Deoxyhexose)2 (G68384KC, G39326PP); (Hex)1 (HexNAc)1 (Deoxyhexose)1 (NeuAc)1 (G47180UC); (Hex)4 (HexNAc)4 (Deoxyhexose)2 (G98518WL); (Hex)2 (HexNAc)4 (Deoxyhexose)2 (G90965BZ); (Hex)2 (HexNAc)5 (G23048PE); (Hex)4 (HexNAc)5 (Deoxyhexose)2 (G82251ZP, G18603ZQ); (Hex)3 (HexNAc)6 (G62609ZF); (Hex)2 (HexNAc)1 (Deoxyhexose)2 (G68200GL); (Hex)4 (HexNAc)4 (Deoxyhexose)3 (G66166BF); (Hex)4 (HexNAc)5 (Deoxyhexose)1 (G92547QZ); (Hex)3 (HexNAc)5 (Deoxyhexose)2 (G28921PH); (Hex)2 (HexNAc)5 (Deoxyhexose)1 (G09396HG); (Hex)6 (HexNAc)5 (Deoxyhexose)3 (G18501TC); (Hex)4 (HexNAc)6 (Deoxyhexose)1 (G32752FJ); (Hex)5 (HexNAc)6 (Deoxyhexose)3 (G65612SS); (Hex)7 (HexNAc)6 (Deoxyhexose)1 (G61898SS); (Hex)5 (HexNAc)4 (Deoxyhexose)2 (G21630AC); (Hex)5 (HexNAc)5 (Deoxyhexose)1 (G25957KN); (Hex)6 (HexNAc)5 (Deoxyhexose)1 (G37901JE, G84713IO); (Hex)6 (HexNAc)7 (Deoxyhexose)2 (G29852ZH); (Hex)4 (HexNAc)6 (Deoxyhexose)2 (G99804SJ); (Hex)5 (HexNAc)5 (Deoxyhexose)3 (G90829NZ); (Hex)6 (HexNAc)5 (Deoxyhexose)2 (G70999YJ, G02681FY); and (Hex)5 (HexNAc)6 (Deoxyhexose)2 (G44467ZE).
47. The method of embodiment 46, wherein the MUC5AC glycan comprises a formula selected from the group consisting of (Hex)1 (HexNAc)1 (G76355TG, G85856KC); (Hex)1 (HexNAc)2 (G00033MO, G61730RY, G56868BH); (Hex)1 (HexNAc)1 (Deoxyhexose)1 (G94435QH); (Hex)1 (Deoxyhexose)1 (G00068MO); (Hex)2 (HexNAc)3 (Deoxyhexose)1 (G91459EI, G26724PP, G52132CU); (Hex)2 (HexNAc)4 (G34764BK, G12074QJ); (Hex)2 (HexNAc)2 (Deoxyhexose)1 (G94514IB, G61216ZY); (Hex)2 (HexNAc)3 (G96915PP, G13483MW); (Hex)2 (HexNAc)2 (G64973KT); (Hex)3 (HexNAc)2 (Deoxyhexose)2 (G20310DM, G78177LC); (Hex)3 (HexNAc)3 (Deoxyhexose)1 (G66741QE, G16458JH); (Hex)3 (HexNAc)4 (G94517VF, G57672ST); (Hex)2 (HexNAc)4 (Deoxyhexose)1 (G23700TV); (Hex)2 (HexNAc)1 (Deoxyhexose)1 (G73318SN, G33986KK); (Hex)1 (HexNAc)2 (Deoxyhexose)1 (G32426JY, G74353FF); (Hex)2 (HexNAc)2 (Deoxyhexose)2 (G23119MJ); (Hex)1 (HexNAc)3 (G68893BQ, G23438NR); (Hex)3 (HexNAc)4 (Deoxyhexose)1 (G02990AF, G29956GF); and (Hex)1 (HexNAc)1 (NeuAc)1 (G65562ZE).
48. The method of embodiment 47, wherein the MUC5AC glycan comprises a formula selected from the group consisting of (Hex)1 (HexNAc)1 (G76355TG, G85856KC); (Hex)1 (HexNAc)2 (G00033MO, G61730RY, G56868BH); (Hex)1 (HexNAc)1 (Deoxyhexose)1 (G94435QH); (Hex)1 (Deoxyhexose)1 (G00068MO); (Hex)2 (HexNAc)3 (Deoxyhexose)1 (G91459EI, G26724PP, G52132CU); (Hex)2 (HexNAc)4 (G34764BK, G12074QJ); (Hex)2 (HexNAc)2 (Deoxyhexose)1 (G94514IB, G61216ZY); (Hex)2 (HexNAc)3 (G96915PP, G13483MW); (Hex)2 (HexNAc)2 (G64973KT); (Hex)3 (HexNAc)2 (Deoxyhexose)2 (G20310DM, G78177LC); (Hex)3 (HexNAc)3 (Deoxyhexose)1 (G66741QE, G16458JH); and (Hex)3 (HexNAc)4 (G94517VF, G57672ST).
49. The method of embodiment 48, wherein the MUC5AC glycan comprises a formula selected from the group consisting of (Hex)1 (HexNAc)1 (G76355TG, G85856KC); (Hex)1 (HexNAc)2 (G00033MO, G61730RY, G56868BH); (Hex)1 (HexNAc)1 (Deoxyhexose)1 (G94435QH); (Hex)1 (Deoxyhexose)1 (G00068MO); (Hex)2 (HexNAc)3 (Deoxyhexose)1 (G91459EI, G26724PP, G52132CU); and (Hex)2 (HexNAc)4 (G34764BK, G12074QJ).
50. The method of embodiment any one of embodiments 34-49, wherein the mucin glycan is a unsulfated mucin glycan.
51. The method of embodiment any one of embodiments 34-49, wherein the mucin glycan is a sulfated mucin glycan.
52. The method of embodiment 51, wherein the sulfated mucin glycan comprises a formula selected from the group consisting of S1 (Hex)1 (HexNAc)1 (G10634LC); S1 (Hex)1 (HexNAc)1 (Deoxyhexose)1 (G08671QK); S1 (Hex)1 (HexNAc)2 (G32406CO); S1 (Hex)2 (HexNAc)1 (Deoxyhexose)1 (G24803MV); S1 (Hex)1 (HexNAc)2 (Deoxyhexose)1 (G10520JC); S1 (Hex)1 (HexNAc)2 (NeuAc)1 (G96888OD); S1 (Hex)2 (HexNAc)2 (Deoxyhexose)1 (G24803MV); S1 (Hex)1 (HexNAc)2 (NeuGc)1 (G60426XC); S1 (Hex)1 (HexNAc)3 (Deoxyhexose)1 (G72091WB, G60644GY); and S1 (Hex)2 (HexNAc)3 (Deoxyhexose)1 (G35891PL).
53. The method of embodiment 51, wherein the sulfated mucin glycan comprises a sulfated MUC2 glycan, a sulfated MUC5AC glycan, a sulfated MUC5B glycan, or a combination thereof.
54. The method of embodiment 53, wherein:
   a) the sulfated MUC2 glycan comprises a formula selected from the group consisting of S1 (Hex)1 (HexNAc)2 (G32406CO); S1 (Hex)1 (HexNAc)2 (Deoxyhexose)1 (G10520JC); S1 (Hex)1 (HexNAc)2 (NeuAc)1 (G96888OD); S1 (Hex)1 (HexNAc)3 (Deoxyhexose)1 (G72091WB, G60644GY); and S1 (Hex)1 (HexNAc)2 (NeuGc)1 (G60426XC);
   b) the sulfated MUC5B glycan comprises a formula selected from the group consisting of S1 (Hex)2 (HexNAc)1 (Deoxyhexose)1 (G24803MV); S1 (Hex)1 (HexNAc)1 (Deoxyhexose)1 (G08671QK); S1 (Hex)1 (HexNAc)1 (G10634LC); S1 (Hex)2 (HexNAc)2 (Deoxyhexose)1 (G24803MV); and S1 (Hex)2 (HexNAc)3 (Deoxyhexose)1 (G35891PL); or
   c) the sulfated MUC5AC glycan comprises a formula selected from the group consisting of S1 (Hex)1 (HexNAc)2 (Deoxyhexose)1 (G10520JC); S1 (Hex)2 (HexNAc)2 (Deoxyhexose)1 (G24803MV); S1 (Hex)2 (HexNAc)1 (Deoxyhexose)1 (G24803MV); S1 (Hex)2 (HexNAc)3 (Deoxyhexose)1 (G35891PL); and S1 (Hex)1 (HexNAc)2 (G32406CO).
55. A composition comprising a synthetic mucin glycan.
56. A composition comprising a mucin glycan, wherein the purity of the mucin glycan is at least about 30%.
57. The composition of embodiment 56, wherein the purity of the mucin glycan is at least about 50%.
58. A defined mucin glycan composition, comprising one or more desired mucin glycans, wherein at least 80% of total mucin glycans present in the composition are of the one or more desired mucin glycans or pharmaceutically acceptable salts thereof.
59. The composition of any one of embodiments 55-58, wherein the hydrogen atom of the hydroxyl at anomeric/C-1 position of the GalNAc residue is unsubstituted or substituted.
60. The composition of embodiment 58 or 59, wherein the defined mucin glycan composition comprises about 1-5 desired mucin glycans or pharmaceutically acceptable salts thereof.
61. The composition of any one of embodiments 58-60, wherein at least 90%, 95%, 99% or 99.5% of the total mucin glycans present in the composition are of the one or more desired mucin glycans or pharmaceutically acceptable salts thereof.
62. The composition of any one of embodiments 58-61, wherein the defined mucin glycan composition is incorporated into a formulation for therapeutic administration.
63. The composition of any one of embodiments 58-61, wherein the defined mucin glycan composition is incorporated into a coating for a surface susceptible for fungal biofilm formation.
64. The composition of any one of embodiments 55-63, wherein the mucin glycan is a secreted gel-forming mucin glycan.
65. The composition of any one of embodiments 55-63, wherein the mucin glycan comprises a MUC2 glycan, a MUC5AC glycan, a MUC5B glycan, or a combination thereof.
66. The composition of any one of embodiments 55-65, wherein the mucin glycan comprises one or more of N-acetylgalactosamine (GalNAc), N-acetylglucosamine (GlcNAc), mannose (Man), fucose (Fuc), N-acetylneuraminic acid (Neu5Ac), galactose (Gal) or a combination thereof.
67. The composition of any one of embodiments 55-66, wherein the mucin glycan comprises a glycan core of the following structural formula:

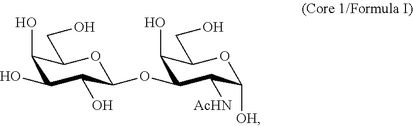

or a tautomer or stereoisomer thereof.
68. The composition of embodiment 67, wherein the mucin glycan comprises a glycan structure selected from the group consisting of 1a, 2, 3a, 4, 9c, 15b, 16b, 18b, 6, 7 and 25b of FIG. 4A.
69. The composition of embodiment 68, wherein the mucin glycan further comprises fucose, sialic acid, or a combination thereof.
70. The composition of embodiment 69, wherein the mucin glycan comprises the following structural formula:

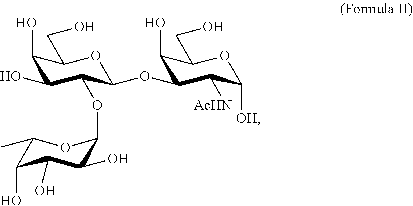

or a tautomer or stereoisomer thereof, or

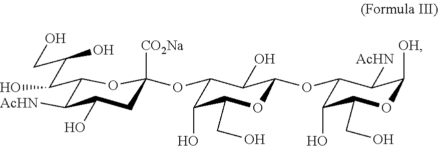

or a tautomer or stereoisomer thereof.
71. The composition of embodiment 67, wherein the mucin glycan comprises a glycan core of the following structural formula:

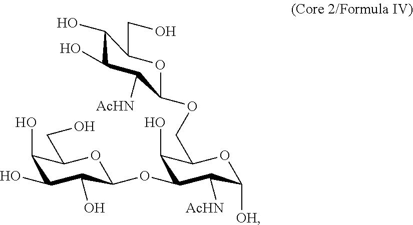

or a tautomer or stereoisomer thereof.
72. The composition of embodiment 71, wherein the mucin glycan comprises a glycan structure selected from the group consisting of 8a, 9a, 9b, 10a, 11a, 12a, 12b, 13, 14a, 15a, 16a, 17, 18a, 19, 20a, 21a, 21b, 22, 23a, 24a, 24b, 25a, 26a, 27, 28, 29, 30, 31, 32, 33a, 34a, 35, 36, 37, 38a, 38b, 39, 40, 41, 42, 43, 44, 45, 46, 47a, 48, 49a, 50, 51, 52, 53a and 54 of FIG. 4A.
73. The composition of embodiment 72, wherein the mucin glycan comprises fucose, galactose, or a combination thereof.
74. The composition of embodiment 73, wherein the mucin glycan comprises the following structural formula:

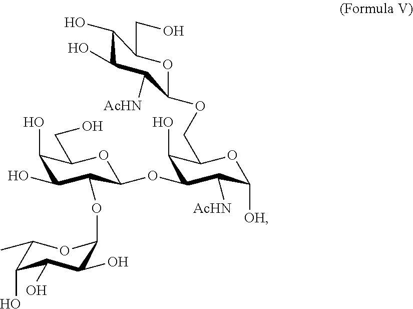

or a tautomer or stereoisomer thereof or

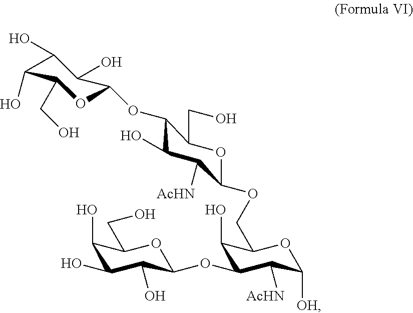

or a tautomer or stereoisomer thereof.
75. The composition of any one of embodiments 55-66, wherein the mucin glycan comprises a glycan core of the following structural formula:

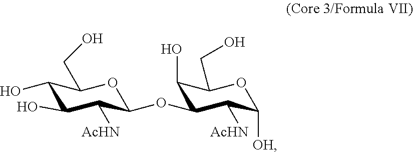

or a tautomer or stereoisomer thereof.
76. The composition of embodiment 75, wherein the mucin glycan comprises a glycan structure selected from the group consisting of 8b, 10b, 20b, 26b and 55 of FIG. 4A.
77. The composition of embodiment 75, wherein the mucin glycan comprises a glycan core of the following structural formula:

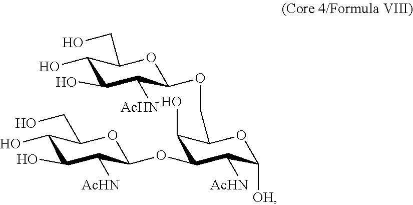

or a tautomer or stereoisomer thereof.
78. The composition of embodiment 77, wherein the mucin glycan comprises a glycan structure selected from the group consisting of 34b and 56 of FIG. 4A.
79. The composition of any one of embodiments 55-66, wherein the mucin glycan comprises a glycan core of the following structural formula:

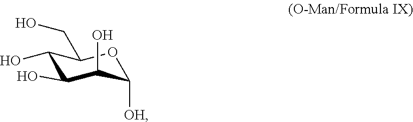

or a tautomer or stereoisomer thereof.
80. The composition of embodiment 79, wherein the mucin glycan comprises a glycan structure selected from the group consisting of 1b, 3b, 11b, 14b, 57, 8c, 23b, 23c, 24c, 33b, 58, 47b, 49b and 53b of FIG. 4A.
81. The composition of any one of embodiments 55-66, wherein the mucin glycan comprises a glycan core of the following structural formula:

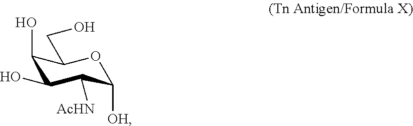

or a tautomer or stereoisomer thereof.
82. The composition of embodiment 81, wherein the mucin glycan comprises a glycan structure of 5 of FIG. 4A.
83. The composition of any one of embodiments 55-82, wherein the mucin glycan is unsulfated.
84. The composition of any one of embodiments 55-82, wherein the mucin glycan is sulfated.
85. The composition of embodiment 84, wherein the sulfated mucin glycan comprises the glycan structure selected from:
   a) the group consisting of 1a, 2, 3a, 9c, 15b and 18b of FIG. 4A;
   b) the group consisting of 8a, 9a, 9b, 11a, 12a, 12b, 13, 14a, 15a, 17, 18a, 19, 20a, 21a, 21b, 23a, 24a, 24b, 30 and 34a of FIG. 4A;
   c) 8b or 20b of FIG. 4A;
   d) 34b of FIG. 4A; or
   e) the group consisting of 11b, 14b, 57, 23b and 23c of FIG. 4A.
86. The composition of any one of embodiments 55-85, wherein the mucin glycan further comprises N-acetylgalactosamine, N-acetylglucosamine, mannose, fucose, N-acetylneuraminic acid, galactose, sulfate, sialic acid, or a combination thereof.
87. The composition of any one of embodiments 55-66, wherein the mucin glycan comprises a formula selected from the group consisting of (HexNAc)1 (G57321FI); (Hex)1 (HexNAc)1 (G76355TG, G85856KC); (Hex)1 (HexNAc)1 (Deoxyhexose)1 (G94435QH); (Hex)1 (HexNAc)1 (NeuAc)1 (G65562ZE); (Hex)2 (HexNAc)1 (Deoxyhexose)1 (G73318SN, G33986KK); (Hex)1 (HexNAc)1 (NeuGc)1 (G64527IJ); (Hex)1 (HexNAc)1 (Deoxyhexose)1 (NeuAc)1 (G47180UC); (Hex)2 (HexNAc)1 (Deoxyhexose)2 (G68200GL); (Hex)1 (HexNAc)1 (NeuAc)2 (G01614ZM); (Hex)2 (HexNAc)1 (Deoxyhexose)3 (G82961CS); (Hex)2 (HexNAc)1 (Deoxyhexose)1 (NeuAc)1 (G49549VN, G95742RK); (Hex)1 (HexNAc)1 (NeuAc)1 (NeuGc)1 (G49527BY); (Hex)1 (HexNAc)2 (Deoxyhexose)1 (NeuAc)1 (G75749JP, G40270LS); (Hex)3 (HexNAc)2 (Deoxyhexose)4 (G93469SN, G15747RC); (Hex)1 (HexNAc)2 (G00033MO, G61730RY, G56868BH); (Hex)1 (HexNAc)2 (Deoxyhexose)1 (G32426JY, G74353FF); (Hex)2 (HexNAc)2 (G64973KT); (Hex)1 (HexNAc)3 (G68893BQ, G23438NR); (Hex)1 (HexNAc)2 (Deoxyhexose)2 (G89748NG, G09520ZQ); (Hex)1 (HexNAc)2 (NeuAc)1 (G85608AG, G64844ET); (Hex)2 (HexNAc)2 (Deoxyhexose)1 (G94514IB, G61216ZY); (Hex)1 (HexNAc)2 (NeuGc)1 (G60426XC); (Hex)3 (HexNAc)2 (G16404NW, G25323VU); (Hex)1 (HexNAc)3 (Deoxyhexose)1 (G28878FC); (Hex)2 (HexNAc)3 (G96915PP, G13483MW); (Hex)1 (HexNAc)4 (G59229NY); (Hex)2 (HexNAc)2 (Deoxyhexose)2 (G23119MJ); (Hex)2 (HexNAc)2 (NeuAc)1 (G46748BU); (Hex)3 (HexNAc)2 (Deoxyhexose)1 (G15849KC, G81911LP, G41486OC); (Hex)2 (HexNAc)3 (Deoxyhexose)1 (G91459EI, G26724PP, G52132CU); (Hex)3 (HexNAc)3 (G86537AD, G89585FG); (Hex)2 (HexNAc)4 (G34764BK, G12074QJ); (Hex)2 (HexNAc)2 (Deoxyhexose)3 (G01532FF); (Hex)2 (HexNAc)2 (Deoxyhexose)1 (NeuAc)1 (G77740PR, G59155GF); (Hex)3 (HexNAc)2 (Deoxyhexose)2 (G20310DM, G78177LC); (Hex)2 (HexNAc)3 (Deoxyhexose)2 (G68384KC, G39326PP); (Hex)3 (HexNAc)3 (Deoxyhexose)1 (G66741QE, G16458JH); (Hex)2 (HexNAc)4 (Deoxyhexose)1 (G23700TV); (Hex)3 (HexNAc)4 (G94517VF, G57672ST); (Hex)3 (HexNAc)2 (Deoxyhexose)3 (G70416EY, G13012GZ); (Hex)2 (HexNAc)5 (G23048PE); (Hex)3 (HexNAc)2 (Deoxyhexose)1 (NeuAc)1 (G74607VK, G81461IK); (Hex)3 (HexNAc)3 (Deoxyhexose)2 (G35949CT, G71094KR, G08426KY); (Hex)2 (HexNAc)4 (Deoxyhexose)2 (G90965BZ); (Hex)3 (HexNAc)4 (Deoxyhexose)1 (G02990AF, G29956GF); (Hex)3 (HexNAc)3 (Deoxyhexose)3 (G23021IW); (Hex)4 (HexNAc)3 (Deoxyhexose)2 (G68308CM); (Hex)3 (HexNAc)4 (Deoxyhexose)2 (G93333OF); (Hex)4 (HexNAc)4 (Deoxyhexose)1 (G94768NG); (Hex)2 (HexNAc)5 (Deoxyhexose)2 (G25612EW); (Hex)3 (HexNAc)3 (Deoxyhexose)4 (G84853WN); (Hex)5 (HexNAc)6 (Deoxyhexose)3 (G65612SS); (Hex)3 (HexNAc)5 (Deoxyhexose)1 (G05252QE); (Hex)7 (HexNAc)6 (Deoxyhexose)1 (G61898SS); (Hex)3 (HexNAc)6 (G62609ZF); (Hex)3 (HexNAc)4 (Deoxyhexose)3 (G59787TQ); (Hex)4 (HexNAc)4 (Deoxyhexose)2 (G98518WL); (Hex)6 (HexNAc)7 (Deoxyhexose)2 (G29852ZH); (Hex)4 (HexNAc)5 (Deoxyhexose)1 (G92547QZ); (Hex)3 (HexNAc)4 (Deoxyhexose)4 (G89469SP); (Hex)5 (HexNAc)4 (Deoxyhexose)2 (G21630AC); (Hex)4 (HexNAc)5 (Deoxyhexose)2 (G82251ZP, G18603ZQ); (Hex)5 (HexNAc)5 (Deoxyhexose)1 (G25957KN); (Hex)4 (HexNAc)6 (Deoxyhexose)1 (G32752FJ); (Hex)6 (HexNAc)5 (Deoxyhexose)1 (G37901JE, G84713IO); (Hex)4 (HexNAc)6 (Deoxyhexose)2 (G99804SJ); (Hex)4 (HexNAc)5 (Deoxyhexose)4 (G11381FO); (Hex)5 (HexNAc)5 (Deoxyhexose)3 (G90829NZ); (Hex)6 (HexNAc)5 (Deoxyhexose)2 (G70999YJ, G02681FY); (Hex)5 (HexNAc)6 (Deoxyhexose)2 (G44467ZE); (Hex)3 (HexNAc)5 (Deoxyhexose)2 (G28921PH); (Hex)6 (HexNAc)5 (Deoxyhexose)3 (G18501TC); (Hex)4 (HexNAc)4 (Deoxyhexose)3 (G66166BF); (HexNAc)2 (G00041MO, G00057MO); (HexNAc)2 (NeuAc)1 (G63334FZ); (HexNAc)2 (NeuGc)1 (G09441IP); (Hex)3 (HexNAc)5 (G00505CR); (Hex)2 (HexNAc)5 (Deoxyhexose)1 (G09396HG); (Hex)2 (HexNAc)1 (G28052FT); (Hex)2 (HexNAc)1 (NeuAc)1 (G59126YU); (Hex)1 (Deoxyhexose)1 (G00068MO); (Hex)1 (NeuAc)1 (G30207PZ, G63069TR); and (Hex)1 (NeuGc)1 (G38557KR, G59867EM).
88. The composition of embodiment 87, wherein the mucin glycan comprises a MUC2 glycan.
89. The composition of embodiment 88, wherein the MUC2 glycan comprises a formula selected from the group consisting of (Hex)1 (HexNAc)1 (Deoxyhexose)1 (G94435QH); (Hex)1 (NeuAc)1 (G30207PZ, G63069TR); (Hex)1 (Deoxyhexose)1 (G00068MO); (Hex)1 (HexNAc)1 (G76355TG, G85856KC); (Hex)1 (NeuGc)1 (G38557KR, G59867EM); (Hex)1 (HexNAc)2 (Deoxyhexose)1 (G32426JY, G74353FF); (Hex)1 (HexNAc)1 (NeuAc)1 (G65562ZE); (Hex)1 (HexNAc)1 (NeuGc)1 (G64527IJ); (Hex)1 (HexNAc)2 (G00033MO, G61730RY, G56868BH); (HexNAc)2 (G00041MO, G00057MO); (HexNAc)1 (G57321FI); (Hex)1 (HexNAc)3 (Deoxyhexose)1 (G28878FC); (Hex)2 (HexNAc)2 (NeuAc)1 (G46748BU); (Hex)2 (HexNAc)2 (G64973KT); (HexNAc)2 (NeuAc)1 (G63334FZ); (Hex)2 (HexNAc)1 (G28052FT); (Hex)2 (HexNAc)2 (Deoxyhexose)1 (G94514IB, G61216ZY); (Hex)1 (HexNAc)3 (G68893BQ, G23438NR); (HexNAc)2 (NeuGc)1 (G09441IP); (Hex)2 (HexNAc)2 (Deoxyhexose)2 (G23119MJ); (Hex)1 (HexNAc)2 (NeuAc)1 (G85608AG, G64844ET); (Hex)2 (HexNAc)3 (Deoxyhexose)2 (G68384KC, G39326PP); (Hex)2 (HexNAc)3 (G96915PP, G13483MW); (Hex)1 (HexNAc)1 (NeuAc)2 (G01614ZM); (Hex)1 (HexNAc)2 (NeuGc)1 (G60426XC); (Hex)1 (HexNAc)1 (Deoxyhexose)1 (NeuAc)1 (G47180UC); (Hex)1 (HexNAc)2 (Deoxyhexose)1 (NeuAc)1 (G75749JP, G40270LS); (Hex)4 (HexNAc)5 (Deoxyhexose)1 (G92547QZ); (Hex)3 (HexNAc)4 (Deoxyhexose)1 (G02990AF, G29956GF); (Hex)2 (HexNAc)4 (Deoxyhexose)2 (G90965BZ); (Hex)3 (HexNAc)5 (Deoxyhexose)1 (G05252QE); (Hex)2 (HexNAc)1 (NeuAc)1 (G59126YU); (Hex)3 (HexNAc)4 (G94517VF, G57672ST); (Hex)4 (HexNAc)4 (Deoxyhexose)1 (G94768NG); (Hex)2 (HexNAc)3 (Deoxyhexose)1 (G91459EI, G26724PP, G52132CU); (Hex)1 (HexNAc)1 (NeuAc)1 (NeuGc)1 (G49527BY); (Hex)2 (HexNAc)4 (Deoxyhexose)1 (G23700TV); (Hex)3 (HexNAc)5 (G00505CR); (Hex)4 (HexNAc)5 (Deoxyhexose)2 (G82251ZP, G18603ZQ); (Hex)5 (HexNAc)5 (Deoxyhexose)1 (G25957KN); (Hex)2 (HexNAc)4 (G34764BK, G12074QJ); (Hex)3 (HexNAc)3 (Deoxyhexose)1 (G66741QE, G16458JH); (Hex)4 (HexNAc)6 (Deoxyhexose)1 (G32752FJ); (Hex)3 (HexNAc)2 (Deoxyhexose)1 (G15849KC, G81911LP, G41486OC); (Hex)2 (HexNAc)1 (Deoxyhexose)2 (G68200GL); (Hex)4 (HexNAc)6 (Deoxyhexose)2 (G99804SJ); (Hex)3 (HexNAc)2 (G16404NW, G25323VU); (Hex)5 (HexNAc)6 (Deoxyhexose)2 (G44467ZE); and (Hex)3 (HexNAc)3 (G86537AD, G89585FG).
90. The composition of embodiment 89, wherein the MUC2 glycan comprises a formula selected from the group consisting of (Hex)1 (HexNAc)1 (Deoxyhexose)1 (G94435QH); (Hex)1 (NeuAc)1 (G30207PZ, G63069TR); (Hex)1 (Deoxyhexose)1 (G00068MO); (Hex)1 (HexNAc)1 (G76355TG, G85856KC); (Hex)1 (NeuGc)1 (G38557KR, G59867EM); (Hex)1 (HexNAc)2 (Deoxyhexose)1 (G32426JY, G74353FF); (Hex)1 (HexNAc)1 (NeuAc)1 (G65562ZE); (Hex)1 (HexNAc)1 (NeuGc)1 (G64527IJ); (Hex)1 (HexNAc)2 (G00033MO, G61730RY, G56868BH); (HexNAc)2 (G00041MO, G00057MO); (HexNAc)1 (G57321FI); (Hex)1 (HexNAc)3 (Deoxyhexose)1 (G28878FC); (Hex)2 (HexNAc)2 (NeuAc)1 (G46748BU); (Hex)2 (HexNAc)2 (G64973KT); (HexNAc)2 (NeuAc)1 (G63334FZ); and (Hex)2 (HexNAc)1 (G28052FT).
91. The composition of embodiment 90, wherein the MUC2 glycan comprises a formula selected from the group consisting of (Hex)1 (HexNAc)1 (Deoxyhexose)1 (G94435QH); (Hex)1 (NeuAc)1 (G30207PZ, G63069TR); (Hex)1 (Deoxyhexose)1 (G00068MO); (Hex)1 (HexNAc)1 (G76355TG, G85856KC); (Hex)1 (NeuGc)1 (G38557KR, G59867EM); (Hex)1 (HexNAc)2 (Deoxyhexose)1 (G32426JY, G74353FF); (Hex)1 (HexNAc)1 (NeuAc)1 (G65562ZE); and (Hex)1 (HexNAc)1 (NeuGc)1 (G64527IJ).
92. The composition of embodiment 91, wherein the MUC2 glycan comprises a formula selected from the group consisting of (Hex)1 (HexNAc)1 (Deoxyhexose)1 (G94435QH); (Hex)1 (NeuAc)1 (G30207PZ, G63069TR); (Hex)1 (Deoxyhexose)1 (G00068MO); and (Hex)1 (HexNAc)1 (G76355TG, G85856KC).
93. The composition of embodiment 87, wherein the mucin glycan comprises a MUC5B glycan.
94. The composition of embodiment 93, wherein the MUC5B glycan comprises a formula selected from the group consisting of (Hex)1 (HexNAc)1 (Deoxyhexose)1 (G94435QH); (Hex)1 (HexNAc)1 (G76355TG, G85856KC); (Hex)1 (HexNAc)2 (Deoxyhexose)1 (G32426JY, G74353FF); (Hex)1 (NeuAc)1 (G30207PZ, G63069TR); (Hex)1 (Deoxyhexose)1 (G00068MO); (Hex)3 (HexNAc)3 (Deoxyhexose)2 (G35949CT, G71094KR, G08426KY); (Hex)2 (HexNAc)2 (Deoxyhexose)2 (G23119MJ); (Hex)1 (HexNAc)1 (NeuAc)1 (G65562ZE); (Hex)3 (HexNAc)2 (Deoxyhexose)2 (G20310DM, G78177LC); (Hex)1 (HexNAc)2 (G00033MO, G61730RY, G56868BH); (Hex)1 (HexNAc)2 (Deoxyhexose)2 (G89748NG, G09520ZQ); (Hex)2 (HexNAc)2 (Deoxyhexose)1 (G94514IB, G61216ZY); (Hex)2 (HexNAc)1 (Deoxyhexose)1 (G73318SN, G33986KK); (Hex)2 (HexNAc)3 (Deoxyhexose)1 (G91459EI, G26724PP, G52132CU); (Hex)2 (HexNAc)3 (Deoxyhexose)2 (G68384KC, G39326PP); (Hex)3 (HexNAc)2 (Deoxyhexose)1 (G15849KC, G81911LP, G41486OC); (Hex)2 (HexNAc)1 (G28052FT); (Hex)1 (HexNAc)3 (Deoxyhexose)1 (G28878FC); (Hex)3 (HexNAc)4 (Deoxyhexose)3 (G59787TQ); (Hex)2 (HexNAc)1 (Deoxyhexose)2 (G68200GL); (Hex)2 (HexNAc)2 (NeuAc)1 (G46748BU); (Hex)3 (HexNAc)2 (Deoxyhexose)3 (G70416EY, G13012GZ); (Hex)2 (HexNAc)4 (Deoxyhexose)2 (G90965BZ); (Hex)3 (HexNAc)3 (Deoxyhexose)1 (G66741QE, G16458JH); (Hex)3 (HexNAc)3 (Deoxyhexose)3 (G23021IW); (Hex)2 (HexNAc)2 (G64973KT); (Hex)2 (HexNAc)3 (G96915PP, G13483MW); (Hex)2 (HexNAc)2 (Deoxyhexose)3 (G01532FF); (Hex)3 (HexNAc)4 (Deoxyhexose)4 (G89469SP); (Hex)3 (HexNAc)2 (G16404NW, G25323VU); (Hex)3 (HexNAc)4 (Deoxyhexose)1 (G02990AF, G29956GF); (HexNAc)2 (NeuAc)1 (G63334FZ); (Hex)3 (HexNAc)4 (Deoxyhexose)2 (G93333OF); (Hex)1 (HexNAc)2 (Deoxyhexose)1 (NeuAc)1 (G75749JP, G40270LS); (Hex)3 (HexNAc)2 (Deoxyhexose)4 (G93469SN, G15747RC); (Hex)1 (HexNAc)2 (NeuAc)1 (G85608AG, G64844ET); (Hex)1 (HexNAc)1 (Deoxyhexose)1 (NeuAc)1 (G47180UC); (Hex)4 (HexNAc)3 (Deoxyhexose)2 (G68308CM); (Hex)4 (HexNAc)4 (Deoxyhexose)2 (G98518WL); (HexNAc)2 (G00041MO, G00057MO); (Hex)4 (HexNAc)4 (Deoxyhexose)3 (G66166BF); (Hex)2 (HexNAc)4 (Deoxyhexose)1 (G23700TV); (Hex)3 (HexNAc)2 (Deoxyhexose)1 (NeuAc)1 (G74607VK, G81461IK); (Hex)2 (HexNAc)1 (Deoxyhexose)1 (NeuAc)1 (G49549VN, G95742RK); (Hex)3 (HexNAc)3 (Deoxyhexose)4 (G84853WN); (Hex)5 (HexNAc)4 (Deoxyhexose)2 (G21630AC); (HexNAc)1 (G57321FI); (Hex)1 (HexNAc)3 (G68893BQ, G23438NR); (Hex)2 (HexNAc)1 (Deoxyhexose)3 (G82961CS); (Hex)2 (HexNAc)5 (Deoxyhexose)2 (G25612EW); (Hex)4 (HexNAc)4 (Deoxyhexose)1 (G94768NG); (Hex)4 (HexNAc)5 (Deoxyhexose)4 (G11381FO); (Hex)4 (HexNAc)5 (Deoxyhexose)2 (G82251ZP, G18603ZQ); (Hex)5 (HexNAc)5 (Deoxyhexose)1 (G25957KN); (Hex)2 (HexNAc)4 (G34764BK, G12074QJ); (Hex)4 (HexNAc)5 (Deoxyhexose)1 (G92547QZ); (Hex)1 (NeuGc)1 (G38557KR, G59867EM); (Hex)5 (HexNAc)6 (Deoxyhexose)2 (G44467ZE); (Hex)6 (HexNAc)5 (Deoxyhexose)2 (G70999YJ, G02681FY); (Hex)2 (HexNAc)2 (Deoxyhexose)1 (NeuAc)1 (G77740PR, G59155GF); (Hex)5 (HexNAc)6 (Deoxyhexose)3 (G65612SS); (Hex)3 (HexNAc)3 (G86537AD, G89585FG); (Hex)3 (HexNAc)5 (Deoxyhexose)1 (G05252QE); (Hex)5 (HexNAc)5 (Deoxyhexose)3 (G90829NZ); (Hex)2 (HexNAc)1 (NeuAc)1 (G59126YU); (Hex)7 (HexNAc)6 (Deoxyhexose)1 (G61898SS); and (Hex)3 (HexNAc)4 (G94517VF, G57672ST).
95. The composition of embodiment 94, wherein the MUC5B glycan comprises a formula selected from the group consisting of (Hex)1 (HexNAc)1 (Deoxyhexose)1 (G94435QH); (Hex)1 (HexNAc)1 (G76355TG, G85856KC); (Hex)1 (HexNAc)2 (Deoxyhexose)1 (G32426JY, G74353FF); (Hex)1 (NeuAc)1 (G30207PZ, G63069TR); (Hex)1 (Deoxyhexose)1 (G00068MO); (Hex)3 (HexNAc)3 (Deoxyhexose)2 (G35949CT, G71094KR, G08426KY); (Hex)2 (HexNAc)2 (Deoxyhexose)2 (G23119MJ); (Hex)1 (HexNAc)1 (NeuAc)1 (G65562ZE); (Hex)3 (HexNAc)2 (Deoxyhexose)2 (G20310DM, G78177LC); (Hex)1 (HexNAc)2 (G00033MO, G61730RY, G56868BH); (Hex)1 (HexNAc)2 (Deoxyhexose)2 (G89748NG, G09520ZQ); (Hex)2 (HexNAc)2 (Deoxyhexose)1 (G94514IB, G61216ZY); (Hex)2 (HexNAc)1 (Deoxyhexose)1 (G73318SN, G33986KK); (Hex)2 (HexNAc)3 (Deoxyhexose)1 (G91459EI, G26724PP, G52132CU); (Hex)2 (HexNAc)3 (Deoxyhexose)2 (G68384KC, G39326PP); (Hex)3 (HexNAc)2 (Deoxyhexose)1 (G15849KC, G81911LP, G41486OC); (Hex)2 (HexNAc)1 (G28052FT); (Hex)1 (HexNAc)3 (Deoxyhexose)1 (G28878FC); (Hex)3 (HexNAc)4 (Deoxyhexose)3 (G59787TQ); (Hex)2 (HexNAc)1 (Deoxyhexose)2 (G68200GL); (Hex)2 (HexNAc)2 (NeuAc)1 (G46748BU); (Hex)3 (HexNAc)2 (Deoxyhexose)3 (G70416EY, G13012GZ); (Hex)2 (HexNAc)4 (Deoxyhexose)2 (G90965BZ); (Hex)3 (HexNAc)3 (Deoxyhexose)1 (G66741QE, G16458JH); (Hex)3 (HexNAc)3 (Deoxyhexose)3 (G23021IW); (Hex)2 (HexNAc)2 (G64973KT); and (Hex)2 (HexNAc)3 (G96915PP, G13483MW).
96. The composition of embodiment 95, wherein the MUC5B glycan comprises a formula selected from the group consisting of (Hex)1 (HexNAc)1 (Deoxyhexose)1 (G94435QH); (Hex)1 (HexNAc)1 (G76355TG, G85856KC); (Hex)1 (HexNAc)2 (Deoxyhexose)1 (G32426JY, G74353FF); (Hex)1 (NeuAc)1 (G30207PZ, G63069TR); (Hex)1 (Deoxyhexose)1 (G00068MO); (Hex)3 (HexNAc)3 (Deoxyhexose)2 (G35949CT, G71094KR, G08426KY); (Hex)2 (HexNAc)2 (Deoxyhexose)2 (G23119MJ); (Hex)1 (HexNAc)1 (NeuAc)1 (G65562ZE); (Hex)3 (HexNAc)2 (Deoxyhexose)2 (G20310DM, G78177LC); (Hex)1 (HexNAc)2 (G00033MO, G61730RY, G56868BH); (Hex)1 (HexNAc)2 (Deoxyhexose)2 (G89748NG, G09520ZQ); (Hex)2 (HexNAc)2 (Deoxyhexose)1 (G94514IB, G61216ZY); and (Hex)2 (HexNAc)1 (Deoxyhexose)1 (G73318SN, G33986KK).
97. The composition of embodiment 96, wherein the MUC5B glycan comprises a formula selected from the group consisting of (Hex)1 (HexNAc)1 (Deoxyhexose)1 (G94435QH); (Hex)1 (HexNAc)1 (G76355TG, G85856KC); (Hex)1 (HexNAc)2 (Deoxyhexose)1 (G32426JY, G74353FF); (Hex)1 (NeuAc)1 (G30207PZ, G63069TR); and (Hex)1 (Deoxyhexose)1 (G00068MO).
98. The composition of embodiment 97, wherein the mucin glycan comprises a MUC5AC glycan.
99. The composition of embodiment 98, wherein the MUC5AC glycan comprises a formula selected from the group consisting of (Hex)1 (HexNAc)1 (G76355TG, G85856KC); (Hex)1 (HexNAc)2 (G00033MO, G61730RY, G56868BH); (Hex)1 (HexNAc)1 (Deoxyhexose)1 (G94435QH); (Hex)1 (Deoxyhexose)1 (G00068MO); (Hex)2 (HexNAc)3 (Deoxyhexose)1 (G91459EI, G26724PP, G52132CU); (Hex)2 (HexNAc)4 (G34764BK, G12074QJ); (Hex)2 (HexNAc)2 (Deoxyhexose)1 (G94514IB, G61216ZY); (Hex)2 (HexNAc)3 (G96915PP, G13483MW); (Hex)2 (HexNAc)2 (G64973KT); (Hex)3 (HexNAc)2 (Deoxyhexose)2 (G20310DM, G78177LC); (Hex)3 (HexNAc)3 (Deoxyhexose)1 (G66741QE, G16458JH); (Hex)3 (HexNAc)4 (G94517VF, G57672ST); (Hex)2 (HexNAc)4 (Deoxyhexose)1 (G23700TV); (Hex)2 (HexNAc)1 (Deoxyhexose)1 (G73318SN, G33986KK); (Hex)1 (HexNAc)2 (Deoxyhexose)1 (G32426JY, G74353FF); (Hex)2 (HexNAc)2 (Deoxyhexose)2 (G23119MJ); (Hex)1 (HexNAc)3 (G68893BQ, G23438NR); (Hex)3 (HexNAc)4 (Deoxyhexose)1 (G02990AF, G29956GF); (Hex)1 (HexNAc)1 (NeuAc)1 (G65562ZE); (Hex)3 (HexNAc)4 (Deoxyhexose)2 (G93333OF); (Hex)3 (HexNAc)2 (Deoxyhexose)1 (G15849KC, G81911LP, G41486OC); (Hex)2 (HexNAc)1 (G28052FT); (Hex)3 (HexNAc)3 (Deoxyhexose)2 (G35949CT, G71094KR, G08426KY); (Hex)3 (HexNAc)3 (G86537AD, G89585FG); (Hex)1 (HexNAc)2 (NeuAc)1 (G85608AG, G64844ET); (Hex)3 (HexNAc)5 (G00505CR); (HexNAc)1 (G57321FI); (Hex)1 (HexNAc)4 (G59229NY); (Hex)4 (HexNAc)3 (Deoxyhexose)2 (G68308CM); (Hex)3 (HexNAc)5 (Deoxyhexose)1 (G05252QE); (Hex)1 (HexNAc)3 (Deoxyhexose)1 (G28878FC); (Hex)2 (HexNAc)1 (NeuAc)1 (G59126YU); (Hex)4 (HexNAc)4 (Deoxyhexose)1 (G94768NG); (Hex)2 (HexNAc)2 (NeuAc)1 (G46748BU); (Hex)3 (HexNAc)2 (G16404NW, G25323VU); (Hex)2 (HexNAc)3 (Deoxyhexose)2 (G68384KC, G39326PP); (Hex)1 (HexNAc)1 (Deoxyhexose)1 (NeuAc)1 (G47180UC); (Hex)4 (HexNAc)4 (Deoxyhexose)2 (G98518WL); (Hex)2 (HexNAc)4 (Deoxyhexose)2 (G90965BZ); (Hex)2 (HexNAc)5 (G23048PE); (Hex)4 (HexNAc)5 (Deoxyhexose)2 (G82251ZP, G18603ZQ); (Hex)3 (HexNAc)6 (G62609ZF); (Hex)2 (HexNAc)1 (Deoxyhexose)2 (G68200GL); (Hex)4 (HexNAc)4 (Deoxyhexose)3 (G66166BF); (Hex)4 (HexNAc)5 (Deoxyhexose)1 (G92547QZ); (Hex)3 (HexNAc)5 (Deoxyhexose)2 (G28921PH); (Hex)2 (HexNAc)5 (Deoxyhexose)1 (G09396HG); (Hex)6 (HexNAc)5 (Deoxyhexose)3 (G18501TC); (Hex)4 (HexNAc)6 (Deoxyhexose)1 (G32752FJ); (Hex)5 (HexNAc)6 (Deoxyhexose)3 (G65612SS); (Hex)7 (HexNAc)6 (Deoxyhexose)1 (G61898SS); (Hex)5 (HexNAc)4 (Deoxyhexose)2 (G21630AC); (Hex)5 (HexNAc)5 (Deoxyhexose)1 (G25957KN); (Hex)6 (HexNAc)5 (Deoxyhexose)1 (G37901JE, G84713IO); (Hex)6 (HexNAc)7 (Deoxyhexose)2 (G29852ZH); (Hex)4 (HexNAc)6 (Deoxyhexose)2 (G99804SJ); (Hex)5 (HexNAc)5 (Deoxyhexose)3 (G90829NZ); (Hex)6 (HexNAc)5 (Deoxyhexose)2 (G70999YJ, G0268IFY); and (Hex)5 (HexNAc)6 (Deoxyhexose)2 (G44467ZE).
100. The composition of embodiment 99, wherein the MUC5AC glycan comprises a formula selected from the group consisting of (Hex)1 (HexNAc)1 (G76355TG, G85856KC); (Hex)1 (HexNAc)2 (G00033MO, G61730RY, G56868BH); (Hex)1 (HexNAc)1 (Deoxyhexose)1 (G94435QH); (Hex)1 (Deoxyhexose)1 (G00068MO); (Hex)2 (HexNAc)3 (Deoxyhexose)1 (G91459EI, G26724PP, G52132CU); (Hex)2 (HexNAc)4 (G34764BK, G12074QJ); (Hex)2 (HexNAc)2 (Deoxyhexose)1 (G94514IB, G61216ZY); (Hex)2 (HexNAc)3 (G96915PP, G13483MW); (Hex)2 (HexNAc)2 (G64973KT); (Hex)3 (HexNAc)2 (Deoxyhexose)2 (G20310DM, G78177LC); (Hex)3 (HexNAc)3 (Deoxyhexose)1 (G66741QE, G16458JH); (Hex)3 (HexNAc)4 (G94517VF, G57672ST); (Hex)2 (HexNAc)4 (Deoxyhexose)1 (G23700TV); (Hex)2 (HexNAc)1 (Deoxyhexose)1 (G73318SN, G33986KK); (Hex)1 (HexNAc)2 (Deoxyhexose)1 (G32426JY, G74353FF); (Hex)2 (HexNAc)2 (Deoxyhexose)2 (G23119MJ); (Hex)1 (HexNAc)3 (G68893BQ, G23438NR); (Hex)3 (HexNAc)4 (Deoxyhexose)1 (G02990AF, G29956GF); and (Hex)1 (HexNAc)1 (NeuAc)1 (G65562ZE).
101. The composition of embodiment 100, wherein the MUC5AC glycan comprises a formula selected from the group consisting of (Hex)1 (HexNAc)1 (G76355TG, G85856KC); (Hex)1 (HexNAc)2 (G00033MO, G61730RY, G56868BH); (Hex)1 (HexNAc)1 (Deoxyhexose)1 (G94435QH); (Hex)1 (Deoxyhexose)1 (G00068MO); (Hex)2 (HexNAc)3 (Deoxyhexose)1 (G91459EI, G26724PP, G52132CU); (Hex)2 (HexNAc)4 (G34764BK, G12074QJ); (Hex)2 (HexNAc)2 (Deoxyhexose)1 (G94514IB, G61216ZY); (Hex)2 (HexNAc)3 (G96915PP, G13483MW); (Hex)2 (HexNAc)2 (G64973KT); (Hex)3 (HexNAc)2 (Deoxyhexose)2 (G20310DM, G78177LC); (Hex)3 (HexNAc)3 (Deoxyhexose)1 (G66741QE, G16458JH); and (Hex)3 (HexNAc)4 (G94517VF, G57672ST).
102. The composition of embodiment 101, wherein the MUC5AC glycan comprises a formula selected from the group consisting of (Hex)1 (HexNAc)1 (G76355TG, G85856KC); (Hex)1 (HexNAc)2 (G00033MO, G61730RY, G56868BH); (Hex)1 (HexNAc)1 (Deoxyhexose)1 (G94435QH); (Hex)1 (Deoxyhexose)1 (G00068MO); (Hex)2 (HexNAc)3 (Deoxyhexose)1 (G91459EI, G26724PP, G52132CU); and (Hex)2 (HexNAc)4 (G34764BK, G12074QJ).
103. The composition of embodiment any one of embodiments 87-102, wherein the mucin glycan is a unsulfated mucin glycan.
104. The composition of embodiment any one of embodiments 87-102, wherein the mucin glycan is a sulfated mucin glycan.
105. The composition of embodiment 104, wherein the sulfated mucin glycan comprises a formula selected from the group consisting of S1 (Hex)1 (HexNAc)1 (G10634LC); S1 (Hex)1 (HexNAc)1 (Deoxyhexose)1 (G08671QK); S1 (Hex)1 (HexNAc)2 (G32406CO); S1 (Hex)2 (HexNAc)1 (Deoxyhexose)1 (G24803MV); S1 (Hex)1 (HexNAc)2 (Deoxyhexose)1 (G10520JC); S1 (Hex)1 (HexNAc)2 (NeuAc)1 (G96888OD); S1 (Hex)2 (HexNAc)2 (Deoxyhexose)1 (G24803MV); S1 (Hex)1 (HexNAc)2 (NeuGc)1 (G60426XC); S1 (Hex)1 (HexNAc)3 (Deoxyhexose)1 (G72091WB, G60644GY); and S1 (Hex)2 (HexNAc)3 (Deoxyhexose)1 (G35891PL).
106. The composition of embodiment 104, wherein the sulfated mucin glycan comprises a sulfated MUC2 glycan, a sulfated MUC5AC glycan, a sulfated MUC5B glycan, or a combination thereof.
107. The composition of embodiment 106, wherein:
   a) the sulfated MUC2 glycan comprises a formula selected from the group consisting of S1 (Hex)1 (HexNAc)2 (G32406CO); S1 (Hex)1 (HexNAc)2 (Deoxyhexose)1 (G10520JC); S1 (Hex)1 (HexNAc)2 (NeuAc)1 (G96888OD); S1 (Hex)1 (HexNAc)3 (Deoxyhexose)1 (G72091WB, G60644GY); and S1 (Hex)1 (HexNAc)2 (NeuGc)1 (G60426XC);
   b) the sulfated MUC5B glycan comprises a formula selected from the group consisting of S1 (Hex)2 (HexNAc)1 (Deoxyhexose)1 (G24803MV); S1 (Hex)1 (HexNAc)1 (Deoxyhexose)1 (G08671QK); S1 (Hex)1 (HexNAc)1 (G10634LC); S1 (Hex)2 (HexNAc)2 (Deoxyhexose)1 (G24803MV); and S1 (Hex)2 (HexNAc)3 (Deoxyhexose)1 (G35891PL); or
   c) the sulfated MUC5AC glycan comprises a formula selected from the group consisting of S1 (Hex)1 (HexNAc)2 (Deoxyhexose)1 (G10520JC); S1 (Hex)2 (HexNAc)2 (Deoxyhexose)1 (G24803MV); S1 (Hex)2 (HexNAc)1 (Deoxyhexose)1 (G24803MV); S1 (Hex)2 (HexNAc)3 (Deoxyhexose)1 (G35891PL); and S1 (Hex)1 (HexNAc)2 (G32406CO).

### EXAMPLES

Candida albicans is an opportunistic fungal pathogen that asymptomatically colonizes the mucosal surfaces of most healthy humans^{1,2}. Alterations to the mucus barrier and microbiota can lead to C. albicans overgrowth and infection, causing conditions such as oral thrush, vulvovaginal candidiasis, and life-threatening systemic candidiasis^{1,3}. The scarcity of antifungal drug classes, their limited efficacy, toxicity, and the development of resistance⁴ contribute to a mortality rate of ˜40% in deep-seated candidiasis³, highlighting an urgent need for alternative treatments to fungal infections.

Targeting pathogenic mechanisms rather than growth represents an attractive approach for developing novel antimicrobial agents. The infection of diverse host niches is supported by a wide range of C. albicans virulence and fitness attributes, including the morphological yeast-to-hyphal transition (filamentation), adhesin expression, biofilm formation, and the secretion of hydrolytic enzymes that damage the underlying epithelium⁵. The yeast-to-hyphal transition is a major virulence factor⁵ and is integral for robust biofilms, which are intrinsically resistant to treatment, posing a significant clinical challenge⁶. Strikingly, despite its potential for pathogenicity, C. albicans is accommodated in healthy mucus⁷, suggesting that mucus may underpin novel strategies for preventing C. albicans virulence.

Mucus is a complex viscoelastic secretion that coats all non-keratinized epithelial surfaces in the body that are exposed to and communicate with the external environment⁸. Much of the microbiota is housed in the mucus layer, serving as a protective barrier and microbial niche^{8,9}. Mucins are the main structural component of mucus and play an integral role in attenuating virulence traits in various cross-kingdom pathogens, including C. albicans^{2,10}. Mucin exposure suppresses C. albicans virulence phenotypes, including the formation of host-cell-penetrating hyphae¹⁰. However, the mechanisms through which mucins attenuate virulence in C. albicans remain unknown, impeding their application for therapeutic intervention.

To close this gap, the mechanism and biochemical motifs of mucins that suppress C. albicans virulence gene expression and phenotypes were characterized. By isolating and characterizing mucin-derived glycans across major mucosal surfaces, it was determined that mucin glycans repress C. albicans virulence traits including filamentation, adhesion, and biofilm formation and alter fungal-bacterial dynamics. It was identified that specific Core-1- and Core-2-modified glycan structures within the mucin polymer suppress filamentation and downregulate filamentation-associated genes in C. albicans. These results elucidate the mechanisms by which healthy mucins attenuate C. albicans pathogenicity, suggesting therapeutic candidates for treating C. albicans infection without disrupting the microbiota (and potential evolution of antifungal resistance) that normally accompanies the killing of cells.

### Example 1. Methods

C. albicans Strains and Media

Strains were maintained on yeast extract peptone dextrose (YPD) agar (2% Bacto peptone, 2% glucose, 1% yeast extract, 2% agar) and grown at 30° C. Single colonies were inoculated into YPD broth and grown with shaking overnight at 30° C. prior to each experiment. Experiments were performed with Gibco RPMI 1640 medium (Life Technologies, Carlsbad, CA; #31800-089) buffered with 165 mM 3-(N-morpholino)propanesulfonic acid (MOPS) and supplemented with 0.2% NaHCO₃ and 2% glucose; YPD medium with 10% fetal bovine serum; GlcNAc medium (0.5% N-acetylglucosamine, 0.5% peptone, 0.3% KH₂PO₄); Spider medium (1% nutrient broth, 1% D-mannitol, 2 g K₂HPO₄, 50 mg/mL arginine, 10 mg/mL histidine, and 50 mg/mL tryptophan); or Lee's medium⁴⁵. Growth curves were performed in Synthetic Defined (SD)+0.004% (w/v) L-Arginine+0.0025% (w/v) L-Leucine media with 2% glucose.

The C. albicans reference strains used in this study were SC5314 and HGFP3. Strain HGFP3 was constructed by inserting the GFP gene next to the promoter of HWP1, a gene encoding a hyphal cell wall protein, in SC5314; this strain was provided by E. Mylonakis (Massachusetts General Hospital, Boston, MA) with the permission of P. Sundstrom. Homozygous deletion strains were obtained from the transcriptional factor deletion collection and were provided by the Fungal Genome Stock Center (www.fgsc.net). The following C. albicans strains used for pathway analyses were gifts from Paul Kauman (University of Massachusetts, Medical School): AV55 (ura3::λimm434/ura3::λimm 434; LEU2::pCK1-efg1-T206E::URA3); DH409 (ura3::λimm434/ura3; ras1-G13V); and CDH72-1 (ura3/ura3 cph1Δ::hisG/cph1Δ::hisG; ADH1prCPH1).

Collection of Human Saliva

Submandibular saliva was collected from healthy human volunteers using a custom vacuum pump, pooled, centrifuged at 2,500×g for 5 min, and phenylmethylsulfonylfluoride (1 mM) was added.

Mucin Purification

This study used native porcine gastric mucins (MUC5AC), porcine intestinal mucins (MUC2), and human salivary mucins (MUC5B), which differ from industrially purified mucins in their rheological properties and bioactivities^{10,46}. Native mucins were purified as described previously^{10,18}. In brief, mucus was scraped from fresh pig stomachs and intestines and solubilized in sodium chloride. Insoluble material was removed via ultracentrifugation at 190,000×g for 1 h at 4° C. (Beckman 50.2 Ti rotor with polycarbonate bottles). Submandibular saliva was collected from human volunteers as described above using a custom vacuum pump, pooled, centrifuged, and protease inhibitors were added¹⁰. Mucins were purified using size-exclusion chromatography on separate Sepharose CL-2B columns. Mucin fractions were then desalted, concentrated, and lyophilized for storage at −80° C. Lyophilized mucins were reconstituted by shaking them gently at 4° C. overnight in the desired medium.

Mass spectrometry is routinely used to monitor the composition of purified mucin extracts. This type of analysis has shown that mucin extracts purified from porcine stomach mucus, for example, are composed predominantly of MUC5AC, with small quantities of MUC2, MUC5B, and MUC6, as well as histones, actin, and albumin⁴⁷.

Isolation of Mucin Oligosaccharides

Non-reductive alkaline β-elimination ammonolysis was applied to dissociate non-reduced glycans from mucins as described previously^{18,48}. Purified mucins were dissolved in ammonium hydroxide saturated with ammonium carbonate and incubated at 60° C. for 40 h to release oligosaccharide glycosylamines and partially deglycosylated mucins. Volatile salts were removed using repeated centrifugal evaporation and the oligosaccharide glycosylamines were separated from residual deglycosylated mucins via centrifugal filtration through 3-5 kDa molecular weight cut-off membranes (Amicon, Miami, FL; Ultracel) in accordance with the manufacturer's instructions. The resulting oligosaccharide glycosylamines were converted to reducing oligosaccharide hemiacetals via treatment with boric acid. Residual boric acid was removed via repeated centrifugal evaporation from methanol. Oligosaccharides were further purified using solid-phase extraction using Hypercarb mini-columns (ThermoFisher, Waltham, MA) and residual solvents were removed through centrifugal evaporation.

Analysis of Mucin O-Glycan Profiles

Glycans released from MUC2, MUC5B, and MUC5AC were permethylated and analyzed by nanospray ionization tandem mass spectrometry (NSI-MS) following direct infusion into a linear/orbital hybrid ion trap instrument (Orbitrap-LTQ Discovery, ThermoFisher) operated in positive ion mode for non-sulfated glycans or in negative mode for the detection of sulfated glycans. The permethylated O-glycans were dissolved in 1 mM sodium hydroxide in methanol/water (1:1) for infusion at a syringe flow rate of 0.60 μl/min and capillary temperature set to 210° C.⁴⁹. For fragmentation by collision-induced dissociation (CID) in MS/MS and MSn, a normalized collision energy of 35-40% was applied. Detection and relative quantification of the prevalence of individual glycans was accomplished using the total ion mapping (TIM) functionality of the Xcalibur software package version 2.0 (ThermoFisher) as previously described⁴⁹. For TIM, the m/z range from 600 to 2000 was automatically scanned in successive 2.8 mass unit windows with a window-to-window overlap of 0.8 mass units, which allowed the naturally occurring isotopes of each glycan species to be summed into a single response, thereby increasing detection sensitivity. Most glycan components were identified as singly, doubly, and/or triply charged, sodiated species (M+Na) in positive mode or as singly or doubly charged (M−H) species in negative mode. Charge states for each glycan were deconvoluted manually and summed for quantification. Structural representations of mucin glycans were based on topologic features detected upon CID fragmentation and knowledge of O-glycan biosynthetic pathways. Approximately 33% of the m/z values reported here were associated with 2 or 3 isomeric glycan structures. NSI-MS/MS and MSⁿ were used as needed to assign isomeric heterogeneity at each of these m/z values. For purposes of representing and comparing the heterogeneity of the glycan profile associated with each mucin, the signal intensity associated with an m/z value comprised of more than one glycan was assigned to the most abundant glycan structure among the isomers. Graphic representations of glycan monosaccharide residues are consistent with the Symbol Nomenclature For Glycans (SNFG) as adopted by the glycomics and glycobiology communities. Glycomics data and metadata were obtained and are presented in accordance with MIRAGE standards and the Athens Guidelines⁵⁰. GlyTouCan accessions were retrieved from the GlyTouCan repository through GlyGen for glycan instances in which accessions already existed. If new accessions were required for glycans not previously placed in the repository, the desired structural representations were generated in GlycoGlyph and submitted directly to GlyTouCan for registration⁵¹. All raw mass spectrometric data related to mucin glycan profiles were deposited at GlycoPost⁵². Heatmap and other data analysis was performed on extracted signal intensities using Prism GraphPad and Excel software.

Filamentation Assay

Hyphal growth of C. albicans was induced by diluting cells to OD₆₀₀=0.05 into prewarmed hyphae-inducing medium as indicated and incubating at 37° C. (200 rpm) in a glass-bottom, 96-well plate. Cells were grown in hyphae-inducing medium for several hours, as described in BRIEF DESCRIPTION OF THE DRAWINGS. Images were acquired with a confocal laser scanning microscope (Zeiss, Oberkochen, Germany; LSM 800) equipped with a ×63/1.4 NA oil-immersion or a 25× objective. Images were analyzed using Zeiss ZEN v.2.1. Representative micrographs are shown.

RNA Extraction

For the extraction of the RNA of C. albicans grown in the presence or absence of mucins, 1 mL of RPMI or 0.5% w/v MUC2, MUC5AC, or MUC5B in RPMI was inoculated with 10 μL of an overnight culture of strain SC5314 and incubated in a culture tube at 37° C. with shaking (180 rpm) for 8 h. Total RNA was extracted using the Epicentre MasterPure Yeast RNA Purification Kit and treated with Sigma-Aldrich (St. Louis, MO) AMPD1 amplification-grade DNase I.

For RNA extraction from C. albicans grown in the presence or absence of mucin glycans, 100 μL of RPMI or 0.1% w/v MUC5AC glycans in RPMI were inoculated with a 1:50 dilution of an overnight culture of strain SC5314 and incubated at 37° C. for the time indicated. Total RNA was extracted with the MasterPure RNA Purification Kit (Lucigen, Middleton, WI) and residual DNA was removed using the Turbo DNA-free kit (Ambion, Austin, TX). The integrity of the total RNA was assessed using an Agilent 2100 Bioanalyzer (Agilent Technologies, Santa Clara, CA). rRNA was removed using the Ribo-Zero® rRNA Removal Kit (Yeast; Epicentre).

RNA Sequencing

For RNA sequencing of C. albicans grown in mucins, poly(A) RNA was isolated from total RNA via two rounds of purification. Samples were run on a MiSeq with a paired-end protocol and read lengths of 150 bp.

For RNA sequencing of C. albicans grown in mucin glycans, Illumina RNA-seq was used. Libraries were produced using the KAPA RNA HyperPrep kit (Kapa Biosystems, Wilmington, MA) and sequenced using the Illumina HiSeq platform with a single-end protocol and read lengths of 40 or 50 nucleotides.

RT-qPCR Analysis

A list of the primers used in this study is provided in Table 1. qPCR with reverse transcription (RT-qPCR) was performed using a two-step method. First-strand cDNA was synthesized from total RNA using the ProtoScript IV First Strand cDNA Synthesis kit (New England Biolabs, Ipswich, MA). The cDNA was used as template for RT-qPCR using a SYBR PowerUp Master Mix kit (Applied Biosystems, Life Technologies) on a Roche LightCycler 480 real-time PCR system. The ACT1 and 18S rRNA genes were used as endogenous controls as specified. The elimination of contaminating DNA was confirmed using qPCR amplification of ACT1 and 18S rRNA genes on control samples that did not have reverse transcriptase added during cDNA synthesis. Changes in gene expression were calculated based on mean change in qPCR cycle threshold (ΔCt) using the ΔΔCₜ method (fold change=2−ΔΔCt−ΔΔCt).

TABLE 1
Primer Sequences

| SEQ ID | | |
|---|---|---|
| NO: 1 | ECE1_F | TGCCATTTGTTGTCAGAGCTG |
| NO: 2 | ECE1_R | TAGCTTGTTGAACAGTTTCCAGG |
| NO: 3 | HWP1_F | GCTGGTTCAGAATCATCCATGC |
| NO: 4 | HWP1_R | AAGGTTCAGTGGCAGGAGCTG |
| NO: 5 | HGC1_F | GTCAGCTTCCTGCACCTCATC |
| NO: 6 | HGC1_R | AAACAGCACGAGAACCAGCG |
| NO: 7 | NRG1_F | GGTTGCACGTTGTCGAAACC |
| NO: 8 | NRG1_R | TGTTGCTGCTGCTGCTTGG |
| NO: 9 | ACT1_F | CCCAGGTATTGCTGAACGTA |
| NO: 10 | ACT1_R | GAACCACCAATCCAGACAGA |
| NO: 11 | YWP1_F | TGCTAGTACTGCTAACAAAGTCAC |
| NO: 12 | YWP1_R | CACCATTAACACCACCAGCA |
| NO: 13 | UME6_F | TCATTCAATCCTACTCGTCCACC |
| NO: 14 | UME6_R | CCAGATCCAGTAGCAGTGCTG |
| NO: 15 | RIP1_F | TGCTGACAGAGTCAAGAAACC |
| NO: 16 | RIP1_R | GAACCAACCACCGAAATCAC |
| NO: 17 | EFG1_F | CATCACAACCAGGTTCTACAACCAAT |
| NO: 18 | EFG1_R | CTACTATTAGCAGCACCACCC |
| NO: 19 | 18S_F | GGATTTACTGAAGACTAACTACTG |
| NO: 20 | 18S_R | GAACAACAACCGATCCCTAGT |
| NO: 21 | ALS3_F | ACTTCCACAGCTGCTTCCACTTCT |
| NO: 22 | ALS3_R | TCCACGGAACCGGTTGTTGCT |
| NO: 23 | HYR1_F | CGGTTCTGGAAGTGGTCATAA |
| NO: 24 | HYR1_R | AGAGTGTGAACCTGCGTTAG |
| NO: 25 | EED1_F | TGCTCTACCACCACAACAAG |
| NO: 26 | EED1_R | TTGCGGTGCTTGCTCATA |

Analysis of RNA Sequencing Data

For mucins RNA sequencing experiments, reads were mapped to the C. albicans SC5314 haplotype A genome, version A22-s05-m05-r03, retrieved from the Candida Genome Database (www.candidagenome.org) using Rsubread v1.28.1⁵³. Read summarization was performed on the gene level using featureCounts⁵⁴ using annotations from a modified version of C. albicans SC5314 haplotype A, which only contained protein coding genes. Multimapping read pairs, read pairs mapping across more than one gene, and read pairs in which ends mapped to different chromosomes were removed from downstream analyses. Genes that had <10 counts per million in at least two samples were discarded. Remaining gene counts were normalized using trimmed mean of M-values⁵⁵. Differential expression analysis was performed using limma v3.34.9⁵⁶ with voom-transformed read counts⁵⁷. Genes were considered differentially expressed when P<0.05 after the false-discovery rate was controlled using Benjamini-Hochberg correction.

For mucin glycans RNA sequencing experiments, reads were mapped to the C. albicans SC5314 haplotype A genome, version A22-s05-m05-r03, retrieved from the Candida Genome Database (www.candidagenome.org) using the Galaxy Server⁵⁸. Read summarization was performed on the gene level with annotations from a modified version of C. albicans SC5314 haplotype A. Multimapping read pairs, read pairs mapping across more than one gene, and read pairs in which ends mapped to different chromosomes were removed from downstream analyses. Differential expression analysis was performed using DESEQ2⁵⁹. Genes were considered differentially expressed when P<0.05 after the false-discovery rate was controlled using Benjamini-Hochberg correction.

Functional category (pathway) assignments were obtained from Candida Genome Database Gene Ontology annotations and assessed using PANTHER⁶⁰. Over-representation of biological pathways in mucin was assessed using one-sided Fisher's exact test followed by a Benjamini-Hochberg procedure for multiple corrections, for differentially expressed genes from n=3 replicates. Enrichment of pathways in MUC5AC glycans was determined based on mean log₂-transformed fold changes from n=3 replicates and calculated with the two-sided Mann-Whitney U-test followed by a Benjamini-Hochberg procedure for multiple corrections. Heat maps and scatter plots of gene expression data were constructed using GraphPad Prism.

Murine Wound C. albicans Protocol

Female, 8-week old, SKH-1 mice were anesthetized with isoflurane and given buprenorphine (0.05 mg/kg) before wounding with a 6-mm punch biopsy to generate two identical full-thickness dermal wounds on the dorsal side of the mouse. Wounds were kept covered with an occlusive dressing (Opsite Flexifix) throughout the duration of the experiment. After a 24 h recovery period, wounds were inoculated with 30 μl volume of PBS containing 10⁸ SC513 Enol-mCherry yeast. Topical treatments of 30 μl of 0.5% MUC2, or PBS were administered to each wound on Day 1, 3, and 5 post-infection. Wounds were gently washed with 500 ul of sterile PBS and bandages changed prior to each treatment. Wound fluorescence was imaged daily with IVIS Lumina II optical imaging system to assess fungal burden. At Day 5 and Day 7, wound biopsy specimens were collected and CFUs were calculated per gram of tissue.

The mice used in this experiment were housed at 72° F. at 30% humidity with a 12-hour light/dark cycle.

Polystyrene Attachment Assay

Strain HGFP3 was pregrown overnight in YPD medium at 30° C., diluted to OD₆₀₀=0.1, added into prewarmed RPMI medium in a polystyrene 96-well plate, and incubated at 37° C. for the time indicated in the figure legend. Medium was decanted and plates were washed three times with phosphate-buffered saline. Images were acquired with a confocal laser scanning microscope (LSM 800) equipped with a ×20/1.4 NA objective. The excitation wavelength for GFP was 488 nm. Four images were recorded for each well and for at least three independent wells. Images were analyzed in Fiji as follows: each image was converted to 8-bit and the contrast was enhanced (0.4% saturated pixels), then thresholded to create a binary image. Each image was analyzed using the Analyze Particles tool to measure the surface area covered by cells as described previously¹⁰. The mean surface area measurements of the images for each condition were calculated.

Biofilm Formation Assays and Visualization

In vitro biofilm growth assays were carried out in RPMI medium by growing the biofilm directly on a 96-well polystyrene plate. Briefly, strain SC5314 was grown overnight in YPD at 30° C., washed twice with phosphate-buffered saline, then diluted to OD₆₀₀=0.5 in 100 μL of RPMI in a 96-well polystyrene plate. The inoculated plate was incubated at 37° C. for 90 min to facilitate attachment of yeast cells to the surface. Nonadherent cells were washed twice with phosphate-buffered saline, and samples were subsequently submerged in fresh RPMI. Biofilms were grown for 24 h at 37° C. For CFU enumeration, the medium containing the planktonic cells was removed and plated on YPD plates. Biofilms were resuspended with phosphate-buffered saline, disrupted by pipetting, serially diluted in phosphate-buffered saline, and plated on YPD plates. Biofilms and planktonic cells were imaged using a Zeiss wide-field fluorescence microscope.

Coculture Viability Assays

An overnight SC5314 culture grown in YPD was diluted 1:100 into RPMI in a 96-well plate (MatTek, Ashland, MA) with or without 0.1% MUC5AC glycans and grown for 4 h with shaking at 37° C. A control well without C. albicans was included. Concurrently, 2 ml of LB were inoculated with 40 l Pseudomonas aeruginosa strain PA14 and grown for 4 h with shaking at 37° C. RPMI was then removed from C. albicans and replaced with 200 μl SLB. P. aeruginosa was added to a final OD₆₀₀=0.25. At 0 h, 24 h, 48 h, and 72 h, the contents of the wells were homogenized and a 5-μl aliquot was serially diluted in phosphate-buffered saline. Dilutions were plated on YPD agar+Gm³⁰+Tet⁶⁰ (to select for C. albicans) and Cetrimide agar (to select for P. aeruginosa) and incubated overnight at 30° C. and 37° C., respectively. Colonies were counted after incubation.

Confocal Imaging of Coculture

Images were acquired with a confocal laser scanning microscope (LSM 800) equipped with a ×63/1.4 NA oil-immersion objective. Images were analyzed using Zeiss ZEN v.2.1. C. albicans was stained with 20 μg/ml calcofluor white. The excitation and emission wavelengths for calcofluor white were 365 nm and 445 nm, respectively; the excitation and emission wavelengths for mCherry were 587 nm and 610 nm, respectively.

Glycan Synthesis and Analysis

All commercial reagents were used as supplied unless otherwise stated, and solvents were dried and distilled using standard techniques. Thin layer chromatography was performed on silica-coated glass plates (TLC Silica Gel 60 F₂₅₄; Merck, Rahway, NJ) with detection via fluorescence, charring with 5% H₂SO_{4(aq)}, or staining with a ceric ammonium molybdate solution. Organic solutions were concentrated and/or evaporated to dry under vacuum in a water bath (<50° C.). Molecular sieves were dried at 400° C. under vacuum for 20-30 min prior to use. Amberlite IR-120H resin was washed extensively with MeOH and dried under vacuum prior to use. Medium-pressure liquid chromatography was performed using a CombiFlash Companion equipped with RediSep normal-phase flash columns, and solvent gradients refer to sloped gradients with concentrations reported as % v/v. Nuclear magnetic resonance spectra were recorded on a Bruker Avance DMX-500 (500 MHz) spectrometer, and assignments achieved with the assistance of 2D gCOSY, 2D gTOCSY, 2D gHSQC, and 2D gHMBC; chemical shifts are expressed in ppm and referenced to either Si(CH₃)₄ (for CDCl₃), residual CHD₂OD (for CD₃OD), or a MeOH internal standard (for D₂O). Low-resolution electron-spray ionization mass spectrometry was performed with a Waters micromass ZQ. High-resolution mass spectrometry was performed with an Agilent 1100 LC equipped with a photodiode array detector, and a Micromass QTOF I equipped with a 4 GHz digital-time converter. Optical rotation was determined in a 10-cm cell at 20° C. using a Perkin-Elmer Model 341 polarimeter. High-performance liquid chromatography was performed with an Agilent 1100 LC equipped with an Atlantis T3 (3 mm, 2.1×100 mm) C18 column and ELSD detection.

Statistical Analysis

Unless noted otherwise, experiments were performed with at least three biological replicates consisting of at least three technical replicates, and results are presented as mean SEM. Microscopy images depicted are representative and similar results were observed in different fields of view across at minimum three independent biological replicates. Raw data are available as Source Data. MUC2, MUC5AC, and MUC5B and their associated glycans were tested from several purification batches with consistent results.

Data and Materials Availability

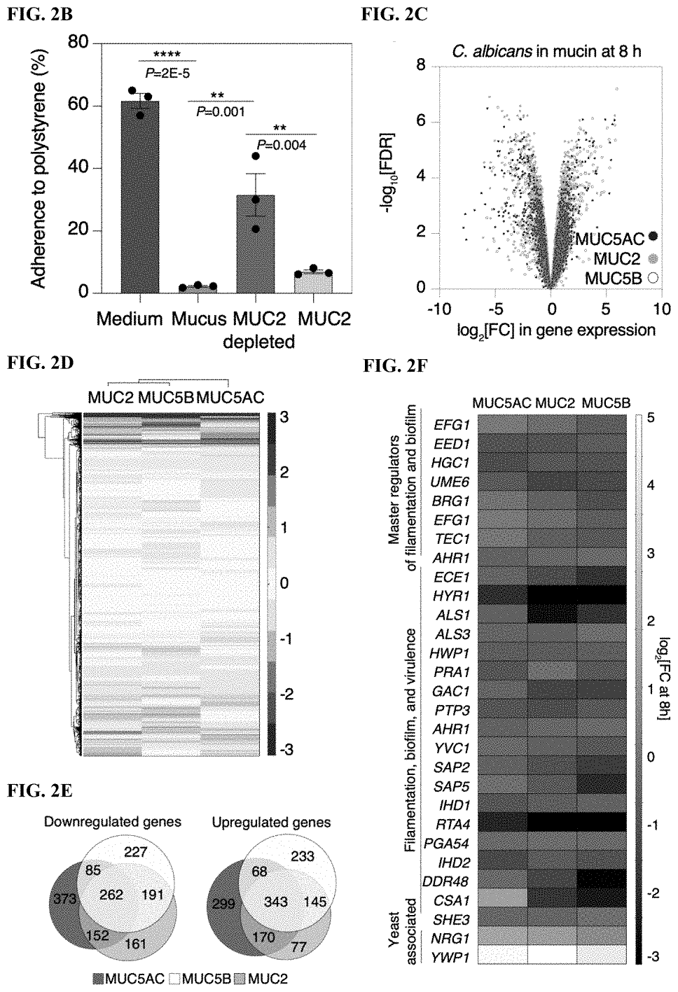

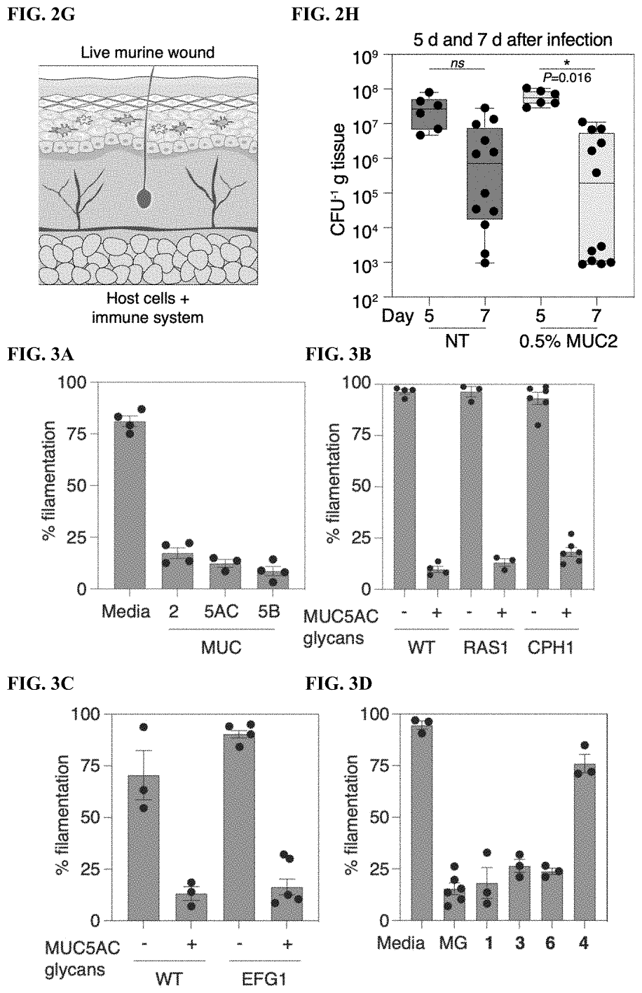

High-throughput sequencing data presented were deposited in the Gene Expression Omnibus (GEO) under accession number GSE197249 (FIGS. 2A-2H) and GSE192826 (FIGS. 4A-4G & 6A-6C). All raw mass spectrometric data related to mucin glycan profiles were deposited at GlycoPost, Accession #GPST000254 for non-sulfated glycans and Accession #GPST000258 for sulfated glycans. Additional information can be found in Takagi et al., Mucin O-glycans are natural inhibitors of Candida albicans pathogenicity, Nat Chem Biol. 18(7):762-73 (2022), www.ncbi.nlm.nih.gov/pmc/articles/PMC7613833/#SD15, the contents of which are incorporated herein by reference in their entirety.

Mucins are large gel-forming polymers inside the mucus barrier that inhibit the yeast to hyphal transition of Candida albicans, a key virulence trait of this important human fungal pathogen. However, the molecular motifs in mucins that inhibit filamentation remain unclear, despite their potential for therapeutic interventions. Here, it was determined that mucins display an abundance of virulence-attenuating molecules in the form of mucin O-glycans. Greater than 100 mucin O-glycans from three major mucosal surfaces were isolated and catalogued, and it was established that they suppress filamentation and related phenotypes relevant to infection, including surface adhesion, biofilm formation, and cross-kingdom competition between C. albicans and the bacterium Pseudomonas aeruginosa. Using synthetic O-glycans, three structures (Core 1, Core 1+fucose, and Core 2+galactose) were identified to be sufficient to inhibit filamentation with potency comparable to the complex O-glycan pool. Overall, Examples 2-6 identify mucin O-glycans as host molecules with untapped therapeutic potential to manage fungal pathogens.

### Example 2. Mucins Share a Conserved Function in Attenuating C. albicans Virulence

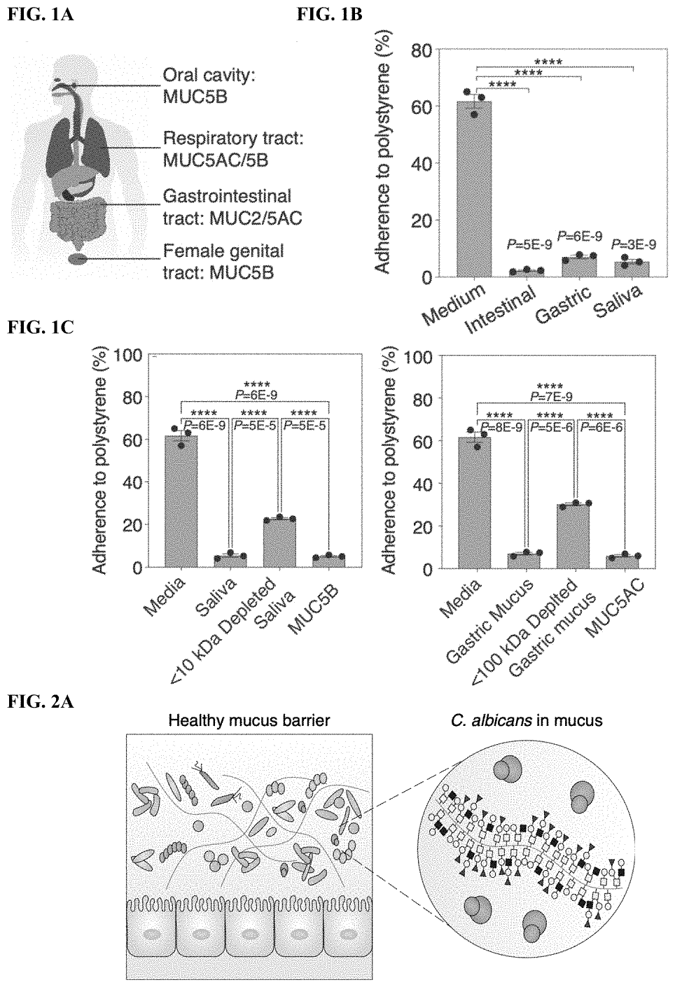

While previous studies identified mucin polymers as candidates for managing C. albicans virulence in vitro¹⁰, it is unclear whether mucin activity persists in native and complex mucus or in the context of an intact immune system, which may indicate whether mucins are viable candidates for therapeutic intervention on mucosal surfaces. Here, mucus from three distinct sources (FIG. 1A), porcine intestinal mucus, porcine gastric mucus, and human saliva—representing model systems¹¹⁻¹³ for mucosal niches colonized by C. albicans (FIG. 2A), were investigated. C. albicans adhesion to a polystyrene surface was quantified using fluorescence microscopy in medium with or without mucus. All three mucus types significantly decreased C. albicans adhesion in vitro (FIG. 1). Cell growth was similar with or without mucus; thus, mucus shifts C. albicans to a planktonic state without affecting viability.

One candidate for mediating this function is mucin polymers, which suppress C. albicans adhesion in vitro¹⁰. To determine whether mucins are the dominant adhesion-suppressing factor in mucus, high-molecular-weight components were removed from porcine intestinal mucus using a centrifugal filter with a 100-kDa cutoff. The filtrate was less effective in preventing adhesion than whole mucus (FIG. 2B). Re-introducing the filtrate with 0.5% w/v MUC2, the predominant mucin in the intestinal tract, restored this effect and significantly decreased adhesion; similar results were observed for all three mucin types (FIGS. 2B & 1C). Thus, mucins from these mucosal surfaces are both necessary and sufficient to recapitulate the adhesion-suppressing effects of mucus.

To clarify how mucins regulate C. albicans physiology, RNA sequencing was performed on cells grown in RPMI medium with or without (0.5% w/v) MUC2 (intestinal mucin), MUC5B (salivary mucin), or MUC5AC (gastric/respiratory mucin), representing mucins secreted on mucosal surfaces abundantly colonized by C. albicans. Each mucin type elicited a specific gene-expression profile (FIGS. 2C-2D), with 262 downregulated and 343 upregulated genes (P<0.05) shared among the three transcriptional profiles (FIG. 2E).

The 262 downregulated genes include, among others, ADH2, ALG1, ALG5, AMS1, AOX1, AOX2, ASM3, ATO1, ATO2, AYR2, BEM1, BMT1, BMT3, BTA1, BUD14, CCT6, CDC3, CDC4, CHS2, CHS3, CHS7, CHS8, CHT2, CIP1, CLA4, CLG1, CSH1, CSP2, CTF1, CTR1, CUP1, DEF1, DFI1, DPP3, ECE1, ERF1, EXG2, FAA4, FAV1, FAV2, FDH1, FRE30, FRE7, FRP3, GAC1, GAD1, GAP4, GCD2, GDT1, GLG2, GLX3, GRE2, GUT1, HAC1, HEX3, HGC1, HGT1, HGT2, HRT1, HSP21, HSP30, HTS1, HWP1, HYR1, IDH2, IFD6, IHD1, IHD2, ISU1, KEX2, KRE1, KRS1, LAC1, LYS143, MAC1, MAL31, MDM34, MDN1, MED7, MET10, MET14, MET3, MEU1, MKC1, MKK2, MNN12, MRV8, MSI3, NAM7, NIP1, PBR1, PCL1, PGA11, PGA13, PGA32, PGA54, PGI1, PHO112, PHO86, PHO89, PIN3, PRN1, PRN2, PRN3, PRN4, PST1, PTP3, RBT7, RFG1, RHB1, RHO2, RLM1, RPS42, RTA4, SAL6, SAP2, SAP5, SIR2, SKO1, SMF12, SMI1B, SOD3, SPO7, STB3, TEN1, THI13, TIF, UAP1, UGA1, UME6, WOR3, WSC1, YHB5, YKE2, YMC2, YOX1, YVC1, ZCF27, ZDS1, and ZFU2.

The 343 upregulated genes include, among others, ACO2, AFP99, AGE1, ALT1, AMO1, AMO2, APE3, ARO8, ARP4, ASN1, ATF1, ATM1, AXL1, BAT21, BAT22, BUB1, BUL1, CAN1, CAR1, CAR2, CAT8, CBF1, CDC14, CDC20, CDC21, CDC5, CHS1, CHT3, CLB4, CMK1, CRD2, CTP1, CWH8, DAG7, DAK2, DAO1, DAO2, DOA1, DOT1, DQD1, DSE1, DUR1,2, EBP7, ECM15, EMP46, ENG1, ERG1, ERG11, ERG13, ERG24, ERG251, ERG26, ERG27, ERG3, ERG5, ERG6, ERG9, FBA1, FCY21, FGR23, FGR29, FGR41, FGR50, FLO9, FMA1, GAL1, GAP1, GAP2, GAT1, GCN4, GCS1, GDH3, GIS2, GLN1, GLO1, GLT1, GOR1, GPD1, GPM2, GYP1, HEM13, HEM14, HEM3, HGT7, HIS1, HIS4, HIS5, HIS7, HMS1, HNM1, HOF1, HOM2, HOM6, HOS3, HRK1, HRQ2, HTA3, HYU1, IDP1, IFE2, IFG3, IFR2, ILV1, ILV3, ILV6, INT1, KIP1, KIP2, KTR4, LAP4, LCB4, LEU1, LEU2, LEU4, LIG1, LIP8, LYS1, LYS12, LYS144, LYS2, LYS22, LYS4, LYS5, LYS9, MBP1, MDM10, MED17, MEP1, MEP2, MIH1, MIS11, MNN22, MPH1, NCP1, NIT3, NPR1, NRG1, NUF2, NUP, OPT3, OPT4, PCL5, PCL7, PDE2, PGA38, PGA45, PGA48, PHO87, PIR1, POS5, PPS1, PRO1, PRO3, PTR22, PUT1, PUT2, QDR1, RAD32, RAD54, RBE1, RBT1, RHD1, RHD3, RIA1, RNH35, RNR1, RNR22, ROD1, RPD31, SCT1, SCW11, SEO1, SHE9, SIM1, SIZ1, SMC2, SMC4, SNF5, SNO1, SNZ1, SSN6, STP1, SUR2, SUT1, SWE1, SWI4, TFA1, TGL99, THI20, THR1, THR4, TNA1, TOA2, TOS4, TPO3, TRP4, TRP5, TYE7, UBP13, UPC2, VPS4, XKS1, YCG1, YCS4, YHB4, YWP1, and ZCF16.

Filamentation- and adhesion-regulating pathways were enriched in the shared downregulated genes. Notably, all three mucins caused the downregulation of various virulence-associated genes (FIG. 2F). Consistent with previous observations¹⁰, inoculation of C. albicans in RPMI medium caused the formation of extensive hyphae, while mucin-exposed cells were predominantly in the round yeast form or formed short chains resembling pseudohyphae (FIG. 3A). The upregulated pathways were enriched in genes encoding cellular amino-acid and small-molecule biosynthetic and metabolic processes. C. albicans metabolism plays a central role in biofilm formation and hyphal morphogenesis¹⁴; thus, metabolic changes may correlate with virulence suppression. Together, these data indicate that mucins across various mucosal surfaces downregulate virulence-associated gene expression and phenotypes, providing rich sources of bioactive molecules for regulating C. albicans.

The roles of mucins were investigated in the context of an intact immune system (FIG. 2G), murine puncture wounds were infected with C. albicans and topically treated with 0.5% MUC2, and the fungal burden over time was quantified in colony-forming units (CFUs). While the fungal burden on day 5 and 7 did not differ between MUC2 and mucin-free treatment, exposure to MUC2, but not mucin-free mock treatment, caused a significant CFU reduction 7 days post-infection compared to day 5 of infection (FIG. 2H). This enhanced C. albicans clearance was likely not due to direct killing by MUC2 because mucins did not alter growth-. Rather, mucins may facilitate fungal clearance by attenuating C. albicans pathogenicity, thus supporting host defense mechanisms to reduce the fungal burden in the wound.

### Example 3. Mucin Glycans Act Via Nrg1 to Prevent Filamentation

Mucin glycans can regulate host-microbe interactions: they serve as nutrients¹⁵, microbial binding sites⁸, and signaling molecules^{18,19}. To determine whether mucin glycans mediate virulence suppression, glycans were isolated via non-reductive, alkaline β-elimination, which preserved the structural heterogeneity of glycan chains, yielding a library of glycans released from MUC5AC. The released glycans were analyzed as permethylated derivatives using nanospray-ionization multi-dimensional mass spectrometry (NSI-MSn, ThermoFisher Orbitrap Discovery) to characterize structural topology features beyond simple monosaccharide composition (FIG. 4A, Table 2). Greater than 80 glycan structures, including isobaric glycans with distinct structural characteristics, were identified. Negative-mode NSI-MSn indicated sulfation on 34 of these glycans. The MUC5AC glycan pool was dominated by Core-1- and Core-2-type O-glycan structures that were partially modified by fucose, possessed multiple LacNAc repeats, and were only sparsely capped by sialic acid (FIG. 4B). Sulfation was most abundant on non-sialylated Core-2-type O-glycans with a single fucose (Table 3).

To determine whether the mucin glycan pool can replicate mucin-induced virulence suppression, RNA sequencing of C. albicans in medium with or without 0.1% w/v MUC5AC glycans was performed. A pooled library of MUC5AC glycans triggered global gene expression changes, with 233 and 308 genes significantly upregulated and downregulated, respectively, compared with cells grown in medium alone (P<0.05). Similar to intact mucins, MUC5AC glycans upregulated the transcription of amino-acid biosynthetic and metabolic processes and downregulated pathways associated with filamentation, biofilm formation, and interspecies interactions (FIG. 4C).

The 233 upregulated genes include, among others, ACO1, ADE12, ADE13, ADE4, ADE5,7, ADE6, AFL1, AGP2, AIP2, AQY1, ARO3, ARO4, ASN1, ASR1, ATF1, BAT22, BIO2, BMT4, BNA31, BNA32, BUL1, CAN1, CAN2, CAR1, CAR2, CAT8, CDC19, CDR4, CHT3, COX15, CTA4, CWH8, DAG7, DAP1, DED81, DQD1, DUR1,2, DUR3, EBP1, EHT1, ENG1, FAD3, FBA1, FDH3, FGR41, FMA1, FRE10, GCN4, GCV1, GCV2, GCV3, GDH2, GDH3, GIS2, GLN1, GLT1, GNP1, GPD2, GPM2, GRS1, HAK1, HEM1, HEM13, HGT7, HIS4, HIS5, HIS7, HOM3, HOM6, HOS3, HRQ2, HSP12, HSP21, HSP60, ICL1, IDP1, IFG3, ILV1, ILV2, ILV3, ILV5, ILV6, IMH3, LEU1, LEU2, LEU4, LEU42, LYS4, MEP1, MIA40, MIS11, MNN1, MNN22, MNN4, NAR1, NRG1, OAC1, OPT1, OPT2, OPT3, OPT4, PDX3, PGA14, PGA38, PGA45, PGA6, PHO87, PIR1, PMA1, PRO3, PTR22, PUT1, PUT2, PYC2, RBE1, RHD1, RHD3, RHR2, RME1, RNH1, RNR1, RNR22, ROA1, RPL10, RPL10A, RPL11, RPL12, RPL15A, RPL17B, RPL2, RPL21A, RPL23A, RPL24A, RPL25, RPL3, RPL30, RPL35, RPL38, RPL42, RPL4B, RPL5, RPL82, RPL8B, RPL9B, RPP2A, RPP2B, RPS12, RPS19A, RPS20, RPS21, RPS24, RPS3, RPS42, RPS4A, RPS6A, RPS7A, RPS9B, RTA3, SAP9, SCW11, SDH12, SDH2, SER1, SHM2, SIM1, SNZ1, SOD1, SOU1, SSA2, STF2, STP1, THR4, TNA1, TOP1, TPO3, TPO4, TRP5, TRX1, TYE7, UBA4, UBI3, URA1, VAS1, WH11, YWP1, and ZPR1.

The 308 downregulated genes include, among others, ADH2, AGO1, AHR1, ALG5, ALS3, AMS1, ARG3, ARO9, ARP1, ASM3, ASR3, ATO1, ATP9, AUT7, AYR2, BEM1, BEM2, BMT1, BMT3, BMT6, BRG1, BUD14, BUD2, CAS4, CCC2, CDC11, CDC12, CDC3, CDC4, CFL2, CFL5, CHS4, CHT2, CIP1, CLA4, CLG1, CPA2, CSA1, CSH1, CSR1, CTR1, CUP1, DCK1, DDR48, DEF1, DFI1, DPP3, ECE1, ECM4, EFG1, FAV1, FDH1, FET31, FET34, FRE30, FRE7, FRP1, FRP2, FTR1, GAC1, GAD1, GAP4, GDB1, GDE1, GDI1, GIN4, GLG2, GLX3, GPH1, GPX2, GRE2, HAC1, HAP3, HAP43, HET1, HGC1, HGT1, HGT18, HGT6, HMX1, HWP1, HYR1, IDH2, IFD6, IHD1, IHD2, IQG1, IRA2, IRO1, IST2, KEX2, KIP4, KRE1, LAP3, LMO1, LSP1, MAC1, MAL31, MCD4, MED15, MET10, MET14, MET15, MET3, MKC1, MKK2, MNN12, MNN15, MNN24, MRF1, MSB2, MYO2, NCE102, PCL1, PEP1, PGA13, PGA4, PGA54, PGA59, PGA63, PGA7, PGI1, PHM7, PHR1, PIN3, PLB3, PLD1, POL93, PRA1, PRN1, PRN2, PRN3, PRN4, PRX1, PST1, PST2, PTP3, RAS1, RAX1, RCT1, RFG1, RFX2, RGA2, RIB3, RIM101, RIM9, ROB1, RTA4, SAL6, SAP5, SEC24, SEP7, SFL2, SHE3, SIP5, SIR2, SKN1, SLK19, SLM2, SMF12, SOD3, SOD5, SRD1, SSD1, SSU81, STB3, STE23, SUN41, SUR7, TEC1, TFS1, TKL1, TSA1, TSA1B, TUB1, TUP1, UME6, VAC8, VPS1, WOR3, WSC1, WSC2, YCK2, YHB5, YKE2, YOX1, YVC1, ZCF20, ZCF27, ZDS1, ZFU2, ZRC1, ZRT1, ZRT2, and ZWF1.

Over 20% of the downregulated genes were associated with filamentous growth, which was suppressed by intact mucins. It was found that isolated MUC5AC glycans suppressed filamentation across the three medium conditions without altering growth, while medium alone supported the formation of extensive hyphae (FIG. 4D). Addition of the monosaccharides found in mucins did not suppress filamentation or alter the expression of signature hyphal-specific (UME6, HGC1)^{20,21} or yeast-specific (YWP1)²² genes relative to medium alone (FIG. 4E). Changes in gene expression were detected 30 min after exposure to MUC5AC glycans, which intensified over 4 h (FIG. 4F). Filamentation suppression was visualized 4 h after hyphal induction and the round yeast morphology was maintained over 8 h (FIG. 4D), suggesting a prolonged glycan response. Further, a dose-dependent effect of mucin glycans was detected, with potent filamentation suppression occurring at concentrations below those of mucosal surfaces (FIG. 4G). Together, these data demonstrate that complex glycan structures are critical for retaining C. albicans in a host-compatible yeast state and may signal a healthy mucosal environment.

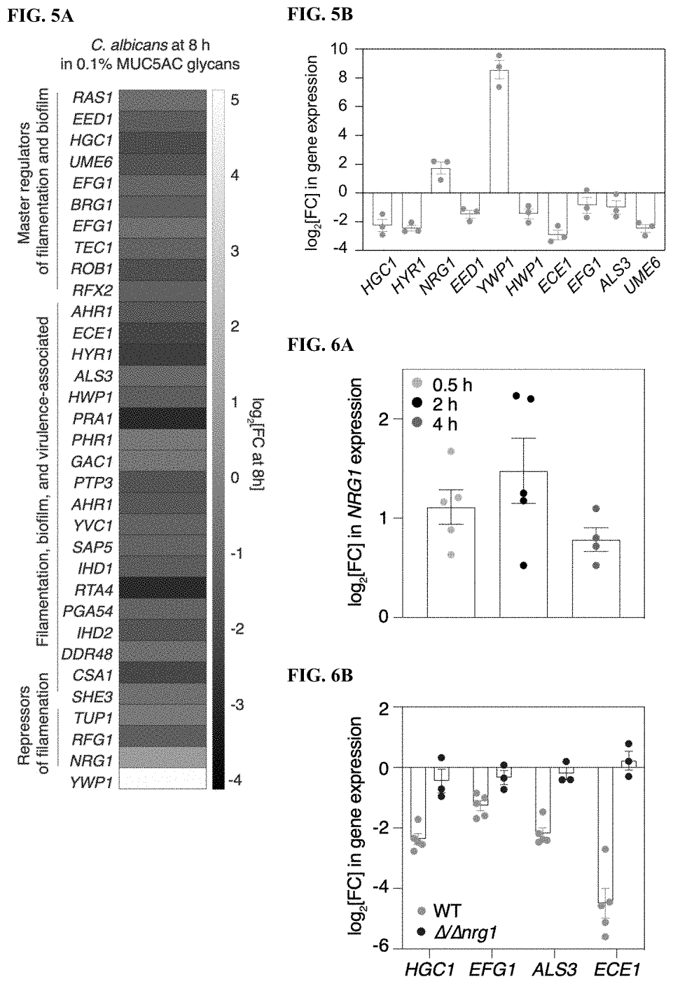

Hyphal morphogenesis in C. albicans is induced by environmental signals acting via multiple signaling cascades, including a cAMP-dependent pathway and a MAPK pathway²³. The RNA-sequencing data revealed that the transcription of many filamentation activators and key outputs of these pathways (including hyphal-specific proteins Ume6, Eed1, and Hgc1²⁴) were significantly downregulated in the presence of mucin glycans (FIG. 5A). Hyphal-specific gene expression is negatively regulated by a protein complex consisting of the general transcriptional corepressor Tup1 and DNA-binding proteins Nrg1 or Rfg1²⁴. The transcription of NRG1, a major transcriptional repressor of filamentation, increased in the presence of mucin glycans. Thus, mucin glycans may inhibit filamentation by regulating transcriptional activators and/or repressors, which play prominent roles in the yeast-to-hyphal transition²³.

To assess whether mucin glycans suppress hyphal formation by preventing activation of the major transcriptional activators of filamentation, mutants that constitutively activate major positive filamentation regulators (Ras1, Cph1, and Efg1) were screened for the filamentation-suppression response to mucin glycans. Ras1 cycles between inactive and active states. The RAS1^{G13V} strain is locked in an active state, leading to hyperfilamentation²⁵. If mucin glycans act upstream or directly via Ras1 activation, the dominant active RAS1^{G13V} strain should remain filamentous in the presence of mucin glycans, being unable to respond to mucin glycans. However, in the presence of mucin glycans, cells from both the RAS1^{G13V} strain and wild-type strain retained the yeast morphology (FIG. 3B), suggesting that this function does not depend on Ras1 activation.

To determine whether mucin glycans act via the cAMP-PKA pathway, whether mucin glycans suppress filamentation was tested in a strain with a phosphomimetic mutation controlled by the glucose-repressible PCK1 promoter^{26,27} (PCKpr-efg1-T206E), which simulates constitutive signaling of Efg1, a downstream transcription factor in the cAMP-PKA pathway²³. In the presence of mucin glycans, cells from a constitutively expressed Efgl transcription factor transitioned to a yeast morphology, as observed for the wild-type strain in Spider medium (FIG. 3C), suggesting that the filamentation suppression of mucin glycans is independent of the cAMP-PKA pathway. To determine whether mucin glycans act via the MAPK pathway, filamentation suppression was tested in a strain over expressing Cph1 (prADH1-CPH1). In the presence of mucin glycans, cells overexpressing Cph1 remained in the yeast form at levels comparable to those of the wild-type strain (FIG. 3B), indicating that mucin glycans suppress filamentation independently of the MAPK pathway. Thus, mucin glycans do not act directly via the major transcriptional activators of hyphal induction to suppress filamentation; instead, they likely act downstream of Cph1 and Efg1 or via alternate pathways.

To explore alternate regulation pathways, it was examined whether mucin glycans act via transcriptional repressors of filamentation, which inhibit the yeast-to-hyphal transition²³. The focus was on NRG1 and TUP1, as their loss leads to constitutive filamentation and upregulation of hyphal genes, even in non-inducing conditions²⁸. Specifically, 30 min after exposure to mucin glycans (FIG. 6A), expression of Nrg1 was upregulated, which continued throughout an 8-h time course. If filamentation suppression depends on Nrg1 or Tup1, those mutant strains should remain filamentous in the presence of mucin glycans. Indeed, opposed to the wild-type strain, upon exposure to mucin glycans, cells lacking NRG1 or TUP1 were constitutively filamentous in the presence or absence of mucin glycans, suggesting that mucin glycans block hyphal formation in an Nrg1/Tup1-dependent manner.

To further explore this function of mucin glycans, RNA sequencing of the wild-type strain and Δ/Δnrg1 mutant strain was performed after 2 h in the presence or absence of mucin glycans to detect early transcriptional changes during hyphal morphogenesis. In the wild-type strain, mucin glycans downregulated 45 and upregulated 64 genes (P<0.05) after 2 h (FIG. 6C).

The 45 downregulated genes include, among others, AHR1, ALS3, AMS1, ASM3, CFL2, CSH1, CTR1, DDR48, DUR1,2, EFG1, FDH1, FET31, FET34, FRE10, FRE30, FRE7, FTR1, HGC1, HMX1, HWP1, IHD1, LAP3, LYS1, LYS2, LYS22, MAC1, MED16, MNN24, OPT4, PGA54, PLB1, POL93, PRA1, RFG1, RIB3, SFL2, SHM1, WOR3, YVC1, ZRT1, and ZRT2.

The 64 upregulated genes include, among others, KCH1, PGA6, RME1, RPL10, RPL10A, RPL11, RPL12, RPL13, RPL15A, RPL16A, RPL17B, RPL18, RPL2, RPL20B, RPL21A, RPL23A, RPL24A, RPL25, RPL27A, RPL28, RPL29, RPL3, RPL30, RPL32, RPL35, RPL37B, RPL43A, RPL4B, RPL6, RPL8B, RPL9B, RPP2A, RPS1, RPS10, RPS12, RPS14B, RPS15, RPS16A, RPS17B, RPS19A, RPS20, RPS21, RPS22A, RPS24, RPS25B, RPS26A, RPS27, RPS5, RPS6A, RPS7A, RPS9B, SOD1, TMA19, UBI3, YST1, and YWP1.

The transcription of several hyphal-specific genes (ALS3, HWP1, EFG1, and HGC1) was downregulated; further, YWP1, a marker for yeast cells, was upregulated (FIG. 6C). Downregulation of genes involved in ion regulation, white-opaque regulation, and amino-peptidase activity, was also detected (FIG. 6C). However, in the Δ/Δnrg1 mutant strain, only 22 genes were differentially expressed (FIG. 6C). The 22 differentially expressed genes include, among others, AMS1, AOX2, ASM3, BLP1, CFL2, CRP1, CSH1, CTR1, FDH1, FRE30, FRE7, ICL1, LAP3, POL93, RIB3, RTA4, SAP5, ZRT2.

Several genes involved in ion homeostasis were differentially downregulated in the Δ/Δnrg1 mutant strain and wild-type strain (FIG. 6C), suggesting that these changes constitute general responses to mucin glycans, independent of morphology. The hyphal-associated genes that were downregulated in the wild-type strain were unchanged in the Δ/Δnrg1 mutant strain (FIG. 6B), consistent with the observation that the Δ/Δnrg1 mutant strain remains filamentous in the presence of mucin glycans. These observations imply that mucin glycans act via Nrg1 to downregulate the expression of hyphal-specific and other virulence-associated genes.

### Example 4. Mucin Glycans Regulate Group-Level Behavior

A virulence attribute of C. albicans is its ability to form robust biofilms⁶. Biofilm cells are highly resistant to conventional antifungal therapeutics and the host immune system, making them highly pathogenic⁶. In C. albicans, biofilm development requires six master transcriptional regulators (Efg1, Tec1, Bcr1, Ndt80, Brg1, and Rob1)²⁹. The first step in biofilm formation involves cell adherence to a surface, mediated by the master regulator Bcr1 and its downstream target genes³⁰.

It was observed that MUC5AC glycans significantly downregulated the transcription of several genes involved in adherence and biofilm initiation, while equivalent amounts of monosaccharides had no effect (FIG. 7A). By inoculating yeast cells into medium with or without (0.1% w/v) mucin glycans and visualizing surface adherence, it was found that mucin glycans significantly reduced cell attachment, while equivalent amounts of monosaccharides had no effect (FIG. 7B).

After initial adherence and biofilm initiation, the next step in biofilm formation is biofilm maturation, where hyphal cells grow and all cells become encased in extracellular matrix²⁹. The RNA-sequencing data revealed significant downregulation of several transcriptional regulators of biofilm maturation, including genes encoding Efg1, Tec1, Brg1, and Rob1 (FIGS. 5A-5B)³⁰. To investigate C. albicans biofilm formation, biofilms formed on the bottom of polystyrene plates were visualized after 24 h of growth. C. albicans typically forms biofilms consisting of yeast, hyphae, and pseudohyphae cells; however, in the presence of MUC5AC glycans, only a layer of yeast-form cells was present on the plate surface (FIG. 7D), with more cells remaining in the yeast non-adhered (planktonic) state compared with the results observed for medium only (FIGS. 7C-7D).

In a host, C. albicans is generally part of a larger multispecies microbial community^{6,31}. It was found that genes involved in microbial interspecies interactions were differentially regulated by mucin glycans, suggesting that mucins influence microbial community dynamics. C. albicans was often found in the presence of the bacterial pathogen Pseudomonas aeruginosa, both as part of the normal microbiota and during infection³¹. In vitro work has shown that these two microbes have an antagonistic relationship when grown together in C. albicans filamentation-inducing conditions³², where P. aeruginosa forms biofilms on C. albicans hyphal cells and secretes small molecules that result in fungal cell death (FIG. 7E). Consistent with previous work, P. aeruginosa did not adhere to or kill C. albicans yeast cells when these species were grown together in non-filamentation-inducing conditions (FIG. 7E)³³.

Because mucins suppressed filamentation¹⁰, it was hypothesized that in filamentation-inducing conditions, mucin glycans would increase the viability of C. albicans in coculture with P. aeruginosa. As previously reported³², cocultures grown under filamentation-inducing conditions in the absence of mucin glycans showed reduced C. albicans CFUs and eventual eradication of C. albicans cells (FIG. 7F). Addition of mucin glycans delayed C. albicans eradication (FIG. 7F), indicating that mucin glycans protect C. albicans against P. aeruginosa. Therefore, mucin glycans may influence microbial population dynamics by modulating C. albicans morphogenesis.

To determine whether filamentation suppression is the dominant factor promoting microbial coexistence, the C. albicans Δ/Δnrg1 mutant strain, which remains filamentous in the presence of mucin glycans, was cocultured with P. aeruginosa in the presence or absence of mucin glycans. The increased viability of C. albicans in the presence of mucin glycans was eliminated in the Δ/Δnrg1 mutant strain (FIG. 7G), indicating that filamentation-suppression effects confer protection from P. aeruginosa. These results suggest that mucin glycans may influence microbial communities and inhibit a range of virulence behaviors.

Research on mucus has traditionally focused on the role of mucins as scaffolding polymers. Here, the findings that mucin O-glycans potently inhibit a range of virulence behaviors, suggest that they can be leveraged for therapeutic applications. Specifically, mucin glycans across three major niches are found to be potent regulators of C. albicans filamentation (FIGS. 4A-4G) that block hyphal formation through Nrg1 (FIGS. 6A-6C) and regulate community behavior (FIGS. 7A-7G). These insights demonstrate the wealth of biochemical information and regulatory power housed within mucus and inform diagnostic strategies for treating and preventing infection.

### Example 5. Mucins Display a Plethora of Glycans with Regulatory Potential

To determine the glycan structures regulating C. albicans physiology and the therapeutic potential of novel glycan-based drugs for C. albicans infection, mucin glycan libraries were screened for filamentation suppression. Glycans isolated from human saliva (MUC5B), porcine gastrointestinal mucus (MUC2), and porcine gastric mucin (MUC5AC) all suppressed filamentation (FIG. 8A), suggesting that these mucin-derived glycans are sufficient to recapitulate the filamentation-suppression response in C. albicans.

To distinguish unique and shared structural features, NSI-MSn was used to analyze released, permethylated glycans from these three mucin pools (Example 1). Glycans at 83 distinct mass/charge (m/z) ratios were identified, approximately ⅓rd of which produced MS fragmentation, indicating the presence of 2-3 isomeric glycan structures (FIG. 8B, Tables 4-6). Thus, the full glycan diversity of these mucin pools exceeds the number of discrete m/z values detected by NSI-MSn. These glycans are predominantly characterized by the presence of a HexNAc (GalNAc) at their reducing terminal. This GalNAc, in O-linkage to serine or threonine amino acids of the mucin protein backbone, provides a foundation upon which a set of structurally distinct glycan core types are built; the core structures are extended to longer, more complex glycan structures depending on the repertoire of glycosyltransferases expressed in the tissue and species of origin. The majority of all three glycan pools consisted of glycans built on Core 1 (Galβ1-3GalNAcα1-Ser/Thr) or Core 2 (Galβ1-3(GlcNAcβ1-6)GalNAcα1-Ser/Thr) structures, while the Tn antigen (GalNAcα1-Ser/Thr) or glycans built on Core 3 (GlcNAcβ1-6GalNAcα1-Ser/Thr) and Core 4 (GlcNAcβ1-3(GlcNAcβ1-6)GalNAcα1-Ser/Thr) structures contributed <10% for each glycan pool (FIG. 8C). Of these 83 glycan compositions, 51 were monosulfated and 20 were disulfated; the most abundant sulfated O-glycans of MUC2 and MUC5AC were Core-2-type, while the most abundant sulfated O-glycans of MUC5B were Core-1- and O-Man-type (Table 3). MUC5AC and MUC5B glycans were more complex than MUC2 glycans (FIG. 8B, Tables 4-6). Specifically, >60% of MUC2 glycans were detected as the Core-1 disaccharide (glycan #2; FIG. 8B, Tables 4-6) or Core 1 modified with a single additional monosaccharide (Fuc or sialic acid; glycans #3 and 4, respectively), compared with <35% for MUC5AC and MUC5B.

Beyond the core structures, 23% and 15% of MUC5AC and MUC5B glycans, respectively, were more than seven sugars long, versus <3% of MUC2 glycans (Tables 4-6). The shortened length of MUC2 glycans may result from degradation through microbial feeding or differences in endogenous glycosyltransferase expression levels in the intestinal tract¹⁵. Consistent with previous reports^{18,34}, mucin glycans from all three sources were heavily fucosylated, with >35% of structures containing at least one fucose, with minimal sialyation (FIG. 8D). MUC5AC yielded the highest abundance of non-fucosylated and asialo glycans (i.e., uncapped glycans), while MUC5B and MUC2 glycans were the most fucosylated and most sialylated, respectively. These capping residues (Fuc and sialic acid) can block the extension of core structures by limiting the addition of N-acetyllactosamine (LacNAc) units. Thus, the glycan collection prepared from MUC5AC had the highest abundance of LacNAc repeats. Several glycan structural elements or motifs with known roles in immunorecognition (e.g., LacNAc, LacdiNAc, Lewis X, GalGal)³⁵ or cell adhesion (e.g., O-Man) were detected as minor components of all three preparations (FIG. 8E); thus, these structures may contribute to immunomodulatory activities. Despite substantial overlap in the glycan structures among the three mucin glycan pools (FIG. 8B, Tables 4-6), differences in mucin glycan identity and length likely arise from niche-specific selective preferences that have coevolved to influence the structural and functional properties of the mucus barrier.

### Example 6. Synthetic Core-1 and Core-2 Structures Suppress Filamentation

Because glycan structure compositions vary across mucin types, glycan structures that were highly abundant across the mucin species examined here were focused on. In total, six glycan structures (FIG. 8F, Tables 4-6) were shared among all three mucin glycan pools and together represented >40% of the total glycan profile in each sample (FIG. 8F), highlighting them as candidates for virulence-attenuating activity. To date, most known mucin glycan structures have not been associated with clearly delineated functions.

Rather than fractionating glycan pools down to the single-glycan level, which poses technical challenges³⁶, a synthetic approach was developed to obtain these six highly abundant mucin glycans (FIG. 9A, Example 7). The initial focus was on Core 1 (1) and Core 2 (2), which constitute 40% of total mucin glycans (Tables 4-6). Neither Core 1 nor Core 2 showed toxic effects, as measured by C. albicans growth at a concentration of 0.1% (w/v), an effective concentration for filamentation suppression in the mucin glycan pool. To determine whether Core 1 and Core 2 have a similar regulatory capacity as the complex glycan pool, their ability to regulate two signature proteins, the yeast-wall protein Ywp1 and the filamentation-associated cytotoxin Ece1, whose genes were strongly up- and downregulated, respectively, by the native mucin glycan pool (see FIGS. 4A-4G), were tested. RT-qPCR revealed that Core 1 and Core 2 individually upregulated YWP1 expression at 0.1% (w/v) to a similar degree as the intact mucin pool, and a concentration increase to 0.4% (w/v) did not significantly alter YWP1 expression (FIG. 9B). Moreover, Core 1 and Core 2 both suppressed ECE1 expression at 0.1% and 0.4%, albeit less strongly than the mucin glycan pool (FIG. 9B). By contrast, an equal-parts mixture of the mucin monosaccharide components did not change gene expression at corresponding concentrations (FIG. 9B). Thus, both Core 1 and Core 2 regulate these two genes with similar potency as the native glycan pool.

To elucidate the role of glycan composition, the effects of four modified Core structures: Core 1+fucose (3), Core 1+sialic acid (4), Core 2+fucose (5), and Core 2+galactose (6) (FIG. 9A) were tested. These four glycans all transcriptionally upregulated YWP1 while downregulating ECE1, similar to their unmodified Core counterparts (FIGS. 9C-9D). The addition of fucose to Core 1 and Core 2 did not measurably alter the ability of these structures to up- or downregulate the two signature genes (FIGS. 9C-9D); the addition of galactose did not significantly alter Core 2 activity (FIG. 9D). However, the addition of sialic acid dampened the gene regulatory response of Core 1 (FIG. 9C). Exploring a broader range of filamentation-associated genes, it was observed that all six synthetic glycans possessed a sliding range of bioactivity, rather than an on/off response: Core 1, Core 1+fucose, and Core 2+galactose showed the strongest suppression of filamentation-associated genes, while Core 1+sialic acid exhibited the weakest effect (FIG. 9E).

The phenotypic filamentation assay confirmed this conclusion: while medium alone supported the formation of extensive hyphal filaments, Core 1, Core 1+fucose, and Core 2+galactose most potently blocked filamentation, as evidenced by the predominance of yeast cells in culture with these structures compared with the monosaccharide pool and medium alone (FIG. 9F, FIG. 4D). Core 1+sialic acid was less effective at suppressing filamentation, despite partially downregulating the transcription of filamentation-associated genes (FIGS. 9C & 9F).

By characterizing bioactive glycans across mucin types, prominent Core-1- and Core-2-modified structures commonly found across mucosal surfaces were identified and synthesized. It was demonstrated that Core 1, Core 2+galactose, and Core 1+fucose individually suppress filamentation at potencies comparable to those of native mucins. These findings highlight that O-glycans can control virulence traits (FIGS. 9A-9F) without killing the microbe, which may prevent the evolution of drug resistance. Many well-known small-molecule drugs, such as antibiotics and anticancer agents³⁷, naturally contain glycans as part of their core structure and/or sugar side chain. Therefore, the discovery of O-glycans that attenuate C. albicans virulence provides the basis for novel glycan-based or glycan-mimetic therapeutics that enhance, or even replace, current antifungals. Native glycans, which are unconjugated to larger macromolecules, are generally ineffective as small-molecule pharmaceuticals due to their inherently poor pharmacokinetic properties³⁸. By identifying the most bioactive mucin O-glycans, the precise glycan epitopes required for activity to design glycomimetic structures that retain activity while exhibiting improved drug-like properties can be established. For example, glycan hydroxyl groups or post-synthetic modifications that are not necessary for activity can be chemically modified or eliminated to improve overall drug-likeness³⁹. Additionally, smaller glycan epitopes should be more amenable to larger-scale production. Frequently, small glycans with biological activity gain potency when presented as multivalent conjugates on a defined backbone. Identifying minimal functional epitopes of mucin glycans provides the basis for conjugates with enhanced efficacies, mimicking the multivalent glycan presentation offered by endogenous mucin proteins.

Given the complexity and diversity of mucin glycans^{18,34} and dynamic glycosylation changes based on cell type⁴⁰, developmental stage⁴¹, and disease state⁴², structural changes in host signals may activate or inhibit the function of specific O-glycans. Accordingly, it was determined that Core 1, Core 1+fucose, and Core 2+galactose effectively suppress filamentation, while Core 1+sialic acid significantly dampens this response. This suggests sialic acid, which is ubiquitously expressed on host cells⁴³, has an unappreciated role in modulating virulence. Changes in glycosylation in disease states may mask or eliminate mucins' protective functions: the presentation of complex glycan structures in mucus contributes to a healthy mucosal environment, while degradation or modification of mucin glycans may trigger C. albicans to transition from commensal to pathogenic.

Nrg1 regulation is temporally coordinated by two central signaling pathways mediating cell growth, leading to transient NRG1 downregulation and degradation of Nrg1 protein followed by occlusion of Nrg1 from hyphal-specific promoters that sustain hyphal development²⁴. Mucin glycans may potentially function as ligands to mimic nutrient signaling pathways or may bind directly to C. albicans adhesins, thus modulating morphogenesis⁴⁴.

### Example 7. Glycan Synthesis

General methods. All commercial reagents were used as supplied unless otherwise stated, and solvents were dried and distilled using standard techniques. Thin layer chromatography was performed on silica-coated glass plates (TLC Silica Gel 60 F₂₅₄, Merck) with detection by fluorescence, charring with 5% H₂SO_{4(aq)}, or staining with a ceric ammonium molybdate solution. Organic solutions were concentrated and/or evaporated to dry under vacuum in a water bath (<50° C.). Molecular sieves were dried at 400° C. under vacuum for 20-30 minutes prior to use. Amberlite IR-120H resin was washed extensively with MeOH and dried under vacuum prior to use. Medium-pressure liquid chromatography (MPLC) was performed using a CombiFlash Companion equipped with RediSep normal-phase flash columns, and solvent gradients refer to sloped gradients with concentrations reported as % v/v. NMR spectra were recorded on a Bruker Avance DMX-500 (500 MHz) spectrometer, and assignments achieved with the assistance of 2D gCOSY, 2D gTOCSY, 2D gHSQC, and 2D gHMBC; chemical shifts are expressed in ppm and referenced to either Si(CH₃)₄ (for CDCl₃), residual CHD₂OD (for CD₃OD), or a MeOH internal standard (for D₂O). Low resolution electron-spray ionization mass spectrometry (ESI-MS) was performed using a Waters micromass ZQ. High resolution mass spectrometry was performed using an Agilent 1100 LC equipped with a photodiode array detector, and a Micromass QTOF I equipped with a 4 GHz digital-time converter. Optical rotation was determined in a 10-cm cell at 20° C. using a Perkin-Elmer Model 341 polarimeter. HPLC analysis was performed using an Agilent 1100 LC equipped with an Atlantis T3 (3 μm, 2.1×100 mm) C18 column and ELSD detection.

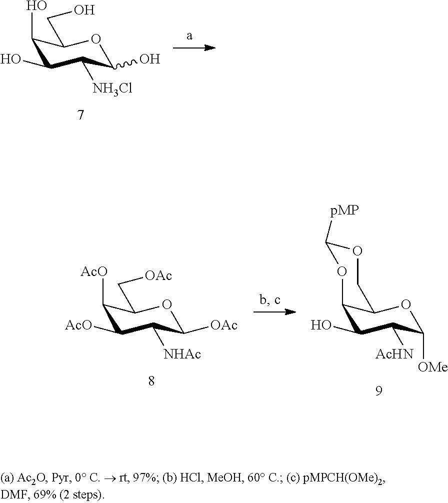

Acetyl 2-acetamido-3,4,6-tri-O-acetyl-2-deoxy-β-D-galactopyranoside (8). D-Galactosamine hydrochloride (7; 12.35 g, 57.27 mmol) in anhydrous pyridine (80 mL) was cooled to 0° C., and then Ac₂O (40 mL) added dropwise over 15 min and the flask slowly warmed to rt. After 16 hours, the reaction mixture was concentrated to a syrup via co-evaporation with toluene (2×50 mL), and then the crude material was purified via MPLC on silica gel using 0→60% acetone-CH₂Cl₂ to afford the pure product as a white solid (21.63 g, 55.55 mmol, 97% yield). R_{f}=0.11 (3:7 acetone:CH₂Cl₂). [α]_{D}²⁰: +9.8° (c 1.0, CHCl₃). ¹H NMR (CDCl₃, 500 MHz): δH 5.70 (d, 1H, J=8.8 Hz, H-1), 5.43 (d, 1H, J=9.5 Hz, NH), 5.38 (dd, 1H, J=3.3, <1 Hz, H-4), 5.09 (dd, 1H, J=11.3, 3.3 Hz, H-3), 4.45 (ddd, 1H, J=11.2, 9.2, 9.2 Hz, H-2), 4.17 (dd, 1H, J=11.3, 6.6 Hz, H-6^{a}), 4.12 (dd, 1H, J=11.3, 6.5 Hz, H-6^{b}), 4.02 (ddd, 1H, J=6.5, 6.5, 1.0 Hz, H-5), 2.17 (s, 3H, OAc), 2.13 (s, 3H, OAc), 2.05 (s, 3H, OAc), 2.02 (s, 3H, OAc), 1.94 (s, 3H, NHAc). ¹³C NMR (CDCl₃, 125 MHz): δC 170.97 (C═O), 170.62 (C═O), 170.47 (C═O), 170.39 (C═O), 169.79 (C═O), 93.28 (C-1), 72.11 (C-5), 70.55 (C-3), 66.56 (C-4), 61.52 (C-6), 50.06 (C-2), 23.54 (NHAc), 21.11 (OAc), 20.89 (2×OAc), 20.86 (OAc). LRMS m/z calc'd for C₁₆H₂₃NNaO₁₀ (M+Na)⁺: 412.12; found: 412.10.

Methyl 2-acetamido-2-deoxy-4,6-O-(p-methoxybenzylidene)-α-D-galactopyranoside (9). The starting material (8; 5.953 g, 15.29 mmol) was dissolved into 2% v/v conc. HCl in MeOH and left heating at 60° C. After 3 days, the mixture was evaporated to dry to afford the desired methyl α-glycoside crude product: R_{f}=0.29 (1:4 CH₃OH:CH₂Cl₂); LRMS m/z calc'd for C₉H₁₇NNaO₆ (M+Na)⁺: 258.10; found: 258.06. The crude material and p-methoxybenzylidene dimethyl acetal (3.2 mL, 19 mmol) were added to anhydrous DMF (35 mL) under Ar (acidity maintained from previous step). After 18 hours of mixing at ambient temperature, the reaction mixture was neutralized with Et₃N (to pH 8), concentrated to a syrup, and then purified via MPLC on silica gel using 0→60% acetone (w/ 0.1% NH₄OH)—CH₂Cl₂ to afford the desired product as a white solid (3.722 g, 10.53 mmol, 69% yield over 2 steps). R_{f}=0.57 (0.01:10:29.99:60 NH₄OH:MeOH:acetone:CH₂Cl₂). [α]_{D}²⁰: +135° (c 1.0, CHCl₃). ¹H NMR (CDCl₃, 500 MHz): δH 7.46-7.43 (m, 2H, Ar), 6.90-6.87 (m, 2H, Ar), 5.91 (d, 1H, J=8.9 Hz, NH), 5.50 (s, 1H, PhCH), 4.83 (d, 1H, J=3.5 Hz, H-1), 4.44 (ddd, 1H, J=10.9, 9.0, 3.5 Hz, H-2), 4.24 (dd, 1H, J=12.5, 1.4 Hz, H-6^{a}), 4.15 (dd, 1H, J=3.4, <1 Hz, H-4), 4.03 (dd, 1H, J=12.5, 1.6 Hz, H-6^{b}), 3.82-3.79 (m, 4H, H-3 and ArOCH₃), 3.60-3.59 (m, 1H, H-5), 3.38 (s, 3H, OCH₃), 2.94 (d, 1H, J=<1 Hz, 3-OH), 2.02 (s, 3H, Ac). ¹³C NMR (CDCl₃, 125 MHz): δC 171.39 (C═O), 160.30 (Ar), 130.30 (Ar), 127.85 (Ar), 113.70 (Ar), 101.30 (PhCH), 99.49 (C-1), 75.59 (C-4), 69.40 (C-6), 68.97 (C-3), 62.86 (C-5), 55.56 (OCH₃), 55.43 (ArOCH₃), 50.54 (C-2), 23.51 (Ac). LRMS m/z calc'd for C₁₇H₂₃NNaO₇ (M+Na)⁺: 376.14; found: 376.08.

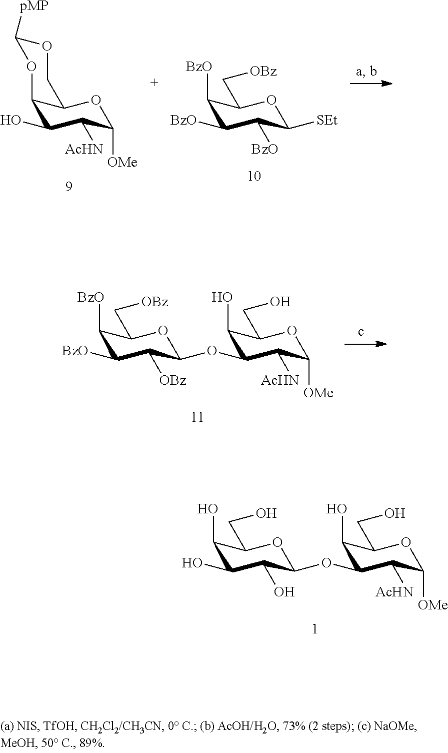

Methyl 2,3,4,6-tetra-O-benzoyl-β-D-galactopyranosyl-(1→3)-2-acetamido-2-deoxy-α-D-galactopyranoside (11). The glycosyl acceptor (9; 602 mg, 1.70 mmol), glycosyl donor⁶¹ (10; 2.726 g, 4.255 mmol), and crushed molecular sieves (3 Å, 1.905 mg) in anhydrous CH₂Cl₂ (10 mL) and anhydrous acetonitrile (20 mL) were left mixing for 1 hour at ambient temperature under Ar. The reaction flask was cooled to 0° C., and then N-iodosuccinimide (NIS) was added (777 mg, 3.45 mmol) followed by the drop-wise addition of triflic acid (TfOH) (23 μL, 0.26 mmol). After 5 hours, the mixture was neutralized with Et₃N (to pH 8), warmed to ambient temperature, filtered over Celite, and diluted with CH₂Cl₂ (100 mL). The organic phase was washed with sat'd Na₂S₂O_{3(aq)} solution (100 mL), sat'd NaCl_{(aq)} solution (100 mL), dried with Na₂SO₄, filtered, and evaporated to dry to afford the crude product: LRMS m/z calc'd for C₅₁H₄₉NNaO₁₆ (M+Na)⁺: 954.30; found: 954.29. The crude mixture was then dissolved into AcOH (16 mL) and H₂O (4 mL). After 3 hours at ambient temperature, the mixture was neutralized with excess sat'd NaHCO_{3(aq)} solution and the product extracted with CH₂Cl₂ (2×100 mL). The combined organic phases were washed with sat'd NaHCO_{3(aq)} (3×100 mL), dried with Na₂SO₄, filtered, and evaporated to dry. The crude material was purified via MPLC on silica gel using 0→10% MeOH—CH₂Cl₂ to afford the pure product as a white solid (1.007 g, 1.237 mmol, 73% yield over 2 steps). R_{f}=0.38 (1:19 MeOH:CH₂Cl₂). [α]_{D}²⁰: +170° (c 1.0, CHCl₃). ¹H NMR (CDCl₃, 500 MHz): δH 8.07-8.05 (m, 2H, Ar), 8.04-8.02 (m, 2H, Ar), 7.99-7.97 (m, 2H, Ar), 7.74-7.71 (m, 2H, Ar), 7.61-7.55 (m, 2H, Ar), 7.53-7.50 (m, 1H, Ar), 7.48-7.44 (m, 2H, Ar), 7.44-7.38 (m, 5H, Ar), 7.22-7.18 (m, 2H, Ar), 5.95 (dd, 1H, J=3.5, <1 Hz, Gal_H4), 5.84 (dd, 1H, J=10.4, 8.0 Hz, Gal_H2), 5.64 (dd, 1H, J=10.4, 3.5 Hz, Gal_H3), 5.45 (d, 1H, J=8.9 Hz, NH), 5.04 (d, 1H, J=8.0 Hz, Gal_H1), 4.73 (d, 1H, J=3.6 Hz, GalN_H1), 4.62-4.54 (m, 3H, Gal_H6^{a}, Gal_H6^{b}, and GalN_H2), 4.44-4.41 (m, 1H, Gal_H5), 4.21-4.19 (m, 1H, GalN_H4), 3.84 (dd, 1H, J=10.8, 3.0 Hz, GalN_H3), 3.77 (ddd, 1H, J=11.2, 6.2, 2.7 Hz, GalN_H6^{a}), 3.71-3.69 (m, 1H, GalN_H5), 3.54 (ddd, 1H, J=11.3, 8.9, 4.3 Hz, GalN_H6^{b}), 3.27 (s, 3H, OCH₃), 3.10 (d, 1H, J=<1 Hz, 4-OH), 2.44 (dd, 1H, J=8.8, 3.0 Hz, 6-OH), 1.34 (s, 3H, Ac). ¹³C NMR (CDCl₃, 125 MHz): δC 170.10 (Ac), 166.27 (C═O), 165.81 (C═O), 165.70 (C═O), 164.94 (C═O), 133.91 (Ar), 133.77 (Ar), 133.71 (Ar), 133.53 (Ar), 130.18 (Ar), 130.00 (Ar), 129.91 (Ar), 129.90 (Ar), 129.44 (Ar), 129.26 (Ar), 128.85 (Ar), 128.81 (Ar), 128.79 (Ar), 128.71 (Ar), 128.47 (Ar), 102.07 (Gal_C1), 98.82 (GalN_C1), 79.86 (GalN_C3), 72.24 (Gal_C5), 71.62 (Gal_C3), 69.90 (Gal_C2), 69.44 (GalN_C5), 68.87 (GalN_C4), 68.36 (Gal_C4), 62.85 (GalN_C6), 62.83 (Gal_C6), 55.30 (OCH₃), 48.05 (GalN_C2), 22.56 (Ac). LRMS m/z calc'd for C₄₃H₄₃NNaO₁₅ (M+Na)⁺: 836.25; found: 836.23.

Methyl β-D-galactopyranosyl-(1→3)-2-acetamido-2-deoxy-α-D-galactopyranoside (1). The starting material (11; 83 mg, 0.10 mmol) was dissolved into anhydrous MeOH (1.5 mL), and then NaOMe solution was added drop-wise (1.5 M NaOMe in MeOH; to pH 10) and the mixture heated at 50° C. After 14 hours, the reaction mixture was cooled to rt, neutralized with acidic resin (Amberlite IR-120H; to pH 6), filtered, and then evaporated to dry. The crude material was purified via RPLC on C-18 silica gel using 0→40% acetonitrile-H₂O to afford the pure product as a white solid (36 mg, 0.091 mmol, 89% yield); data characterization is in agreement with that previously published⁶². [α]_{D}²⁰: +95° (c 1.0, H₂O). ¹H NMR (D₂O, 500 MHz): δH 4.77-4.76 (m, 1H, GalN_H1), 4.45 (d, 1H, J=7.8 Hz, Gal_H1), 4.32 (dd, 1H, J=11.1, 3.7 Hz, GalN_H2), 4.22 (dd, 1H, J=2.9, <1 Hz, GalN_H4), 3.99 (dd, 1H, J=11.1, 3.1 Hz, GalN_H3), 3.95 (ddd, 1H, J=7.4, 4.9, <1 Hz, GalN_H5), 3.90 (dd, 1H, J=3.4, <1 Hz, Gal_H4), 3.77 (dd, 1H, J=11.7, 7.4 Hz, Gal_H6^{a}), 3.78-3.72 (m, 2H, Gal_H6^{a} and GalN_H6^{b}), 3.72 (dd, 1H, J=11.7, 4.5 Hz, Gal_H6^{b}), 3.64 (ddd, 1H, J=7.7, 4.5, <1 Hz, Gal_H5), 3.60 (dd, 1H, J=9.9, 3.4 Hz, Gal_H3), 3.50 (dd, 1H, J=9.9, 7.8 Hz, Gal_H2), 3.38 (s, 3H, OCH₃), 2.01 (s, 3H, Ac). ¹³C NMR (D₂O, 125 MHz): δC 175.23 (C═O), 105.34 (Gal_C1), 98.95 (GalN_C1), 77.92 (GalN_C3), 75.59 (Gal_C5), 73.17 (Gal_C3), 71.24 (Gal_C2), 71.07 (GalN_C5), 69.38 (GalN_C4), 69.21 (Gal_C4), 61.86 (GalN_C6), 61.59 (Gal_C6), 55.72 (OCH₃), 49.22 (GalN_C2), 22.64 (Ac). ESI-HRMS m/z calc'd for C₁₅H₂₇NNaO₁₁ (M+Na)⁺: 420.1482; found: 420.1482. HPLC purity analysis: 99.1%, Rₜ 4.54 minutes, Atlantis T3 C18 column.

Methyl 2,3,4,6-tetra-O-benzoyl-β-D-galactopyranosyl-(1→3)-[3,4,6-tri-O-acetyl-2-deoxy-2-phthalimido-β-D-glucopyranosyl-(1→6)]-2-acetamido-2-deoxy-α-D-galactopyranoside (13). The glycosyl acceptor (11; 135 mg, 0.166 mmol), glycosyl donor⁶⁶ (12; 87 mg, 0.18 mmol), and crushed molecular sieves (3 Å, 190 mg) in anhydrous CH₂Cl₂ (1.0 mL) and anhydrous acetonitrile (1.0 mL) were left mixing for 1 hour at ambient temperature under Ar. The reaction flask was cooled to 0° C., and then NIS was added (67 mg, 0.30 mmol) followed by the drop-wise addition of TfOH solution (15% v/v in CH₂Cl₂; 10 μL, 0.017 mmol). After 3 hours, the mixture was neutralized with Et₃N (to pH 8), warmed to ambient temperature, and diluted with CH₂Cl₂ (60 mL). The organic phase was washed with saturated Na₂S₂O_{3(aq)} solution (60 mL), saturated NaCl_{(aq)} solution (60 mL), dried with Na₂SO₄, filtered, and evaporated to dry.

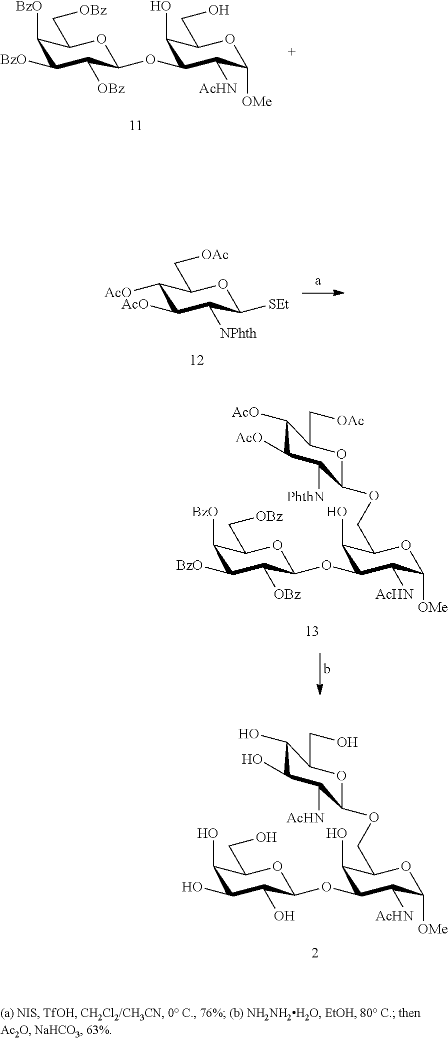

The crude material was purified via MPLC on silica gel using 0→20% acetone (containing 0.05% v/v NH₄OH_{(aq)})—CH₂Cl₂ to afford the pure product as a white solid (156 mg, 0.127 mmol, 76% yield). R_{f}=0.58 (0.01:19.99:80 NH₄OH:acetone:CH₂Cl₂). [α]_{D}²⁰: +123° (c 1.0, CHCl₃). ¹H NMR (CDCl₃, 500 MHz): δH 8.09-8.06 (m, 2H, Ar), 8.02-7.99 (m, 2H, Ar), 7.93-7.90 (m, 2H, Ar), 7.87-7.71 (m, 6H, Ar), 7.65-7.59 (m, 2H, Ar), 7.52-7.46 (m, 5H, Ar), 7.43-7.40 (m, 1H, Ar), 7.39-7.35 (m, 2H, Ar), 7.24-7.20 (m, 2H, Ar), 5.93 (dd, 1H, J=3.2, <1 Hz, Gal_H4), 5.82 (dd, 1H, J=10.7, 9.1 Hz, GlcN_H3), 5.76 (dd, 1H, J=10.4, 8.0 Hz, Gal_H2), 5.56 (dd, 1H, J=10.4, 3.4 Hz, Gal_H3), 5.33 (d, 1H, J=8.4 Hz, GlcN_H1), 5.18 (dd, 1H, J=9.6, 9.6 Hz, GlcN_H4), 5.06 (d, 1H, J=8.9 Hz, NH), 4.85 (d, 1H, J=8.0 Hz, Gal_H1), 4.53-4.50 (m, 2H, Gal_H6^{a} and Gal_H6^{b}), 4.35 (dd, 1H, J=12.2, 4.2 Hz, GlcN_H6^{a}), 4.35-4.29 (m, 2H, GalN_H2 and Gal_H5), 4.29 (dd, 1H, J=10.8, 8.5 Hz, GlcN_H2), 4.22 (d, 1H, J=3.5 Hz, GalN_H1), 4.19 (dd, 1H, J=12.2, 2.0 Hz, GlcN_H6^{b}), 3.96-3.95 (m, 1H, GalN_H4), 3.87 (ddd, 1H, J=10.1, 4.3, 2.3 Hz, GlcN_H5), 3.76 (dd, 1H, J=10.6, 2.4 Hz, GalN_H6^{a}), 3.69 (dd, 1H, J=10.7, 8.5 Hz, GalN_H6^{b}), 3.65-3.61 (m, 2H, GalN_H3 and GalN_H5), 2.81 (s, 3H, OCH₃), 2.76 (d, 1H, J=<1 Hz, GalN_4-OH), 2.10 (s, 3H, OAc), 2.04 (s, 3H, OAc), 1.86 (s, 3H, OAc), 1.28 (s, 3H, NHAc). ¹³C NMR (CDCl₃, 125 MHz): δC 170.91 (C═O), 170.28 (C═O), 169.87 (C═O), 169.77 (C═O), 167.71 (C═O), 166.18 (C═O), 165.79 (C═O), 165.72 (C═O), 164.84 (C═O), 134.52 (Ar), 134.01 (Ar), 133.74 (Ar), 133.71 (Ar), 133.60 (Ar), 131.68 (Ar), 130.27 (Ar), 129.96 (Ar), 129.38 (Ar), 129.26 (Ar), 128.94 (Ar), 128.86 (Ar), 128.76 (Ar), 128.72 (Ar), 128.53 (Ar), 102.02 (Gal_C1), 99.13 (GlcN_C1), 98.23 (Fuc_C1), 79.92 (GalN_C3), 72.19 (Gal_C5), 72.05 (GlcN_C5), 71.61 (Gal_C3), 71.09 (GalN_C6), 70.83 (GalN_C3), 69.82 (Gal_C2), 69.19 (GlcN_C4), 68.54 (GalN_C5), 68.25 (GalN_C4), 68.16 (Gal_C4), 62.57 (Gal_C6), 62.24 (GlcN_C6), 54.89 (GlcN_C2), 54.50 (OCH₃), 47.88 (GalN_C2), 22.62 (NHAc), 20.99 (OAc), 20.86 (OAc), 20.67 (OAc). LRMS m/z calc'd for C₆₃H₆₂N₂NaO₂₄ (M+Na)⁺: 1253.36; found: 1253.32.

Methyl β-D-galactopyranosyl-(1→3)-[2-acetamido-2-deoxy-β-D-glucopyranosyl-(1→6)]-2-acetamido-2-deoxy-α-D-galactopyranoside (2). The protected trisaccharide (13; 146 mg, 0.119 mmol) and NH₂NH₂·H₂O (47 μL, 0.98 mmol) were added to EtOH (3.0 mL) and left mixing at 80° C. After 16 hours, the mixture was cooled to ambient temperature and then NaHCO₃ (406 mg, 4.83 mmol) and Ac₂O added (0.23 mL, 2.4 mmol). After another 4 hours, the solution was evaporated to dry and the crude mixture purified via RPLC on C-18 silica gel using 0→30% acetonitrile-H₂O to afford the pure product as a white solid (45 mg, 0.075 mmol, 63% yield over 2 steps). [α]_{D}²⁰: +54° (c 0.3, H₂O). ¹H NMR (D₂O, 500 MHz): δH 4.75 (d, 1H, J=3.8 Hz, GalN_H1), 4.52 (d, 1H, J=8.5 Hz, GlcN_H1), 4.44 (d, 1H, J=7.8 Hz, Gal_H1), 4.31 (dd, 1H, J=11.1, 3.7 Hz, GalN_H2), 4.20 (dd, 1H, J=3.0, <1 Hz, GalN_H4), 4.06 (dd, 1H, J=10.6, 3.0 Hz, GalN_H6^{a}), 4.03 (ddd, 1H, J=11.0, 2.6, <1 Hz, GalN_H5), 3.99 (dd, 1H, J=11.1, 3.1 Hz, GalN_H3), 3.93 (dd, 1H, J=12.3, 1.8 Hz, GlcN_H6^{a}), 3.90 (dd, 1H, J=3.3, <1 Hz, Gal_H4), 3.77-3.70 (m, 4H, Gal_H6^{a}, GlcN_H6^{b}, Gal_H6^{b}, and GalN_H6^{b}), 3.71 (dd, 1H, J=10.4, 8.5 Hz, GlcN_H2), 3.63 (ddd, 1H, J=7.6, 4.7, <1 Hz, Gal_H5), 3.60 (dd, 1H, J=9.9, 3.4 Hz, Gal_H3), 3.53 (dd, 1H, J=10.3, 8.3 Hz, GlcN_H3), 3.50 (dd, 1H, J=10.0, 7.9 Hz, Gal_H2), 3.46-3.41 (m, 2H, GlcN_H5 and GlcN_H4), 3.35 (s, 3H, OCH₃), 2.00 (s, 6H, 2×Ac). ¹³C NMR (D₂O, 125 MHz): δC 175.25 (C═O), 175.09 (C═O), 105.34 (Gal_C1), 102.27 (GlcN_C1), 98.82 (GalN_C1), 77.73 (GalN_C3), 76.49 (GlcN_C5), 75.61 (Gal_C5), 74.43 (GlcN_C3), 73.17 (Gal_C3), 71.25 (Gal_C2), 70.67 (GalN_C6), 70.59 (GlcN_C4), 69.93 (GalN_C5), 69.62 (GalN_C4), 69.22 (Gal_C4), 61.60 (Gal_C6), 61.36 (GlcN_C6), 56.15 (GlcN_C2), 55.51 (OCH₃), 49.17 (GalN_C2), 22.81 (Ac), 22.65 (Ac). ESI-HRMS m/z calc'd for C₂₃H₄₀N₂NaO₁₆ (M+Na)⁺: 623.2276; found: 623.2276. HPLC purity analysis: >99.5%, Rₜ 5.39 minutes, Atlantis T3 C18 column.

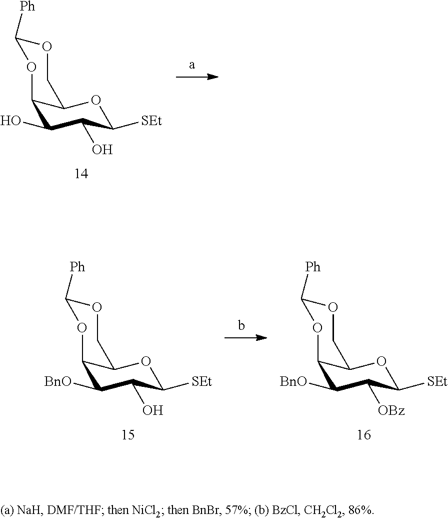

Ethyl 3-O-benzyl-4,6-O-benzylidene-1-thio-β-D-galactopyranoside (15). The starting material⁶³ (14; 506 mg, 1.62 mmol) and NaH (60% oil dispersion; 138 mg, 3.45 mmol) were added to anhydrous THE (20 mL) and anhydrous DMF (2 mL) and left mixing at ambient temperature for 1 hour. Anhydrous NiCl₂ was added⁶⁴ (209 mg, 1.6 mmol), and after another hour benzyl bromide was added drop-wise over 5 min (212 μL, 1.78 mmol). After 20 hours the reaction was quenched via the slow addition of MeOH (2 mL), 3 drops of AcOH were added, and then the mixture evaporated to dry. The crude material was redissolved into CH₂Cl₂ (120 mL) and then washed with sat'd NaCl_{(aq)} solution (2×120 mL), dried with Na2SO4, filtered, and then evaporated to dry. The crude material was purified via MPLC using 0→30% EtOAc-toluene to afford the pure product as a white solid (374 mg, 0.929 mmol, 57% yield). R_{f}=0.53 (1:4 acetone:toluene). [α]_{D}²⁰: +6.7° (c 1.0, CHCl₃). ¹H NMR (CDCl₃, 500 MHz): δH 7.52-7.48 (m, 2H, Ar), 7.41-7.27 (m, 8H, Ar), 5.44 (s, 1H, PhCH), 4.79 (d, 1H, J=12.3 Hz, PhCH^{a}H^{b}), 4.76 (d, 1H, J=12.3 Hz, PhCH^{a}H^{b}), 4.35 (d, 1H, J=9.6 Hz, H-1), 4.31 (dd, 1H, J=12.4, 1.5 Hz, H-6^{a}), 4.18 (dd, 1H, J=3.4, <1 Hz, H-4), 4.06 (ddd, 1H, J=9.4, 9.4, 1.6 Hz, H-2), 3.97 (dd, 1H, J=12.4, 1.8 Hz, H-6^{b}), 3.50 (dd, 1H, J=9.2, 3.4 Hz, H-3), 3.41-3.40 (m, 1H, H-5), 2.83 (dq, 1H, J=12.6, 7.5 Hz, SCH^{a}H^{b}CH₃), 2.75 (dq, 1H, J=12.6, 7.5 Hz, SCH^{a}H^{b}CH₃), 2.55 (d, 1H, J=1.6 Hz, 2-OH), 1.33 (dd, 3H, J=7.5, 7.5 Hz, SCH₂CH₃). ¹³C NMR (CDCl₃, 125 MHz): δC 138.29 (Ar), 138.05 (Ar), 129.21 (Ar), 128.68 (Ar), 128.38 (Ar), 128.08 (Ar), 126.60 (Ar), 101.48 (PhCH), 85.52 (C-1), 80.54 (C-3), 73.75 (C-4), 71.76 (PhCH₂), 70.36 (C-5), 69.65 (C-6), 68.24 (C-2), 23.15 (SCH₂CH₃), 15.50 (SCH₂CH₃). LRMS m/z calc'd for C₂₂H₂₆NaO₅S (M+Na)⁺: 425.14; found: 425.22.

Ethyl 2-O-benzoyl-3-O-benzyl-4,6-O-benzylidene-1-thio-β-D-galactopyranoside (16). The starting material (15; 1.112 g, 2.763 mmol) and benzoyl chloride (0.42 mL, 3.6 mmol) were added to anhydrous pyridine (2.0 mL) and anhydrous CH₂Cl₂ (8.0 mL). After 3 hours, the mixture was quenched via the dropwise addition of MeOH (1 mL), evaporated to dry, and then purified via MPLC using 0→10% EtOAc-toluene to afford the pure product as a white solid (1.207 g, 2.382 mmol, 86% yield). R_{f}=0.47 (1:9 acetone:toluene). [α]_{D}²⁰: +25° (c 1.0, CHCl₃). ¹H NMR (CDCl₃, 500 MHz): δH 8.05-8.02 (m, 2H, Ar), 7.60-7.53 (m, 3H, Ar), 7.47-7.44 (m, 2H, Ar), 7.40-7.34 (m, 3H, Ar), 7.24-7.16 (m, 5H, Ar), 5.73 (dd, 1H, J=9.7, 9.7 Hz, H-2), 5.51 (s, 1H, PhCH), 4.68 (d, 1H, J=12.8 Hz, PhCH^{a}H^{b}), 4.61 (d, 1H, J=12.8 Hz, PhCH^{a}H^{b}), 4.53 (d, 1H, J=9.8 Hz, H-1), 4.35 (dd, 1H, J=12.3, 1.5 Hz, H-6^{a}), 4.27 (dd, 1H, J=3.4, <1 Hz, H-4), 4.01 (dd, 1H, J=12.3, 1.7 Hz, H-6^{b}), 3.74 (dd, 1H, J=9.6, 3.4 Hz, H-3), 3.46-3.45 (m, 1H, H-5), 2.91 (dq, 1H, J=12.3, 7.5 Hz, SCH^{a}H^{b}CH₃), 2.76 (dq, 1H, J=12.3, 7.5 Hz, SCH^{a}H^{b}CH₃), 1.27 (dd, 3H, J=7.5, 7.5 Hz, SCH₂CH₃). ¹³C NMR (CDCl₃, 125 MHz): δC 165.46 (C═O), 138.01 (Ar), 137.94 (Ar), 133.21 (Ar), 130.33 (Ar), 130.07 (Ar), 129.25 (Ar), 128.53 (Ar), 128.51 (Ar), 128.41 (Ar), 127.92 (Ar), 127.88 (Ar), 126.68 (Ar), 101.56 (PhCH), 83.09 (C-1), 78.32 (C-3), 73.63 (C-4), 71.20 (PhCH₂), 70.34 (C-5), 69.58 (C-6), 68.95 (C-2), 22.92 (SCH₄₂CH₃), 15.05 (SCH₂CH₃). LRMS m/z calc'd for C₂₉H₃₀NaO₆S (M+Na)⁺: 529.17; found: 529.22.

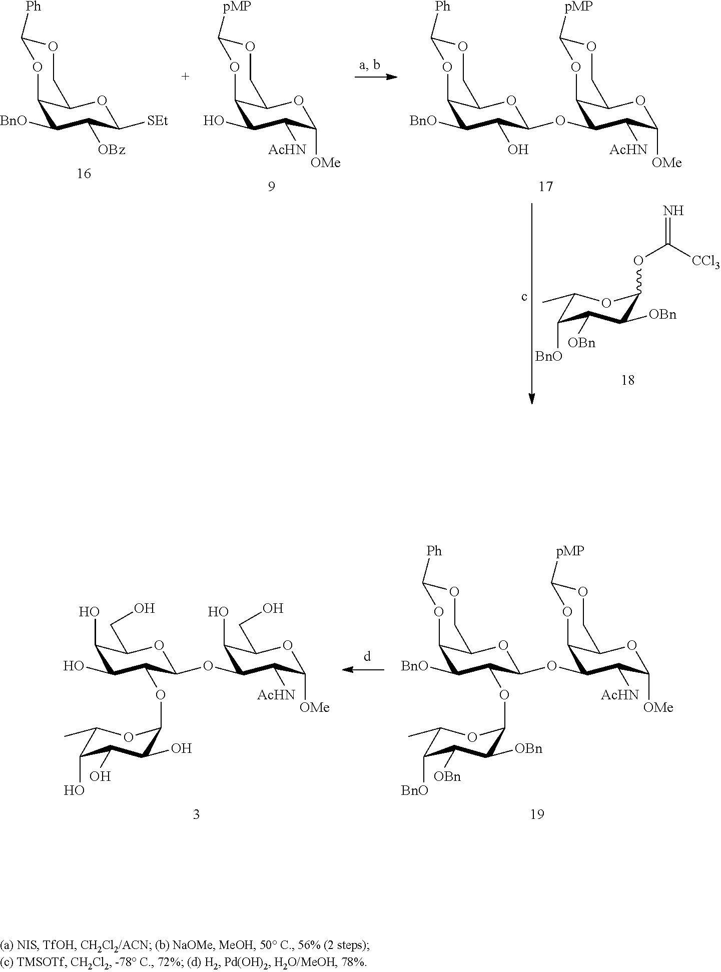

Methyl 3-O-benzyl-4,6-O-benzylidene-β-D-galactopyranosyl-(1→3)-2-acetamido-2-deoxy-4,6-O-(p-methoxybenzylidene)-α-D-galactopyranoside (17). The glycosyl acceptor (9; 209 mg, 0.591 mmol), glycosyl donor (16; 359 mg, 0.709 mmol), and molecular sieves (3 Å; 343 mg) in anhydrous CH₂Cl₂ (4.0 mL) and anhydrous acetonitrile (2.0 mL) were left mixing at rt under Ar. After 1 hour, the mixture was cooled to −40° C. and then N-iodosuccinimide (239 mg, 1.06 mmol) and dropwise triflic acid (7 μL, 0.08 mmol) were added. Three equivalent batches were prepared in parallel (2.527 mmol glycosyl donor combined), and after 4 hours, the mixture was neutralized with Et₃N, warmed to ambient temperature, filtered over Celite, combined and diluted with CH₂Cl₂ (250 mL). The organic phase was washed with sat'd Na₂S₂O_{3(aq)} solution (250 mL), sat'd NaCl_{(aq)} solution (250 mL), dried with Na₂SO₄, filtered, and evaporated to dry. The crude material was purified via MPLC on silica gel using 0→30% acetone (w/ 0.1% NH₄OH)—CH₂Cl₂ to afford the semi-pure product: R_{f}=0.28 (1:4 acetone w/ 0.1% NH₄OH:CH₂Cl₂). LRMS m/z calc'd for C₄₄H₄₇NNaO₁₃ (M+Na)⁺: 820.29; found: 820.15. The mixture was then added to anhydrous MeOH (5.0 mL), and NaOMe solution added (1.5 M NaOMe in MeOH; to pH 10). After 14 hours at 50° C., the mixture was neutralized with acidic resin (Amberlite IR-120H; to pH 6), filtered, and evaporated to dry. The crude material was purified via MPLC on silica gel using 0→30% acetone (w/ 0.1% NH₄OH)—CH₂Cl₂ to afford the pure product as a white solid (989 mg, 1.43 mmol, 56% yield over 2 steps). R_{f}=0.13 (1:4 acetone w/ 0.1% NH₄OH_{(aq)}:CH₂Cl₂). [α]_{D}²⁰: +127° (c 0.67, CHCl₃). ¹H NMR (CDCl₃, 500 MHz): δH 7.52-7.46 (m, 4H, Ar), 7.39-7.37 (m, 2H, Ar), 7.35-7.26 (m, 6H, Ar), 6.88-6.85 (m, 2H, Ar), 5.87 (d, 1H, J=8.4 Hz, NH), 5.56 (s, 1H, pMPCH), 5.45 (s, 1H, PhCH), 4.95 (d, 1H, J=3.6 Hz, GalNAc_H1), 4.81 (d, 1H, J=12.4 Hz, PhCH^{a}H^{b}), 4.77 (d, 1H, J=12.4 Hz, PhCH^{a}H^{b}), 4.67 (ddd, 1H, J=11.0, 8.5, 3.6 Hz, GalNAc_H2), 4.39 (d, 1H, J=7.7 Hz, Gal_H1), 4.38-4.37 (m, 1H, GalNAc_H4), 4.25 (dd, 1H, J=12.3, 1.5 Hz, Gal_H6^{a}), 4.23 (dd, 1H, J=12.4, 1.5 Hz, GalNAc_H6^{a}), 4.09-4.08 (m, 1H, Gal_H4), 4.06 (ddd, 1H, J=9.7, 7.8, 1.6 Hz, Gal_H2), 4.02 (dd, 1H, J=12.3, 1.7 Hz, Gal_H6^{b}), 4.02 (dd, 1H, J=12.4, 1.5 Hz, GalNAc_H6^{b}), 3.94 (dd, 1H, J=11.0, 3.2 Hz, GalNAc_H3), 3.80 (s, 3H, OCH₃), 3.62-3.61 (m, 1H, GalNAc_H5), 3.43 (dd, 1H, J=9.8, 3.5 Hz, Gal_H3), 3.41 (s, 3H, OCH₃), 3.34-3.33 (m, 1H, Gal_H5), 2.85 (d, 1H, J=1.7 Hz, 2-OH), 2.00 (s, 3H, Ac). ¹³C NMR (CDCl₃, 125 MHz): δC 171.13 (C═O), 160.23 (Ar), 138.61 (Ar), 137.99 (Ar), 130.64 (Ar), 129.27 (Ar), 128.56 (Ar), 128.45 (Ar), 128.11 (Ar), 128.07 (Ar), 127.90 (Ar), 126.63 (Ar), 113.73 (Ar), 105.05 (Gal_C1), 101.47 (PhCH), 101.20 (pMPCH), 99.65 (GalNAc_C1), 78.68 (Gal_C3), 76.13 (GalNAc_C4), 75.67 (GalNAc_C3), 73.96 (Gal_C4), 71.88 (PhCH₂), 69.63 (Gal_C2), 69.50, 69.38 (Gal_C6 and GalNAc_C6), 67.03 (Gal_C5), 63.31 (GalNAc_C5), 55.64 (OCH₃), 55.53 (OCH₃), 48.73 (GalNAc_C2), 23.87 (Ac). LRMS m/z calc'd for C₃₇H₄₃NNaO₁₂ (M+Na)⁺: 716.27; found: 716.26.

Methyl 2,3,4-tri-O-benzyl-α-L-fucopyranosyl-(1→2)-3-O-benzyl-4,6-O-benzylidene-β-D-galactopyranosyl-(1→3)-2-acetamido-2-deoxy-4,6-O-(p-methoxybenzylidene)-α-D-galactopyranoside (19). The glycosyl acceptor (17; 198 mg, 0.285 mmol), glycosyl donor⁶⁵ (18; 212 mg, 0.366 mmol), and molecular sieves (3 Å; 246 mg) in anhydrous CH₂Cl₂ (5.0 mL) were left mixing at rt under Ar. After 1 hour, the mixture was cooled to −78° C. and then trimethylsilyl triflate was added dropwise (7.5 μL, 0.041 mmol). Three equivalent batches were prepared in parallel (0.836 mmol glycosyl donor combined), and after 3 hours the mixtures were neutralized with Et₃N, filtered over Celite, combined and then diluted with CH₂Cl₂ (500 mL). The organic phase was washed with sat'd NaHCO_{3(aq)} solution (500 mL), sat'd NaCl_{(aq)} solution (500 mL), dried with Na₂SO₄, filtered, and evaporated to dry. The crude material was purified via MPLC on silica gel using 0→30% acetone (w/ 0.1% NH₄OH)—CH₂Cl₂ to afford the pure product as a white solid (670 mg, 0.603 mmol, 72% yield). R_{f}=0.45 (1:4 acetone w/ 0.1% NH₄OH:CH₂Cl₂). [α]_{D}²⁰: +34° (c 1.0, CHCl₃). ¹H NMR (CDCl₃, 500 MHz): δH 7.54-7.50 (m, 2H, Ar), 7.48-7.45 (m, 2H, Ar), 7.35-7.32 (m, 5H, Ar), 7.29-7.20 (m, 13H, Ar), 7.19-7.12 (m, 5H, Ar), 6.78-6.75 (m, 2H, Ar), 6.11 (d, 1H, J=8.3 Hz, NH), 5.50 (s, 1H, pMPCH), 5.48 (d, 1H, J=3.8 Hz, Fuc_H1), 5.43 (s, 1H, PhCH), 4.89 (d, 1H, J=11.4 Hz, PhCH^{a}H^{b}), 4.89 (d, 1H, J=3.5 Hz, GalNAc_H1), 4.81 (d, 1H, J=11.9 Hz, PhCH^{a}H^{b}), 4.76 (d, 1H, J=11.9 Hz, PhCH^{a}H^{b}), 4.70 (d, 1H, J=12.0 Hz, PhCH^{a}H^{b}), 4.64-4.59 (m, 4H, PhCH^{a}H^{b}, GalNAc_H2, PhCH^{a}H^{b}, and Gal_H1), 4.54 (d, 1H, J=11.4 Hz, PhCH^{a}H^{b}), 4.53 (d, 1H, J=12.1 Hz, PhCH^{a}H^{b}), 4.39 (dd, 1H, J=3.0, <1 Hz, GalNAc_H4), 4.25-4.18 (m, 4H, Gal_H6^{a}, GalNAc_H6^{a}, Gal_H2, and Fuc_H5), 4.12 (dd, 1H, J=3.6, <1 Hz, Gal_H4), 4.04-3.96 (m, 3H, Gal_H6^{b}, GalNAc_H6^{b}, and GalNAc_H3), 3.94 (dd, 1H, J=10.1, 3.8 Hz, Fuc_H2), 3.89 (dd, 1H, J=10.2, 2.6 Hz, Fuc_H3), 3.74 (s, 3H, OCH₃), 3.73-3.72 (m, 1H, Fuc_H4), 3.66 (dd, 1H, J=9.5, 3.7 Hz, Gal_H3), 3.58-3.57 (m, 1H, GalNAc_H5), 3.37 (s, 3H, OCH₃), 3.34-3.32 (m, 1H, Gal_H5), 1.87 (s, 3H, Ac), 0.92 (d, 3H, J=6.4 Hz, Fuc_H6). ¹³C NMR (CDCl₃, 125 MHz): δC 170.03 (C═O), 160.09 (Ar), 139.51 (Ar), 139.41 (Ar), 138.86 (Ar), 138.73 (Ar), 138.05 (Ar), 130.71 (Ar), 129.15 (Ar), 128.41 (Ar), 128.39 (Ar), 128.24 (Ar), 128.21 (Ar), 128.11 (Ar), 128.02 (Ar), 127.96 (Ar), 127.60 (Ar), 127.41 (Ar), 127.38 (Ar), 127.35 (Ar), 126.54 (Ar), 113.54 (Ar), 103.35 (Gal_C1), 101.34 (pMPCH), 101.09 (PhCH), 99.60 (GalNAc_C1), 97.66 (Fuc_C1), 80.51 (Gal_C3), 79.28 (Fuc_C3), 78.53 (Fuc_C4), 76.43 (GalNAc_C4), 76.10 (Fuc_C2), 75.12 (PhCH₂), 74.54 (Gal_C2), 73.73 (GalNAc_C3), 73.26 (Gal_C4), 72.70 (PhCH₂), 72.47 (PhCH₂), 71.31 (PhCH₂), 69.41 (Gal_C6 and GalNAc_C6), 67.17 (Fuc_C5), 66.54 (Gal_C5), 63.32 (GalNAc_C5), 55.65 (OCH₃), 55.47 (OCH₃), 48.85 (GalNAc_C2), 23.65 (Ac), 16.41 (Fuc_C6). LRMS m/z calc'd for C₆₄H₇₁NNaO₁₆ (M+Na)⁺: 1132.46; found: 1132.38.

Methyl α-L-fucopyranosyl-(1→2)-β-D-galactopyranosyl-(1→3)-2-acetamido-2-deoxy-α-D-galactopyranoside (3). The starting material (19; 37 mg, 0.033 mmol) and Pd(OH)₂ (20% w/w on carbon, 13 mg) in MeOH (0.8 mL) and H₂O (0.8 mL) were left mixing under H₂ at atmospheric pressure. After 48 hours, the solid catalyst was removed via filtration and the solution evaporated to dry. The crude material was purified via RPLC on C-18 silica gel using 0→60% acetonitrile-H₂O to afford the pure product as a white solid (14 mg, 0.026 mmol, 78% yield); data characterization is in agreement with that previously published⁶². [α]_{D}²⁰: +42° (c 0.5, H₂O). ¹H NMR (D₂O, 500 MHz): δH 5.22 (d, 1H, J=4.1 Hz, Fuc_H1), 4.75 (m, 1H, GalN_H1), 4.62 (d, 1H, J=7.7 Hz, Gal_H1), 4.21 (dq, 1H, J=6.6, <1 Hz, Fuc_H5), 4.13-4.09 (m, 2H, GalN_H2 and GalN_H4), 4.11 (dd, 1H, J=11.2, 2.9 Hz, GalN_H3), 3.96 (ddd, 1H, J=6.2, 6.2, <1 Hz, GalN_H5), 3.89 (dd, 1H, J=3.4, <1 Hz, Gal_H4), 3.82 (dd, 1H, J=9.7, 3.4 Hz, Gal_H3), 3.79-3.71 (m, 5H, GalN_H6^{a}, Fuc_H2, GalN_H6^{b}, Gal_H6^{a}, and Gal_H6^{b}), 3.67-3.60 (m, 4H, Fuc_H4, Fuc_H3, Gal_H5, and Gal_H2), 3.35 (s, 3H, OCH₃), 2.03 (s, 3H, Ac), 1.18 (d, 3H, J=6.6 Hz, Fuc_H6). ¹³C NMR (D₂O, 125 MHz): δC 174.31 (C═O), 102.63 (Gal_C1), 99.94 (Fuc_C1), 98.53 (GalN_C1), 76.93 (Gal_C2), 75.66 (Gal_C5), 74.41 (GalN_C3), 74.21 (Gal_C3), 72.51 (Fuc_C4), 71.06 (GalN_C5), 70.21 (Fuc_C3), 69.71 (Gal_C4 and GalN_C4), 68.72 (Fuc_C2), 67.44 (Fuc_C5), 61.88 (GalN_C6), 61.56 (Gal_C6), 55.74 (OCH₃), 50.06 (GalN_C2), 22.59 (Ac), 16.00 (Fuc_C6). ESI-HRMS m/z calc'd for C₂₁H₃₇NNaO₁₅ (M+Na)⁺: 566.2061; found: 566.2061. HPLC purity analysis: >99.5%, Rₜ 5.55 minutes, Atlantis T3 C18 column.

Ethyl 2,3-di-O-acetyl-4,6-O-benzylidene-1-thio-β-D-galactopyranoside (20). The starting material (14; 1.071 g, 3.429 mmol) was dissolved into anhydrous pyridine (6.0 mL) and Ac₂O (6.0 mL), and left mixing at rt. After 3 hours, the reaction mixture was evaporated to dry via co-evaporation with toluene (3×10 mL), and then the crude material was purified via MPLC on silica gel using 0→40% acetone-toluene to afford the pure product as a white solid (1.306 g,

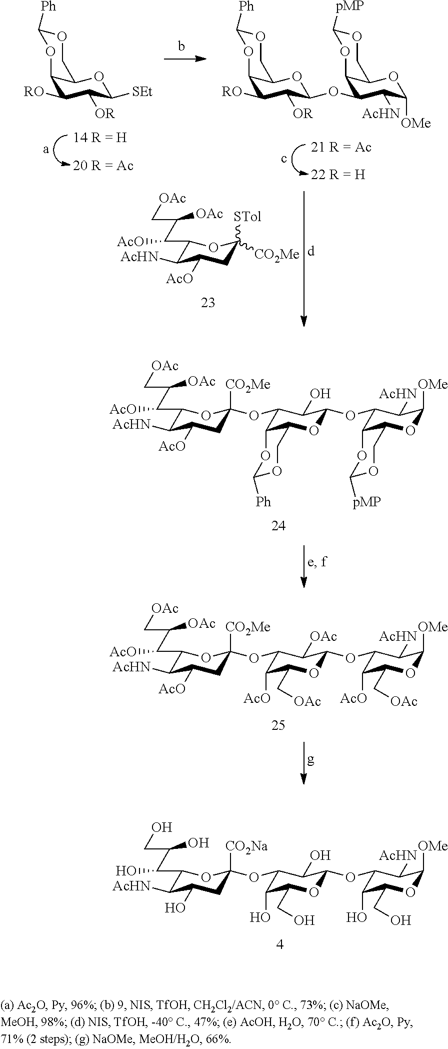

3.294 mmol, 96% yield). R_{f}=0.64 (1:4 acetone:toluene). [α]_{D}²⁰: +28° (c 1.0, CHCl₃). ¹H NMR (CDCl₃, 500 MHz): δH 7.51-7.46 (m, 2H, Ar), 7.40-7.34 (m, 3H, Ar), 5.48 (s, 1H, PhCH), 5.46 (dd, 1H, J=9.9, 9.9 Hz, H-2), 4.98 (dd, 1H, J=10.0, 3.5 Hz, H-3), 4.45 (d, 1H, J=9.8 Hz, H-1), 4.39 (dd, 1H, J=3.5, <1 Hz, H-4), 4.31 (dd, 1H, J=12.5, 1.5 Hz, H-6^{a}), 3.99 (dd, 1H, J=12.5, 1.6 Hz, H-6^{b}), 3.53-3.52 (m, 1H, H-5), 2.87 (dq, 1H, J=12.3, 7.5 Hz, SCH^{a}H^{b}CH₃), 2.72 (dq, 1H, J=12.3, 7.5 Hz, SCH^{a}H^{b}CH₃), 2.06 (s, 3H, Ac), 2.05 (s, 3H, Ac), 1.28 (dd, 3H, J=7.5, 7.5 Hz, SCH₂CH₃). ¹³C NMR (CDCl₃, 125 MHz): δC 170.69 (C═O), 169.53 (C═O), 137.72 (Ar), 129.21 (Ar), 128.30 (Ar), 126.46 (Ar), 101.20 (PhCH), 82.86 (C-1), 73.71 (C-4), 73.10 (C-3), 69.80 (C-5), 69.18 (C-6), 66.70 (C-2), 22.90 (SCH₄₂CH₃), 20.96 (Ac), 20.95 (Ac), 14.88 (SCH₂CH₃). LRMS m/z calc'd for C₁₉H₂₄NaO₇S (M+Na)⁺: 419.11; found: 419.11.

Methyl 2,3-di-O-acetyl-4,6-O-benzylidene-β-D-galactopyranosyl-(1→3)-2-acetamido-2-deoxy-4,6-O-(p-methoxybenzylidene)-α-D-galactopyranoside (21). The glycosyl acceptor (9; 504 mg, 1.43 mmol), glycosyl donor (20; 1.119 g, 2.823 mmol), and crushed molecular sieves (3 Å, 1.476 g) in anhydrous CH₂Cl₂ (10 mL) and anhydrous acetonitrile (5 mL) were left mixing for 1 hour at ambient temperature under Ar. The reaction flask was cooled to 0° C., and then NIS added (641 mg, 2.85 mmol) followed by the drop-wise addition of TfOH (12 μL, 0.14 mmol). After 5 hours, the mixture was neutralized with Et₃N (to pH 8), warmed to ambient temperature, diluted with CH₂Cl₂ (100 mL), and filtered over Celite. The organic phase was washed with saturated Na₂S₂O_{3(aq)} solution (100 mL), saturated NaCl_{(aq)} solution (100 mL), dried with Na₂SO₄, filtered, and evaporated to dry. The crude material was purified via MPLC on silica gel using 0→40% acetone (containing 0.05% v/v conc'd NH₄OH_{(aq)})—CH₂Cl₂ to afford the mostly pure intermediate as a white solid (722 mg, 1.05 mmol, 73% yield). R_{f}=0.39 (3:7 acetone w/ 0.1% NH₄OH:CH₂Cl₂). [α]_{D}²⁰: +122° (c 1.0, CH₃OH). ¹H NMR (CD₃OD, 500 MHz): δH 7.53-7.49 (m, 2H, Ar), 7.46-7.43 (m, 2H, Ar), 7.36-7.33 (m, 3H, Ar), 6.85-6.82 (m, 2H, Ar), 5.59 (s, 1H, PhCH), 5.57 (s, 1H, pMPCH), 5.27 (dd, 1H, J=10.4, 8.0 Hz, Gal_H2), 5.05 (dd, 1H, J=10.4, 3.7 Hz, Gal_H3), 4.88 (d, 1H, J=8.0 Hz, Gal_H1), 4.72 (d, 1H, J=3.5 Hz, GalNAc_H1), 4.56 (dd, 1H, J=11.2, 3.5 Hz, GalNAc_H2), 4.49 (dd, 1H, J=3.3, <1 Hz, GalNAc_H4), 4.42 (dd, 1H, J=3.7, 0.7 Hz, Gal_H4), 4.25 (dd, 1H, J=12.4, 1.5 Hz, Gal_H6^{a}), 4.17 (dd, 1H, J=12.5, 1.6 Hz, Gal_H6^{b}), 4.16-4.13 (m, 1H, GalNAc_H6^{a}), 4.12-4.09 (m, 2H, GalNAc_H6^{b} and GalNAc_H3), 3.77 (s, 3H, ArOCH₃), 3.71-3.70 (m, 1H, GalNAc_H5), 3.67-3.66 (m, 1H, Gal_H5), 3.42 (s, 3H, OCH₃), 2.03 (s, 3H, NHAc), 2.01 (s, 3H, OAc), 2.00 (s, 3H, OAc). ¹³C NMR (CD₃OD, 125 MHz): δC 173.24 (C═O), 172.03 (C═O), 171.69 (C═O), 161.52 (Ar), 139.58 (Ar), 132.31 (Ar), 130.16 (Ar), 129.25 (Ar), 128.96 (Ar), 127.71 (Ar), 114.40 (Ar), 102.94 (Gal_C1), 102.32 (ArCH), 102.05 (ArCH), 101.12 (GalNAc_C1), 77.34 (GalNAc_C4), 75.49 (GalNAc_C3), 75.09 (Gal_C4), 73.39 (Gal_C3), 70.51 (Gal_C2), 70.38 (GalNAc_C6), 70.12 (Gal_C6), 67.95 (Gal_C5), 64.62 (GalNAc_C5), 55.98 (OCH₃), 55.83 (ArOCH₃), 50.05 (GalNAc_C2), 23.09 (NHAc), 21.08 (OAc), 20.73 (OAc). LRMS m/z calc'd for C₃₄H₄₁NNaO₁₄ (M+Na)⁺: 710.24; found: 710.21.

Methyl 4,6-O-benzylidene-β-D-galactopyranosyl-(1→3)-2-acetamido-2-deoxy-4,6-O-(p-methoxybenzylidene)-α-D-galactopyranoside (22). The starting material (21; 548 mg, 0.797 mmol) was dissolved into anhydrous MeOH (6.0 mL), and then NaOMe solution was added drop-wise (1.5 M NaOMe in MeOH; to pH 10). After 2 hours at ambient temperature, the reaction mixture was neutralized with acidic resin (Amberlite IR-120H; to pH 6), filtered, and then evaporated to dry. The crude material was purified via MPLC on silica gel using 0→100% acetone (w/ 0.1% NH₄OH)—CH₂Cl₂ to afford the pure product as a white solid (470 mg, 0.779 mmol, 98% yield). R_{f}=0.07 (1:1 acetone w/ 0.1% NH₄OH:CH₂Cl₂). [α]_{D}²⁰: +104° (c 1.0, CH₃OH). ¹H NMR (CD₃OD, 500 MHz): δH 7.59-7.55 (m, 2H, Ar), 7.49-7.46 (m, 2H, Ar), 7.39-7.33 (m, 3H, Ar), 6.90-6.86 (m, 2H, Ar), 5.65 (s, 1H, PhCH), 5.61 (s, 1H, pMPCH), 4.82 (d, 1H, J=3.4 Hz, GalNAc_H1), 4.62 (dd, 1H, J=11.2, 3.4 Hz, GalNAc_H2), 4.55 (dd, 1H, J=3.2, <1 Hz, GalNAc_H4), 4.52 (d, 1H, J=7.4 Hz, Gal_H1), 4.26 (dd, 1H, J=12.4, 1.4 Hz, Gal_H6^{a}), 4.22 (dd, 1H, J=3.5, 0.8 Hz, Gal_H4), 4.19 (dd, 1H, J=12.4, 1.7 Hz, Gal_H6^{b}), 4.17 (dd, 1H, J=12.5, 1.4, GalNAc_H6^{a}), 4.15-4.11 (m, 2H, GalNAc_H6^{b} and GalNAc_H3), 3.81 (s, 3H, ArOCH₃), 3.75-3.74 (m, 1H, GalNAc_H5), 3.67 (dd, 1H, J=9.9, 7.4 Hz, Gal_H2), 3.61 (dd, 1H, J=9.9, 3.5 Hz, Gal_H3), 3.58-3.57 (m, 1H, Gal_H5), 3.46 (s, 3H, OCH₃), 2.00 (s, 3H, NHAc). ¹³C NMR (CD₃OD, 125 MHz): δC 174.61 (C═O), 162.06 (Ar), 140.19 (Ar), 132.69 (Ar), 130.34 (Ar), 129.50 (Ar), 129.49 (Ar), 128.10 (Ar), 114.78 (Ar), 106.75 (Gal_C1), 102.83, 102.76 (PhCH and pMPCH), 101.50 (GalNAc_C1), 78.01 (GalNAc_C4), 77.92 (Gal_C4), 76.14 (GalNAc_C3), 74.01 (Gal_C3), 72.23 (Gal_C2), 70.83 (Gal_C6), 70.80 (GalNAc_C6), 68.64 (Gal_C5), 64.99 (GalNAc_C5), 56.35 (OCH₃), 56.20 (ArOCH₃), 50.74 (GalNAc_C2), 23.33 (NHAc). LRMS m/z calc'd for C₃₀H₃₇NNaO₁₂ (M+Na)⁺: 626.22; found: 626.18.

Methyl O-[methyl 5-acetamido-4,7,8,9-tetra-O-acetyl-3,5-dideoxy-D-glycero-α-D-galacto-2-nonulopyranosylonate)-(2→3)-4,6-O-benzylidene-β-D-galactopyranosyl-(1→3)-2-acetamido-2-deoxy-4,6-O-(p-methoxybenzylidene)-α-D-galactopyranoside (24). The glycosyl acceptor (22; 399 mg, 0.661 mmol), glycosyl donor (23; 601 mg, 1.01 mmol), and crushed molecular sieves (3 Å, 866 mg) in anhydrous CH₂Cl₂ (5.0 mL) and anhydrous acetonitrile (5.0 mL) were left mixing for 1 hour at ambient temperature under Ar. The reaction flask was cooled to −40° C., and then NIS (298 mg, 1.32 mmol) and TfOH (9 μL, 0.10 mmol) were added. After 24 hours, the mixture was neutralized with Et₃N (to pH 8), warmed to ambient temperature, filtered over Celite, and evaporated to dry. The crude material was redissolved into CH₂Cl₂ (30 mL), washed with saturated NaHCO_{3(aq)} solution (2×30 mL), saturated NaCl_{(aq)} solution (30 mL), dried with Na₂SO₄, filtered, and evaporated to dry. The crude material was purified via MPLC on silica gel using 0→30% acetone (w/ 0.1% NH₄OH)—CH₂Cl₂ to afford the pure product as a white solid (333 mg, 0.309 mmol, 47% yield). ¹H NMR (CDCl₃, 500 MHz) δ 7.49-7.46 (m, 4H, Ar), 7.34-7.31 (m, 3H, Ar), 6.85-6.82 (m, 2H, Ar), 6.50 (d, 1H, J=7.8 Hz, GalNAc_NH), 5.55 (s, 1H, ArCH), 5.51 (ddd, 1H, J=9.7, 7.1, 2.5 Hz, Neu5Ac_H8), 5.40-5.37 (m, 2H, Neu5Ac_NH and ArCH), 5.24 (dd, 1H, J=9.4, 1.3 Hz, Neu5Ac_H7), 5.04 (d, 1H, J=3.3 Hz, GalNAc_H1), 4.87 (ddd, 1H, J=12.2, 9.8, 4.5 Hz, Neu5Ac_H4), 4.64 (ddd, 1H, J=11.1, 7.8, 3.3 Hz, GalNAc_H2), 4.55 (d, 1H, J=7.7 Hz, Gal_H1), 4.41 (dd, 1H, J=2.8, <1 Hz, GalNAc_H4), 4.38 (dd, 1H, J=12.2, 2.4 Hz, Neu5Ac_H9^{a}), 4.25-4.20 (m, 3H, GalNAc_H6^{a}, Gal_H6^{a}, and Gal_H3), 4.11 (dd, 1H, J=12.4, <2 Hz, Gal_H6^{b}), 4.07-4.01 (m, 4H, Neu5Ac_H5, GalNAc_H3, GalNAc_H6^{b}, and Neu5Ac_H6), 3.95-3.89 (m, 3H, Neu5Ac_H9^{b}, Gal_H4, and Gal_H2), 3.78 (s, 3H, OCH₃), 3.64-3.62 (s, 4H, GalNAc_H5 and OCH₃), 3.50-3.49 (m, 1H, Gal_H5), 3.40 (s, 3H, OCH₃), 2.91-2.89 (broad s, 1H, Gal_2-OH), 2.71 (dd, 1H, J=12.9, 4.5 Hz, Neu5Ac_H3_{eq}), 2.19 (s, 3H, OAc), 2.15 (s, 3H, OAc), 2.05-2.00 (m, 10H, OAc, NHAc, Neu5Ac_H3ₐₓ, and OAc), 1.88 (s, 3H, NHAc). LRMS m/z calc'd for C₅₀H₆₄N₂NaO₂₄ (M+Na)⁺: 1099.37; found: 1099.27.

Methyl 2,4,6-tri-O-acetyl-3-O-(methyl 5-acetamido-4,7,8,9-tetra-O-acetyl-3,5-dideoxy-D-glycero-α-D-galacto-2-nonulopyranosylonate)-β-D-galactopyranosyl-(1→3)-2-acetamido-4,6-di-O-acetyl-2-deoxy-α-D-galactopyranoside (25). The starting material (24; 96 mg, 0.089 mmol) was dissolved into AcOH (1.2 mL) and H₂O (0.3 mL) and left mixing at 70° C. After 7 hours, the mixture was evaporated to dry via co-evaporation with toluene (3×2 mL), and then redissolved into pyridine (1.0 mL) and Ac₂O (1.0 mL). After 18 hours, the reaction mixture was concentrated to a syrup via co-evaporation with toluene (3×2 mL), and then the crude material was purified via MPLC on silica gel using 0→15% MeOH—CH₂Cl₂ to afford the pure product as a white solid (68 mg, 0.063 mmol, 71% yield over 2 steps). ¹H NMR (CDCl₃, 500 MHz): δH 6.40 (d, 1H, J=8.1 Hz, GalNAc_NH), 5.70 (ddd, 1H, J=9.3, 7.7, 2.6 Hz, Neu5Ac_H8), 5.42 (dd, 1H, J=2.8, <1 Hz, GalNAc_H4), 5.31 (dd, 1H, J=9.4, 2.7 Hz, Neu5Ac_H7), 5.23 (d, 1H, J=10.2 Hz, Neu5Ac_NH), 5.00 (dd, 1H, J=10.1, 8.1 Hz, Gal_H2), 4.91 (d, 1H, J=3.5 Hz, GalNAc_H1), 4.89-4.83 (m, 2H, Gal_H4 and Neu5Ac_H4), 4.68 (d, 1H, J=8.0 Hz, Gal_H1), 4.50-4.45 (m, 2H, Gal_H3 and GalNAc_H2), 4.39 (dd, 1H, J=12.1, 2.6 Hz, Neu5Ac_H9^{a}), 4.21 (dd, 1H, J=11.4, 4.6 Hz, GalNAc_H6^{a}), 4.11-4.04 (m, 3H, Neu5Ac_H5, GalNAc_H5, and Gal_H6^{a}), 4.01 (dd, 1H, J=11.4, 6.5 Hz, Gal_H6^{b}), 3.95 (dd, 1H, J=11.4, 7.6 Hz, GalNAc_H6^{b}), 3.93-3.84 (m, 6H, GalNAc_H3, Neu5Ac_H9^{b}, Gal_H5, and OCH₃), 3.67 (dd, 1H, J=10.7, 2.7 Hz, Neu5Ac_H6), 3.35 (s, 3H, OCH₃), 2.59 (dd, 1H, J=12.6, 4.5 Hz, Neu5Ac_H3_{eq}), 2.27 (s, 3H, OAc), 2.20 (s, 3H, OAc), 2.13 (s, 3H, OAc), 2.10 (s, 3H, OAc), 2.10 (s, 3H, OAc), 2.09 (s, 3H, OAc), 2.07 (s, 3H, OAc), 2.06 (s, 3H, OAc), 2.01 (s, 6H, OAc and NHAc), 1.86 (s, 3H, NHAc), 1.69 (dd, 1H, J=12.4, 12.4 Hz, Neu5Ac_H3ₐₓ). ¹³C NMR (CDCl₃, 125 MHz): δC 171.50 (Ac), 171.11 (Ac), 171.03 (Ac), 170.81 (Ac), 170.55 (Ac), 170.51 (Ac), 170.49 (Ac), 170.26 (Ac), 170.24 (Ac), 170.07 (Ac), 170.06 (Ac), 168.12 (Neu5Ac_C1), 101.63 (Gal_C1), 98.66 (GalNAc_C1), 96.93 (Neu5Ac_C2), 74.42 (GalNAc_C3), 72.07 (Neu5Ac_C6), 71.51 (Gal_C3), 70.95 (Gal_C5), 69.58 (GalNAc_C4), 69.41 (Neu5Ac_C4), 69.02 (Gal_C2), 67.75 (Gal_C4), 67.61 (Neu5Ac_C7), 67.49 (GalNAc_C5), 67.40 (Neu5Ac_C8), 63.47 (Neu5Ac_C9), 63.24 (GalNAc_C6), 62.03 (Gal_C6), 55.38 (OCH₃), 53.35 (OCH₃), 49.35 (GalNAc_C2), 49.15 (Neu5Ac_C5), 37.59 (Neu5Ac_C3), 23.30 (NHAc), 23.28 (NHAc), 21.62 (OAc), 21.25 (OAc), 21.02 (2×OAc), 20.93 (4×OAc), 20.85 (OAc). LRMS m/z calc'd for C₄₅H₆₄N₂NaO₂₈ (M+Na)⁺: 1103.35; found: 1103.29.

Methyl 3-O-(sodium 5-acetamido-3,5-dideoxy-D-glycero-α-D-galacto-2-nonulopyranosylonate)-β-D-galactopyranosyl-(1→3)-2-acetamido-2-deoxy-α-D-galactopyranoside (4). The starting material (25; 55 mg, 0.051 mmol) was dissolved into 50% aqueous MeOH (1.0 mL), and then NaOMe solution was added drop-wise (1.5 M NaOMe in MeOH; to pH 10). After 14 hours, the reaction mixture was neutralized with acidic resin (Amberlite IR-120H; to pH 8), filtered, and evaporated to dry. The crude material was purified via RPLC on C-18 silica gel using 0→20% acetonitrile-H₂O to afford the pure product as a white solid (24 mg, 0.034 mmol, 66% yield). [α]_{D}²⁰: +32° (c 1.0, H₂O). ¹H NMR (D₂O, 500 MHz): δH 4.79 (d, 1H, J=3.7 Hz, GalNAc_H1), 4.52 (d, 1H, J=7.9 Hz, Gal_H1), 4.31 (dd, 1H, J=11.1, 3.7 Hz, GalNAc_H2), 4.22 (dd, 1H, J=2.7, <1 Hz, GalNAc_H4), 4.06 (dd, 1H, J=9.8, 3.2 Hz, Gal_H3), 4.00 (dd, 1H, J=11.1, 3.0 Hz, GalNAc_H3), 3.96-3.92 (m, 2H, GalNAc_H5 and Gal_H4), 3.87 (ddd, 1H, J=8.9, 6.3, 2.5 Hz, NeuNAc_H8), 3.86-3.81 (m, 2H, NeuNAc_H9^{a} and NeuNAc_H5), 3.79-3.60 (m, 8H, GalNAc_H6^{a}, GalNAc_H6^{b}, Gal_H6^{a}, Gal_H6^{b}, NeuNAc_H4, NeuNAc_H9^{b}, Gal_H5, and NeuNAc_H6), 3.58 (dd, 1H, J=8.9, 1.7 Hz, NeuNAc_H7), 3.53 (dd, 1H, J=9.8, 7.9 Hz, Gal_H2), 3.38 (s, 3H, OCH₃), 2.75 (dd, 1H, J=12.4, 4.6 Hz, NeuNAc_H3_{eq}), 2.02 (s, 3H, Ac), 2.01 (s, 3H, Ac), 1.77 (dd, 1H, J=12.2, 12.2 Hz, NeuNAc_H3ₐₓ). ¹³C NMR (D₂O, 125 MHz): δC 175.64 (C═O), 175.26 (C═O), 174.55 (NeuNAc_C1), 105.12 (Gal_C1), 100.33 (NeuNAc_C2), 98.93 (GalNAc_C1), 78.07 (GalNAc_C3), 76.30 (Gal_C3), 75.41 (Gal_C5), 73.44 (NeuNAc_C6), 72.48 (NeuNAc_C8), 71.09 (GalNAc_C5), 69.68 (Gal_C2), 69.23 (GalNAc_C4), 69.02 (NeuNAc_C4), 68.72 (NeuNAc_C7), 68.00 (Gal_C4), 63.16 (NeuNAc_C9), 61.90 (GalNAc_C6), 61.60 (Gal_C6), 55.73 (OCH₃), 52.30 (NeuNAc_C5), 49.22 (GalNAc_C2), 40.39 (NeuNAc_C3), 22.71 (Ac), 22.68 (Ac). ESI-HRMS m/z calc'd for C₂₆H₄₃N₂NaO₁₉ (M+Na)⁺: 733.2255; found: 733.2250. HPLC purity analysis: 98.2%, Rₜ 5.40 minutes, Atlantis T3 C18 column.

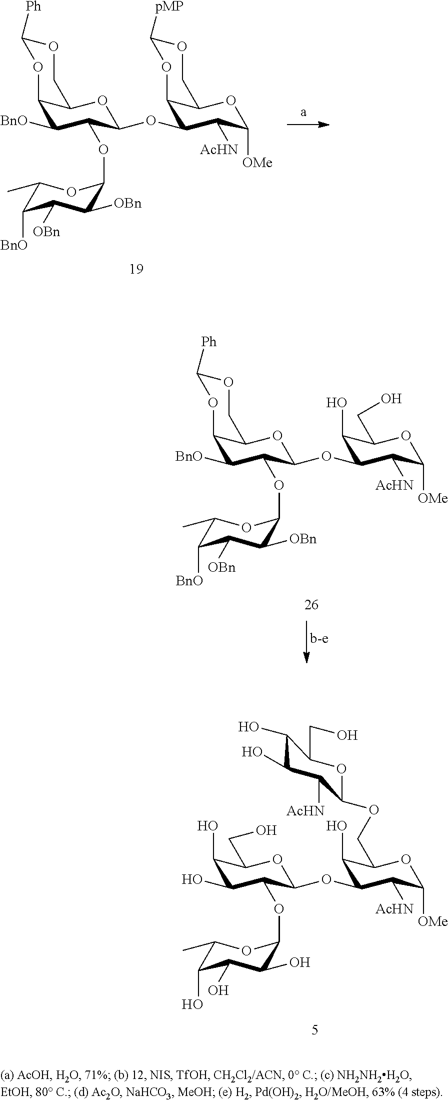

Methyl 2,3,4-tri-O-benzyl-α-L-fucopyranosyl-(1→2)-3-O-benzyl-4,6-O-benzylidene-β-D-galactopyranosyl-(1→3)-2-acetamido-2-deoxy-α-D-galactopyranoside (26). The starting material (19; 440 mg, 0.396 mmol) was dissolved into AcOH (3.2 mL) and H₂O (0.8 mL) and left mixing at ambient temperature. After 3 hours, the mixture was diluted with CH₂Cl₂ (50 mL) and sat'd NaHCO_{3(aq)} solution (50 mL), and then solid NaHCO₃ added until gas evolution subsided. The aqueous layer was removed and re-extracted with CH₂Cl₂ (2×20 mL), and then the combined organic layers washed with sat'd NaHCO_{3(aq)} solution (50 mL), dried with Na₂SO₄, filtered, and evaporated to dry. The crude material was purified via MPLC on silica gel using 0→100% acetone-CH₂Cl₂ to afford the pure product as a white solid (280 mg, 0.282 mmol, 71% yield). R_{f}=0.49 (1:1 acetone:CH₂Cl₂). [α]_{D}²⁰: +20° (c 1.0, CHCl₃). ¹H NMR (CDCl₃, 500 MHz): δH 7.53-7.50 (m, 2H, Ar), 7.38-7.20 (m, 20H, Ar), 7.19-7.14 (m, 3H, Ar), 6.95 (d, 1H, J=7.6 Hz, NH), 5.45 (s, 1H, PhCH), 5.29 (d, 1H, J=3.8 Hz, Fuc_H1), 5.00 (d, 1H, J=11.3 Hz, PhCH^{a}H^{b}), 4.85 (d, 1H, J=3.4 Hz, GalNAc_H1), 4.82 (d, 1H, J=12.0 Hz, PhCH^{a}H^{b}), 4.78 (d, 1H, J=12.2 Hz, PhCH^{a}H^{b}), 4.73 (d, 1H, J=12.1 Hz, PhCH^{a}H^{b}), 4.72-4.67 (m, 2H, PhCH^{a}H^{b} and PhCH^{a}H^{b}), 4.64 (d, 1H, J=11.9 Hz, PhCH^{a}H^{b}), 4.60 (d, 1H, J=11.4 Hz, PhCH^{a}H^{b}), 4.54 (d, 1H, J=7.3 Hz, Gal_H1), 4.40 (ddd, 1H, J=11.1, 7.7, 3.4 Hz, GalNAc_H2), 4.19-4.15 (m, 2H, Gal_H6^{a} and Fuc_H5), 4.13-4.10 (m, 3H, Gal_H2, Gal_H4, and GalNAc_H4), 4.00 (dd, 1H, J=10.2, 3.8 Hz, Fuc_H2), 3.99 (dd, 1H, J=12.4, 1.4 Hz, Gal_H6^{b}), 3.92-3.86 (m, 3H, Fuc_H3, GalNAc_H6^{a}, and GalNAc_H3), 3.79-3.73 (m, 3H, Fuc_H4, GalNAc_H5, and GalNAc_H6^{b}), 3.60 (dd, 1H, J=9.5, 3.4 Hz, Gal_H3), 3.38-3.37 (m, 1H, Gal_H5), 3.28 (s, 3H, OCH₃), 3.26-3.25 (m, 1H, 4-OH), 2.87-2.84 (m, 1H, 6-OH), 1.80 (s, 3H, Ac), 1.24 (d, 3H, J=6.5 Hz, Fuc_H6). ¹³C NMR (CDCl₃, 125 MHz): δC 170.45 (C═O), 139.10 (Ar), 139.05 (Ar), 138.75 (Ar), 138.48 (Ar), 137.77 (Ar), 129.18 (Ar), 128.44 (Ar), 128.36 (Ar), 128.26 (Ar), 128.12 (Ar), 127.93 (Ar), 127.63 (Ar), 127.60 (Ar), 127.58 (Ar), 127.54 (Ar), 127.50 (Ar), 127.35 (Ar), 126.47 (Ar), 103.24 (Gal_C1), 101.08 (PhCH), 98.61, 98.53 (GalNAc_C1 and Fuc_C1), 78.89 (Fuc_C3), 78.51 (Gal_C3), 78.22 (Fuc_C4), 78.16 (Gal_C2), 77.07 (GalNAc_C3), 76.32 (Fuc_C2), 75.05 (PhCH₂), 73.89 (Gal_C4), 73.15 (PhCH₁₂), 72.94 (PhCH₄₂), 72.25 (PhCH₄₂), 69.34 (GalNAc_C5), 69.30 (Gal_C6), 69.20 (GalNAc_C4), 67.92 (Fuc_C5), 66.74 (Gal_C5), 63.03 (GalNAc_C6), 55.42 (OCH₃), 48.99 (GalNAc_C2), 23.06 (Ac), 16.78 (Fuc_C6). LRMS m/z calc'd for C₅₆H₆₅NNaO₁₅ (M+Na)⁺: 1014.43; found: 1014.54.

Methyl α-L-fucopyranosyl-(1→2)-β-D-galactopyranosyl-(1→3)-[2-acetamido-2-deoxy-β-D-glucopyranosyl-(1→6)]-2-acetamido-2-deoxy-α-D-galactopyranoside (5). The glycosyl acceptor (26; 188 mg, 0.189 mmol), glycosyl donor⁶⁶ (12; 118 mg, 0.246 mmol), and crushed molecular sieves (3 Å, 171 mg) in anhydrous CH₂Cl₂ (1.5 mL) and anhydrous acetonitrile (1.5 mL) were left mixing for 1 hour at ambient temperature under Ar. The reaction flask was cooled to 0° C., and then NIS was added (78 mg, 0.35 mmol) followed by the drop-wise addition of TfOH (10 μL, 0.02 mmol). After 2 hours, the mixture was neutralized with Et₃N (to pH 8), warmed to ambient temperature, and diluted with CH₂Cl₂ (60 mL). The organic phase was washed with saturated Na₂S₂O_{3(aq)} solution (60 mL), saturated NaCl_{(aq)} solution (60 mL), dried with Na₂SO₄, filtered, and evaporated to dry. The crude material was purified via MPLC on silica gel using 0→40% acetone (containing 0.1% v/v NH₄OH_{(aq)})—CH₂Cl₂ to afford a mostly pure product (containing a small amount of a lower R_{f} by-product): R_{f}=0.75 (0.01:19.99:80 NH₄OH:acetone:CH₂Cl₂). LRMS m/z calc'd for C₇₆H₈₄N₂NaO₂₄ (M+Na)⁺: 1431.53; found: 1431.54. The product and NH₂NH₂·H₂O (112 μL, 2.34 mmol) were then added to EtOH (4.0 mL) and left mixing at 80° C. After 20 hours, the mixture was evaporated to dry, redissolved into MeOH (4.0 mL), and then NaHCO₃ (788 mg, 9.38 mmol) and Ac₂O added (0.44 mL, 4.7 mmol). After another 4 hours, the solution was evaporated to dry and the crude mixture purified via MPLC on silica gel using 0→30% MeOH—CH₂Cl₂ to afford the partially deprotected product: R_{f}=0.07 (3:7 acetone:CH₂Cl₂). LRMS m/z calc'd for C₅₇H₇₂N₂NaO₂₀ (M+Na)⁺: 1127.46; found: 1127.28. The starting material and Pd(OH)₂ (20% w/w on carbon; 40 mg) were added to 50% aqueous MeOH (2.0 mL), the atmosphere evacuated, and the flask flushed with H_{2(g)} (via balloon). After 24 hours, the catalyst was removed via filtration, the mixture evaporated to dry, and the crude material purified via RPLC on C-18 silica gel using 0→60% acetonitrile-H₂O to afford the pure product as a white solid (92 mg, 0.12 mmol, 63% yield over 4 steps). [α]_{D}²⁰: +5.4° (c 0.5, H₂O). ¹H NMR (D₂O, 500 MHz): δH 5.21 (d, 1H, J=4.0 Hz, Fuc_H1), 4.73 (d, 1H, J=3.4 Hz, GalN_H1), 4.61 (d, 1H, J=7.7 Hz, Gal_H1), 4.53 (d, 1H, J=8.5 Hz, GlcN_H1), 4.20 (dq, 1H, J=6.6, <1 Hz, Fuc_H5), 4.16 (dd, 1H, J=11.0, 3.4 Hz, GalN_H2), 4.14 (dd, 1H, J=2.8, <1 Hz, GalN_H4), 4.11 (dd, 1H, J=11.1, 2.9 Hz, GalN_H3), 4.08-4.03 (m, 2H, GalN_H6^{a} and GalN_H5), 3.93 (dd, 1H, J=12.5, 1.6 Hz, GlcN_H6^{a}), 3.88 (dd, 1H, J=3.4, <1 Hz, Gal_H4), 3.82 (dd, 1H, J=9.7, 3.4 Hz, Gal_H3), 3.78-3.71 (m, 5H, Fuc_H2, Gal_H6^{a}, GlcN_H6^{b}, Gal_H6^{b}, and GalN_H6^{b}), 3.71 (dd, 1H, J=10.2, 8.5 Hz, GlcN_H2), 3.67-3.61 (m, 3H, Fuc_H4, Fuc_H3, and Gal_H5), 3.61 (dd, 1H, J=9.6, 7.8 Hz, Gal_H2), 3.53 (dd, 1H, J=10.3, 8.2 Hz, GlcN_H3), 3.48-3.41 (m, 2H, GlcN_H5 and GlcN_H4), 3.32 (s, 3H, OCH₃), 2.03 (s, 3H, Ac), 2.00 (s, 3H, Ac), 1.18 (d, 3H, J=6.6 Hz, Fuc_H6). ¹³C NMR (D₂O, 125 MHz): δC 175.11 (C═O), 174.31 (C═O), 102.60 (Gal_C1), 102.29 (GlcN_C1), 99.93 (Fuc_C1), 98.37 (GalN_C1), 76.94 (Gal_C2), 76.49 (GlcN_C5), 75.67 (Gal_C5), 74.45 (GlcN_C3), 74.21 (GalN_C3 and Gal_C3), 72.50 (Fuc_C4), 70.82 (GalN_C6), 70.58 (GlcN_C4), 70.20 (Fuc_C3), 69.98, 69.93 (GalN_C4 and GalN_C5), 69.72 (Gal_C4), 68.72 (Fuc_C2), 67.43 (Fuc_C5), 61.57 (Gal_C6), 61.35 (GlcN_C6), 56.15 (GlcN_C2), 55.48 (OCH₃), 49.97 (GalN_C2), 22.81 (Ac), 22.58 (Ac), 15.99 (Fuc_C6). ESI-HRMS m/z calc'd for C₂₉H₅₀N₂NaO₂₀ (M+Na)⁺: 769.2855; found: 769.2853. HPLC purity analysis: >99.5%, Rₜ 5.49 minutes, Atlantis T3 C18 column.

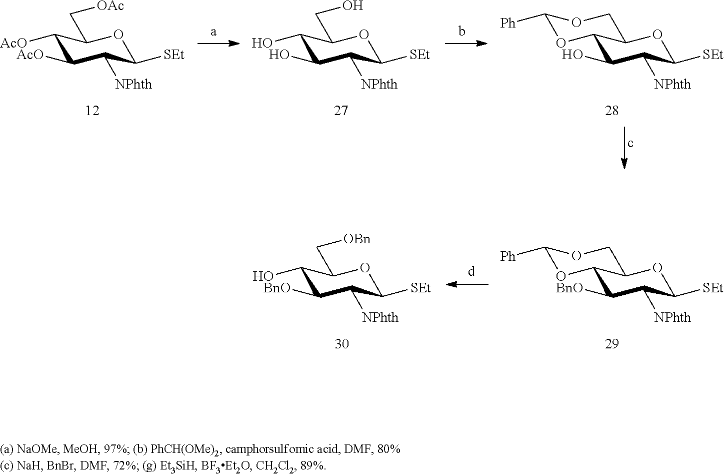

Ethyl 2-deoxy-2-phthalimido-1-thio-β-D-glucopyranoside (27). The starting material (12; 6.98 g, 14.6 mmol) was dissolved into anhydrous MeOH (50 mL) and anhydrous CH₂Cl₂ (20 mL), and then NaOMe solution was added drop-wise (1.5 M NaOMe in MeOH; to pH 10). After 4 hours at ambient temperature, the reaction mixture was neutralized with acidic resin (Amberlite IR-120H; to pH 6), filtered, and then evaporated to dry to afford the pure product as a white solid (4.97 g, 14.1 mmol, 97% yield). R_{f}=0.21 (2:3 acetone:toluene). [α]_{D}²⁰: +8.8° (c 1.0, CH₃OH). ¹H NMR (CD₃OD, 500 MHz): δH 7.90-7.81 (m, 4H, Ar), 5.32 (d, 1H, J=10.5 Hz, H-1), 4.27 (dd, 1H, J=10.2, 8.4 Hz, H-3), 4.05 (dd, 1H, J=10.3, 10.3 Hz, H-2), 3.92 (dd, 1H, J=12.0, 2.0 Hz, H-6^{a}), 3.73 (dd, 1H, J=12.0, 5.6 Hz, H-6^{b}), 3.45 (ddd, 1H, J=9.8, 5.5, 2.0 Hz, H-5), 3.40 (dd, 1H, J=9.8, 8.4 Hz, H-4), 2.73 (dq, 1H, J=12.7, 7.4 Hz, SCH^{a}H^{b}CH₃), 2.63 (dq, 1H, J=12.7, 7.5 Hz, CH^{a}H^{b}CH₃), 1.17 (dd, 3H, J=7.4, 7.4 Hz, SCH₂CH₃). ¹³C NMR (CD₃OD, 125 MHz): δC 169.86 (C═O), 169.58 (C═O), 135.77 (Ar), 135.69 (Ar), 133.36 (Ar), 133.09 (Ar), 124.57 (Ar), 124.24 (Ar), 82.73 (C-5), 82.62 (C-1), 73.87 (C-3), 72.61 (C-4), 63.09 (C-6), 57.88 (C-2), 25.00 (SCH₂CH₃), 15.44 (SCH₂CH₃). LRMS m/z calc'd for C₁₆H₁₉NNaO₆S (M+Na)⁺: 376.08; found: 376.01.

Ethyl 4,6-O-benzylidene-2-deoxy-2-phthalimido-1-thio-β-D-glucopyranoside (28). The starting material (27; 4.974 g, 14.08 mmol), benzylidene dimethyl acetal (2.6 mL, 17 mmol), and camphorsulfonic acid (to pH 3) were added to anhydrous DMF (40 mL) under Ar. After 16 hours of mixing at ambient temperature, the reaction mixture was neutralized with Et₃N (to pH 8), evaporated to dry, and then purified via MPLC on silica gel using 0→70% acetone-toluene to afford the desired product as a white solid (4.977 g, 11.27 mmol, 80% yield). R_{f}=0.56 (1:4 acetone:toluene). [α]_{D}²⁰: −4.2° (c 1.0, CHCl₃). ¹H NMR (CDCl₃, 500 MHz): δH 7.83-7.79 (m, 2H, Ar), 7.69-7.64 (m, 2H, Ar), 7.50-7.46 (m, 2H, Ar), 7.36-7.32 (m, 3H, Ar), 5.54 (s, 1H, PhCH), 5.36 (d, 1H, J=10.6 Hz, H-1), 4.60 (ddd, 1H, J=9.6, 9.6, 3.8 Hz, H-3), 4.35 (dd, 1H, J=10.4, 4.9 Hz, H-6^{a}), 4.27 (dd, 1H, J=10.3, 10.3 Hz, H-2), 3.76 (dd, 1H, J=10.2, 10.2 Hz, H-6^{b}), 3.62 (ddd, 1H, J=9.6, 9.6, 4.9 Hz, H-5), 3.54 (dd, 1H, J=9.2, 9.2 Hz, H-4), 3.05 (d, 1H, J=3.8 Hz, 3-OH), 2.68 (dq, 1H, J=12.5, 7.4 Hz, SCH^{a}H^{b}CH₃), 2.63 (dq, 1H, J=12.5, 7.4 Hz, SCH^{a}H^{b}CH₃), 1.17 (dd, 3H, J=7.4, 7.4 Hz, SCH₂CH₃). ¹³C NMR (CDCl₃, 125 MHz): δC 168.35 (C═O), 167.82 (C═O), 137.10 (Ar), 134.29 (Ar), 131.74 (Ar), 131.60 (Ar), 129.41 (Ar), 128.43 (Ar), 126.44 (Ar), 123.93 (Ar), 123.41 (Ar), 101.93 (PhCH), 82.14 (C-4), 81.94 (C-1), 70.46 (C-5), 69.53 (C-3), 68.67 (C-6), 55.69 (C-2), 24.27 (SCH₂CH₃), 14.98 (SCH₂CH₃). LRMS m/z calc'd for C₂₃H₂₃NNaO₆S (M+Na)⁺: 464.11; found: 464.11.

Ethyl 3-O-benzyl-4,6-O-benzylidene-2-deoxy-2-phthalimido-1-thio-β-D-glucopyranoside (29). The starting material (28; 3.001 g, 6.797 mmol) and benzyl bromide (1.1 mL, 9.3 mmol) were added to anhydrous DMF (30 mL), and then NaH added portion-wise (60% oil dispersion; 385 mg, 9.62 mmol). After 1 hour, the reaction was quenched via the drop-wise addition of H₂O and the mixture diluted with EtOAc (200 mL) and saturated NaCl_{(aq)} solution (500 mL). The aqueous layer was removed and then re-extracted with EtOAc (200 mL). The combined organic phases were washed with sat'd NaCl_{(aq)} solution (2×500 mL), dried with Na₂SO₄, filtered, and then evaporated to dry. The crude material was purified via MPLC using 0→10% EtOAc-toluene to afford the pure product as a white solid (2.604 g, 4.899 mmol, 72% yield). R_{f}=0.71 (1:4 EtOAc:toluene). [α]_{D}²⁰: +54° (c 1.0, CHCl₃). ¹H NMR (CDCl₃, 500 MHz): δH 7.85-7.82 (m, 1H, Ar), 7.73-7.63 (m, 3H, Ar), 7.54-7.51 (m, 2H, Ar), 7.42-7.35 (m, 3H, Ar), 7.01-6.98 (m, 2H, Ar), 6.94-6.86 (m, 3H, Ar), 5.63 (s, 1H, PhCH), 5.35 (d, 1H, J=10.6 Hz, H-1), 4.80 (d, 1H, J=12.3 Hz, PhCH^{a}H^{b}), 4.51 (d, 1H, J=12.3 Hz, PhCH^{a}H^{b}), 4.46 (dd, 1H, J=9.9, 9.0 Hz, H-3), 4.41 (dd, 1H, J=10.4, 4.9 Hz, H-6^{a}), 4.30 (dd, 1H, J=10.5, 10.0 Hz, H-2), 3.83 (dd, 1H, J=10.2, 10.2 Hz, H-6^{b}), 3.82 (dd, 1H, J=9.2, 9.2 Hz, H-4), 3.70 (ddd, 1H, J=9.6, 9.6, 4.9 Hz, H-5), 2.68 (dq, 1H, J=12.5, 7.4 Hz, SCH^{a}H^{b}CH₃), 2.62 (dq, 1H, J=12.5, 7.4 Hz, SCH^{a}H^{b}CH₃), 1.16 (dd, 3H, J=7.4, 7.4 Hz, SCH₂CH₃). ¹³C NMR (CDCl₃, 125 MHz): δC 167.90 (C═O), 167.58 (C═O), 137.99 (Ar), 137.49 (Ar), 134.13 (Ar), 134.01 (Ar), 131.80 (Ar), 131.74 (Ar), 129.20 (Ar), 128.46 (Ar), 128.27 (Ar), 128.21 (Ar), 127.59 (Ar), 126.23 (Ar), 123.72 (Ar), 123.49 (Ar), 101.48 (PhCH), 83.22 (C-4), 81.96 (C-1), 75.60 (C-3), 74.37 (PhCH₂), 70.63 (C-5), 68.90 (C-6), 54.85 (C-2), 24.24 (SCH₂CH₃), 15.03 (SCH₂CH₃). LRMS m/z calc'd for C₃₀H₂₉NNaO₆S (M+Na)⁺: 554.16; found: 554.09.

Ethyl 3,6-di-O-benzyl-2-deoxy-2-phthalimido-1-thio-β-D-glucopyranoside (30). A solution of the starting material (29; 2.726 g, 5.128 mmol) in anhydrous CH₂Cl₂ (25 mL) was cooled to 0° C. under Ar, and then Et₃SiH (4.1 mL, 26 mmol) and BF₃·Et₂O (1.3 mL, 10 mmol) were slowly added. After 5 hours, the reaction mixture was neutralized with Et₃N (to pH 8), warmed back to ambient temperature, and then quenched via the slow addition of MeOH (10 mL). The crude mixture was evaporated to dry, and then purified via MPLC on silica gel using 0→30% EtOAc-toluene to afford the pure product as a white solid (2.430 g, 4.554 mmol, 89% yield). R_{f}=0.43 (1:4 EtOAc:toluene). [α]_{D}²⁰: +38° (c 1.0, CHCl₃). ¹H NMR (CDCl₃, 500 MHz): δH 7.82-7.80 (m, 1H, Ar), 7.72-7.66 (m, 3H, Ar), 7.38-7.29 (m, 5H, Ar), 7.06-7.02 (m, 2H, Ar), 6.98-6.92 (m, 3H, Ar), 5.27 (d, 1H, J=10.0 Hz, H-1), 4.75 (d, 1H, J=12.2 Hz, PhCH^{a}H^{b}), 4.63 (d, 1H, J=11.9 Hz, PhCH^{a}H^{b}), 4.58 (d, 1H, J=11.9 Hz, PhCH^{a}H^{b}), 4.54 (d, 1H, J=12.2 Hz, PhCH^{a}H^{b}), 4.28 (dd, 1H, J=10.2, 7.9 Hz, H-3), 4.23 (dd, 1H, J=10.1, 10.1 Hz, H-2), 3.85 (dd, 1H, J=10.1, 4.8 Hz, H-6^{a}), 3.85-3.81 (m, 1H, H-4), 3.77 (dd, 1H, J=10.1, 5.2 Hz, H-6^{b}), 3.68 (ddd, 1H, J=9.6, 5.0, 5.0 Hz, H-5), 2.97 (d, 1H, J=2.5 Hz, 4-OH), 2.66 (dq, 1H, J=12.5, 7.4 Hz, SCH^{a}H^{b}CH₃), 2.59 (dq, 1H, J=12.5, 7.4 Hz, SCH^{a}H^{b}CH₃), 1.16 (dd, 3H, J=7.4, 7.4 Hz, SCH₂CH₃). ¹³C NMR (CDCl₃, 125 MHz): δC 168.28 (C═O), 167.72 (C═O), 138.33 (Ar), 137.79 (Ar), 134.12 (Ar), 134.02 (Ar), 131.86 (Ar), 128.72 (Ar), 128.36 (Ar), 128.12 (Ar), 128.10 (Ar), 128.02 (Ar), 127.65 (Ar), 123.74 (Ar), 123.48 (Ar), 81.38 (C-1), 79.78 (C-3), 77.77 (C-5), 74.77 (C-4), 74.67 (PhCH₂), 74.00 (PhCH₄₂), 71.13 (C-6), 54.60 (C-2), 24.17 (SCH₄₂CH₃), 15.11 (SCH₂CH₃). LRMS m/z calc'd for C₃₀H₃₁NNaO₆S (M+Na)⁺: 556.18; found: 556.11.

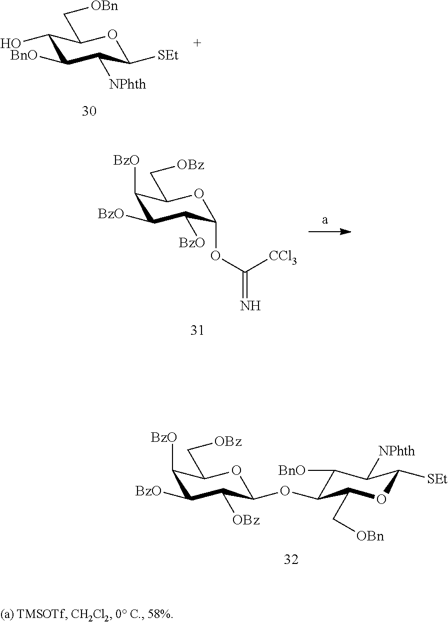

Ethyl 2,3,4,6-tetra-O-benzoyl-β-D-galactopyranosyl-(1→4)-3,6-di-O-benzyl-2-deoxy-2-phthalimido-1-thio-β-D-glucopyranoside (32). The glycosyl acceptor (30; 621 mg, 1.16 mmol), glycosyl donor⁶⁷ (31; 1.715 g, 2.315 mmol), and crushed molecular sieves (3 Å, 1.006 mg) in anhydrous CH₂Cl₂ (25 mL) were left mixing at ambient temperature under Ar. After 1 hour, the reaction flask was cooled to 0° C., and then trimethylsilyl triflate (32 μL, 0.18 mmol) was added dropwise. The flask was slowly warmed to rt, and after 6 hours was neutralized with Et₃N (to pH 8), filtered over Celite, and diluted with CH₂Cl₂ (250 mL). The organic phase was washed with saturated NaHCO_{3(aq)} solution (250 mL), saturated NaCl_{(aq)} solution (250 mL), dried with Na₂SO₄, filtered, and evaporated to dry. The crude material was purified via MPLC on silica gel using 0→10% EtOAc-toluene to afford the pure product as a white solid (747 mg, 0.672 mmol, 58% yield). R_{f}=0.58 (1:4 EtOAc:toluene). [α]_{D}²⁰: +51° (c 1.0, CHCl₃). ¹H NMR (CDCl₃, 500 MHz): δH 8.06-8.03 (m, 2H, Ar), 7.95-7.91 (m, 4H, Ar), 7.81-7.76 (m, 4H, Ar), 7.70-7.34 (m, 15H, Ar), 7.32-7.28 (m, 2H, Ar), 7.25-7.21 (m, 2H, Ar), 7.10-7.06 (m, 2H, Ar), 6.85-6.82 (m, 3H, Ar), 5.88 (dd, 1H, J=3.4, <1 Hz, Gal_H4), 5.79 (dd, 1H, J=10.4, 8.0 Hz, Gal_H2), 5.43 (dd, 1H, J=10.4, 3.5 Hz, Gal_H3), 5.16 (d, 1H, J=10.5 Hz, GlcN_H1), 5.02 (d, 1H, J=12.2 Hz, PhCH^{a}H^{b}), 4.97 (d, 1H, J=8.0 Hz, Gal_H1), 4.76 (d, 1H, J=12.1 Hz, PhCH^{a}H^{b}), 4.69 (d, 1H, J=12.3 Hz, PhCH^{a}H^{b}), 4.41 (d, 1H, J=12.2 Hz, PhCH^{a}H^{b}), 4.40 (dd, 1H, J=11.3, 6.7 Hz, Gal_H6^{a}), 4.37 (dd, 1H, J=10.2, 8.6 Hz, GlcN_H3), 4.30 (dd, 1H, J=11.3, 6.9 Hz, Gal_H6^{b}), 4.26 (dd, 1H, J=10.4, 10.4 Hz, GlcN_H2), 4.24 (dd, 1H, J=9.8, 8.7 Hz, GlcN_H4), 4.03 (ddd, 1H, J=6.8, 6.8, <1 Hz, Gal_H5), 3.73 (dd, 1H, J=11.2, 3.1 Hz, GlcN_H6^{a}), 3.60 (dd, 1H, J=11.2, 1.4 Hz, GlcN_H6^{b}), 3.43 (ddd, 1H, J=9.9, 2.8, 1.4 Hz, GlcN_H5), 2.63 (dq, 1H, J=12.5, 7.4 Hz, SCH^{a}H^{b}CH₃), 2.55 (dq, 1H, J=12.5, 7.5 Hz, SCH^{a}H^{b}CH₃), 1.14 (dd, 3H, J=7.4, 7.4 Hz, SCH₂CH₃). ¹³C NMR (CDCl₃, 125 MHz): δC 168.12 (C═O), 167.65 (C═O), 166.20 (C═O), 165.67 (C═O), 165.53 (C═O), 165.23 (C═O), 138.74 (Ar), 138.27 (Ar), 134.02 (Ar), 133.88 (Ar), 133.64 (Ar), 133.45 (Ar), 131.87 (Ar), 129.71 (Ar), 129.28 (Ar), 129.27 (Ar), 129.01 (Ar), 128.89 (Ar), 128.76 (Ar), 128.74 (Ar), 128.48 (Ar), 128.38 (Ar), 128.30 (Ar), 128.11 (Ar), 128.01 (Ar), 127.28 (Ar), 123.67 (Ar), 123.44 (Ar), 100.64 (Gal_C1), 81.28 (GlcN_C1), 79.02 (GlcN_C5), 77.96 (GlcN_C4), 77.72 (GlcN_C3), 74.76 (PhCH₂), 73.74 (PhCH₂), 71.96 (Gal_C3), 71.27 (Gal_C5), 70.55 (Gal_C2), 68.24 (Gal_C4), 67.91 (GlcN_C6), 61.78 (Gal_C6), 54.95 (GlcN_C2), 24.09 (SCH₂CH₃), 15.09 (SCH₂CH₃). LRMS m/z calc'd for C₆₄H₅₇NNaO₁₅S (M+Na)⁺: 1134.33; found: 1134.27.

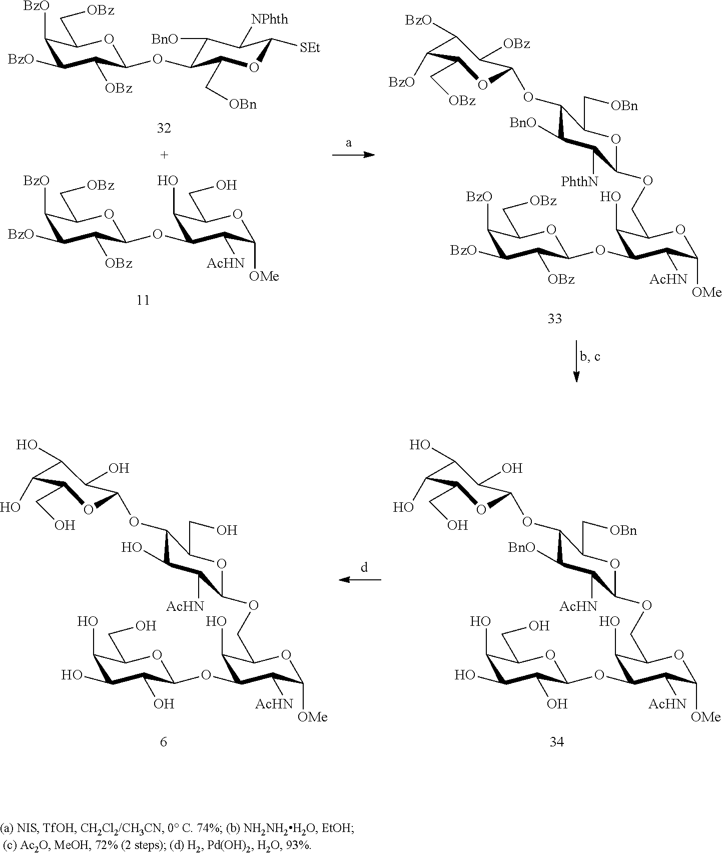

Methyl 2,3,4,6-tetra-O-benzoyl-β-D-galactopyranosyl-(1→4)-3,6-di-O-benzyl-2-deoxy-2-phthalimido-β-D-glucopyranosyl-(1→6)-[2,3,4,6-tetra-O-benzoyl-β-D-galactopyranosyl-(1→3)]-2-acetamido-2-deoxy-α-D-galactopyranoside (33). The glycosyl acceptor (11; 132 mg, 0.162 mmol), glycosyl donor (32; 203 mg, 0.183 mmol), and crushed molecular sieves (3 Å, 273 mg) in anhydrous CH₂Cl₂ (2.0 mL) and anhydrous acetonitrile (2.0 mL) were left mixing for 1 hour at ambient temperature under Ar. The reaction flask was cooled to 0° C., and then N-iodosuccinimide was added (67 mg, 0.30 mmol) followed by the drop-wise addition of triflic acid (3 μL, 0.017 mmol; in 20 μL CH₂Cl₂). After 6 hours, the mixture was neutralized with Et₃N (to pH 8), warmed to ambient temperature, filtered over Celite, and diluted further with CH₂Cl₂ (60 mL). The organic phase was washed with sat'd Na₂S₂O_{3(aq)} solution (60 mL), sat'd NaCl_{(aq)} solution (2×60 mL), dried with Na₂SO₄, filtered, and evaporated to dry. The crude material was purified via MPLC on silica gel using 0→30% acetone (w/ 0.1% NH₄OH)—CH₂Cl₂ to afford the pure product as a white solid (224 mg, 0.120 mmol, 74% yield). R_{f}=0.21 (1:9 acetone w/ 0.1% NH₄OH:CH₂Cl₂). [α]_{D}²⁰: +110° (c 1.0, CHCl₃). ¹H NMR (CDCl₃, 500 MHz): δH 8.08-8.03 (m, 4H, Ar), 7.96-7.89 (m, 8H, Ar), 7.79-7.76 (m, 2H, Ar), 7.74-7.72 (m, 2H, Ar), 7.68-7.64 (m, 2H, Ar), 7.64-7.60 (m, 1H, Ar), 7.58-7.54 (m, 2H, Ar), 7.53-7.30 (m, 24H, Ar), 7.26-7.20 (m, 4H, Ar), 7.10-7.06 (m, 2H, Ar), 6.84-6.81 (m, 3H, Ar), 5.92 (dd, 1H, J=3.5, <1 Hz, Gal_H4′), 5.87 (dd, 1H, J=3.5, <1 Hz, Gal_H4), 5.78 (dd, 1H, J=10.4, 8.0 Hz, Gal_H2), 5.75 (dd, 1H, J=10.4, 8.0 Hz, Gal_H2′), 5.53 (dd, 1H, J=10.4, 3.5 Hz, Gal_H3′), 5.41 (dd, 1H, J=10.4, 3.5 Hz, Gal_H3), 5.04-5.00 (m, 2H, NH and PhCH^{a}H^{b}), 4.99 (d, 1H, J=8.5 Hz, GlcN_H1), 4.90 (d, 1H, J=8.0 Hz, Gal_H1), 4.79-4.76 (m, 2H, Gal_H1′ and PhCH^{a}H^{b}), 4.68 (d, 1H, J=12.3 Hz, PhCH^{a}H^{b}), 4.51 (dd, 1H, J=11.7, 5.0 Hz, Gal_H6^{a}), 4.45 (dd, 1H, J=11.7, 7.7 Hz, Gal_H6^{b}′), 4.39 (dd, 1H, J=11.2, 6.7 Hz, Gal_H6^{a}), 4.38 (d, 1H, J=12.2 Hz, PhCH^{a}H^{b}), 4.37 (dd, 1H, J=10.7, 8.9 Hz, GlcN_H3), 4.34-4.28 (m, 2H, GalNAc_H2 and Gal_H6^{b}), 4.26-4.22 (m, 2H, Gal_H5′ and GlcN_H4), 4.21 (d, 1H, J=3.6 Hz, GalNAc_H1), 4.15 (dd, 1H, J=10.8, 8.5 Hz, GlcN_H2), 4.02 (ddd, 1H, J=6.8, 6.8, <1 Hz, Gal_H5), 3.91-3.89 (m, 1H, GalNAc_H4), 3.75 (dd, 1H, J=10.8, 2.8 Hz, GlcN_H6^{a}), 3.71-3.69 (m, 1H, GalNAc_H6^{a}), 3.59-3.49 (m, 4H, GalNAc_H3, GalNAc_H5, GalNAc_H6^{b}, and GlcN_H6^{b}), 3.38-3.35 (m, 1H, GlcN_H5), 2.77-2.74 (m, 4H, OCH₃ and GalNAc_4-OH), 1.28 (s, 3H, Ac). ¹³C NMR (CDCl₃, 125 MHz): δC 169.85 (C═O, Ac), 167.98 (C═O, Phth), 167.75 (C═O, Phth), 166.22 (C═O, Bz), 166.20 (C═O, Bz), 165.78 (C═O, Bz), 165.73 (C═O, Bz), 165.67 (C═O, Bz), 165.52 (C═O, Bz), 165.18 (C═O, Bz), 164.82 (C═O, Bz), 138.84 (Ar), 138.11 (Ar), 133.98 (Ar), 133.88 (Ar), 133.70 (Ar), 133.66 (Ar), 133.59 (Ar), 133.45 (Ar), 131.95 (Ar), 130.27 (Ar), 130.05 (Ar), 129.95 (Ar), 129.90 (Ar), 129.71 (Ar), 129.28 (Ar), 129.26 (Ar), 129.01 (Ar), 128.97 (Ar), 128.92 (Ar), 128.87 (Ar), 128.85 (Ar), 128.78 (Ar), 128.76 (Ar), 128.73 (Ar), 128.71 (Ar), 128.58 (Ar), 128.52 (Ar), 128.49 (Ar), 128.43 (Ar), 127.25 (Ar), 123.50 (Ar), 123.21 (Ar), 101.92 (Gal_C1′), 100.68 (Gal_C1), 99.16 (GlcN_C1), 98.13 (GalNAc_C1), 80.07 (GalNAc_C3), 78.06 (GlcN_C4), 76.67 (GlcN_C3), 74.64 (PhCH₂), 74.56 (GlcN_C5), 73.85 (PhCH₁₂), 72.14 (Gal_C5′), 71.88 (Gal_C3), 71.60 (Gal_C3′), 71.23 (Gal_C5), 70.50 (Gal_C2), 70.29 (GalNAc_C6), 69.77 (Gal_C2′), 68.44 (GalNAc_C5), 68.21 (Gal_C4), 68.14 (Gal_C4′ and GalNAc_C4), 67.73 (GlcN_C6), 62.52 (Gal_C6′), 61.78 (Gal_C6), 55.96 (GlcN_C2), 54.45 (OCH₃), 47.86 (GalNAc_C2), 22.62 (Ac). LRMS m/z calc'd for C₁₀₅H₉₄N₂NaO₃₀ (M+Na)⁺: 1886.58; found: 1886.29.

Methyl β-D-galactopyranosyl-(1→4)-2-acetamido-3,6-di-O-benzyl-2-deoxy-β-D-glucopyranosyl-(1→6)-[β-D-galactopyranosyl-(1→3)]-2-acetamido-2-deoxy-α-D-galactopyranoside (34). The starting material (33; 124 mg, 0.0665 mmol) and NH₂NH₂·H₂O (48 μL, 1.0 mmol) in EtOH (1.5 mL) were left heating at 80° C. After 24 hours, additional NH₂NH₂·H₂O was added (16 μL, 0.33 mmol), and after another 24 hours the mixture was evaporated to dry to afford the crude product: LRMS m/z calc'd for C₄₁H₆₁N₂O₂₀ (M+H)⁺: 901.38; found: 901.44. The mixture was redissolved into MeOH (2.0 mL) and Ac₂O added (157 μL, 1.66 mmol), and after 4 hours the mixture was evaporated to dry and purified via RPLC on C-18 silica gel using 0→100% acetonitrile-H₂O to afford the pure product as a white solid (45 mg, 0.048 mmol, 72% yield over 2 steps) [α]_{D}²⁰: +35° (c 1.0, H₂O). ¹H NMR (D₂O, 500 MHz): δH 7.49-7.35 (m, 10H, Ar), 4.91 (d, 1H, J=11.4 Hz, PhCH^{a}H^{b}), 4.73 (d, 1H, J=4.0 Hz, GalNAc_H1), 4.72 (d, 1H, J=11.8 Hz, PhCH^{a}H^{b}), 4.64 (d, 1H, J=11.4 Hz, PhCH^{a}H^{b}), 4.55 (d, 1H, J=11.9 Hz, PhCH^{a}H^{b}), 4.50 (d, 1H, J=8.4 Hz, GlcNAc_H1), 4.42 (d, 1H, J=7.8 Hz, Gal_H1′), 4.30 (dd, 1H, J=11.0, 3.7 Hz, GalNAc_H2), 4.20-4.17 (m, 2H, Gal_H1 and GalNAc_H4), 4.03-4.00 (m, 2H, GalNAc_H5 and GalNAc_H6^{a}), 3.97-3.91 (m, 4H, GalNAc_H3, GlcNAc_H6^{a}, GlcNAc_H6^{b}, and GlcNAc_H4), 3.89 (dd, 1H, J=3.4, <1 Hz, Gal_H4′), 3.84 (dd, 1H, J=3.5, <1 Hz, Gal_H4), 3.77 (dd, 1H, J=10.5, 8.5 Hz, GlcNAc_H2), 3.76-3.58 (m, 9H, Gal_H6^{a}′, Gal_H6^{b}′, GalNAc_H6^{b}, Gal_H6^{a}, GlcNAc_H3, GlcNAc_H5, Gal_H6^{b}, Gal_H5′, and Gal_H3′), 3.51 (dd, 1H, J=7.8, 7.8 Hz, Gal_H2), 3.50 (dd, 1H, J=7.8, 7.8 Hz, Gal_H2′), 3.39-3.35 (m, 2H, Gal_H3 and Gal_H5), 3.31 (s, 3H, OCH₃), 2.00 (s, 3H, Ac), 1.83 (s, 3H, Ac). ¹³C NMR (D₂O, 125 MHz): δC 175.23 (C═O), 174.53 (C═O), 138.23 (Ar), 137.92 (Ar), 129.50 (Ar), 129.39 (Ar), 129.29 (Ar), 129.23 (Ar), 129.03 (Ar), 128.89 (Ar), 105.33 (Gal_C1′), 103.17 (Gal_C1), 102.25 (GlcNAc_C1), 98.77 (GalNAc_C1), 80.56 (GlcNAc_C3), 77.79 (GalNAc_C3), 76.57 (GlcNAc_C4), 75.96 (Gal_C5), 75.58 (Gal_C5′), 75.14 (PhCH₂), 74.58 (GlcNAc_C5), 73.62 (PhCH₂), 73.16 (Gal_C3′), 73.08 (Gal_C3), 71.71 (Gal_C2), 71.24 (Gal_C2′), 70.92 (GalNAc_C6), 69.92 (GalNAc_C5), 69.61 (GalNAc_C4), 69.31 (Gal_C4), 69.18 (Gal_C4′), 68.23 (GlcNAc_C6), 61.84 (Gal_C6), 61.55 (Gal_C6′), 55.45 (OCH₃), 54.98 (GlcNAc_C2), 49.14 (GalNAc_C2), 22.78 (Ac), 22.64 (Ac). LRMS m/z calc'd for C₄₃H₆₂N₂NaO₂₁ (M+Na)⁺: 965.37; found: 965.40.

Methyl β-D-galactopyranosyl-(1→4)-2-acetamido-2-deoxy-β-D-glucopyranosyl-(1→6)-[β-D-galactopyranosyl-(1→3)]-2-acetamido-2-deoxy-α-D-galactopyranoside (6). The starting material (34; 36 mg, 0.038 mmol) and Pd(OH)₂ (20% w/w on carbon, 9 mg) in H₂O (1.5 mL) were left mixing under H₂ at atmospheric pressure. After 24 hours, the solid catalyst was removed via filtration and the solution evaporated to dry. The crude material was purified via RPLC on C-18 silica gel using 0→60% acetonitrile-H₂O to afford the pure product as a white solid (27 mg, 0.035 mmol, 93% yield). [α]_{D}²⁰: +29° (c 0.7, H₂O). ¹H NMR (D₂O, 500 MHz): δH 4.74 (d, 1H, J=3.8 Hz, GalNAc_H1), 4.54 (d, 1H, J=8.2 Hz, GlcNAc_H1), 4.46 (d, 1H, J=7.8 Hz, Gal_H1), 4.43 (d, 1H, J=7.8 Hz, Gal_H1′), 4.31 (dd, 1H, J=11.0, 3.7 Hz, GalNAc_H2), 4.19 (dd, 1H, J=3.2, <1 Hz, GalNAc_H4), 4.07-3.97 (m, 4H, GalNAc_H6^{a}, GalNAc_H5, GalNAc_H3, and GlcNAc_H6^{a}), 3.91 (dd, 1H, J=3.4, <1 Hz, Gal_H4), 3.89 (dd, 1H, J=3.4, <1 Hz, Gal_H4′), 3.83 (dd, 1H, J=12.3, 5.1 Hz, GlcNAc_H6^{b}), 3.79-3.69 (m, 9H, GlcNAc_H2, Gal_H6^{a}, Gal_H6^{a}′, Gal_H6^{b}, Gal_H6^{b}′, GalNAc_H6^{b}, Gal_H5, GlcNAc_H4, and GlcNAc_H3), 3.65 (dd, 1H, J=10.0, 3.4 Hz, Gal_H3), 3.65-3.58 (m, 3H, Gal_H5′, Gal_H3′, and GlcNAc_H5), 3.53 (dd, 1H, J=9.9, 7.8 Hz, Gal_H2), 3.49 (dd, 1H, J=9.9, 7.8 Hz, Gal_H2′), 3.34 (s, 3H, OCH₃), 2.00 (s, 3H, Ac), 2.00 (s, 3H, Ac). ¹³C NMR (D₂O, 125 MHz): δC 175.25 (C═O), 175.05 (C═O), 105.35 (Gal_C1′), 103.55 (Gal_C1), 102.17 (GlcNAc_C1), 98.83 (GalNAc_C1), 79.15 (GlcNAc_C4), 77.72 (GalNAc_C3), 76.01 (Gal_C5), 75.62 (Gal_C5′), 75.38 (GlcNAc_C5), 73.17, 73.16 (Gal_C3 and Gal_C3′), 73.04 (GlcNAc_C3), 71.61 (Gal_C2), 71.26 (Gal_C2′), 70.68 (GalNAc_C6), 69.93 (GalNAc_C5), 69.63 (GalNAc_C4), 69.23, 69.20 (Gal_C4 and Gal_C4′), 61.67, 61.61 (Gal_C6 and Gal_C6′), 60.69 (GlcNAc_C6), 55.67 (GlcNAc_C2), 55.53 (OCH₃), 49.17 (GalNAc_C2), 22.83 (Ac), 22.66 (Ac). ESI-HRMS m/z calc'd for C₂₉H₅₀N₂NaO₂₁ (M+Na)⁺: 785.2804; found: 785.2806. HPLC purity analysis: >99.5%, Rₜ 5.34 minutes, Atlantis T3 C18 column.

### Notes

Table 2: Glycans released from each mucin were permethylated and analyzed by NSI-MS. Table 3 presents details (glycan reference number, structural representation, composition, GlyTouCan accession, theoretical m/z, detected m/z, amount, and relative abundance) for the glycans released from MUC5AC only.

Table 3: Glycans released from each mucin were permethylated and analyzed by NSI-MS. Table 3 presents details (glycan reference number, structural representation, composition, GlyTouCan accession, theoretical m/z, detected m/z, amount, and relative abundance) for the top 5 sulfated glycans detected from each mucin preparation by negative mode NSI-MS.
□ N-acetylgalactosamine ● Mannose
▪ N-acetylglucosamine ◯ Galactose
Ⓢ Sulfate ⋄ N-Glycolylneuraminic acid
▴ Fucose ♦ N-Acetylneuraminic acid

In Tables 2-3:

Tables 4-6: Glycans released from each mucin were permethylated and analyzed by NSI-MS. Tables 4-6 present details (glycan reference number, structural representation, composition, GlyTouCan accession, theoretical m/z, detected m/z, amount, and relative abundance) for the glycans released from all three mucins for cross-comparison.

TABLE 2
Glycan Profiles for MUC5AC Preparations.

| | | | | | pmol/mg | | |
|---|---|---|---|---|---|---|---|
| Gly- | | | m/z (M + Na)¹⁺ | | of total | % Total Profile | |
| can | | | Theoretic- | | released | | w/o |
| # | Composition (Representative Structures) | GlyTouCan Accession(s) | al | Detected | glycan | | Peeling |
| 1 | 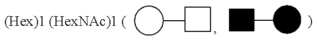 | G76355TG, T85856KC | 518.257 | 518.26 | 91.72 | 16.83 | 17.91 |
| 2 | 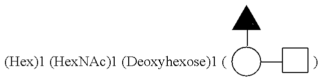 | G94435QH | 692.346 | 692.35 | 73.00 | 13.40 | 14.26 |
| 3 | 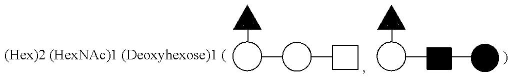 | G73318SN, G33986KK | 896.446 | 896.45 | 10.38 | 1.91 | 2.03 |
| 4 | 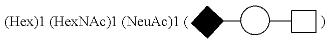 | G65562ZE | 879.431 | 879.44 | 5.99 | 1.10 | 1.17 |
| 5 | 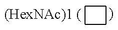 | G57321FI | 314.157 | 314.16 | 2.50 | 0.46 | 0.49 |
| 6 | 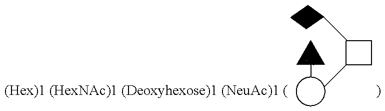 | G47180UC | 1053.520 | 1053.53 | 1.37 | 0.25 | 0.27 |
| 7 | 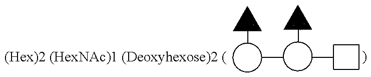 | G68200GL | 1070.535 | 1070.54 | 0.88 | 0.16 | 0.17 |
| 8 | 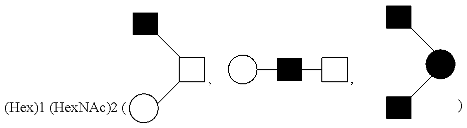 | G00033MO, G61730RY, G56868BH | 763.384 | 763.39 | 87.06 | 15.98 | 17.00 |
| 9 | 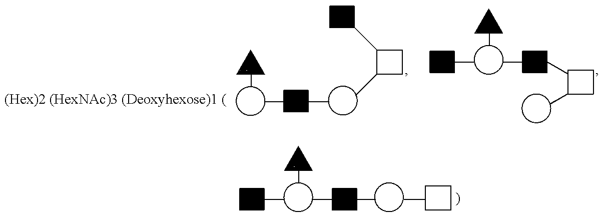 | G91459EI, G26724PP, G52132CU | 1386.699 | 1386.71 | 24.48 | 4.49 | 4.78 |
| 10 | 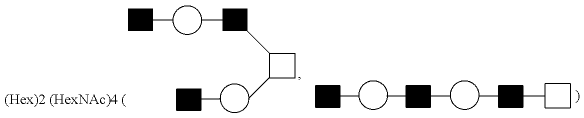 | G34764BK, G12074QJ | 1457.736 | 1457.74 | 23.39 | 4.29 | 4.57 |
| 11 | 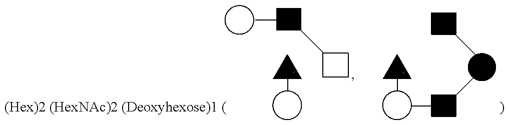 | G94514IB, G61216ZY | 1141.573 | 1141.58 | 20.85 | 3.83 | 4.07 |
| 12 | 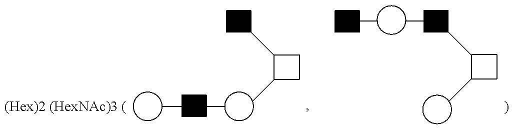 | G96915PP, G13483MW | 1212.610 | 1212.62 | 17.89 | 3.28 | 3.49 |
| 13 | 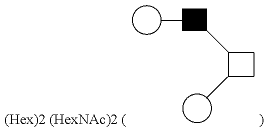 | G64973KT | 967.483 | 967.49 | 17.03 | 3.13 | 3.33 |
| 14 | 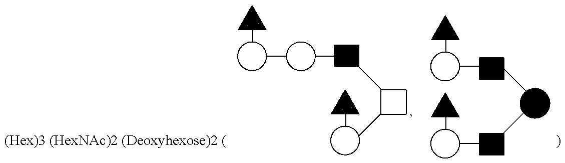 | G20310DM, G78177LC | 1519.762 | 1519.77 | 12.29 | 2.26 | 2.40 |
| 15 | 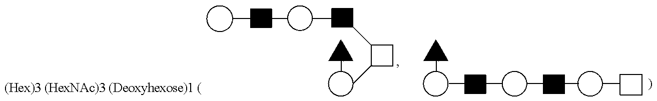 | G66741QE, G16458JH | 1590.799 | 1590.81 | 12.21 | 2.24 | 2.38 |
| 16 | 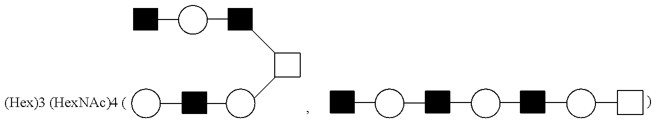 | G94517VF, G57672ST | 1661.836 | 1661.84 | 11.04 | 2.03 | 2.15 |
| 17 | 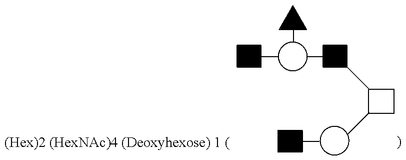 | G23700TV | 1631.825 | 1631.83 | 10.85 | 1.99 | 2.12 |
| 18 | 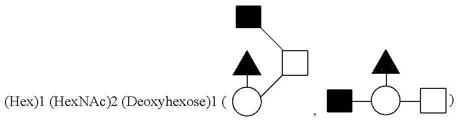 | G32426JY, G74353FF | 937.473 | 937.48 | 10.15 | 1.86 | 1.98 |
| 19 | 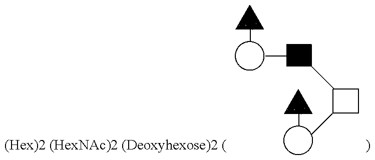 | G23119MJ | 1315.662 | 1315.67 | 7.98 | 1.46 | 1.56 |
| 20 | 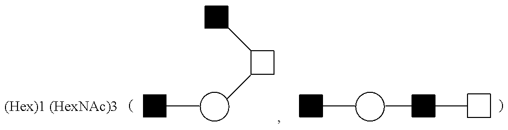 | G68893BQ, G23438NR | 1008.510 | 1008.51 | 7.48 | 1.37 | 1.46 |
| 21 | 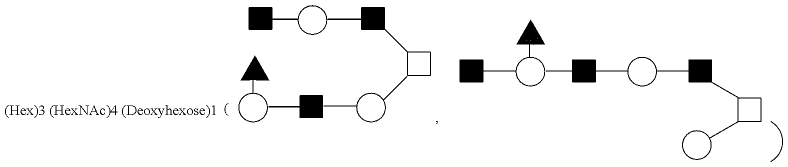 | G02990AF, G29956GF | 1835.925 | 1835.93 | 7.29 | 1.34 | 1.42 |
| 22 | 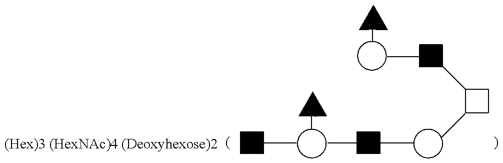 | G93333OF | 1016.502 | 1016.51 | 5.07 | 0.93 | 0.99 |
| 23 | 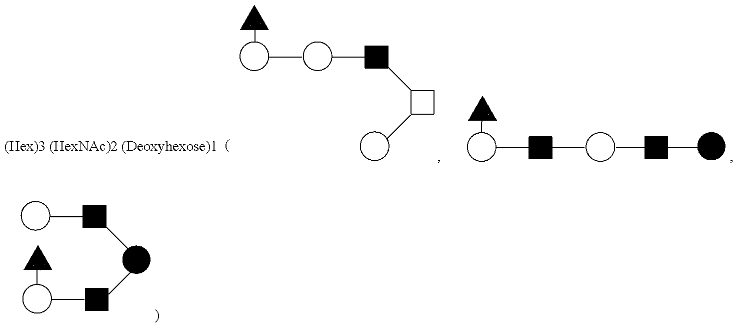 | G15849KC, G81911LP, G41486OC | 1345.672 | 1345.68 | 4.93 | 0.90 | 0.96 |
| 24 | 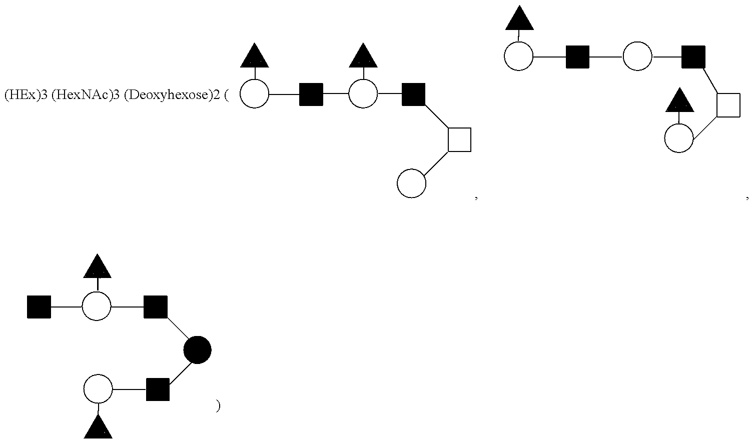 | G35949CT, G71094KR, G08426KY | 1764.888 | 1764.90 | 4.80 | 0.88 | 0.94 |
| 25 | 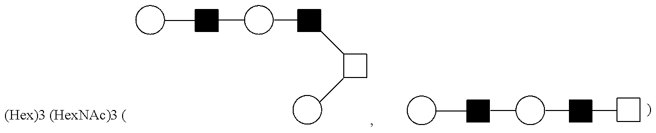 | G86537AD, G89585FG | 1416.709 | 1416.72 | 3.25 | 0.60 | 0.64 |
| 26 | 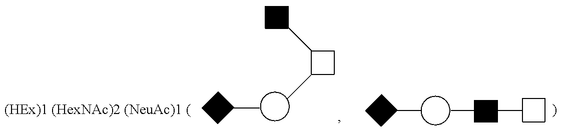 | G85608AG, G64844ET | 1124.557 | 1124.56 | 2.52 | 0.46 | 0.49 |
| 27 | 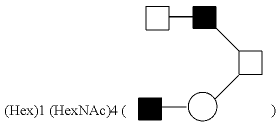 | G59229NY | 1253.636 | 1253.64 | 2.25 | 0.41 | 0.44 |
| 28 | 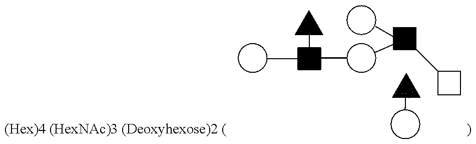 | G68308CM | 1968.988 | 1968.99 | 2.02 | 0.37 | 0.39 |
| 29 | 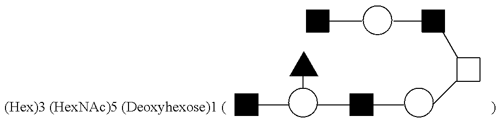 | G05252QE | 1052.020 | 1052.03 | 2.00 | 0.37 | 0.39 |
| 30 | 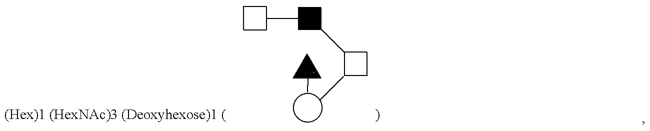 | G28878FC | 1182.599 | 1182.61 | 1.86 | 0.34 | 0.36 |
| 31 | 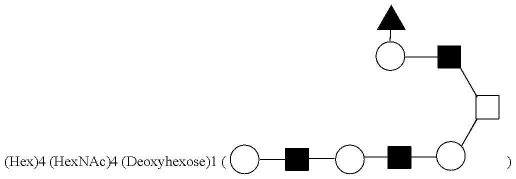 | G94768NG | 1031.507 | 1031.51 | 1.75 | 0.32 | 0.34 |
| 32 | 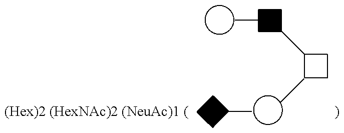 | G46748BU | 1328.657 | 1328.66 | 1.72 | 0.31 | 0.34 |
| 33 | 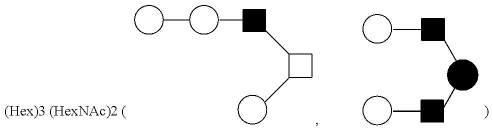 | G16404NW, G25323VU | 1171.583 | 1171.59 | 1.60 | 0.29 | 0.31 |
| 34 | 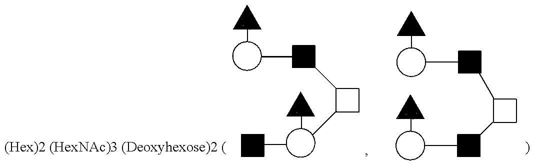 | G68384KC, G39326PP | 1560.788 | 1560.79 | 1.51 | 0.28 | 0.29 |
| 35 | 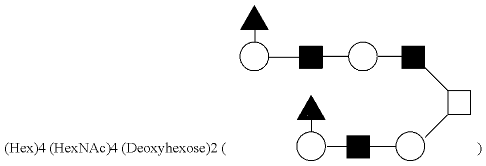 | G98518WL | 1118.552 | 1118.56 | 1.35 | 0.25 | 0.26 |
| 36 | 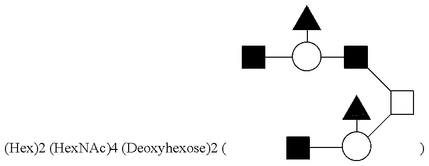 | G90965BZ | 1805.914 | 1805.92 | 1.21 | 0.22 | 0.24 |
| 37 | 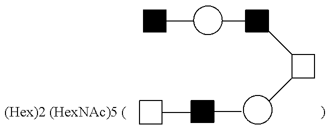 | G23048PE | 1702.862 | 1702.87 | 0.98 | 0.18 | 0.19 |
| 38 | 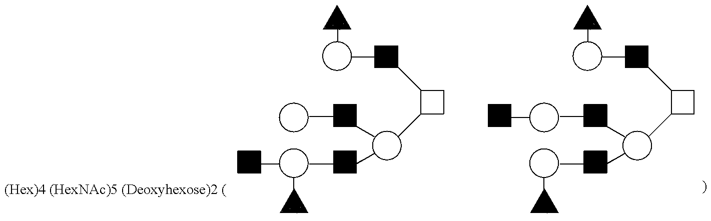 | G82251ZP, G18603ZQ | 1241.115 | 1241.12 | 0.93 | 0.17 | 0.18 |
| 39 | 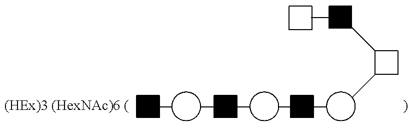 | G62609ZF | 1087.539 | 1087.54 | 0.91 | 0.17 | 0.18 |
| 40 | 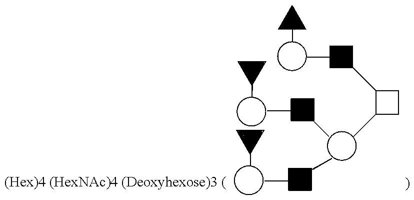 | G66166BF | 1205.596 | 1205.60 | 0.84 | 0.15 | 0.16 |
| 41 | 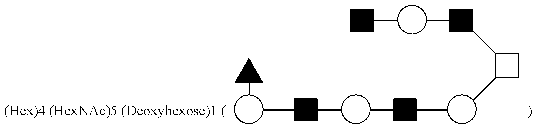 | G92547QZ | 1154.070 | 1154.08 | 0.82 | 0.15 | 0.16 |
| 42 | 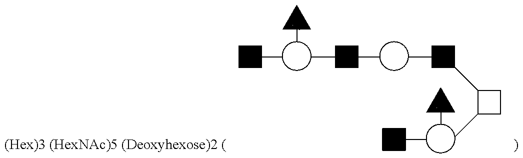 | G28921PH | 1139.065 | 1139.07 | 0.64 | 0.12 | 0.13 |
| 43 | 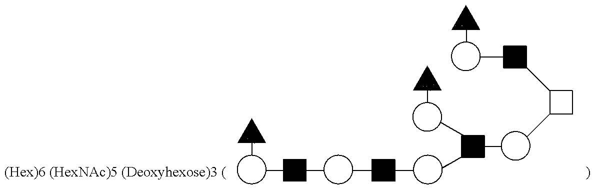 | G18501TC | 1532.259 | 1532.27 | 0.56 | 0.10 | 0.11 |
| 44 | 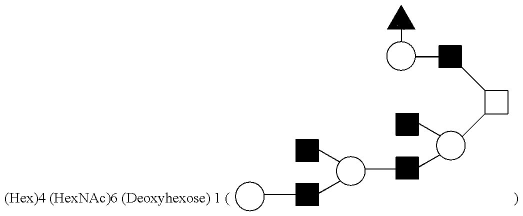 | G32752FJ | 1276.633 | 1276.64 | 0.51 | 0.09 | 0.10 |
| 45 | 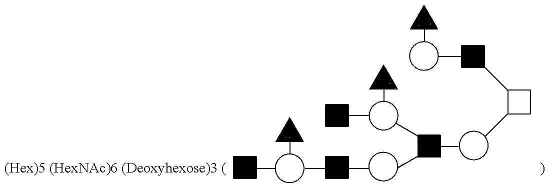 | G65612SS | 1552.772 | 1552.78 | 0.39 | 0.07 | 0.08 |
| 46 | 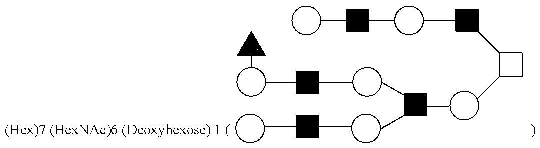 | G61898SS | 1582.783 | 1582.80 | 0.39 | 0.07 | 0.08 |
| 47 | 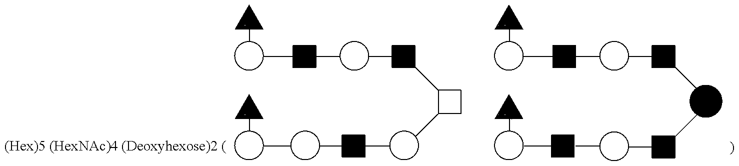 | G21630AC | 1220.601 | 1220.61 | 0.31 | 0.06 | 0.06 |
| 48 | 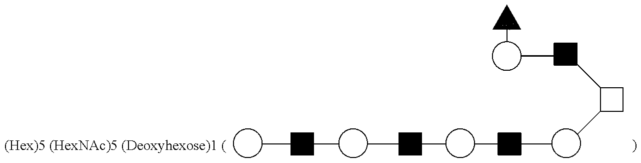 | G25957KN | 1256.120 | 1256.13 | 0.29 | 0.05 | 0.06 |
| 49 | 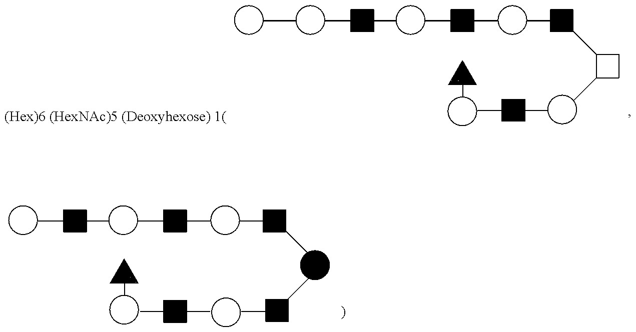 | G37901JE, G84713IO | 1358.170 | 1358.18 | 0.25 | 0.05 | 0.05 |
| 50 | 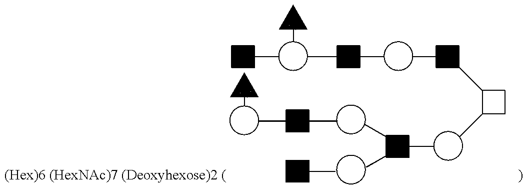 | G29852ZH | 1690.341 | 1690.36 | 0.25 | 0.05 | 0.05 |
| 51 | 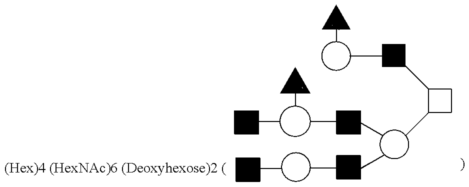 | G99804SJ | 1363.678 | 1363.69 | 0.21 | 0.04 | 0.04 |
| 52 | 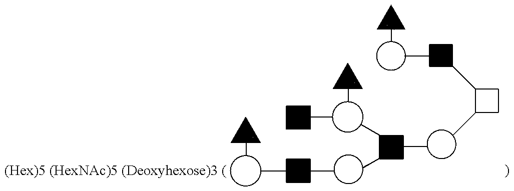 | G90829NZ | 1430.209 | 1430.22 | 0.14 | 0.03 | 0.03 |
| 53 | 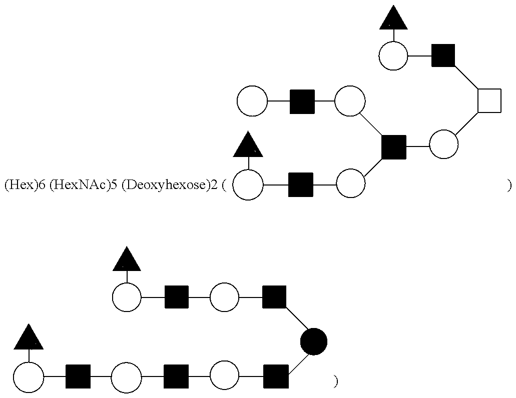 | G70999YJ, G02681FY | 1445.215 | 1445.22 | 0.13 | 0.02 | 0.03 |
| 54 | 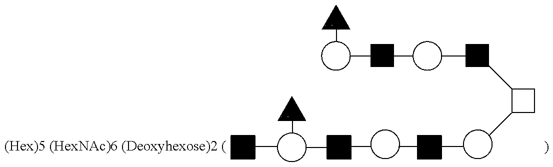 | G44467ZE | 1465.728 | 1465.73 | 0.11 | 0.02 | 0.02 |
| 55 | 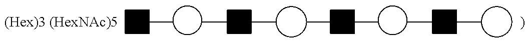 | G00505CR | 1906.962 | 1906.97 | 2.51 | 0.46 | 0.49 |
| 56 | 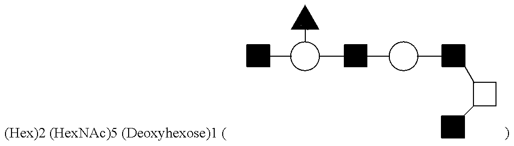 | G09396HG | 949.970 | 949.98 | 0.62 | 0.11 | 0.12 |
| 57 | 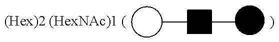 | G28052FT | 722.357 | 722.36 | 4.80 | 0.88 | 0.94 |
| 58 | 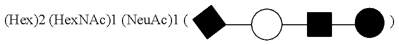 | G59126YU | 1083.531 | 1083.54 | 1.84 | 0.34 | 0.36 |
| 59 | 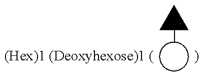 | G00068MO | 447.220 | 447.22 | 32.79 | 6.02 | na |

| | | | |
|---|---|---|---|
| Total w/ Peel: | 544.90 | 100.00 | 100.00 |
| Total w/o Peel: | 512.11 | | |

Glycan 59 is the expected product of peeling reactions

TABLE 3
Sulfated Glycan Profiles For MUC2, MUC5B, and MUC5AC Preparations

| | | | | | | | % of | |
|---|---|---|---|---|---|---|---|---|
| | | | Theo- | | | | Total | |
| | | | retical | | | | sul- | % of |
| | | | m/z | | | | fated | Top 5 |
| | Composition | GlyTouCan | (M − | | | Signal | gly- | sulfated |
| # | (Representative Structure) | Accession(s) | Na)¹⁻ | | Detected | Intensity | cans | glycans |
| S1 | 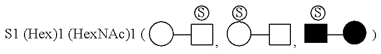 | G10634LC | 560.202 | MUC5B | 560.201 | 887059 | 8.36 | 21.96 |
| S2 | 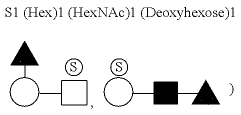 | G08671QK | 734.291 | MUC5B | 734.290 | 1025409 | 9.67 | 25.39 |
| S3 | 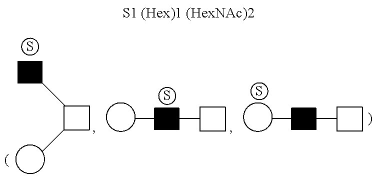 | G32406CO | 805.328 | MUC2 MUC5AC | 805.33 805.33 | 4010391 360757 | 19.68 6.34 | 34.30 9.97 |
| S4 | 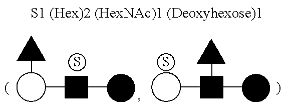 | G24803MV | 938.391 | MUC5B MUC5AC | 938.389 938.39 | 1172196 543225 | 11.05 9.55 | 29.02 15.01 |
| S5 | 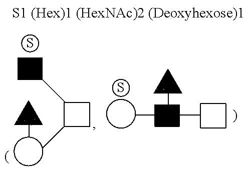 | G10520JC | 979.417 | MUC2 MUC5AC | 979.41 979.42 | 2695001 1275559 | 13.22 22.43 | 23.05 35.25 |
| S6 | 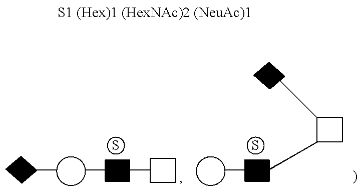 | G96888OD | 1166.502 | MUC2 | 1166.50 | 1909453 | 9.37 | 16.33 |
| S7 | 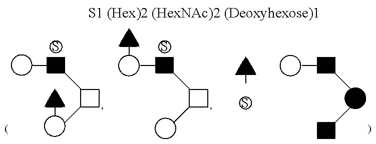 | G24803MV | 1183.517 | MUC5B MUC5AC | 1183.515 1183.52 | 478521 972794 | 4.51 17.11 | 11.85 26.88 |
| S8 | 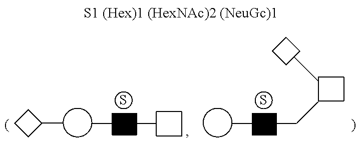 | * | 1196.512 | MUC2 | 1196.51 | 1486979 | 7.30 | 12.72 |
| S9 | 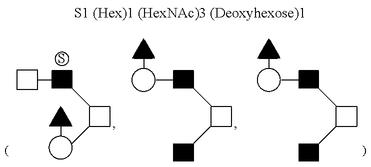 | G72091WB, G60644GY | 1124.544 | MUC2 | 1224.540 | 1588826 | 7.80 | 13.59 |
| S10 | 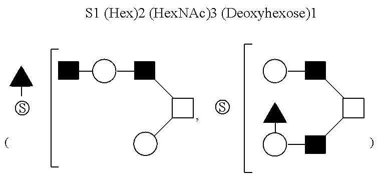 | G35891PL | 1428.644 | MUC5B MUC5AC | 1428.641 1428.64 | 476006 466419 | 4.49 8.20 | 11.78 12.89 |
| | | | | Total | MUC2 | 11690650 | | 100.00 |
| | | | | subset | MUC5B | 4039191 | | 100.00 |
| | | | | | MUC5AC | 3618754 | | 100.00 |
| | | | | Total | MUC2 | 20381869 | 57.36 | |
| | | | | sulfated | MUC5B | 10606784 | 38.08 | |
| | | | | | MUC5AC | 5686243 | 63.64 | |

GlyTouCan Accessions are for the indicated compositions
*not currently supported by repository

TABLE 4
Glycan Profile for MUC2 Preparation

| | | | | | pmol/mg of | % Total Profile | |
|---|---|---|---|---|---|---|---|
| Glycan | | | m/z (M + Na)¹⁺ | | total released | | w/o |
| # | Composition | Gly TouCan Accession(s) | Theoretical | Detected | glycan | | Peeling |
| 1 | (HexNAc)1 | G57321FI | 314.157 | 314.16 | 6.23 | 1.68 | 2.42 |
| 2 | (Hex)1 (HexNAc)1 | G76355TG, G85856KC | 518.257 | 518.26 | 37.53 | 10.10 | 14.57 |
| 3 | (Hex)1 (HexNAc)1 (Deoxyhexose)1 | G94435QH | 692.346 | 692.35 | 89.58 | 24.11 | 34.77 |
| 4 | (Hex)1 (HexNAc)1 (NeuAc)1 | G65562ZE | 879.431 | 879.43 | 17.29 | 4.65 | 6.71 |
| 5 | (Hex)2 (HexNAc)1 (Deoxyhexose)1 | G73318SN, G33986KK | 896.446 | | | | |
| 6 | (Hex)1 (HexNAc)1 (NeuGc)1 | G64527IJ | 909.441 | 909.444 | 11.40 | 3.07 | 4.42 |
| 7 | (Hex)1 (HexNAc)1 (Deoxyhexose)1 (NeuAc)1 | G47180UC | 1053.520 | 1053.523 | 1.25 | 0.34 | 0.49 |
| 8 | (Hex)2 (HexNAc)1 (Deoxyhexose)2 | G68200GL | 1070.535 | 1070.540 | 0.20 | 0.05 | 0.08 |
| 9 | (Hex)1 (HexNAc)1 (NeuAc)2 | G01614ZM | 1240.605 | 1240.608 | 1.86 | 0.50 | 0.72 |
| 10 | (Hex)2 (HexNAc)1 (Deoxyhexose)3 | G82961CS | 1244.625 | | | | |
| 11 | (Hex)2 (HexNAc)1 (Deoxyhexose)1 (NeuAc)1 | G49549VN, G95742RK | 1257.620 | | | | |
| 12 | (Hex)1 (HexNAc)1 (NeuAc)1 (NeuGc)1 | G49527BY | 1270.615 | 1270.616 | 0.41 | 0.11 | 0.16 |
| 13 | (Hex)1 (HexNAc)2 (Deoxyhexose)1 (NeuAc)1 | G75749JP, G40270LS | 1298.646 | 1298.649 | 1.21 | 0.32 | 0.47 |
| 14 | (Hex)3 (HexNAc)2 (Deoxyhexose)4 | G93469SN, G15747RC | 1867.940 | | | | |
| 15 | (Hex)1 (HexNAc)2 | G00033MO, G61730RY, | 763.384 | 763.386 | 9.26 | 2.49 | 3.59 |
| | | G56868BH | | | | | |
| 16 | (Hex)1 (HexNAc)2 (Deoxyhexose)1 | G32426JY, G74353FF | 937.473 | 937.475 | 22.56 | 6.07 | 8.76 |
| 17 | (Hex)2 (HexNAc)2 | G64973KT | 967.483 | 967.486 | 4.19 | 1.13 | 1.63 |
| 18 | (Hex)1 (HexNAc)3 | G68893BQ, G23438NR | 1008.510 | 1008.514 | 2.78 | 0.75 | 1.08 |
| 19 | (Hex)1 (HexNAc)2 (Deoxyhexose)2 | G89748NG, G09520ZQ | 1111.562 | | | | |
| 20 | (Hex)1 (HexNAc)2 (NeuAc)1 | G85608AG, G64844ET | 1124.557 | 1124.559 | 2.50 | 0.67 | 0.97 |
| 21 | (Hex)2 (HexNAc)2 (Deoxyhexose)1 | G94514IB, G61216ZY | 1141.573 | 1141.576 | 3.00 | 0.81 | 1.16 |
| 22 | (Hex)1 (HexNAc)2 (NeuGc)1 | G60426XC | 1154.568 | 1154.573 | 1.56 | 0.42 | 0.61 |
| 23 | (Hex)3 (HexNAc)2 | G16404NW, G25323VU | 1171.583 | 1171.586 | 0.14 | 0.04 | 0.05 |
| 24 | (Hex)1 (HexNAc)3 (Deoxyhexose)1 | G28878FC | 1182.599 | 1182.602 | 4.86 | 1.31 | 1.89 |
| 25 | (Hex)2 (HexNAc)3 | G96915PP, G13483MW | 1212.610 | 1212.612 | 1.94 | 0.52 | 0.75 |
| 26 | (Hex)1 (HexNAc)4 | G59229NY | 1253.636 | | | | |
| 27 | (Hex)2 (HexNAc)2 (Deoxyhexose)2 | G23119MJ | 1315.662 | 1315.666 | 2.54 | 0.68 | 0.99 |
| 28 | (Hex)2 (HexNAc)2 (NeuAc)1 | G46748BU | 1328.657 | 1328.659 | 4.55 | 1.23 | 1.77 |
| 29 | (Hex)3 (HexNAc)2 (Deoxyhexose)1 | G15849KC, G81911LP, | 1345.672 | 1345.676 | 0.25 | 0.07 | 0.10 |
| | | G41486OC | | | | | |
| 30 | (Hex)2 (HexNAc)3 (Deoxyhexose)1 | G91459EI, G26724PP, | 1386.699 | 1386.703 | 0.41 | 0.11 | 0.16 |
| | | G52132CU | | | | | |
| 31 | (Hex)3 (HexNAc)3 | G86537AD, G89585FG | 1416.709 | 1416.713 | 0.10 | 0.03 | 0.04 |
| 32 | (Hex)2 (HexNAc)4 | G34764BK, G12074QJ | 1457.736 | 1457.729 | 0.32 | 0.09 | 0.12 |
| 33 | (Hex)2 (HexNAc)2 (Deoxyhexose)3 | G01532FF | 1489.751 | | | | |
| 34 | (Hex)2 (HexNAc)2 (Deoxyhexose)1 (NeuAc)1 | G77740PR, G59155GF | 1502.750 | | | | |
| 35 | (Hex)3 (HexNAc)2 (Deoxyhexose)2 | G20310DM, G78177LC | 1519.762 | | | | |
| 36 | (Hex)2 (HexNAc)3 (Deoxyhexose)2 | G68384KC, G39326PP | 1560.788 | 1560.792 | 2.48 | 0.67 | 0.96 |
| 37 | (Hex)3 (HexNAc)3 (Deoxyhexose)1 | G66741QE, G16458JH | 1590.799 | 1590.806 | 0.32 | 0.09 | 0.12 |
| 38 | (Hex)2 (HexNAc)4 (Deoxyhexose)1 | G23700TV | 1631.825 | 1631.822 | 0.40 | 0.11 | 0.15 |
| 39 | (Hex)3 (HexNAc)4 | G94517VF, G57672ST | 1661.836 | 1661.831 | 0.49 | 0.13 | 0.19 |
| 40 | (Hex)3 (HexNAc)2 (Deoxyhexose)3 | G70416EY, G13012GZ | 1693.851 | | | | |
| 4. | (Hex)2 (HexNAc)5 | G23048PE | 1702.862 | | | | |
| 42 | (Hex)3 (HexNAc)2 (Deoxyhexose)1 (NeuAc)1 | G74607VK, G81461IK | 1706.846 | | | | |
| 43 | (Hex)3 (HexNAc)3 (Deoxyhexose)2 | G35949CT, G71094KR, | 1764.888 | | | | |
| | | G08426KY | | | | | |
| 44 | (Hex)2 (HexNAc)4 (Deoxyhexose)2 | G90965BZ | 1805.914 | 1805.921 | 0.95 | 0.26 | 0.37 |
| 45 | (Hex)3 (HexNAc)4 (Deoxyhexose)1 | G02990AF, G29956GF | 1835.925 | 1835.927 | 1.03 | 0.28 | 0.40 |
| 46 | (Hex)3 (HexNAc)3 (Deoxyhexose)3 | G23021IW | 1938.977 | | | | |
| 47 | (Hex)4 (HexNAc)3 (Deoxyhexose)2 | G68308CM | 1968.988 | | | | |
| 48 | (Hex)3 (HexNAc)4 (Deoxyhexose)2 | G93333OF | 1016.502 | | | | |
| 49 | (Hex)4 (HexNAc)4 (Deoxyhexose)1 | G94768NG | 1031.507 | 1031.509 | 0.47 | 0.13 | 0.18 |
| 50 | (Hex)2 (HexNAc)5 (Deoxyhexose)2 | G25612EW | 1037.015 | | | | |
| 51 | (Hex)3 (HexNAc)3 (Deoxyhexose)4 | G84853WN | 1068.028 | | | | |
| 52 | (Hex)5 (HexNAc)6 (Deoxyhexose)3 | G65612SS | 1552.772 | | | | |
| 53 | (Hex)3 (HexNAc)5 (Deoxyhexose)1 | G05252QE | 1052.020 | 1052.023 | 0.94 | 0.25 | 0.36 |
| 54 | (Hex)7 (HexNAc)6 (Deoxyhexose)1 | G61898SS | 1582.783 | | | | |
| 55 | (Hex)3 (HexNAc)6 | G62609ZF | 1087.539 | | | | |
| 56 | (Hex)3 (HexNAc)4 (Deoxyhexose)3 | G59787TQ | 1103.546 | | | | |
| 57 | (Hex)4 (HexNAc)4 (Deoxyhexose)2 | G98518WL | 1118.552 | | | | |
| 58 | (Hex)6 (HexNAc)7 (Deoxyhexose)2 | G29852ZH | 1690.341 | | | | |
| 59 | (Hex)4 (HexNAc)5 (Deoxyhexose)1 | G92547QZ | 1154.070 | 1154.073 | 1.18 | 0.32 | 0.46 |
| 60 | (Hex)3 (HexNAc)4 (Deoxyhexose)4 | G89469SP | 1190.591 | | | | |
| 51 | (Hex)5 (HexNAc)4 (Deoxyhexose)2 | G21630AC | 1220.601 | | | | |
| 62 | (Hex)4 (HexNAc)5 (Deoxyhexose)2 | G82251ZP, G18603ZQ | 1241.115 | 1241.118 | 0.35 | 0.09 | 0.13 |
| 63 | (Hex)5 (HexNAc)5 (Deoxyhexose)1 | G25957KN | 1256.120 | 1256.123 | 0.32 | 0.09 | 0.13 |
| 64 | (Hex)4 (HexNAc)6 (Deoxyhexose)1 | G32752FJ | 1276.633 | 1276.637 | 0.30 | 0.08 | 0.12 |
| 65 | (Hex)6 (HexNAc)5 (Deoxyhexose)1 | G37901JE, G84713IO | 1358.170 | | | | |
| 66 | (Hex)4 (HexNAc)6 (Deoxyhexose)2 | G99804SJ | 1363.678 | 1363.682 | 0.17 | 0.05 | 0.07 |
| 67 | (Hex)4 (HexNAc)5 (Deoxyhexose)4 | G11381FO | 1415.204 | | | | |
| 68 | (Hex)5 (HexNAc)5 (Deoxyhexose)3 | G90829NZ | 1430.209 | | | | |
| 69 | (Hex)6 (HexNAc)5 (Deoxyhexose)2 | G70999YJ, G02681FY | 1445.215 | | | | |
| 70 | (Hex)5 (HexNAc)6 (Deoxyhexose)2 | G44467ZE | 1465.728 | 1465.730 | 0.11 | 0.03 | 0.04 |
| 71 | (Hex)3 (HexNAc)5 (Deoxyhexose)2 | G28921PH | 1139.065 | | | | |
| 72 | (Hex)6 (HexNAc)5 (Deoxyhexose)3 | G18501TC | 1532.259 | | | | |
| 73 | (Hex)4 (HexNAc)4 (Deoxyhexose)3 | G66166BF | 1205.596 | | | | |
| 74 | (HexNAc)2 | G00041MO, G00057MO | 559.284 | 559.285 | 8.86 | 2.38 | 3.44 |
| 75 | (HexNAc)2 (NeuAc)1 | G63334FZ | 920.457 | 920.460 | 3.84 | 1.03 | 1.49 |
| 76 | (HexNAc)2 (NeuGc)1 | G09441IP | 950.468 | 950.471 | 2.76 | 0.74 | 1.07 |
| 77 | (Hex)3 (HexNAc)5 | G00505CR | 1906.962 | 1906.970 | 0.38 | 0.10 | 0.15 |
| 78 | (Hex)2 (HexNAc)5 (Deoxyhexose)1 | G09396HG | 949.970 | | | | |
| 79 | (Hex)2 (HexNAc)1 | G28052FT | 722.357 | 722.359 | 3.79 | 1.02 | 1.47 |
| 80 | (Hex)2 (HexNAc)1 (NeuAc)1 | G59126YU | 1083.531 | 1083.530 | 0.59 | 0.16 | 0.23 |
| 81 | (Hex)1 (Deoxyhexose)1 | G00068MO | 447.220 | 447.221 | 38.66 | 10.40 | na |
| 82 | (Hex)1 (NeuAc)1 | G30207PZ, G63069TR | 634.305 | 634.306 | 43.37 | 11.67 | na |
| 83 | (Hex)1 (NeuGc)1 | G38557KR, G59867EM | 664.315 | 664.317 | 31.92 | 8.59 | na |
| | | | | Total w/Peel: | 371.58 | 100.00 | 100.00 |
| | | | | Total w/o Peel: | 257.62 | | |

Glycans 81, 82, 83 are the expected products of peeling reactions

TABLE 5
Glycan Profile for MUC5B Preparation

| | | | | | pmol/mg of | % Total Profile | |
|---|---|---|---|---|---|---|---|
| Glycan | | | m/z (M + Na)¹⁺ | | total released | | w/o |
| # | Composition | Gly TouCan Accession(s) | Theoretical | Detected | glycan | | Peeling |
| 1 | (HexNAc)1 | G57321FI | 314.157 | 314.158 | 0.73 | 0.40 | 0.44 |
| 2 | (Hex)1 (HexNAc)1 | G76355TG, G85856KC | 518.257 | 518.258 | 14.30 | 7.90 | 8.66 |
| 3 | (Hex)1 (HexNAc)1 (Deoxyhexose)1 | G94435QH | 692.346 | 692.349 | 38.27 | 21.15 | 23.16 |
| 4 | (Hex)1 (HexNAc)1 (NeuAc)1 | G65562ZE | 879.431 | 879.434 | 4.38 | 2.42 | 2.65 |
| 5 | (Hex)2 (HexNAc)1 (Deoxyhexose) 1 | G73318SN, G33986KK | 896.446 | 896.449 | 3.64 | 2.01 | 2.21 |
| 6 | (Hex)1 (HexNAc)1 (NeuGc)1 | G64527IJ | 909.441 | | | | |
| 7 | (Hex)1 (HexNAc)1 (Deoxyhexose)1 (NeuAc)1 | G47180UC | 1053.520 | 1053.522 | 1.15 | 0.63 | 0.69 |
| 8 | (Hex)2 (HexNAc)1 (Deoxyhexose)2 | G68200GL | 1070.535 | 1070.539 | 2.73 | 1.51 | 1.65 |
| 9 | (Hex)1 (HexNAc)1 (NeuAc)2 | G01614ZM | 1240.605 | | | | |
| 10 | (Hex)2 (HexNAc)1 (Deoxyhexose)3 | G82961CS | 1244.625 | 1244.628 | 0.60 | 0.33 | 0.36 |
| 11 | (Hex)2 (HexNAc)1 (Deoxyhexose)1 (NeuAc)1 | G49549VN, G95742RK | 1257.620 | 1257.625 | 0.87 | 0.48 | 0.53 |
| 12 | (Hex)1 (HexNAc)1 (NeuAc)1 (NeuGc)1 | G49527BY | 1270.615 | | | | |
| 13 | (Hex)1 (HexNAc)2 (Deoxyhexose)1 (NeuAc)1 | G75749JP, G40270LS | 1298.646 | 1298.650 | 1.33 | 0.73 | 0.80 |
| 14 | (Hex)3 (HexNAc)2 (Deoxyhexose)4 | G93469SN, G15747RC | 1867.940 | 1867.946 | 1.30 | 0.72 | 0.79 |
| 15 | (Hex)1 (HexNAc)2 | G00033MO, G61730RY, | 763.384 | 763.388 | 4.25 | 2.35 | 2.57 |
| | | G56868BH | | | | | |
| 16 | (Hex)1 (HexNAc)2 (Deoxyhexose)1 | G32426JY, G74353FF | 937.473 | 937.476 | 11.24 | 6.21 | 6.80 |
| 17 | (Hex)2 (HexNAc)2 | G64973KT | 967.483 | 967.486 | 1.89 | 1.05 | 1.15 |
| 18 | (Hex)1 (HexNAc)3 | G68893BQ, G23438NR | 1008.510 | 1008.514 | 0.72 | 0.40 | 0.44 |
| 19 | (Hex)1 (HexNAc)2 (Deoxyhexose)2 | G89748NG, G09520ZQ | 1111.562 | 1111.565 | 4.05 | 2.24 | 2.45 |
| 20 | (Hex)1 (HexNAc)2 (NeuAc)1 | G85608AG, G64844ET | 1124.557 | 1124.562 | 1.22 | 0.67 | 0.74 |
| 21 | (Hex)2 (HexNAc)2 (Deoxyhexose)1 | G94514IB, G61216ZY | 1141.573 | 1141.575 | 3.99 | 2.20 | 2.41 |
| 22 | (Hex) 1 (HexNAc)2 (NeuGc)1 | G60426XC | 1154.568 | | | | |
| 23 | (Hex)3 (HexNAc)2 | G16404NW, G25323VU | 1171.583 | 1171.586 | 1.65 | 0.91 | 1.00 |
| 24 | (Hex)1 (HexNAc)3 (Deoxyhexose)1 | G28878FC | 1182.599 | 1182.602 | 2.98 | 1.65 | 1.81 |
| 25 | (Hex)2 (HexNAc)3 | G96915PP, G13483MW | 1212.610 | 1212.612 | 1.87 | 1.03 | 1.13 |
| 26 | (Hex)1 (HexNAc)4 | G59229NY | 1253.636 | | | | |
| 27 | (Hex)2 (HexNAc)2 (Deoxyhexose)2 | G23119MJ | 1315.662 | 1315.666 | 4.94 | 2.73 | 2.99 |
| 28 | (Hex)2 (HexNAc)2 (NeuAc)1 | G46748BU | 1328.657 | 1328.659 | 2.42 | 1.34 | 1.46 |
| 29 | (Hex)3 (HexNAc)2 (Deoxyhexose)1 | G15849KC, G81911LP, | 1345.672 | 1345.676 | 3.30 | 1.82 | 2.00 |
| | | G41486OC | | | | | |
| 30 | (Hex)2 (HexNAc)3 (Deoxyhexose)1 | G91459EI, G26724PP, | 1386.699 | 1386.702 | 3.40 | 1.88 | 2.06 |
| | | G52132CU | | | | | |
| 31 | (Hex)3 (HexNAc)3 | G86537AD, G89585FG | 1416.709 | 1416.714 | 0.17 | 0.09 | 0.10 |
| 32 | (Hex)2 (HexNAc)4 | G34764BK, G12074QJ | 1457.736 | 1457.734 | 0.27 | 0.15 | 0.17 |
| 33 | (Hex)2 (HexNAc)2 (Deoxyhexose)3 | G01532FF | 1489.751 | 1489.755 | 1.78 | 0.98 | 1.07 |
| 34 | (Hex)2 (HexNAc)2 (Deoxyhexose)1 (NeuAc)1 | G77740PR, G59155GF | 1502.750 | 1502.751 | 0.22 | 0.12 | 0.13 |
| 35 | (Hex)3 (HexNAc)2 (Deoxyhexose)2 | G20310DM, G78177LC | 1519.762 | 1519.766 | 4.35 | 2.40 | 2.63 |
| 36 | (Hex)2 (HexNAc)3 (Deoxyhexose)2 | G68384KC, G39326PP | 1560.788 | 1560.792 | 3.40 | 1.88 | 2.06 |
| 37 | (Hex)3 (HexNAc)3 (Deoxyhexose)1 | G66741QE, G16458JH | 1590.799 | 1590.803 | 2.15 | 1.19 | 1.30 |
| 38 | (Hex)2 (HexNAc)4 (Deoxyhexose)1 | G23700TV | 1631.825 | 1631.830 | 1.05 | 0.58 | 0.64 |
| 39 | (Hex)3 (HexNAc)4 | G94517VF, G57672ST | 1661.836 | 1661.837 | 0.05 | 0.03 | 0.03 |
| 40 | (Hex)3 (HexNAc)2 (Deoxyhexose)3 | G70416EY, G13012GZ | 1693.851 | 1693.856 | 2.22 | 1.23 | 1.34 |
| 41 | (Hex)2 (HexNAc)5 | G23048PE | 1702.862 | | | | |
| 42 | (Hex)3 (HexNAc)2 (Deoxyhexose)1 (NeuAc)1 | G74607VK, G81461IK | 1706.846 | 1706.851 | 0.91 | 0.50 | 0.55 |
| 43 | (Hex)3 (HexNAc)3 (Deoxyhexose)2 | G35949CT, G71094KR, | 1764.888 | 1764.897 | 5.23 | 2.89 | 3.17 |
| | | G08426KY | | | | | |
| 44 | (Hex)2 (HexNAc)4 (Deoxyhexose)2 | G90965BZ | 1805.914 | 1805.919 | 2.17 | 1.20 | 1.31 |
| 45 | (Hex)3 (HexNAc)4 (Deoxyhexose)1 | G02990AF, G29956GF | 1835.925 | 1835.927 | 1.45 | 0.80 | 0.88 |
| 46 | (Hex)3 (HexNAc)3 (Deoxyhexose)3 | G23021IW | 1938.977 | 1938.979 | 2.11 | 1.17 | 1.28 |
| 47 | (Hex)4 (HexNAc)3 (Deoxyhexose)2 | G68308CM | 1968.988 | 1968.990 | 1.14 | 0.63 | 0.69 |
| 48 | (Hex)3 (HexNAc)4 (Deoxyhexose)2 | G93333OF | 1016.502 | 1016.504 | 1.34 | 0.74 | 0.81 |
| 49 | (Hex)4 (HexNAc)4 (Deoxyhexose)1 | G94768NG | 1031.507 | 1031.510 | 0.57 | 0.32 | 0.35 |
| 50 | (Hex)2 (HexNAc)5 (Deoxyhexose)2 | G25612EW | 1037.015 | 1037.018 | 0.58 | 0.32 | 0.35 |
| 51 | (Hex)3 (HexNAc)3 (Deoxyhexose)4 | G84853WN | 1068.028 | 1068.031 | 0.84 | 0.47 | 0.51 |
| 52 | (Hex)5 (HexNAc)6 (Deoxyhexose)3 | G65612SS | 1552.772 | 1552.776 | 0.19 | 0.10 | 0.11 |
| 53 | (Hex)3 (HexNAc)5 (Deoxyhexose)1 | G05252QE | 1052.020 | 1052.023 | 0.13 | 0.07 | 0.08 |
| 54 | (Hex)7 (HexNAc)6 (Deoxyhexose)1 | G61898SS | 1582.783 | 1582.796 | 0.05 | 0.03 | 0.03 |
| 55 | (Hex)3 (HexNAc)6 | G62609ZF | 1087.539 | | | | |
| 56 | (Hex)3 (HexNAc)4 (Deoxyhexose)3 | G59787TQ | 1103.546 | 1103.549 | 2.94 | 1.62 | 1.78 |
| 57 | (Hex)4 (HexNAc)4 (Deoxyhexose)2 | G98518WL | 1118.552 | 1118.555 | 1.13 | 0.62 | 0.68 |
| 58 | (Hex)6 (HexNAc)7 (Deoxyhexose)2 | G29852ZH | 1690.341 | | | | |
| 59 | (Hex)4 (HexNAc)5 (Deoxyhexose)1 | G92547QZ | 1154.070 | 1154.073 | 0.25 | 0.14 | 0.15 |
| 60 | (Hex)3 (HexNAc)4 (Deoxyhexose)4 | G89469SP | 1190.591 | 1190.594 | 1.72 | 0.95 | 1.04 |
| 61 | (Hex)5 (HexNAc)4 (Deoxyhexose)2 | G21630AC | 1220.601 | 1220.604 | 0.84 | 0.46 | 0.51 |
| 62 | (Hex)4 (HexNAc)5 (Deoxyhexose)2 | G82251ZP, G18603ZQ | 1241.115 | 1241.118 | 0.49 | 0.27 | 0.30 |
| 63 | (Hex)5 (HexNAc)5 (Deoxyhexose)1 | G25957KN | 1256.120 | 1256.123 | 0.46 | 0.25 | 0.28 |
| 64 | (Hex)4 (HexNAc)6 (Deoxyhexose)1 | G32752FJ | 1276.633 | | | | |
| 65 | (Hex)6 (HexNAc)5 (Deoxyhexose)1 | G37901JE, G84713IO | 1358.170 | | | | |
| 66 | (Hex)4 (HexNAc)6 (Deoxyhexose)2 | G99804SJ | 1363.678 | | | | |
| 67 | (Hex)4 (HexNAc)5 (Deoxyhexose)4 | G11381FO | 1415.204 | 1415.208 | 0.51 | 0.28 | 0.31 |
| 68 | (Hex)5 (HexNAc)5 (Deoxyhexose)3 | G90829NZ | 1430.209 | 1430.213 | 0.08 | 0.04 | 0.05 |
| 69 | (Hex)6 (HexNAc)5 (Deoxyhexose)2 | G70999YJ, G02681FY | 1445.215 | 1445.215 | 0.22 | 0.12 | 0.14 |
| 70 | (Hex)5 (HexNAc)6 (Deoxyhexose)2 | G44467ZE | 1465.728 | 1465.729 | 0.22 | 0.12 | 0.14 |
| 71 | (Hex)3 (HexNAc)5 (Deoxyhexose)2 | G28921PH | 1139.065 | | | | |
| 72 | (Hex)6 (HexNAc)5 (Deoxyhexose)3 | G18501TC | 1532.259 | | | | |
| 73 | (Hex)4 (HexNAc)4 (Deoxyhexose)3 | G66166BF | 1205.596 | 1205.599 | 1.07 | 0.59 | 0.65 |
| 74 | (HexNAc)2 | G00041MO, G00057MO | 559.284 | 559.285 | 1.11 | 0.62 | 0.67 |
| 75 | (HexNAc)2 (NeuAc)1 | G63334FZ | 920.457 | 920.460 | 1.37 | 0.76 | 0.83 |
| 76 | (HexNAc)2 (NeuGc)1 | G09441IP | 950.468 | | | | |
| 77 | (Hex)3 (HexNAc)5 | G00505CR | 1906.962 | | | | |
| 78 | (Hex)2 (HexNAc)5 (Deoxyhexose) 1 | G09396HG | 949.970 | | | | |
| 79 | (Hex)2 (HexNAc)1 | G28052FT | 722.357 | 722.359 | 3.13 | 1.73 | 1.89 |
| 80 | (Hex)2 (HexNAc)1 (NeuAc)1 | G59126YU | 1083.531 | 1083.530 | 0.07 | 0.04 | 0.04 |
| 81 | (Hex) 1 (Deoxyhexose)1 | G00068MO | 447.220 | 447.221 | 7.66 | 4.23 | na |
| 82 | (Hex)1 (NeuAc)1 | G30207PZ, G63069TR | 634.305 | 634.307 | 7.83 | 4.33 | na |
| 83 | (Hex)1 (NeuGc)1 | G38557KR, G59867EM | 664.315 | 664.317 | 0.24 | 0.13 | na |
| | | | | Total w/Peel: | 180.95 | 100.00 | 100.00 |
| | | | | Total w/o Peel: | 165.23 | | |

Glycans 81, 82, 83 are the expected products of peeling reactions

TABLE 6
Glycan Profile for MUC5AC Preparation

| | | | | | pmol/mg of | % Total Profile | |
|---|---|---|---|---|---|---|---|
| Glycan | | | m/z (M + Na)¹⁺ | | total released | | w/o |
| # | Composition | Gly TouCan Accession(s) | Theoretical | Detected | glycan | | Peeling |
| 1 | (HexNAc)1 | G57321FI | 314.157 | 314.159 | 2.50 | 0.46 | 0.49 |
| 2 | (Hex)1 (HexNAc)1 | G76355TG, G85856KC | 518.257 | 518.26 | 91.72 | 16.83 | 17.91 |
| 3 | (Hex)1 (HexNAc)1 (Deoxyhexose)1 | G94435QH | 692.346 | 692.35 | 73.00 | 13.40 | 14.26 |
| 4 | (Hex)1 (HexNAc)1 (NeuAc)1 | G65562ZE | 879.431 | 879.44 | 5.99 | 1.10 | 1.17 |
| 5 | (Hex)2 (HexNAc)1 (Deoxyhexose)1 | G73318SN, G33986KK | 896.446 | 896.45 | 10.38 | 1.91 | 2.03 |
| 6 | (Hex)1 (HexNAc)1 (NeuGc)1 | G64527IJ | 909.441 | | | | |
| 7 | (Hex)1 (HexNAc)1 (Deoxyhexose)1 (NeuAc)1 | G47180UC | 1053.520 | 1053.53 | 1.37 | 0.25 | 0.27 |
| 8 | (Hex)2 (HexNAc)1 (Deoxyhexose)2 | G68200GL | 1070.535 | 1070.54 | 0.88 | 0.16 | 0.17 |
| 9 | (Hex)1 (HexNAc)1 (NeuAc)2 | G01614ZM | 1240.605 | | | | |
| 10 | (Hex)2 (HexNAc)1 (Deoxyhexose)3 | G82961CS | 1244.625 | | | | |
| 11 | (Hex)2 (HexNAc)1 (Deoxyhexose)1 (NeuAc)1 | G49549VN, G95742RK | 1257.620 | | | | |
| 12 | (Hex)1 (HexNAc)1 (NeuAc)1 (NeuGc)1 | G49527BY | 1270.615 | | | | |
| 13 | (Hex)1 (HexNAc)2 (Deoxyhexose)1 (NeuAc)1 | G75749JP, G40270LS | 1298.646 | | | | |
| 14 | (Hex)3 (HexNAc)2 (Deoxyhexose)4 | G93469SN, G15747RC | 1867.940 | | | | |
| 15 | (Hex)1 (HexNAc)2 | G00033MO, G61730RY, | 763.384 | 763.39 | 87.06 | 15.98 | 17.00 |
| | | G56868BH | | | | | |
| 16 | (Hex)1 (HexNAc)2 (Deoxyhexose)1 | G32426JY, G74353FF | 937.473 | 937.48 | 10.15 | 1.86 | 1.98 |
| 17 | (Hex)2 (HexNAc)2 | G64973KT | 967.483 | 967.49 | 17.03 | 3.13 | 3.33 |
| 18 | (Hex)1 (HexNAc)3 | G68893BQ, G23438NR | 1008.510 | 1008.51 | 7.48 | 1.37 | 1.46 |
| 19 | (Hex)1 (HexNAc)2 (Deoxyhexose)2 | G89748NG, G09520ZQ | 1111.562 | | | | |
| 20 | (Hex)1 (HexNAc)2 (NeuAc)1 | G85608AG, G64844ET | 1124.557 | 1124.56 | 2.52 | 0.46 | 0.49 |
| 21 | (Hex)2 (HexNAc)2 (Deoxyhexose)1 | G94514IB, G61216ZY | 1141.573 | 1141.58 | 20.85 | 3.83 | 4.07 |
| 22 | (Hex)1 (HexNAc)2 (NeuGc)1 | G60426XC | 1154.568 | | | | |
| 23 | (Hex)3 (HexNAc)2 | G16404NW, G25323VU | 1171.583 | 1171.59 | 1.60 | 0.29 | 0.31 |
| 24 | (Hex)1 (HexNAc)3 (Deoxyhexose)1 | G28878FC | 1182.599 | 1182.61 | 1.86 | 0.34 | 0.36 |
| 25 | (Hex)2 (HexNAc)3 | G96915PP, G13483MW | 1212.610 | 1212.62 | 17.89 | 3.28 | 3.49 |
| 26 | (Hex)1 (HexNAc)4 | G59229NY | 1253.636 | 1253.64 | 2.25 | 0.41 | 0.44 |
| 27 | (Hex)2 (HexNAc)2 (Deoxyhexose)2 | G23119MJ | 1315.662 | 1315.67 | 7.98 | 1.46 | 1.56 |
| 28 | (Hex)2 (HexNAc)2 (NeuAc)1 | G46748BU | 1328.657 | 1328.66 | 1.72 | 0.31 | 0.34 |
| 29 | (Hex)3 (HexNAc)2 (Deoxyhexose)1 | G15849KC, G81911LP, | 1345.672 | 1345.68 | 4.93 | 0.90 | 0.96 |
| | | G41486OC | | | | | |
| 30 | (Hex)2 (HexNAc)3 (Deoxyhexose)1 | G91459EI, G26724PP, | 1386.699 | 1386.71 | 24.48 | 4.49 | 4.78 |
| | | G52132CU | | | | | |
| 31 | (Hex)3 (HexNAc)3 | G86537AD, G89585FG | 1416.709 | 1416.72 | 3.25 | 0.60 | 0.64 |
| 32 | (Hex)2 (HexNAc)4 | G34764BK, G12074QJ | 1457.736 | 1457.74 | 23.39 | 4.29 | 4.57 |
| 33 | (Hex)2 (HexNAc)2 (Deoxyhexose)3 | G01532FF | 1489.751 | | | | |
| 34 | (Hex)2 (HexNAc)2 (Deoxyhexose)1 (NeuAc)1 | G77740PR, G59155GF | 1502.750 | | | | |
| 35 | (Hex)3 (HexNAc)2 (Deoxyhexose)2 | G20310DM, G78177LC | 1519.762 | 1519.77 | 12.29 | 2.26 | 2.40 |
| 36 | (Hex)2 (HexNAc)3 (Deoxyhexose)2 | G68384KC, G39326PP | 1560.788 | 1560.79 | 1.51 | 0.28 | 0.29 |
| 37 | (Hex)3 (HexNAc)3 (Deoxyhexose)1 | G66741QE, G16458JH | 1590.799 | 1590.81 | 12.21 | 2.24 | 2.38 |
| 38 | (Hex)2 (HexNAc)4 (Deoxyhexose)1 | G23700TV | 1631.825 | 1631.83 | 10.85 | 1.99 | 2.12 |
| 39 | (Hex)3 (HexNAc)4 | G94517VF, G57672ST | 1661.836 | 1661.84 | 11.04 | 2.03 | 2.15 |
| 40 | (Hex)3 (HexNAc)2 (Deoxyhexose)3 | G70416EY, G13012GZ | 1693.851 | | | | |
| 41 | (Hex)2 (HexNAc)5 | G23048PE | 1702.862 | 1702.87 | 0.98 | 0.18 | 0.19 |
| 42 | (Hex)3 (HexNAc)2 (Deoxyhexose)1 (NeuAc)1 | G74607VK, G81461IK | 1706.846 | | | | |
| 43 | (Hex)3 (HexNAc)3 (Deoxyhexose)2 | G35949CT, G71094KR, | 1764.888 | 1764.90 | 4.80 | 0.88 | 0.94 |
| | | G08426KY | | | | | |
| 44 | (Hex)2 (HexNAc)4 (Deoxyhexose)2 | G90965BZ | 1805.914 | 1805.92 | 1.21 | 0.22 | 0.24 |
| 45 | (Hex)3 (HexNAc)4 (Deoxyhexose)1 | G02990AF, G29956GF | 1835.925 | 1835.93 | 7.29 | 1.34 | 1.42 |
| 46 | (Hex)3 (HexNAc)3 (Deoxyhexose)3 | G23021IW | 1938.977 | | | | |
| 47 | (Hex)4 (HexNAc)3 (Deoxyhexose)2 | G68308CM | 1968.988 | 1968.99 | 2.02 | 0.37 | 0.39 |
| 48 | (Hex)3 (HexNAc)4 (Deoxyhexose)2 | G93333OF | 1016.502 | 1016.51 | 5.07 | 0.93 | 0.99 |
| 49 | (Hex)4 (HexNAc)4 (Deoxyhexose)1 | G94768NG | 1031.507 | 1031.51 | 1.75 | 0.32 | 0.34 |
| 50 | (Hex)2 (HexNAc)5 (Deoxyhexose)2 | G25612EW | 1037.015 | | | | |
| 51 | (Hex)3 (HexNAc)3 (Deoxyhexose)4 | G84853WN | 1068.028 | | | | |
| 52 | (Hex)5 (HexNAc)6 (Deoxyhexose)3 | G65612SS | 1552.772 | 1552.78 | 0.39 | 0.07 | 0.08 |
| 53 | (Hex)3 (HexNAc)5 (Deoxyhexose)1 | G05252QE | 1052.020 | 1052.03 | 2.00 | 0.37 | 0.39 |
| 54 | (Hex)7 (HexNAc)6 (Deoxyhexose)1 | G61898SS | 1582.783 | 1582.80 | 0.39 | 0.07 | 0.08 |
| 55 | (Hex)3 (HexNAc)6 | G62609ZF | 1087.539 | 1087.54 | 0.91 | 0.17 | 0.18 |
| 56 | (Hex)3 (HexNAc)4 (Deoxyhexose)3 | G59787TQ | 1103.546 | | | | |
| 57 | (Hex)4 (HexNAc)4 (Deoxyhexose)2 | G98518WL | 1118.552 | 1118.56 | 1.35 | 0.25 | 0.26 |
| 58 | (Hex)6 (HexNAc)7 (Deoxyhexose)2 | G29852ZH | 1690.341 | 1690.36 | 0.25 | 0.05 | 0.05 |
| 59 | (Hex)4 (HexNAc)5 (Deoxyhexose)1 | G92547QZ | 1154.070 | 1154.08 | 0.82 | 0.15 | 0.16 |
| 60 | (Hex)3 (HexNAc)4 (Deoxyhexose)4 | G89469SP | 1190.591 | | | | |
| 61 | (Hex)5 (HexNAc)4 (Deoxyhexose)2 | G21630AC | 1220.601 | 1220.61 | 0.31 | 0.06 | 0.06 |
| 62 | (Hex)4 (HexNAc)5 (Deoxyhexose)2 | G82251ZP, G18603ZQ | 1241.115 | 1241.12 | 0.93 | 0.17 | 0.18 |
| 63 | (Hex)5 (HexNAc)5 (Deoxyhexose)1 | G25957KN | 1256.120 | 1256.13 | 0.29 | 0.05 | 0.06 |
| 64 | (Hex)4 (HexNAc)6 (Deoxyhexose)1 | G32752FJ | 1276.633 | 1276.64 | 0.51 | 0.09 | 0.10 |
| 65 | (Hex)6 (HexNAc)5 (Deoxyhexose)1 | G37901JE, G84713IO | 1358.170 | 1358.18 | 0.25 | 0.05 | 0.05 |
| 66 | (Hex)4 (HexNAc)6 (Deoxyhexose)2 | G99804SJ | 1363.678 | 1363.69 | 0.21 | 0.04 | 0.04 |
| 67 | (Hex)4 (HexNAc)5 (Deoxyhexose)4 | G11381FO | 1415.204 | | | | |
| 68 | (Hex)5 (HexNAc)5 (Deoxyhexose)3 | G90829NZ | 1430.209 | 1430.22 | 0.14 | 0.03 | 0.03 |
| 69 | (Hex)6 (HexNAc)5 (Deoxyhexose)2 | G70999YJ, G02681FY | 1445.215 | 1445.22 | 0.13 | 0.02 | 0.03 |
| 70 | (Hex)5 (HexNAc)6 (Deoxyhexose)2 | G44467ZE | 1465.728 | 1465.73 | 0.11 | 0.02 | 0.02 |
| 71 | (Hex)3 (HexNAc)5 (Deoxyhexose)2 | G28921PH | 1139.065 | 1139.07 | 0.64 | 0.12 | 0.13 |
| 72 | (Hex)6 (HexNAc)5 (Deoxyhexose)3 | G18501TC | 1532.259 | 1532.27 | 0.56 | 0.10 | 0.11 |
| 73 | (Hex)4 (HexNAc)4 (Deoxyhexose)3 | G66166BF | 1205.596 | 1205.60 | 0.84 | 0.15 | 0.16 |
| 74 | (HexNAc)2 | G00041MO, G00057MO | 559.284 | | | | |
| 75 | (HexNAc)2 (NeuAc)1 | G63334FZ | 920.457 | | | | |
| 76 | (HexNAc)2 (NeuGc)1 | G09441IP | 950.468 | | | | |
| 77 | (Hex)3 (HexNAc)5 | G00505CR | 1906.962 | 1906.97 | 2.51 | 0.46 | 0.49 |
| 78 | (Hex)2 (HexNAc)5 (Deoxyhexose)1 | G09396HG | 949.970 | 949.98 | 0.62 | 0.11 | 0.12 |
| 79 | (Hex)2 (HexNAc)1 | G28052FT | 722.357 | 722.36 | 4.80 | 0.88 | 0.94 |
| 80 | (Hex)2 (HexNAc)1 (NeuAc)1 | G59126YU | 1083.531 | 1083.54 | 1.84 | 0.34 | 0.36 |
| 81 | (Hex)1 (Deoxyhexose)1 | G00068MO | 447.220 | 447.22 | 32.79 | 6.02 | na |
| 82 | (Hex)1 (NeuAc)1 | G30207PZ, G63069TR | 634.305 | | | | |
| 83 | (Hex)1 (NeuGc)1 | G38557KR, G59867EM | 664.315 | | | | |
| | | | | Total w/Peel: | 544.90 | 100.00 | 100.00 |
| | | | | Total w/o Peel: | 512.11 | | |

Glycans 81, 82, 83 are the expected products of peeling reactions

### Representative Structures in Tables 4-6

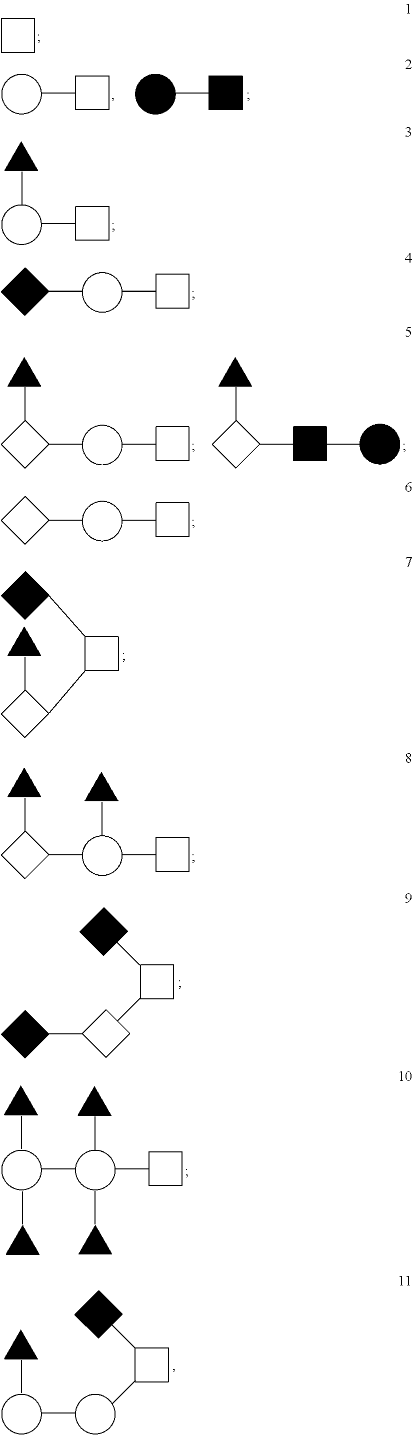

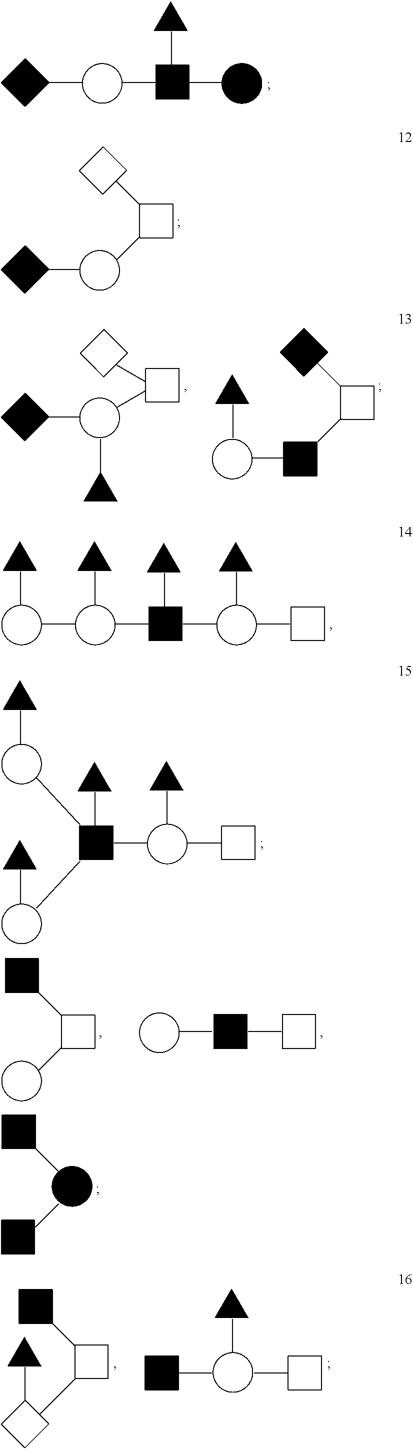

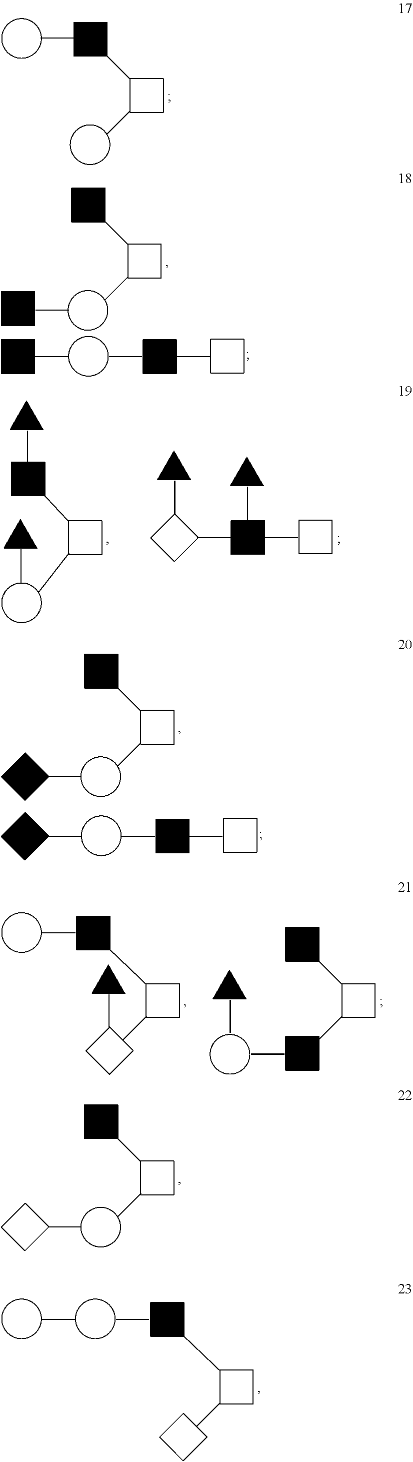

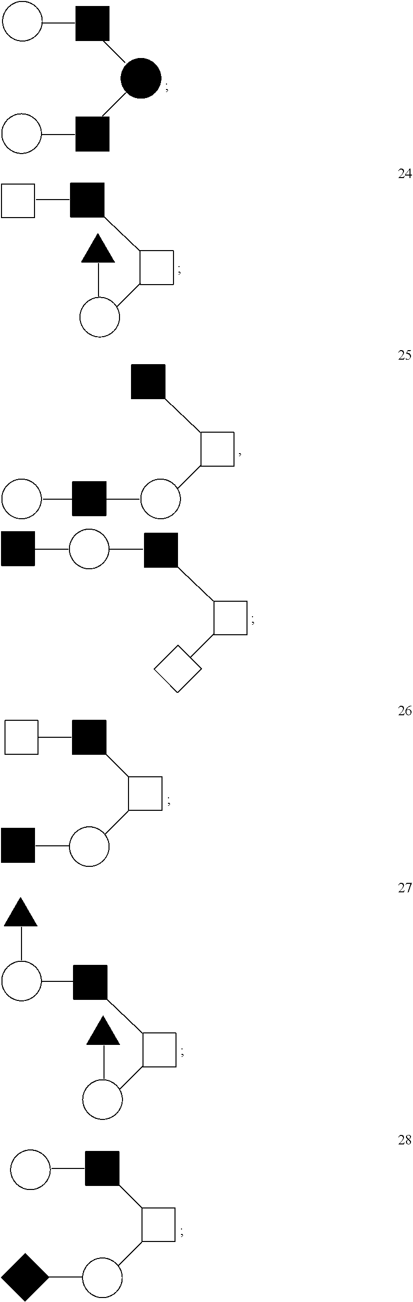

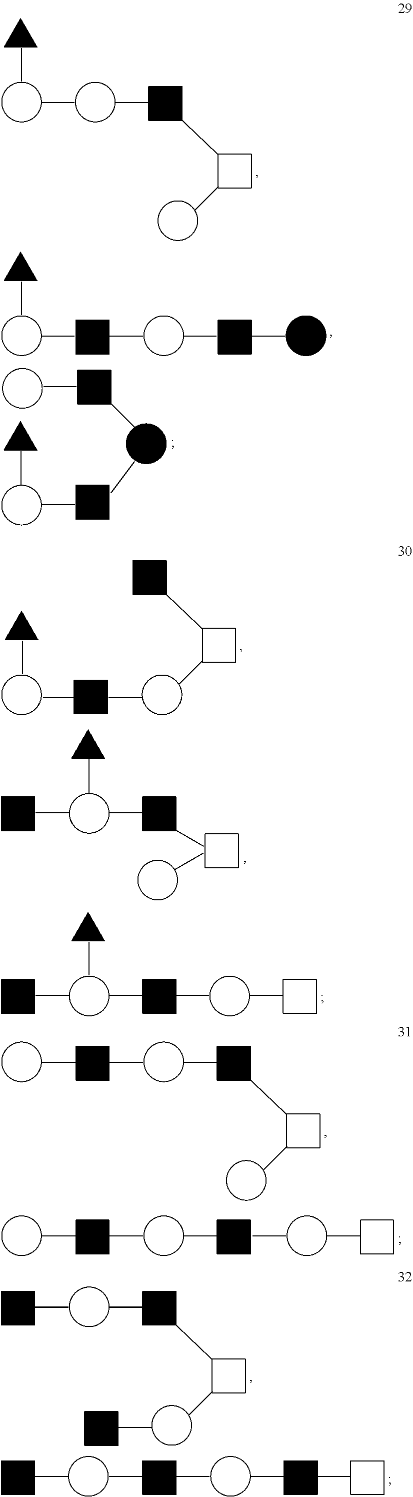

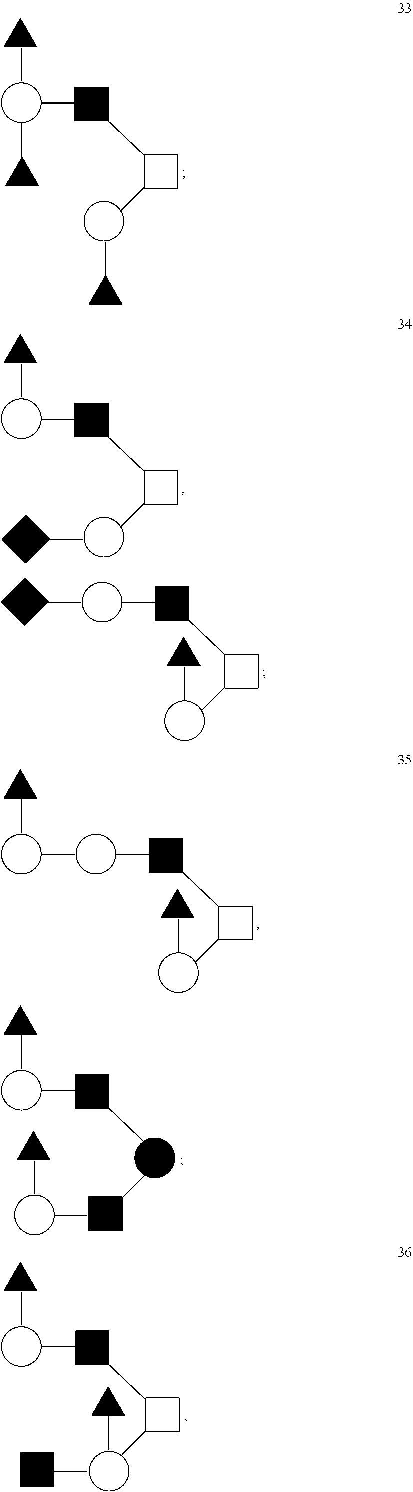

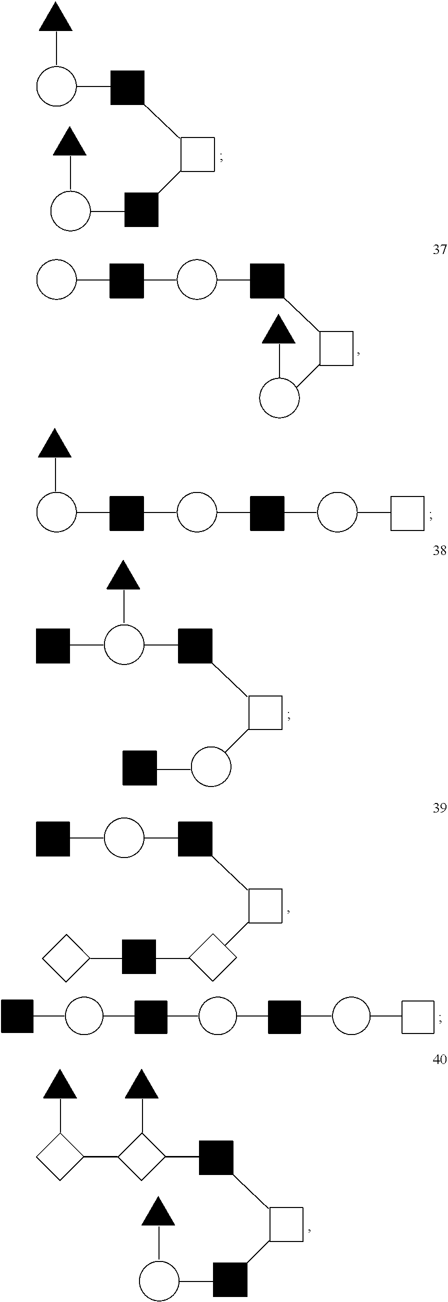

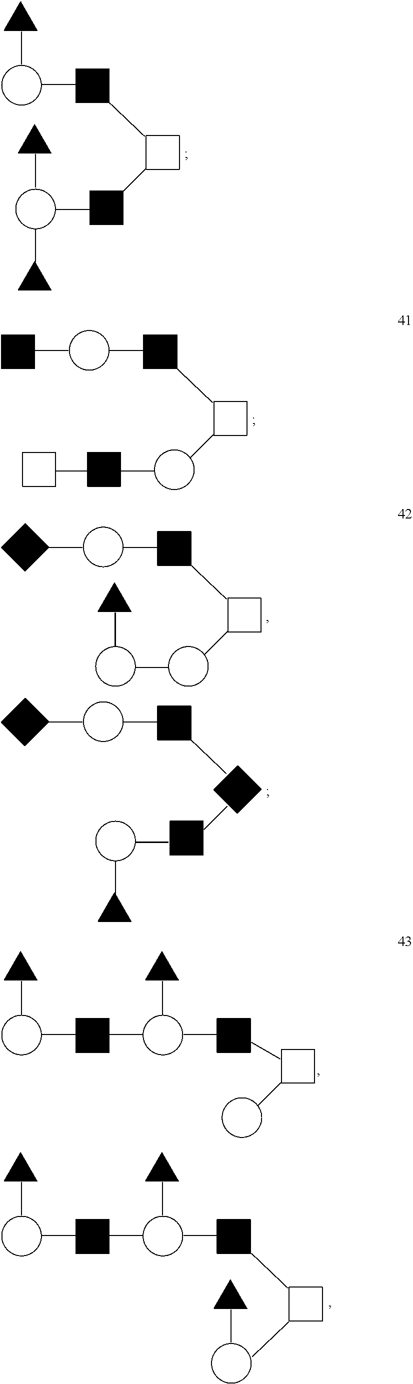

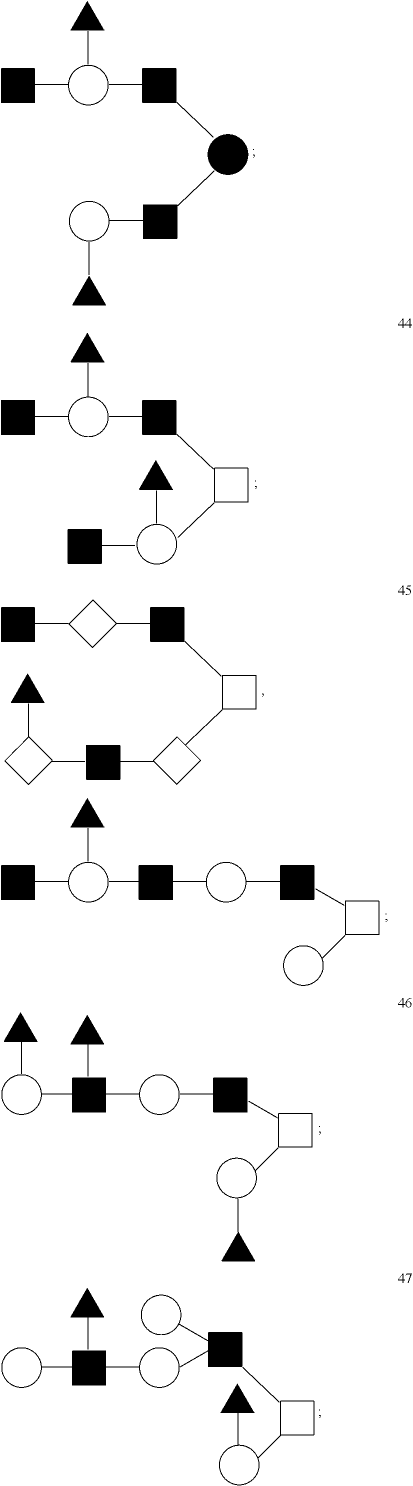

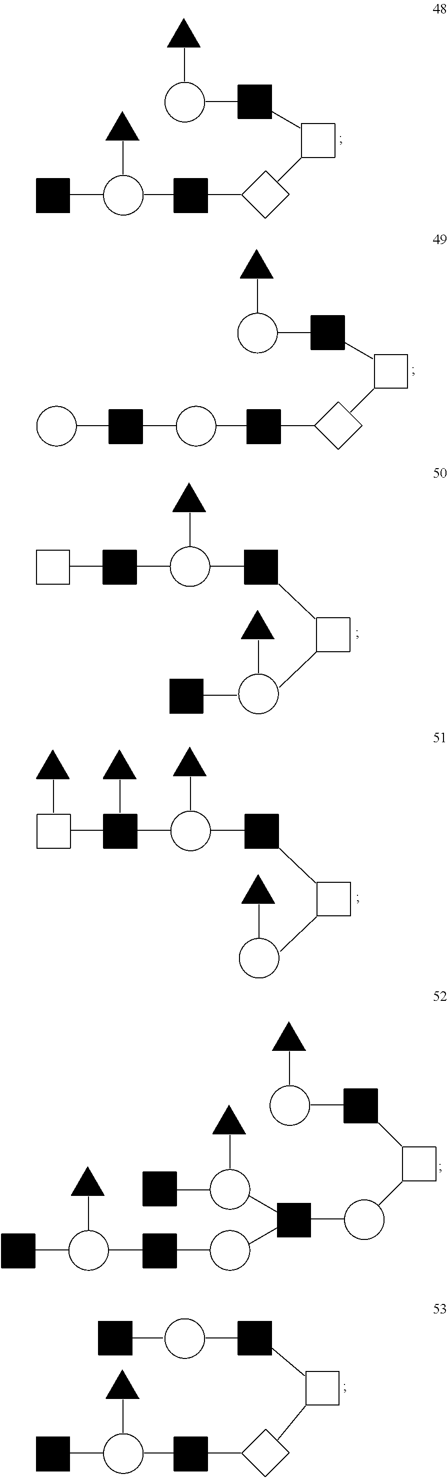

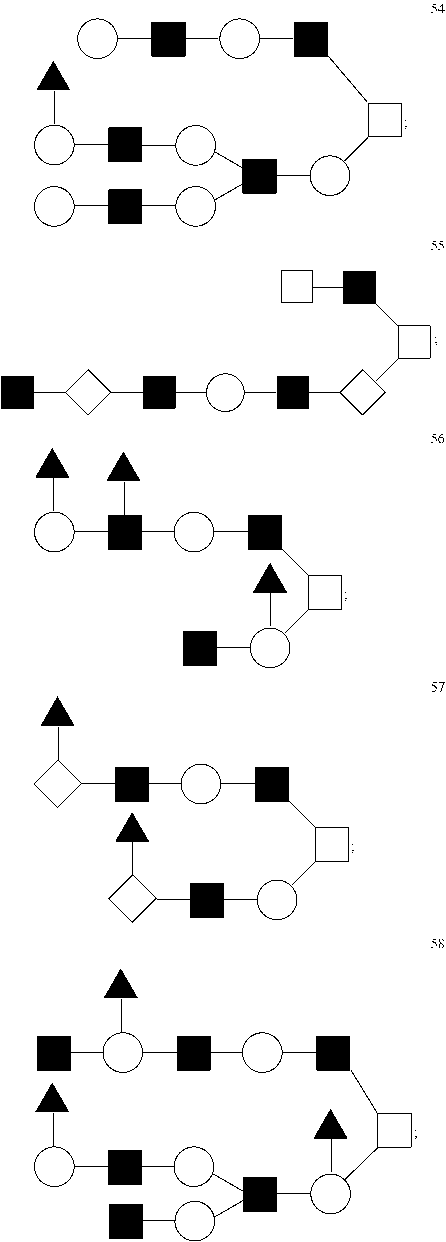

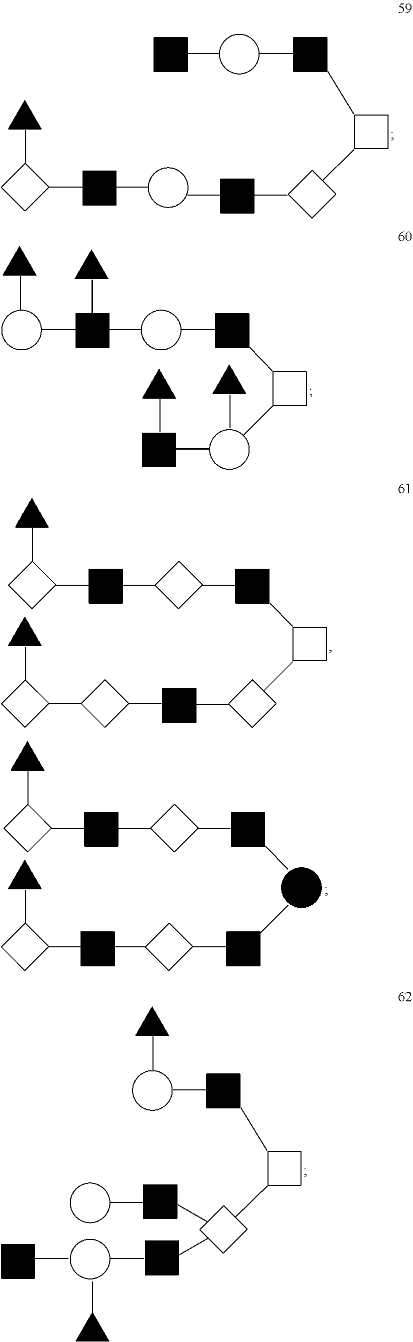

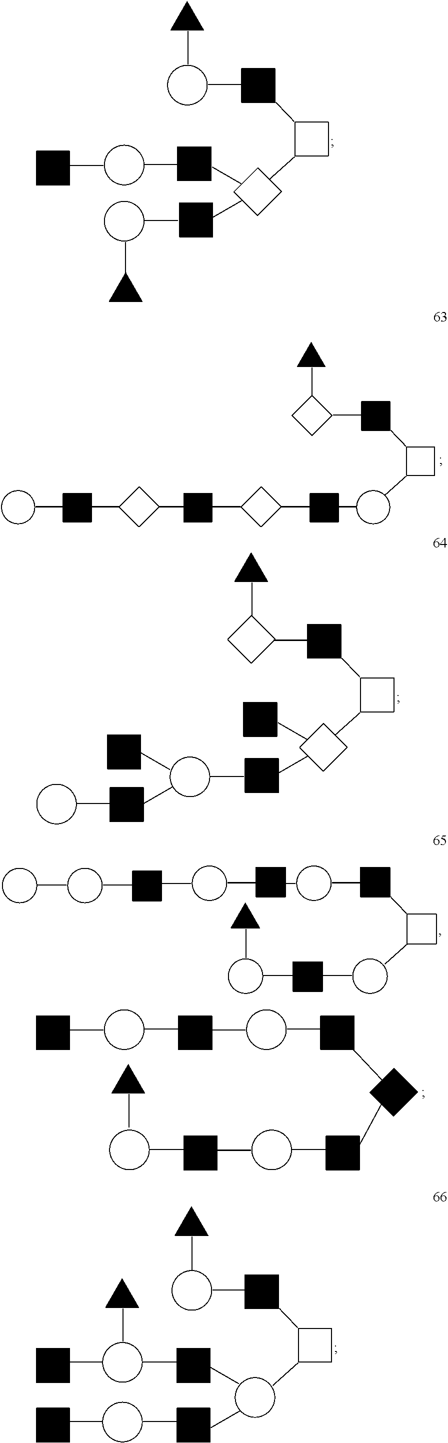

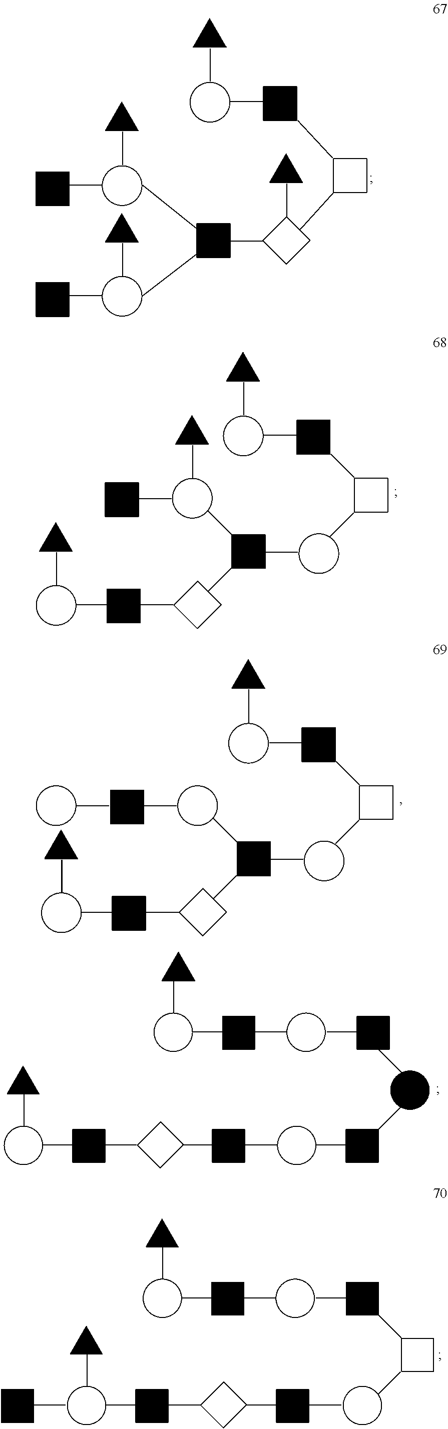

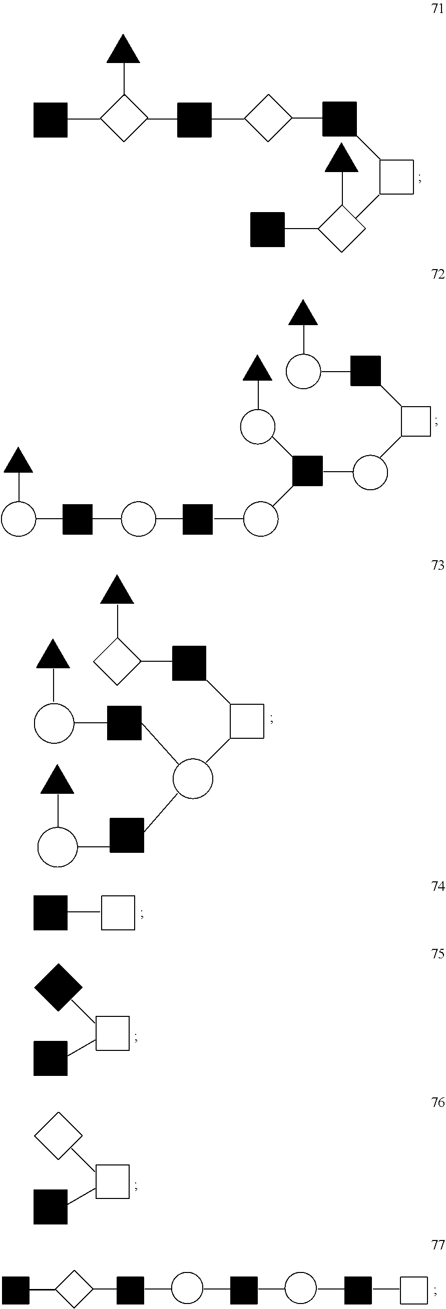

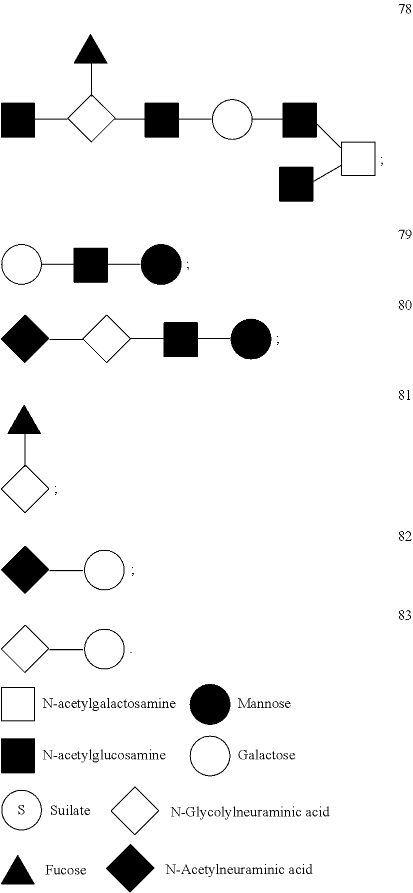

### REFERENCES

1. Ganguly & Mitchell, Mucosal biofilms of Candida albicans, Curr Opin Microbiol. 14:380-85 (2011).
2. Valle & Nobile, Interactions of microorganisms with host mucins: a focus on Candida albicans, FEMS microbiology reviews 44:645-54 (2020).
3. Pappas et al., Invasive candidiasis, Nat Rev Dis Primers 4:1-20 (2018).
4. Perlin, Antifungals: From Genomics to Resistance and the Development of Novel Agents, Clinical Infectious Diseases. 61:1213-14 (2015).
5. Calderone & Fonzi, Virulence factors of Candida albicans, Trends in Microbiology 9:327-35 (2001).
6. Nobile & Johnson, Candida albicans Biofilms and Human Disease, Annu Rev Microbiol. 69:71-92 (2015).
7. Kennedy & Volz, Ecology of Candida albicans gut colonization: inhibition of Candida adhesion, colonization, and dissemination from the gastrointestinal tract by bacterial antagonism, Infect Immun. 49:654-63 (1985).
8. Corfield, Mucins: A biologically relevant glycan barrier in mucosal protection, Biochimica et Biophysica Acta (BBA)—General Subjects, 1850:236-52 (2015).
9. Wang et al., Home, sweet home: how mucus accommodates our microbiota, The FEBS Journal 288:1789-99 (2021).
10. Kavanaugh et al., Mucins Suppress Virulence Traits of Candida albicans, mBio. 5:e01911-14 (2014).
11. Turner et al., Cysteine-rich regions of pig gastric mucin contain von Willebrand factor and cystine knot domains at the carboxyl terminal, Biochimica et Biophysica Acta (BBA)—Gene Structure and Expression 1447:77-92 (1999).
12. Krupa et al., Comparing the permeability of human and porcine small intestinal mucus for particle transport studies, Sci Rep. 10:20290 (2020).
13. Celli et al., Viscoelastic Properties and Dynamics of Porcine Gastric Mucin, Biomacromolecules 6:1329-33 (2005).
14. Han et al., The metabolic basis of Candida albicans morphogenesis and quorum sensing, Fungal Genetics and Biology 48:747-63 (2011).
15. Tailford et al., Juge N. Mucin glycan foraging in the human gut microbiome, Front Genet 6:81 (2015).
16. Lindén et al., MUC1 limits Helicobacter pylori infection both by steric hindrance and by acting as a releasable decoy, PLoS Pathog. 5:e1000617 (2009).
17. Chatterjee et al., Understanding the adhesion mechanism of a mucin binding domain from Lactobacillus fermentum and its role in enteropathogen exclusion, International Journal of Biological Macromolecules 110:598-607 (2018).
18. Wheeler et al., Mucin glycans attenuate the virulence of Pseudomonas aeruginosa in infection, Nature Microbiology 4:2146-54 (2019).
19. Bergstrom & Xia, Mucin-type O-glycans and their roles in intestinal homeostasis, Glycobiology 23:1026-37 (2013).
20. Banerjee et al., UME6, a Novel Filament-specific Regulator of Candida albicans Hyphal Extension and Virulence, Mol Biol Cell 19:1354-65 (2008).
21. Zheng et al., Hgc1, a novel hypha-specific G1 cyclin-related protein regulates Candida albicans hyphal morphogenesis, EMBO J 23:1845-56 (2004).
22. Granger et al., Yeast wall protein 1 of Candida albicans, Microbiology (Reading) 151:1631-44 (2005).
23. Sudbery, Growth of Candida albicans hyphae, Nature Reviews Microbiology 9:737-48 (2011).
24. Lu et al., Candida albicans hyphal initiation and elongation, Trends in Microbiology 22:707-14 (2014).
25. Feng et al., Ras signaling is required for serum-induced hyphal differentiation in Candida albicans, J Bacteriol 181:6339-46 (1999).
26. Bockmühl & Ernst, A Potential Phosphorylation Site for an A-Type Kinase in the Efg1 Regulator Protein Contributes to Hyphal Morphogenesis of Candida albicans, Genetics 157:1523-30 (2001).
27. Leuker et al., Sequence and promoter regulation of the PCK1 gene encoding phosphoenolpyruvate carboxykinase of the fungal pathogen Candida albicans, Gene 192:235-40 (1997).
28. Shapiro et al., Regulatory Circuitry Governing Fungal Development, Drug Resistance, and Disease, Microbiol Mol Biol Rev 75:213-67 (2011).
29. Nobile et al., A Recently Evolved Transcriptional Network Controls Biofilm Development in Candida albicans, Cell 148:126-38 (2012).
30. Lohse et al., Development and regulation of single-and multi-species Candida albicans biofilms, Nature Reviews Microbiology 16:19-31 (2018).
31. Morales & Hogan, Candida albicans interactions with bacteria in the context of human health and disease, PLoS Pathog 6:e1000886 (2010).
32. Hogan & Kolter, Pseudomonas-Candida Interactions: An Ecological Role for Virulence Factors, Science 296:2229-32 (2002).
33. Brand et al., Cell wall glycans and soluble factors determine the interactions between the hyphae of Candida albicans and Pseudomonas aeruginosa, FEMS Microbiol Lett. 287:48-55 (2008).
34. Jin et al., Structural Diversity of Human Gastric Mucin Glycans, Mol Cell Proteomics 16:743-58 (2017).
35. Amon et al., Glycans in immune recognition andresponse, Carbohydr Res. 389:115-22 (2014).
36. Cummings & Pierce, The Challenge and Promise of Glycomics, Chemistry & Biology 21:1-15 (2014).
37. Dube & Bertozzi, Glycans in cancer and inflammation—potential for therapeutics and diagnostics, Nat Rev Drug Discov. 4:477-88 (2005).
38. Hevey, Strategies for the Development of Glycomimetic Drug Candidates, Pharmaceuticals 12:55 (2019).
39. Hevey, Bioisosteres of Carbohydrate Functional Groups in Glycomimetic Design, Biomimetics 4:53 (2019).
40. Brockhausen, Mucin-type O-glycans in human colon and breast cancer: glycodynamics and functions, EMBO reports 7:599-604 (2006).
41. Tabak, The role of mucin-type O-glycans in eukaryotic development, Seminars in Cell & Developmental Biology 21:616-21 (2010).
42. Kudelka et al., Simple Sugars to Complex Disease—Mucin-Type O-Glycans in Cancer, Adv Cancer Res. 126:53-135 (2015).
43. Gianchecchi et al., Sialic Acid-Siglec Axis in Human Immune Regulation, Involvement in Autoimmunity and Cancer and Potential Therapeutic Treatments, IJMS. 22:5774 (2021).
44. Donohue et al., The N-terminal part of Als1 protein from Candida albicans specifically binds fucose-containing glycans, Molecular Microbiology 80:1667-79 (2011).
45. Lee et al., An amino acid liquid synthetic medium for the development of mycelial and yeast forms of Candida Albicans, Sabouraudia 13:148-53 (1975).
46. Wagner et al., A rheological study of the association and dynamics of MUC5AC gels, Biomacromolecules 18:3654-64 (2017).
47. Lieleg et al., Mucin biopolymers as broad-spectrum antiviral agents, Biomacromolecules 13:1724-32 (2012).
48. Huang et al., Microscale nonreductive release of O-linked glycans for subsequent analysis through MALDI mass spectrometry and capillary electrophoresis, Anal Chem. 73:6063-69 (2001).
49. Aoki et al., Dynamic developmental elaboration of N-linked glycan complexity in the Drosophila melanogaster embryo, J Biol Chem. 282:9127-42 (2007).
50. Liu et al., The minimum information required for a glycomics experiment (MIRAGE) project: improving the standards for reporting glycan microarray-based data, Glycobiology 27:280-84 (2017).
51. Mehta & Cummings, GlycoGlyph: a glycan visualizing, drawing and naming application, Bioinformatics 36:3613-14 (2020).
52. Watanabe et al., GlycoPOST realizes FAIR principles for glycomics mass spectrometry data, Nucleic Acids Res. 49:D1523-28 (2021).
53. Liao et al., The Subread aligner: fast, accurate and scalable read mapping by seed-and-vote, Nucleic Acids Research 41:e108 (2013).
54. Liao et al., featureCounts: an efficient general purpose program for assigning sequence reads to genomicfeatures, Bioinformatics 30:923-30 (2014).
55. Robinson & Oshlack, A scaling normalization method for differential expression analysis of RNA-seq data, Genome Biology. 11:R25 (2010).
56. Ritchie et al., limma powers differential expression analyses for RNA-sequencing and microarray studies, Nucleic Acids Research 43:e47 (2015).
57. Law et al., voom: precision weights unlock linear model analysis tools for RNA-seq read counts, Genome Biology 15:R29 (2014).
58. Afgan et al., The Galaxy platform for accessible, reproducible and collaborative biomedical analyses: 2018 update, Nucleic Acids Res. 46:W537-44 (2018).
59. Love et al., Moderated estimation of fold change and dispersion for RNA-seq data with DESeq2, Genome Biol. 15:550 (2014).
60. Mi et al., Protocol Update for large-scale genome and gene function analysis with the PANTHER classification system (v.14.0), Nature Protocols 14:703-21 (2019).
61. Sail & Kovac, Benzoylated ethyl 1-thioglycosides: direct preparation from per-Obenzylated sugars, Carbohydr. Res. 357, 47-52 (2012).
62. Yi et al., Escherichia coli O86 O-antigen biosynthetic gene cluster and stepwise enzymatic synthesis of human blood group B antigen tetrasaccharide, J. Am. Chem. Soc. 127, 2040-41 (2005).
63. Calosso et al., Acyclic tethers mimicking subunits of polysaccharide ligands: selectin antagonists, ACS Med. Chem. Lett. 5:1054-59 (2014).
64. Gangadharmath & Demchenko, Nickel(II) chloride-mediated regioselective benzylation and benzoylation of diequatorial vicinal diols, Synlett 2191-93 (2004).
65. Verez-Bencomo et al., Glycosides of 8-hydroxy-3,6-dioxaoctanal. A synthesis of a new spacer for synthetic oligosaccharides, Carbohydr. Res. 217:263-67 (1991).
66. Minuth et al., Sweets for catalysis—facile optimisation of carbohydrate-based bis(oxazoline) ligands, Eur. J. Org. Chem. 997-1008 (2009).
67. Lafont et al., Syntheses and interfacial behaviour of neoglycolipid analogues of glycosyl ceramides, Carbohydr. Res. 336:181-94 (2001).

The teachings of all patents, published applications and references cited herein are incorporated by reference in their entirety. For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, etc.), are hereby incorporated by reference in their entirety.

1. A method of attenuating virulence of a fungus, the method comprising contacting the fungus with a mucin glycan, tautomer, stereoisomer and/or a pharmaceutically acceptable salt thereof.

2. A method of treating a fungal infection and/or attenuating virulence of a fungus in a subject in need thereof, the method comprising administering to the subject a therapeutically effective amount of a mucin glycan, tautomer, stereoisomer and/or a pharmaceutically acceptable salt thereof.

3. The method of claim 1, wherein attenuating virulence of a fungus comprises modulating a virulence-associated gene of the fungus, reducing the fungus's surface adhesion, inhibiting the fungus's morphological transition to an invasive or virulent cell type, inhibiting fungal biofilm formation, reducing the fungus's secretion of a hydrolytic enzyme, or a combination thereof, and optionally, wherein modulating a virulence-associated gene comprises:
a) downregulating expression of RAS1, EED1, HGC1, UME6, EFG1, BRG1, EFG1, TEC1, ROB1, RFX2, AHR1, ECE1, HYR1, ALS3, HWP1, PRA1, PHR1, GAC1, PTP3, AHR1, YVC1, SAP5, IHD1, RTA4, PGA54, IHD2, DDR48, CSA1, SHE3, or TUP1, or a combination thereof;
b) increasing expression of NRG1, YWP1, or both; or
both a) and b).

4. The method of claim 1, wherein the fungus comprises Candida albicans.

5. A composition comprising:
a) a synthetic mucin glycan; or
b) a mucin glycan, wherein the purity of the mucin glycan is at least about 30%; or
both a) and b).

6. The composition of claim 5, wherein the mucin glycan comprises a GalNAc residue, and wherein the hydrogen atom of the hydroxyl at anomeric/C-1 position of the GalNAc residue is unsubstituted or substituted.

7. The method of claim 1, wherein the mucin glycan:
a) is a secreted gel-forming mucin glycan;
b) comprises a MUC2 glycan, a MUC5AC glycan, a MUC5B glycan, or a combination thereof; or
c) comprises one or more monosaccharides selected from N-acetylgalactosamine (GalNAc), N-acetylglucosamine (GlcNAc), mannose (Man), fucose (Fuc), N-acetylneuraminic acid (Neu5Ac), or galactose (Gal), or a combination thereof;
or any combination of a) to c).

8. The method of claim 1, wherein the mucin glycan further comprises N-acetylgalactosamine, N-acetylglucosamine, mannose, fucose, N-acetylneuraminic acid, galactose, sulfate, sialic acid, or a combination thereof.

9. The method of claim 1, wherein the mucin glycan comprises a glycan core of the following structural formula, or a tautomer or stereoisomer thereof:

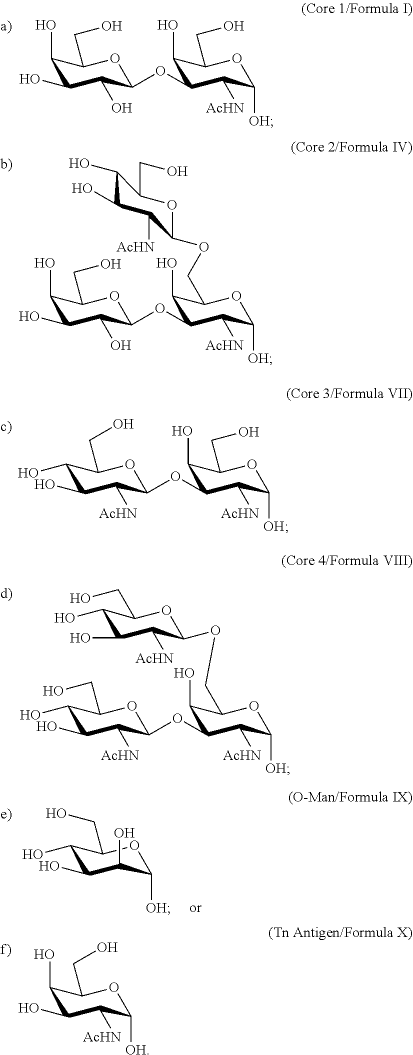

10. The method of claim 9, wherein the mucin glycan comprises a glycan structure selected from the group consisting of: 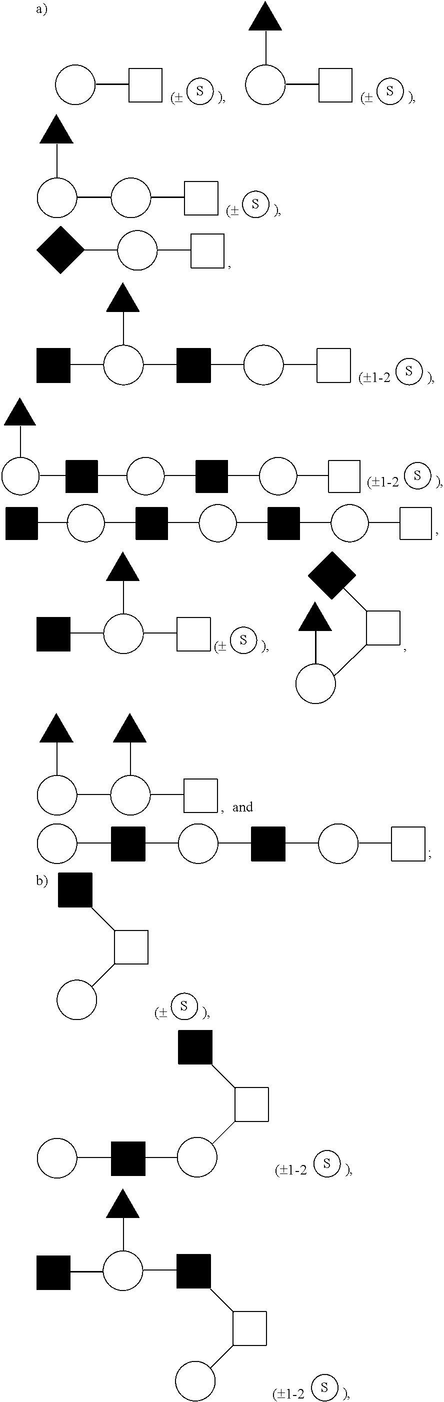 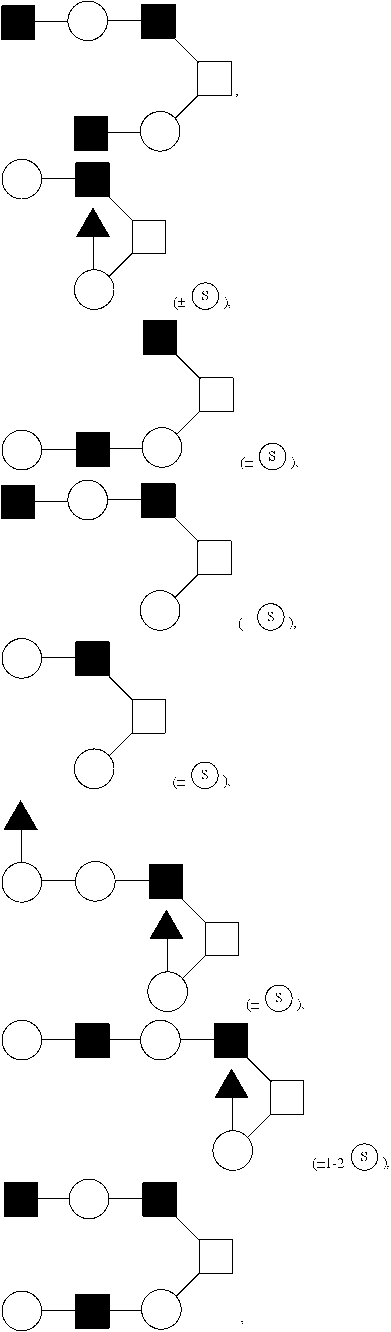 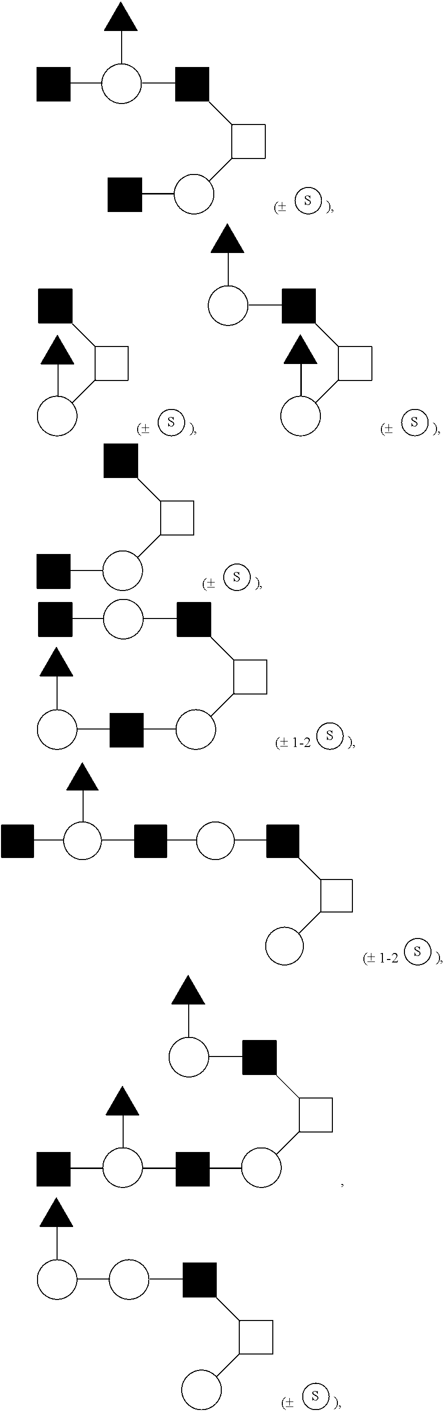 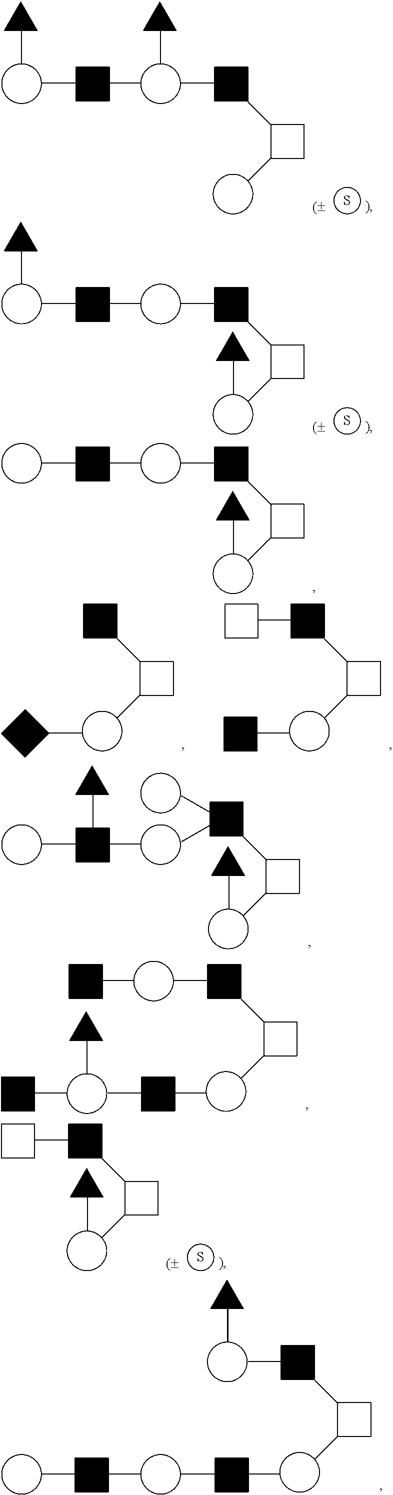 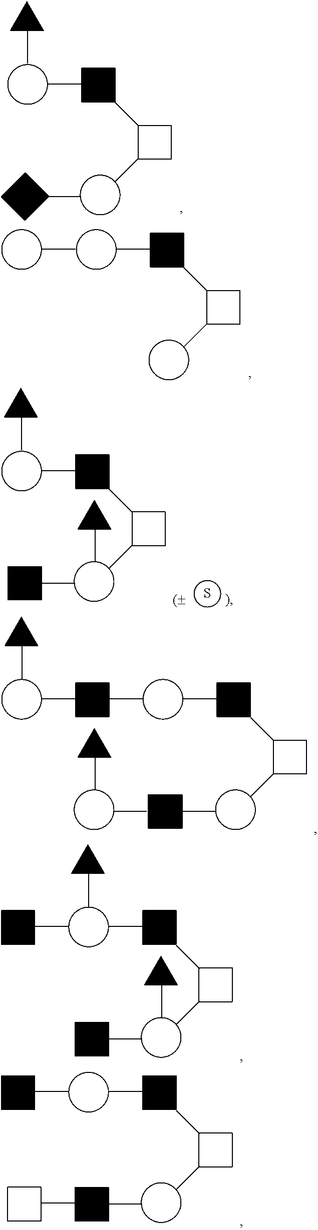 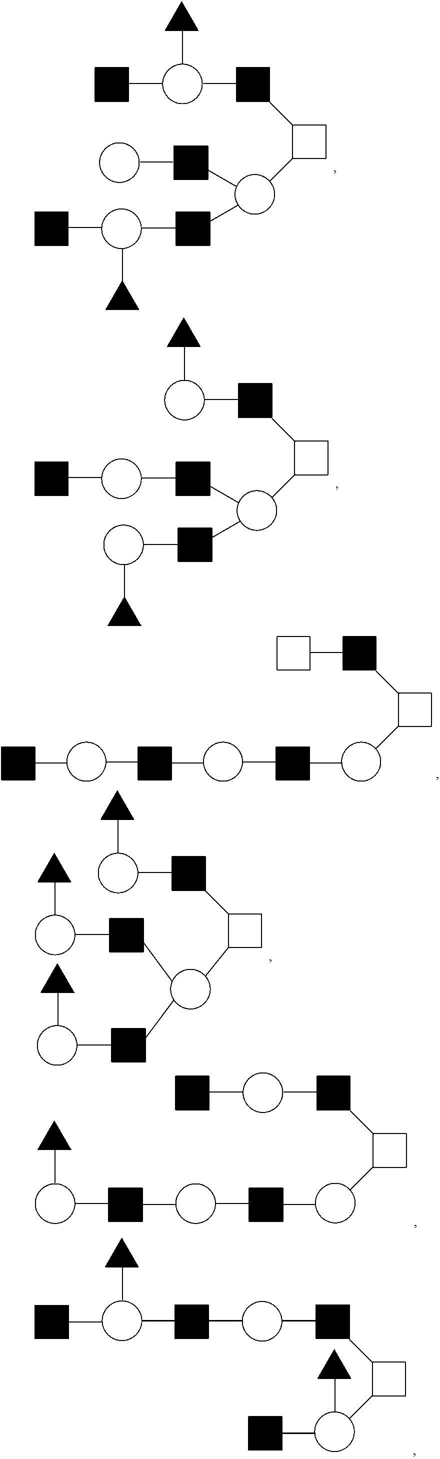 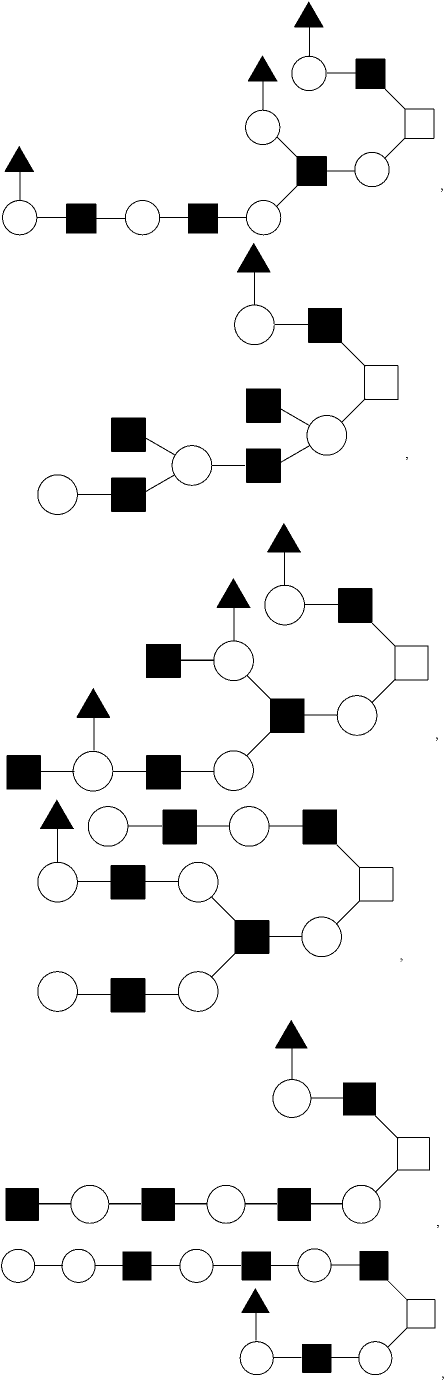 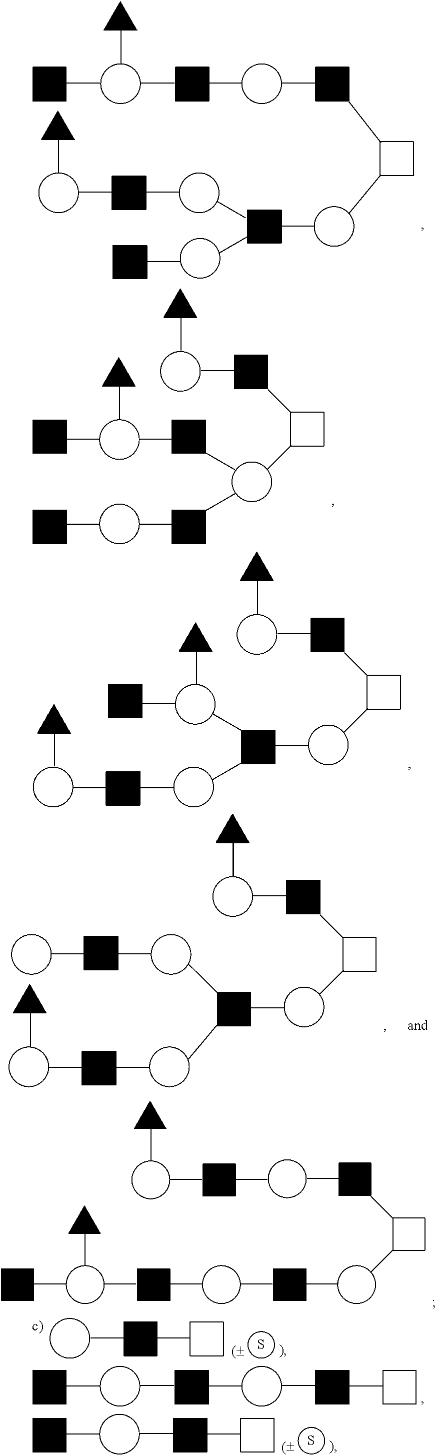 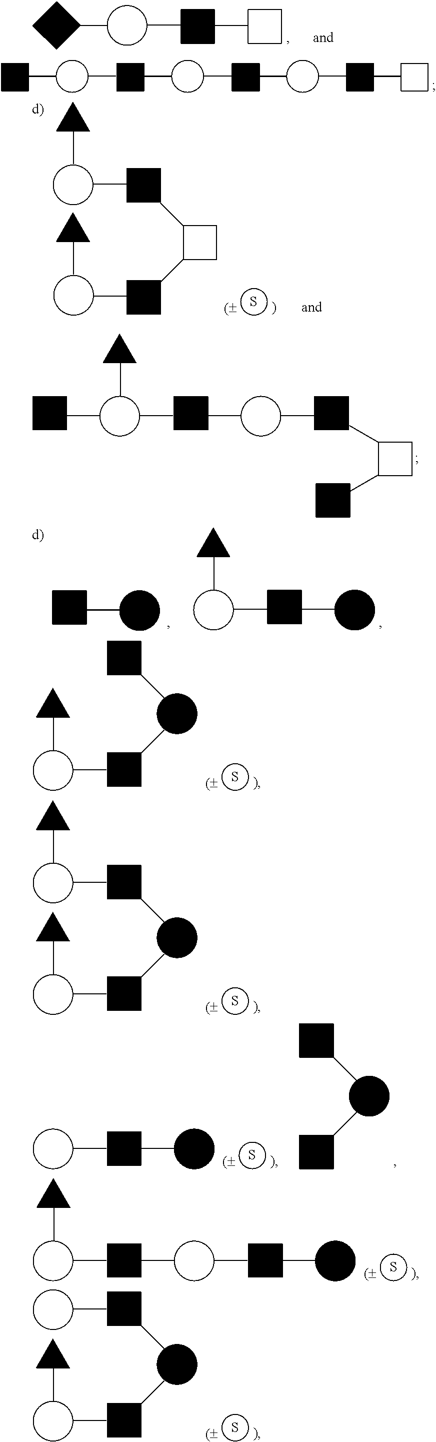 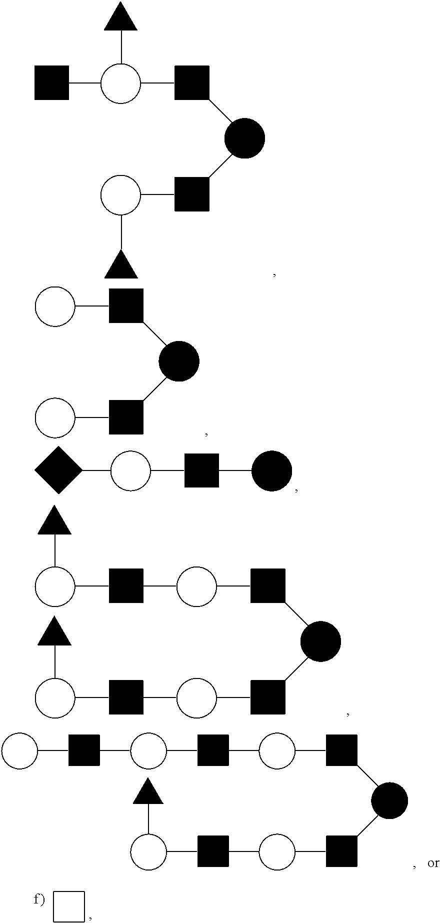
wherein □ is N-acetylgalactosamine, ▪ is N-acetylglucosamine, ● is mannose, ◯ is galactose, ♦ is N-acetylneuraminic acid, ▴ is fucose, and Ⓢ is sulfate.

11. The method of claim 10, wherein:
a) the mucin glycan comprises a glycan structure selected from the group consisting of 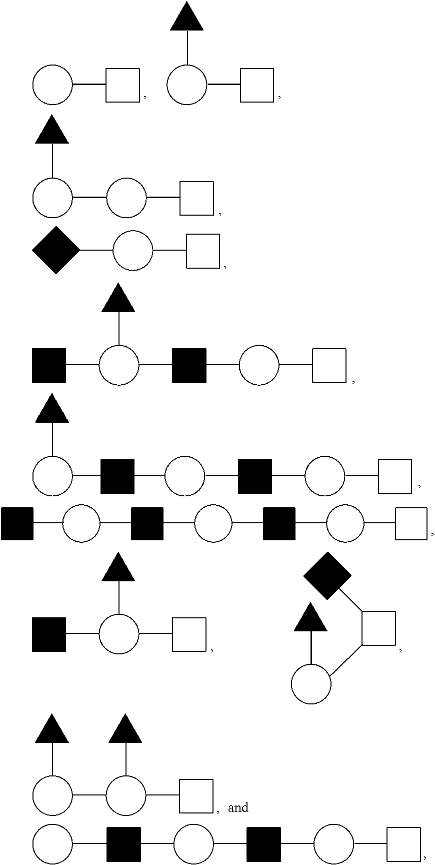
the mucin glycan further comprises fucose, sialic acid, or a combination thereof, and optionally, the mucin glycan comprises the following structural formula, or a tautomer or stereoisomer thereof: 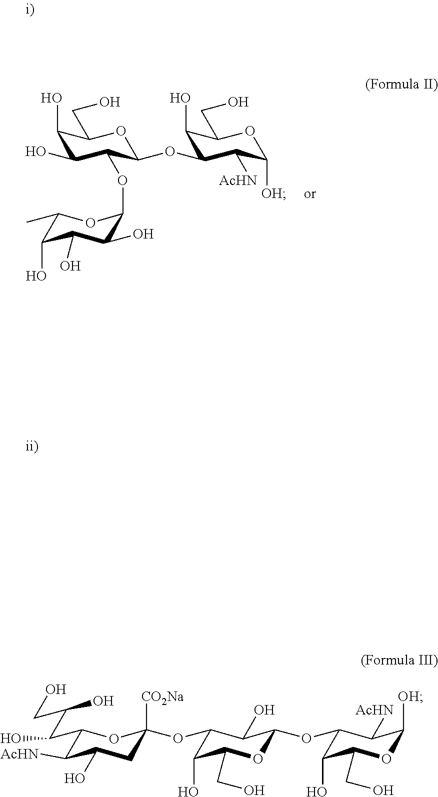
b) the mucin glycan comprises a glycan structure selected from the group consisting 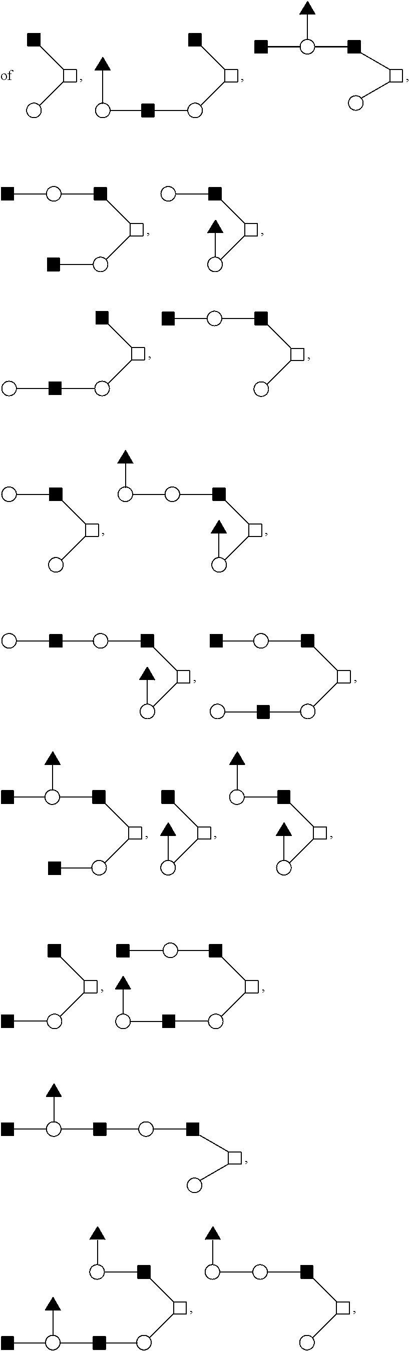 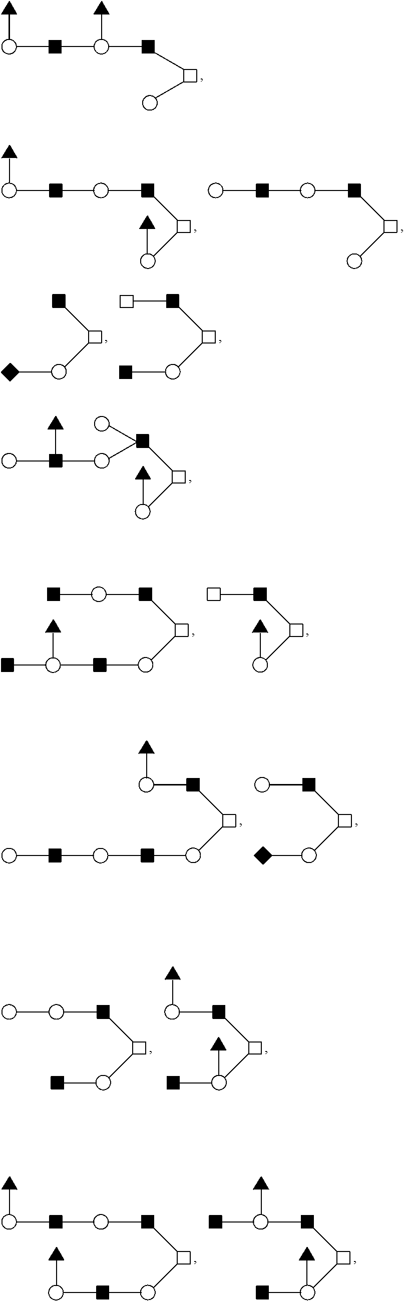 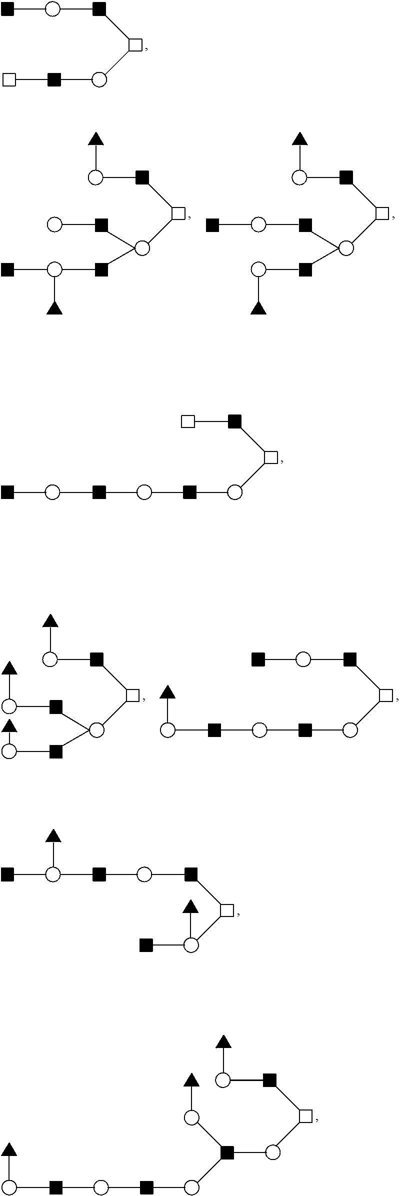 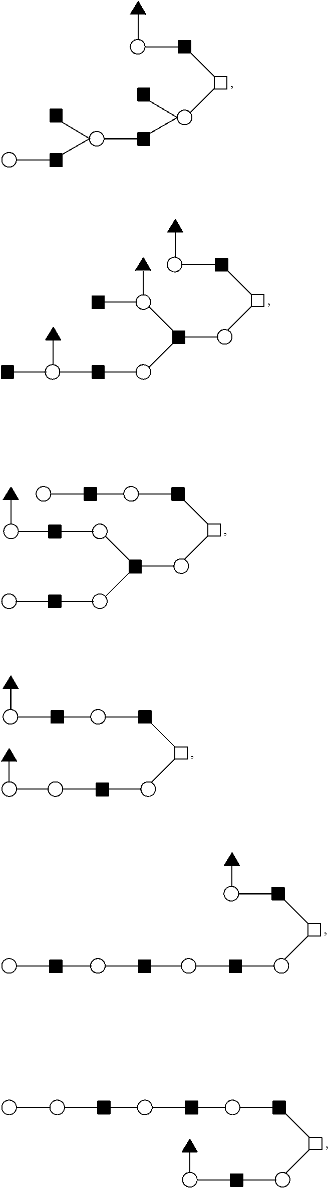 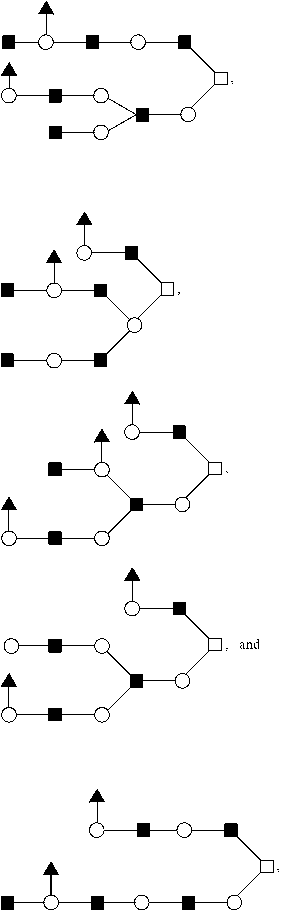
the mucin glycan further comprises fucose, galactose, or a combination thereof, and optionally, the mucin glycan comprises the following structural formula, or a tautomer or stereoisomer thereof:

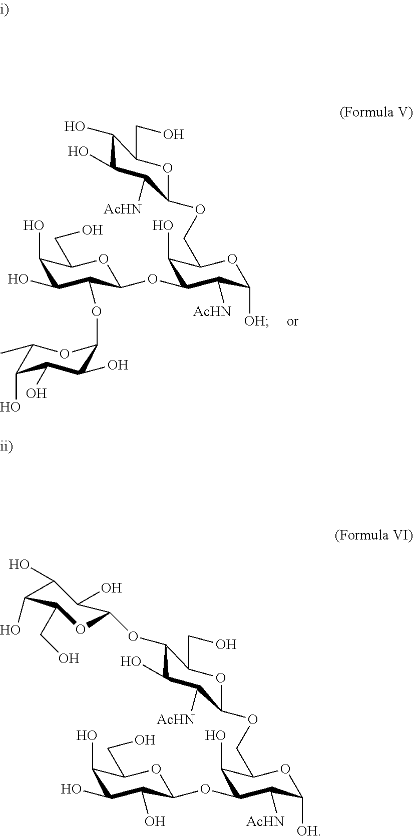

12. The method of claim 1, wherein the mucin glycan comprises a formula selected from the group consisting of (HexNAc)1; (Hex)1 (HexNAc)1; (Hex)1 (HexNAc)1 (Deoxyhexose)1; (Hex)1 (HexNAc)1 (NeuAc)1; (Hex)2 (HexNAc)1 (Deoxyhexose)1; (Hex)1 (HexNAc)1 (NeuGc)1; (Hex)1 (HexNAc)1 (Deoxyhexose)1 (NeuAc)1; (Hex)2 (HexNAc)1 (Deoxyhexose)2; (Hex)1 (HexNAc)1 (NeuAc)2; (Hex)2 (HexNAc)1 (Deoxyhexose)3; (Hex)2 (HexNAc)1 (Deoxyhexose)1 (NeuAc)1; (Hex)1 (HexNAc)1 (NeuAc)1 (NeuGc)1; (Hex)1 (HexNAc)2 (Deoxyhexose)1 (NeuAc)1; (Hex)3 (HexNAc)2 (Deoxyhexose)4; (Hex)1 (HexNAc)2; (Hex)1 (HexNAc)2 (Deoxyhexose)1; (Hex)2 (HexNAc)2; (Hex)1 (HexNAc)3; (Hex)1 (HexNAc)2 (Deoxyhexose)2; (Hex)1 (HexNAc)2 (NeuAc)1; (Hex)2 (HexNAc)2 (Deoxyhexose)1; (Hex)1 (HexNAc)2 (NeuGc)1; (Hex)3 (HexNAc)2; (Hex)1 (HexNAc)3 (Deoxyhexose)1; (Hex)2 (HexNAc)3; (Hex)1 (HexNAc)4; (Hex)2 (HexNAc)2 (Deoxyhexose)2; (Hex)2 (HexNAc)2 (NeuAc)1; (Hex)3 (HexNAc)2 (Deoxyhexose)1; (Hex)2 (HexNAc)3 (Deoxyhexose)1; (Hex)3 (HexNAc)3; (Hex)2 (HexNAc)4; (Hex)2 (HexNAc)2 (Deoxyhexose)3; (Hex)2 (HexNAc)2 (Deoxyhexose)1 (NeuAc)1; (Hex)3 (HexNAc)2 (Deoxyhexose)2; (Hex)2 (HexNAc)3 (Deoxyhexose)2; (Hex)3 (HexNAc)3 (Deoxyhexose)1; (Hex)2 (HexNAc)4 (Deoxyhexose)1; (Hex)3 (HexNAc)4; (Hex)3 (HexNAc)2 (Deoxyhexose)3; (Hex)2 (HexNAc)5; (Hex)3 (HexNAc)2 (Deoxyhexose)1 (NeuAc)1; (Hex)3 (HexNAc)3 (Deoxyhexose)2; (Hex)2 (HexNAc)4 (Deoxyhexose)2; (Hex)3 (HexNAc)4 (Deoxyhexose)1; (Hex)3 (HexNAc)3 (Deoxyhexose)3; (Hex)4 (HexNAc)3 (Deoxyhexose)2; (Hex)3 (HexNAc)4 (Deoxyhexose)2; (Hex)4 (HexNAc)4 (Deoxyhexose)1; (Hex)2 (HexNAc)5 (Deoxyhexose)2; (Hex)3 (HexNAc)3 (Deoxyhexose)4; (Hex)5 (HexNAc)6 (Deoxyhexose)3; (Hex)3 (HexNAc)5 (Deoxyhexose)1; (Hex)7 (HexNAc)6 (Deoxyhexose)1; (Hex)3 (HexNAc)6; (Hex)3 (HexNAc)4 (Deoxyhexose)3; (Hex)4 (HexNAc)4 (Deoxyhexose)2; (Hex)6 (HexNAc)7 (Deoxyhexose)2; (Hex)4 (HexNAc)5 (Deoxyhexose)1; (Hex)3 (HexNAc)4 (Deoxyhexose)4; (Hex)5 (HexNAc)4 (Deoxyhexose)2; (Hex)4 (HexNAc)5 (Deoxyhexose)2; (Hex)5 (HexNAc)5 (Deoxyhexose)1; (Hex)4 (HexNAc)6 (Deoxyhexose)1; (Hex)6 (HexNAc)5 (Deoxyhexose)1; (Hex)4 (HexNAc)6 (Deoxyhexose)2; (Hex)4 (HexNAc)5 (Deoxyhexose)4; (Hex)5 (HexNAc)5 (Deoxyhexose)3; (Hex)6 (HexNAc)5 (Deoxyhexose)2; (Hex)5 (HexNAc)6 (Deoxyhexose)2; (Hex)3 (HexNAc)5 (Deoxyhexose)2; (Hex)6 (HexNAc)5 (Deoxyhexose)3; (Hex)4 (HexNAc)4 (Deoxyhexose)3; (HexNAc)2; (HexNAc)2 (NeuAc)1; (HexNAc)2 (NeuGc)1; (Hex)3 (HexNAc)5; (Hex)2 (HexNAc)5 (Deoxyhexose)1; (Hex)2 (HexNAc)1; (Hex)2 (HexNAc)1 (NeuAc)1; (Hex)1 (Deoxyhexose)1; (Hex)1 (NeuAc)1; and (Hex)1 (NeuGc)1.

13. The method of claim 12, wherein the mucin glycan comprises a MUC2 glycan, a MUC5AC glycan, a MUC5B glycan, or a combination thereof.

14. The method of claim 13, wherein:
a) the MUC2 glycan comprises a formula selected from the group consisting of (Hex)1 (HexNAc)1 (Deoxyhexose)1; (Hex)1 (NeuAc)1; (Hex)1 (Deoxyhexose)1; (Hex)1 (HexNAc)1; (Hex)1 (NeuGc)1; (Hex)1 (HexNAc)2 (Deoxyhexose)1; (Hex)1 (HexNAc)1 (NeuAc)1; (Hex)1 (HexNAc)1 (NeuGc)1; (Hex)1 (HexNAc)2; (HexNAc)2; (HexNAc)1; (Hex)1 (HexNAc)3 (Deoxyhexose)1; (Hex)2 (HexNAc)2 (NeuAc)1; (Hex)2 (HexNAc)2; (HexNAc)2 (NeuAc)1; (Hex)2 (HexNAc)1; (Hex)2 (HexNAc)2 (Deoxyhexose)1; (Hex)1 (HexNAc)3; (HexNAc)2 (NeuGc)1; (Hex)2 (HexNAc)2 (Deoxyhexose)2; (Hex)1 (HexNAc)2 (NeuAc)1; (Hex)2 (HexNAc)3 (Deoxyhexose)2; (Hex)2 (HexNAc)3; (Hex)1 (HexNAc)1 (NeuAc)2; (Hex)1 (HexNAc)2 (NeuGc)1; (Hex)1 (HexNAc)1 (Deoxyhexose)1 (NeuAc)1; (Hex)1 (HexNAc)2 (Deoxyhexose)1 (NeuAc)1; (Hex)4 (HexNAc)5 (Deoxyhexose)1; (Hex)3 (HexNAc)4 (Deoxyhexose)1; (Hex)2 (HexNAc)4 (Deoxyhexose)2; (Hex)3 (HexNAc)5 (Deoxyhexose)1; (Hex)2 (HexNAc)1 (NeuAc)1; (Hex)3 (HexNAc)4; (Hex)4 (HexNAc)4 (Deoxyhexose)1; (Hex)2 (HexNAc)3 (Deoxyhexose)1; (Hex)1 (HexNAc)1 (NeuAc)1 (NeuGc)1; (Hex)2 (HexNAc)4 (Deoxyhexose)1; (Hex)3 (HexNAc)5; (Hex)4 (HexNAc)5 (Deoxyhexose)2; (Hex)5 (HexNAc)5 (Deoxyhexose)1; (Hex)2 (HexNAc)4; (Hex)3 (HexNAc)3 (Deoxyhexose)1; (Hex)4 (HexNAc)6 (Deoxyhexose)1; (Hex)3 (HexNAc)2 (Deoxyhexose)1; (Hex)2 (HexNAc)1 (Deoxyhexose)2; (Hex)4 (HexNAc)6 (Deoxyhexose)2; (Hex)3 (HexNAc)2; (Hex)5 (HexNAc)6 (Deoxyhexose)2; and (Hex)3 (HexNAc)3;
b) the MUC5B glycan comprises a formula selected from the group consisting of (Hex)1 (HexNAc)1 (Deoxyhexose)1; (Hex)1 (HexNAc)1; (Hex)1 (HexNAc)2 (Deoxyhexose)1; (Hex)1 (NeuAc)1; (Hex)1 (Deoxyhexose)1; (Hex)3 (HexNAc)3 (Deoxyhexose)2; (Hex)2 (HexNAc)2 (Deoxyhexose)2; (Hex)1 (HexNAc)1 (NeuAc)1; (Hex)3 (HexNAc)2 (Deoxyhexose)2; (Hex)1 (HexNAc)2; (Hex)1 (HexNAc)2 (Deoxyhexose)2; (Hex)2 (HexNAc)2 (Deoxyhexose)1; (Hex)2 (HexNAc)1 (Deoxyhexose)1; (Hex)2 (HexNAc)3 (Deoxyhexose)1; (Hex)2 (HexNAc)3 (Deoxyhexose)2; (Hex)3 (HexNAc)2 (Deoxyhexose)1; (Hex)2 (HexNAc)1; (Hex)1 (HexNAc)3 (Deoxyhexose)1; (Hex)3 (HexNAc)4 (Deoxyhexose)3; (Hex)2 (HexNAc)1 (Deoxyhexose)2; (Hex)2 (HexNAc)2 (NeuAc)1; (Hex)3 (HexNAc)2 (Deoxyhexose)3; (Hex)2 (HexNAc)4 (Deoxyhexose)2; (Hex)3 (HexNAc)3 (Deoxyhexose)1; (Hex)3 (HexNAc)3 (Deoxyhexose)3; (Hex)2 (HexNAc)2; (Hex)2 (HexNAc)3; (Hex)2 (HexNAc)2 (Deoxyhexose)3; (Hex)3 (HexNAc)4 (Deoxyhexose)4; (Hex)3 (HexNAc)2; (Hex)3 (HexNAc)4 (Deoxyhexose)1; (HexNAc)2 (NeuAc)1; (Hex)3 (HexNAc)4 (Deoxyhexose)2; (Hex)1 (HexNAc)2 (Deoxyhexose)1 (NeuAc)1; (Hex)3 (HexNAc)2 (Deoxyhexose)4; (Hex)1 (HexNAc)2 (NeuAc)1; (Hex)1 (HexNAc)1 (Deoxyhexose)1 (NeuAc)1; (Hex)4 (HexNAc)3 (Deoxyhexose)2; (Hex)4 (HexNAc)4 (Deoxyhexose)2; (HexNAc)2; (Hex)4 (HexNAc)4 (Deoxyhexose)3; (Hex)2 (HexNAc)4 (Deoxyhexose)1; (Hex)3 (HexNAc)2 (Deoxyhexose)1 (NeuAc)1; (Hex)2 (HexNAc)1 (Deoxyhexose)1 (NeuAc)1; (Hex)3 (HexNAc)3 (Deoxyhexose)4; (Hex)5 (HexNAc)4 (Deoxyhexose)2 (G21630AC); (HexNAc)1; (Hex)1 (HexNAc)3; (Hex)2 (HexNAc)1 (Deoxyhexose)3; (Hex)2 (HexNAc)5 (Deoxyhexose)2; (Hex)4 (HexNAc)4 (Deoxyhexose)1; (Hex)4 (HexNAc)5 (Deoxyhexose)4; (Hex)4 (HexNAc)5 (Deoxyhexose)2; (Hex)5 (HexNAc)5 (Deoxyhexose)1; (Hex)2 (HexNAc)4; (Hex)4 (HexNAc)5 (Deoxyhexose)1; (Hex)1 (NeuGc)1; (Hex)5 (HexNAc)6 (Deoxyhexose)2; (Hex)6 (HexNAc)5 (Deoxyhexose)2; (Hex)2 (HexNAc)2 (Deoxyhexose)1 (NeuAc)1; (Hex)5 (HexNAc)6 (Deoxyhexose)3; (Hex)3 (HexNAc)3; (Hex)3 (HexNAc)5 (Deoxyhexose)1; (Hex)5 (HexNAc)5 (Deoxyhexose)3); (Hex)2 (HexNAc)1 (NeuAc)1; (Hex)7 (HexNAc)6 (Deoxyhexose)1; and (Hex)3 (HexNAc)4; or
c) the MUC5AC glycan comprises a formula selected from the group consisting of (Hex)1 (HexNAc)1; (Hex)1 (HexNAc)2; (Hex)1 (HexNAc)1 (Deoxyhexose)1; (Hex)1 (Deoxyhexose)1; (Hex)2 (HexNAc)3 (Deoxyhexose)1; (Hex)2 (HexNAc)4; (Hex)2 (HexNAc)2 (Deoxyhexose)1; (Hex)2 (HexNAc)3; (Hex)2 (HexNAc)2; (Hex)3 (HexNAc)2 (Deoxyhexose)2; (Hex)3 (HexNAc)3 (Deoxyhexose)1; (Hex)3 (HexNAc)4; (Hex)2 (HexNAc)4 (Deoxyhexose)1; (Hex)2 (HexNAc)1 (Deoxyhexose)1; (Hex)1 (HexNAc)2 (Deoxyhexose)1; (Hex)2 (HexNAc)2 (Deoxyhexose)2; (Hex)1 (HexNAc)3; (Hex)3 (HexNAc)4 (Deoxyhexose)1; (Hex)1 (HexNAc)1 (NeuAc)1; (Hex)3 (HexNAc)4 (Deoxyhexose)2; (Hex)3 (HexNAc)2 (Deoxyhexose)1; (Hex)2 (HexNAc)1; (Hex)3 (HexNAc)3 (Deoxyhexose)2; (Hex)3 (HexNAc)3; (Hex)1 (HexNAc)2 (NeuAc)1; (Hex)3 (HexNAc)5; (HexNAc)1; (Hex)1 (HexNAc)4; (Hex)4 (HexNAc)3 (Deoxyhexose)2; (Hex)3 (HexNAc)5 (Deoxyhexose)1; (Hex)1 (HexNAc)3 (Deoxyhexose)1; (Hex)2 (HexNAc)1 (NeuAc)1; (Hex)4 (HexNAc)4 (Deoxyhexose)1; (Hex)2 (HexNAc)2 (NeuAc)1; (Hex)3 (HexNAc)2; (Hex)2 (HexNAc)3 (Deoxyhexose)2; (Hex)1 (HexNAc)1 (Deoxyhexose)1 (NeuAc)1; (Hex)4 (HexNAc)4 (Deoxyhexose)2; (Hex)2 (HexNAc)4 (Deoxyhexose)2; (Hex)2 (HexNAc)5; (Hex)4 (HexNAc)5 (Deoxyhexose)2; (Hex)3 (HexNAc)6; (Hex)2 (HexNAc)1 (Deoxyhexose)2; (Hex)4 (HexNAc)4 (Deoxyhexose)3; (Hex)4 (HexNAc)5 (Deoxyhexose)1; (Hex)3 (HexNAc)5 (Deoxyhexose)2; (Hex)2 (HexNAc)5 (Deoxyhexose)1; (Hex)6 (HexNAc)5 (Deoxyhexose)3; (Hex)4 (HexNAc)6 (Deoxyhexose)1; (Hex)5 (HexNAc)6 (Deoxyhexose)3; (Hex)7 (HexNAc)6 (Deoxyhexose)1; (Hex)5 (HexNAc)4 (Deoxyhexose)2; (Hex)5 (HexNAc)5 (Deoxyhexose)1; (Hex)6 (HexNAc)5 (Deoxyhexose)1; (Hex)6 (HexNAc)7 (Deoxyhexose)2; (Hex)4 (HexNAc)6 (Deoxyhexose)2; (Hex)5 (HexNAc)5 (Deoxyhexose)3; (Hex)6 (HexNAc)5 (Deoxyhexose)2; and (Hex)5 (HexNAc)6 (Deoxyhexose)2.

15. The method of claim 14, wherein:
a) the MUC2 glycan comprises a formula selected from the group consisting of (Hex)1 (HexNAc)1 (Deoxyhexose)1; (Hex)1 (NeuAc)1; (Hex)1 (Deoxyhexose)1; (Hex)1 (HexNAc)1; (Hex)1 (NeuGc)1; (Hex)1 (HexNAc)2 (Deoxyhexose)1; (Hex)1 (HexNAc)1 (NeuAc)1; (Hex)1 (HexNAc)1 (NeuGc)1; (Hex)1 (HexNAc)2; (HexNAc)2; (HexNAc)1; (Hex)1 (HexNAc)3 (Deoxyhexose)1; (Hex)2 (HexNAc)2 (NeuAc)1; (Hex)2 (HexNAc)2; (HexNAc)2 (NeuAc)1; and (Hex)2 (HexNAc)1;
b) the MUC5B glycan comprises a formula selected from the group consisting of (Hex)1 (HexNAc)1 (Deoxyhexose)1; (Hex)1 (HexNAc)1; (Hex)1 (HexNAc)2 (Deoxyhexose)1; (Hex)1 (NeuAc)1; (Hex)1 (Deoxyhexose)1; (Hex)3 (HexNAc)3 (Deoxyhexose)2; (Hex)2 (HexNAc)2 (Deoxyhexose)2; (Hex)1 (HexNAc)1 (NeuAc)1; (Hex)3 (HexNAc)2 (Deoxyhexose)2; (Hex)1 (HexNAc)2; (Hex)1 (HexNAc)2 (Deoxyhexose)2; (Hex)2 (HexNAc)2 (Deoxyhexose)1; (Hex)2 (HexNAc)1 (Deoxyhexose)1; (Hex)2 (HexNAc)3 (Deoxyhexose)1; (Hex)2 (HexNAc)3 (Deoxyhexose)2; (Hex)3 (HexNAc)2 (Deoxyhexose)1; (Hex)2 (HexNAc)1; (Hex)1 (HexNAc)3 (Deoxyhexose)1; (Hex)3 (HexNAc)4 (Deoxyhexose)3; (Hex)2 (HexNAc)1 (Deoxyhexose)2; (Hex)2 (HexNAc)2 (NeuAc)1; (Hex)3 (HexNAc)2 (Deoxyhexose)3; (Hex)2 (HexNAc)4 (Deoxyhexose)2; (Hex)3 (HexNAc)3 (Deoxyhexose)1; (Hex)3 (HexNAc)3 (Deoxyhexose)3; (Hex)2 (HexNAc)2; and (Hex)2 (HexNAc)3; or
c) the MUC5AC glycan comprises a formula selected from the group consisting of (Hex)1 (HexNAc)1; (Hex)1 (HexNAc)2; (Hex)1 (HexNAc)1 (Deoxyhexose)1; (Hex)1 (Deoxyhexose)1; (Hex)2 (HexNAc)3 (Deoxyhexose)1; (Hex)2 (HexNAc)4; (Hex)2 (HexNAc)2 (Deoxyhexose)1; (Hex)2 (HexNAc)3; (Hex)2 (HexNAc)2; (Hex)3 (HexNAc)2 (Deoxyhexose)2; (Hex)3 (HexNAc)3 (Deoxyhexose)1; (Hex)3 (HexNAc)4; (Hex)2 (HexNAc)4 (Deoxyhexose)1; (Hex)2 (HexNAc)1 (Deoxyhexose)1; (Hex)1 (HexNAc)2 (Deoxyhexose)1; (Hex)2 (HexNAc)2 (Deoxyhexose)2; (Hex)1 (HexNAc)3; (Hex)3 (HexNAc)4 (Deoxyhexose)1; and (Hex)1 (HexNAc)1 (NeuAc)1.

16. The method of claim 15, wherein:
a) the MUC2 glycan comprises a formula selected from the group consisting of (Hex)1 (HexNAc)1 (Deoxyhexose)1; (Hex)1 (NeuAc)1; (Hex)1 (Deoxyhexose)1; (Hex)1 (HexNAc)1; (Hex)1 (NeuGc)1; (Hex)1 (HexNAc)2 (Deoxyhexose)1; (Hex)1 (HexNAc)1 (NeuAc)1; and (Hex)1 (HexNAc)1 (NeuGc)1;
b) the MUC5B glycan comprises a formula selected from the group consisting of (Hex)1 (HexNAc)1 (Deoxyhexose)1; (Hex)1 (HexNAc)1; (Hex)1 (HexNAc)2 (Deoxyhexose)1; (Hex)1 (NeuAc)1; (Hex)1 (Deoxyhexose)1; (Hex)3 (HexNAc)3 (Deoxyhexose)2; (Hex)2 (HexNAc)2 (Deoxyhexose)2; (Hex)1 (HexNAc)1 (NeuAc)1; (Hex)3 (HexNAc)2 (Deoxyhexose)2; (Hex)1 (HexNAc)2; (Hex)1 (HexNAc)2 (Deoxyhexose)2; (Hex)2 (HexNAc)2 (Deoxyhexose)1; and (Hex)2 (HexNAc)1 (Deoxyhexose)1; or
c) the MUC5AC glycan comprises a formula selected from the group consisting of (Hex)1 (HexNAc)1; (Hex)1 (HexNAc)2; (Hex)1 (HexNAc)1 (Deoxyhexose)1; (Hex)1 (Deoxyhexose)1; (Hex)2 (HexNAc)3 (Deoxyhexose)1; (Hex)2 (HexNAc)4; (Hex)2 (HexNAc)2 (Deoxyhexose)1; (Hex)2 (HexNAc)3; (Hex)2 (HexNAc)2; (Hex)3 (HexNAc)2 (Deoxyhexose)2; (Hex)3 (HexNAc)3 (Deoxyhexose)1; and (Hex)3 (HexNAc)4.

17. The method of claim 16, wherein:
a) wherein the MUC2 glycan comprises a formula selected from the group consisting of (Hex)1 (HexNAc)1 (Deoxyhexose)1; (Hex)1 (NeuAc)1; (Hex)1 (Deoxyhexose)1; and (Hex)1 (HexNAc)1;
b) the MUC5B glycan comprises a formula selected from the group consisting of (Hex)1 (HexNAc)1 (Deoxyhexose)1; (Hex)1 (HexNAc)1; (Hex)1 (HexNAc)2 (Deoxyhexose)1; (Hex)1 (NeuAc)1; and (Hex)1 (Deoxyhexose)1; or
c) the MUC5AC glycan comprises a formula selected from the group consisting of (Hex)1 (HexNAc)1; (Hex)1 (HexNAc)2; (Hex)1 (HexNAc)1 (Deoxyhexose)1; (Hex)1 (Deoxyhexose)1; (Hex)2 (HexNAc)3 (Deoxyhexose)1; and (Hex)2 (HexNAc)4.

18. The method of claim 12, wherein the mucin glycan is a sulfated mucin glycan.

19. The method of claim 18, wherein the sulfated mucin glycan comprises a formula selected from the group consisting of S1 (Hex)1 (HexNAc)1; S1 (Hex)1 (HexNAc)1 (Deoxyhexose)1; S1 (Hex)1 (HexNAc)2; S1 (Hex)2 (HexNAc)1 (Deoxyhexose)1; S1 (Hex)1 (HexNAc)2 (Deoxyhexose)1; S1 (Hex)1 (HexNAc)2 (NeuAc)1; S1 (Hex)2 (HexNAc)2 (Deoxyhexose)1; S1 (Hex)1 (HexNAc)2 (NeuGc)1 (G60426XC); S1 (Hex)1 (HexNAc)3 (Deoxyhexose)1; and S1 (Hex)2 (HexNAc)3 (Deoxyhexose)1.

20. The method of claim 19, wherein:
a) the sulfated mucin glycan comprises a sulfated MUC2 glycan, and optionally, the sulfated MUC2 glycan comprises a formula selected from the group consisting of S1 (Hex)1 (HexNAc)2; S1 (Hex)1 (HexNAc)2 (Deoxyhexose)1; S1 (Hex)1 (HexNAc)2 (NeuAc)1; S1 (Hex)1 (HexNAc)3 (Deoxyhexose)1; and S1 (Hex)1 (HexNAc)2 (NeuGc)1;
b) the sulfated mucin glycan comprises a sulfated MUC5B glycan, and optionally, the sulfated MUC5B glycan comprises a formula selected from the group consisting of S1 (Hex)2 (HexNAc)1 (Deoxyhexose)1; S1 (Hex)1 (HexNAc)1 (Deoxyhexose)1; S1 (Hex)1 (HexNAc)1; S1 (Hex)2 (HexNAc)2 (Deoxyhexose)1; and S1 (Hex)2 (HexNAc)3 (Deoxyhexose)1; or
c) the sulfated mucin glycan comprises a sulfated MUC5AC glycan, and optionally, the sulfated MUC5AC glycan comprises a formula selected from the group consisting of S1 (Hex)1 (HexNAc)2 (Deoxyhexose)1; S1 (Hex)2 (HexNAc)2 (Deoxyhexose)1; S1 (Hex)2 (HexNAc)1 (Deoxyhexose)1; S1 (Hex)2 (HexNAc)3 (Deoxyhexose)1; and S1 (Hex)1 (HexNAc)2.


SEQUENCE LISTING (26 sequences)

SEQ ID NO: 1 - DNA, 21 bp, synthetic construct
  misc_feature 1..21: note = Primer ECE1_F
  source 1..21: mol_type = other DNA
tgccatttgttgtcagagctg

SEQ ID NO: 2 - DNA, 23 bp, synthetic construct
  misc_feature 1..23: note = Primer ECE1_R
  source 1..23: mol_type = other DNA
tagcttgttgaacagtttccagg

SEQ ID NO: 3 - DNA, 22 bp, synthetic construct
  misc_feature 1..22: note = Primer HWP1_F
  source 1..22: mol_type = other DNA
gctggttcagaatcatccatgc

SEQ ID NO: 4 - DNA, 21 bp, synthetic construct
  misc_feature 1..21: note = Primer HWP1_R
  source 1..21: mol_type = other DNA
aaggttcagtggcaggagctg

SEQ ID NO: 5 - DNA, 21 bp, synthetic construct
  misc_feature 1..21: note = Primer HGC1_F
  source 1..21: mol_type = other DNA
gtcagcttcctgcacctcatc

SEQ ID NO: 6 - DNA, 20 bp, synthetic construct
  misc_feature 1..20: note = Primer HGC1_R
  source 1..20: mol_type = other DNA
aaacagcacgagaaccagcg

SEQ ID NO: 7 - DNA, 20 bp, synthetic construct
  misc_feature 1..20: note = Primer NRG1_F
  source 1..20: mol_type = other DNA
ggttgcacgttgtcgaaacc

SEQ ID NO: 8 - DNA, 19 bp, synthetic construct
  misc_feature 1..19: note = Primer NRG1_R
  source 1..19: mol_type = other DNA
tgttgctgctgctgcttgg

SEQ ID NO: 9 - DNA, 20 bp, synthetic construct
  misc_feature 1..20: note = Primer ACT1_F
  source 1..20: mol_type = other DNA
cccaggtattgctgaacgta

SEQ ID NO: 10 - DNA, 20 bp, synthetic construct
  misc_feature 1..20: note = Primer ACT1_R
  source 1..20: mol_type = other DNA
gaaccaccaatccagacaga

SEQ ID NO: 11 - DNA, 24 bp, synthetic construct
  misc_feature 1..24: note = Primer YWP1_F
  source 1..24: mol_type = other DNA
tgctagtactgctaacaaagtcac

SEQ ID NO: 12 - DNA, 20 bp, synthetic construct
  misc_feature 1..20: note = Primer YWP1_R
  source 1..20: mol_type = other DNA
caccattaacaccaccagca

SEQ ID NO: 13 - DNA, 23 bp, synthetic construct
  misc_feature 1..23: note = Primer UME6_F
  source 1..23: mol_type = other DNA
tcattcaatcctactcgtccacc

SEQ ID NO: 14 - DNA, 21 bp, synthetic construct
  misc_feature 1..21: note = Primer UME6_R
  source 1..21: mol_type = other DNA
ccagatccagtagcagtgctg

SEQ ID NO: 15 - DNA, 21 bp, synthetic construct
  misc_feature 1..21: note = Primer RIP1_F
  source 1..21: mol_type = other DNA
tgctgacagagtcaagaaacc

SEQ ID NO: 16 - DNA, 20 bp, synthetic construct
  misc_feature 1..20: note = Primer RIP1_R
  source 1..20: mol_type = other DNA
gaaccaaccaccgaaatcac

SEQ ID NO: 17 - DNA, 26 bp, synthetic construct
  misc_feature 1..26: note = Primer EFG1_F
  source 1..26: mol_type = other DNA
catcacaaccaggttctacaaccaat

SEQ ID NO: 18 - DNA, 21 bp, synthetic construct
  misc_feature 1..21: note = Primer EFG1_R
  source 1..21: mol_type = other DNA
ctactattagcagcaccaccc

SEQ ID NO: 19 - DNA, 24 bp, synthetic construct
  misc_feature 1..24: note = Primer 18S_F
  source 1..24: mol_type = other DNA
ggatttactgaagactaactactg

SEQ ID NO: 20 - DNA, 21 bp, synthetic construct
  misc_feature 1..21: note = Primer 18S_R
  source 1..21: mol_type = other DNA
gaacaacaaccgatccctagt

SEQ ID NO: 21 - DNA, 24 bp, synthetic construct
  misc_feature 1..24: note = Primer ALS3_F
  source 1..24: mol_type = other DNA
acttccacagctgcttccacttct

SEQ ID NO: 22 - DNA, 21 bp, synthetic construct
  misc_feature 1..21: note = Primer ALS3_R
  source 1..21: mol_type = other DNA
tccacggaaccggttgttgct

SEQ ID NO: 23 - DNA, 21 bp, synthetic construct
  misc_feature 1..21: note = Primer HYR1_F
  source 1..21: mol_type = other DNA
cggttctggaagtggtcataa

SEQ ID NO: 24 - DNA, 20 bp, synthetic construct
  misc_feature 1..20: note = Primer HYR1_R
  source 1..20: mol_type = other DNA
agagtgtgaacctgcgttag

SEQ ID NO: 25 - DNA, 20 bp, synthetic construct
  misc_feature 1..20: note = Primer EED1_F
  source 1..20: mol_type = other DNA
tgctctaccaccacaacaag

SEQ ID NO: 26 - DNA, 18 bp, synthetic construct
  misc_feature 1..18: note = Primer EED1_R
  source 1..18: mol_type = other DNA
ttgcggtgcttgctcata